(19) United States
(12) Patent Application Publication
(10) Pub. No.: US 2022/0047621 A1
(43) Pub. Date: Feb. 17, 2022

(54) RNAi CONSTRUCTS AND METHODS FOR INHIBITING MARC1 EXPRESSION

(71) Applicant: AMGEN INC., Thousand Oaks, CA (US)

(72) Inventors: Justin K. MURRAY, Moorpark, CA (US); Jun ZHANG, Foster City, CA (US); Oliver HOMANN, Berkeley, CA (US); Jason C. LONG, Newbury Park, CA (US); Bryan MEADE, Westlake Village, CA (US)

(73) Assignee: AMGEN INC., Thousand Oaks, CA (US)

(21) Appl. No.: 17/401,114

(22) Filed: Aug. 12, 2021

Related U.S. Application Data
(60) Provisional application No. 63/065,190, filed on Aug. 13, 2020.
(60) Provisional application No. 63/214,016, filed on Jun. 23, 2021.

(51) Int. Cl. A61K 31/7125; C12Q 1/6869; C12N 15/113; A61P 1/16

(52) U.S. Cl. CPC A61K 31/7125; A61P 1/16; C12N 15/113; C12Q 1/6869

(57) ABSTRACT

The present invention relates to RNAi constructs for reducing expression of the MARC1 gene. Methods of using such RNAi constructs to treat or prevent liver fibrosis and fatty liver diseases, such as nonalcoholic fatty liver disease and nonalcoholic steatohepatitis, are also described.

### CROSS-REFERENCE TO RELATED APPLICATIONS

This application claims the benefit of U.S. Provisional Application No. 63/065,190, filed Aug. 13, 2020, and U.S. Provisional Application No. 63/214,016, filed Jun. 23, 2021, both of which are hereby incorporated by reference in their entireties.

### DESCRIPTION OF THE TEXT FILE SUBMITTED ELECTRONICALLY

The present application contains a Sequence Listing, which has been submitted electronically in ASCII format and is hereby incorporated by reference in its entirety. The computer readable format copy of the Sequence Listing, which was created on Aug. 9, 2021, is named A-2664-US-NP ST25 and is 1,064 kilobytes in size.

### FIELD OF THE INVENTION

The present invention relates to compositions and methods for modulating liver expression of mitochondrial amidoxime-reducing component 1 (mARC1) protein. In particular, the present invention relates to nucleic acid-based therapeutics for reducing MARC1 gene expression via RNA interference and methods of using such nucleic acid-based therapeutics to reduce circulating lipid levels and to treat or prevent fatty liver disease and liver fibrosis.

### BACKGROUND OF THE INVENTION

Comprising a spectrum of hepatic pathologies, nonalcoholic fatty liver disease (NAFLD) is the most common chronic liver disease in the world, the prevalence of which doubled in the last 20 years and now is estimated to affect approximately 20-30% of the world population. In some individuals the accumulation of ectopic fat in the liver, called steatosis, triggers inflammation and hepatocellular injury leading to a more advanced stage of disease called, nonalcoholic steatohepatitis (NASH). NASH is defined as lipid accumulation with evidence of cellular damage, inflammation, and different degrees of scarring or fibrosis. As of 2015, 75-100 million Americans are predicted to have NAFLD, whereas NASH accounts for approximately 10-30% of NAFLD diagnoses.

The mARC1 protein is a molybdenum-containing protein in the mitochondrial outer membrane that catalyzes the reduction of N-oxygenated molecules (Klein et al., J Biol Chem, Vol. 287(51):42795-42803, 2012; Ott et al., J Biol Inorg Chem, Vol. 20(2):265-275, 2015). It is a highly effective counterpart to one of the most prominent biotransformation enzymes, CYP450, and is involved in activation of amidoxime prodrugs as well as inactivation of other drugs containing N-hydroxylated functional groups (Neve et al., PLoS One, Vol. 10(9):e0138487, 2015; Ott et al., 2015, supra). Recently, predicted loss-of-function variants in the MARC1 gene have been reported to be associated with decreased blood levels of cholesterol and liver enzymes, reduced liver fat, and protection from cirrhosis. See Emdin et al., bioRxiv 594523; //doi.org/10.1101/594523, 2019; and Emdin et al., PLoS Genet, Vol. 16(4): e1008629, 2020. Specifically, the A165T missense variant in the mARC1 coding region was associated with protection from all-cause cirrhosis, lower levels of hepatic fat on computed tomographic imaging and lower odds of physician-diagnosed fatty liver as well as lower blood levels of alanine transaminase, alkaline phosphatase, total cholesterol, and LDL cholesterol levels in an analysis of 12,361 all-cause cirrhosis cases and 790,095 controls from eight cohorts (Emdin et al., 2020, supra). Additional MARC1 alleles (M187K missense mutation and R200Ter truncation mutation) that associated with lower cholesterol levels, liver enzyme levels and reduced risk of cirrhosis were also identified (Emdin et al., 2020, supra). These data suggest that deficiency of the mARC1 enzyme protects against chronic liver disease and cirrhosis. Accordingly, therapeutics targeting mARC1 function represent a novel approach to reducing cholesterol levels (e.g. non-HDL cholesterol or LDL-cholesterol levels) and liver fibrosis, and treating or preventing liver diseases, particularly NAFLD and NASH.

### SUMMARY OF THE INVENTION

The present invention is based, in part, on the design and generation of RNAi constructs that target the MARC1 gene and reduce its expression in liver cells. The sequence-specific inhibition of MARC1 gene expression is useful for treating or preventing conditions associated with elevated lipid levels and liver fat, such as cardiovascular disease and fatty liver disease. Accordingly, in one embodiment, the present invention provides an RNAi construct comprising a sense strand and an antisense strand, wherein the antisense strand comprises a region having a sequence that is substantially complementary to a mARC1 mRNA sequence. For instance, in some embodiments, the antisense strand comprises a sequence that is substantially complementary to the sequence of at least 15 contiguous nucleotides of a region of the human mARC1 mRNA sequence (SEQ ID NO: 1) with no more than 1, 2, or 3 mismatches. In certain embodiments, the antisense strand comprises a region having at least 15 contiguous nucleotides from an antisense sequence listed in Table 1 or Table 2.

In some embodiments, the sense strand of the RNAi constructs described herein comprises a sequence that is sufficiently complementary to the sequence of the antisense strand to form a duplex region of about 15 to about 30 base pairs in length. In these and other embodiments, the sense and antisense strands are each independently about 19 to about 30 nucleotides in length. In some embodiments, the RNAi constructs comprise one or two blunt ends. In other embodiments, the RNAi constructs comprise one or two nucleotide overhangs. Such nucleotide overhangs may comprise 1 to 6 unpaired nucleotides and can be located at the 3′ end of the sense strand, the 3′ end of the antisense strand, or the 3′ end of both the sense and antisense strand. In certain embodiments, the RNAi constructs comprise an overhang of two unpaired nucleotides at the 3′ end of the sense strand and the 3′ end of the antisense strand. In other embodiments, the RNAi constructs comprise an overhang of two unpaired nucleotides at the 3′ end of the antisense strand and a blunt end at the 3′ end of the sense strand/5′ end of the antisense strand.

The RNAi constructs of the invention may comprise one or more modified nucleotides, including nucleotides having modifications to the ribose ring, nucleobase, or phosphodiester backbone. In some embodiments, the RNAi constructs comprise one or more 2′-modified nucleotides. Such 2′-modified nucleotides can include 2′-fluoro modified nucleotides, 2′-O-methyl modified nucleotides, 2′-O-methoxyethyl modified nucleotides, 2′-O-alkyl modified nucleotides, 2′-O-allyl modified nucleotides, bicyclic nucleic acids (BNA), deoxyribonucleotides, or combinations thereof. In one particular embodiment, the RNAi constructs comprise one or more 2′-fluoro modified nucleotides, 2′-O-methyl modified nucleotides, or combinations thereof. In some embodiments, all of the nucleotides in the sense and antisense strand of the RNAi construct are modified nucleotides. Abasic nucleotides may be incorporated into the RNAi constructs of the invention, for example, as the terminal nucleotide at the 3′ end, the 5′ end, or both the 3′ end and the 5′ end of the sense strand. In such embodiments, the abasic nucleotide may be inverted, e.g. linked to the adjacent nucleotide through a 3′-3′ internucleotide linkage or a 5′-5′ internucleotide linkage.

In some embodiments, the RNAi constructs comprise at least one backbone modification, such as a modified internucleotide or internucleoside linkage. In certain embodiments, the RNAi constructs described herein comprise at least one phosphorothioate internucleotide linkage. In particular embodiments, the phosphorothioate internucleotide linkages may be positioned at the 3′ or 5′ ends of the sense and/or antisense strands. For instance, in some embodiments, the antisense strand comprises two consecutive phosphorothioate internucleotide linkages between the terminal nucleotides at both the 3′ and 5′ ends. In some such embodiments, the sense strand comprises one or two phosphorothioate internucleotide linkages between the terminal nucleotides at its 3′ end.

In certain embodiments, the antisense strand and/or the sense strand of the RNAi constructs of the invention may comprise or consist of a sequence from the antisense and sense sequences listed in Table 1 or Table 2. In certain such embodiments, the RNAi construct may be any one of the duplex compounds listed in any one of Tables 1 to 24. In some embodiments, the RNAi construct is D-1044, D-1061, D-1062, D-1067, D-1083, D-1090, D-1092, D-1093, D-1095, D-1138, D-1139, D-1143, D-1170, D-1177, D-1180, D-1191, D-1245, D-2000, D-2002, D-2003, D-2004, D-2011, D-2026, D-2028, D-2032, D-2033, D-2034, D-2035, D-2036, D-2042, D-2044, D-2045, D-2046, D-2050, D-2078, D-2079, D-2081, D-2182, D-2196, D-2238, D-2241, D-2243, D-2246, D-2255, D-2356, D-2258, D-2301, D-2316, D-2317, D-2329, D-2332, D-2341, D-2344, D-2357, D-2399, or D-2510. In certain embodiments, the RNAi construct is D-2079, D-2081, D-2196, D-2238, D-2241, D-2255, D-2258, D-2317, D-2332, D-2357, or D-2399.

In some embodiments, the RNAi constructs of the invention may target a particular region of the human mARC1 mRNA transcript (e.g. the human mARC1 mRNA transcript sequence set forth in SEQ ID NO: 1). For instance, in certain embodiments, the RNAi constructs comprise a sense strand and an antisense strand, wherein the antisense strand comprises a region having a sequence that is substantially complementary to the sequence of at least 15 contiguous nucleotides of nucleotides 1205 to 1250 of SEQ ID NO: 1. In other embodiments, the antisense strand comprises a region having a sequence that is substantially complementary to the sequence of at least 15 contiguous nucleotides of nucleotides 1209 to 1239 of SEQ ID NO: 1. In yet other embodiments, the antisense strand comprises a region having a sequence that is substantially complementary to the sequence of at least 15 contiguous nucleotides of nucleotides 1345 to 1375 of SEQ ID NO: 1. In still other embodiments, the antisense strand comprises a region having a sequence that is substantially complementary to the sequence of at least 15 contiguous nucleotides of nucleotides 2039 to 2078 of SEQ ID NO: 1. In certain other embodiments, the antisense strand comprises a region having a sequence that is substantially complementary to the sequence of at least 15 contiguous nucleotides of nucleotides 2048 to 2074 of SEQ ID NO: 1. In any of the above embodiments, the sequence of the antisense strand may be substantially complementary to the sequence of at least 15 contiguous nucleotides of the specific regions of the human mARC1 transcript (SEQ ID NO: 1) with no more than 1, 2, or 3 mismatches between the sequence of the antisense strand and the sequence of the specific regions of the human mARC1 transcript. In some such embodiments in which a mismatch occurs between the sequence of the antisense strand and the sequence of the target mARC1 mRNA sequence, the mismatch may be located between the target mARC1 mRNA sequence and the nucleotide at position 6 and/or position 8 from the 5′ end of the antisense strand. In other embodiments, the sequence of the antisense strand may be fully complementary to the sequence of at least 15 contiguous nucleotides of the specific regions of the human mARC1 transcript (SEQ ID NO: 1).

The RNAi constructs of the invention may further comprise a ligand to facilitate delivery or uptake of the RNAi constructs to specific tissues or cells, such as liver cells. In certain embodiments, the ligand targets delivery of the RNAi constructs to hepatocytes. In these and other embodiments, the ligand may comprise galactose, galactosamine, or N-acetyl-galactosamine (GalNAc). In certain embodiments, the ligand comprises a multivalent galactose or multivalent GalNAc moiety, such as a trivalent or tetravalent galactose or GalNAc moiety. The ligand may be covalently attached to the 5′ or 3′ end of the sense strand of the RNAi construct, optionally through a linker. In some embodiments, the RNAi constructs comprise a ligand and linker having a structure according to any one of Formulas I to IX described herein. In certain embodiments, the RNAi constructs comprise a ligand and linker having a structure according to Formula VII. In other embodiments, the RNAi constructs comprise a ligand and linker having a structure according to Formula IV.

The present invention also provides pharmaceutical compositions comprising any of the RNAi constructs described herein and a pharmaceutically acceptable carrier, excipient, or diluent. Such pharmaceutical compositions are particularly useful for reducing expression of the MARC1 gene in the cells (e.g. liver cells) of a patient in need thereof. Patients who may be administered a pharmaceutical composition of the invention can include patients diagnosed with or at risk of cardiovascular disease, fatty liver disease, liver fibrosis, or cirrhosis and patients with elevated blood levels of cholesterol (e.g. total cholesterol, non-HDL cholesterol, or LDL-cholesterol). Accordingly, the present invention includes methods of treating, preventing, or reducing the risk of developing fatty liver disease (e.g. NAFLD, NASH, alcoholic fatty liver disease, or alcoholic steatohepatitis), liver fibrosis, or cardiovascular disease in a patient in need thereof comprising administering an RNAi construct or pharmaceutical composition described herein. In certain embodiments, the present invention provides methods for reducing blood levels (serum or plasma) of cholesterol (e.g. total cholesterol, non-HDL cholesterol, or LDL-cholesterol) in a patient in need thereof comprising administering an RNAi construct or pharmaceutical composition described herein.

The use of mARC1-targeting RNAi constructs in any of the methods described herein or for preparation of medicaments for administration according to the methods described herein is specifically contemplated. For instance, the present invention includes a mARC1-targeting RNAi construct for use in a method for treating, preventing, or reducing the risk of developing fatty liver disease (e.g. NAFLD, NASH, alcoholic fatty liver disease, or alcoholic steatohepatitis), liver fibrosis, or cardiovascular disease in a patient in need thereof. The present invention also includes a mARC1-targeting RNAi construct for use in a method for reducing blood levels (serum or plasma) of cholesterol (e.g. total cholesterol, non-HDL cholesterol, or LDL-cholesterol) in a patient in need thereof.

The present invention also encompasses the use of a mARC1-targeting RNAi construct in the preparation of a medicament for treating, preventing, or reducing the risk of developing fatty liver disease (e.g. NAFLD, NASH, alcoholic fatty liver disease, or alcoholic steatohepatitis), liver fibrosis, or cardiovascular disease in a patient in need thereof. In certain embodiments, the present invention provides the use of a mARC1-targeting RNAi construct in the preparation of a medicament for reducing blood levels (serum or plasma) of cholesterol (e.g. total cholesterol, non-HDL cholesterol, or LDL-cholesterol) in a patient in need thereof.

### BRIEF DESCRIPTION OF THE DRAWINGS

FIG. 1 shows the nucleotide sequence of a transcript of the human MARC1 gene (Ensembl transcript no. ENST00000366910.9; SEQ ID NO: 1). The transcript sequence is depicted as the complementary DNA (cDNA) sequence with thymine bases replacing uracil bases.

FIGS. 2A and 2B are bar graphs showing liver expression of mARC1 mRNA (FIG. 2A) and mARC2 mRNA (FIG. 2B) in ob/ob mice receiving subcutaneous injections of buffer, mARC1 siRNA (duplex no. D-1000), or a control siRNA (duplex no. D-1002) once every two weeks for six weeks. mRNA levels were assessed by qPCR at six weeks and are expressed relative to mRNA levels in animals receiving buffer only injections.

FIGS. 3A-3H are graphs depicting serum levels of total cholesterol (CHOL; FIG. 3A), LDL cholesterol (LDL; FIG. 3B), HDL cholesterol (HDL; FIG. 3C), triglycerides (TG; FIG. 3D), alanine aminotransferase (ALT; FIG. 3E), aspartate aminotransferase (AST; FIG. 3F), C-reactive protein (CRP; FIG. 3G), and tissue inhibitor of metalloproteinases-1 (TIMP-1; FIG. 3H) in ob/ob mice receiving subcutaneous injections of buffer, mARC1 siRNA (duplex no. D-1000), or a control siRNA (duplex no. D-1002) once every two weeks for six weeks. Serum levels of the different analytes were measured using a clinical analyzer at the six-week time point. Mean values±standard error of the mean (SEM) are shown. *=p<0.05;**=p<0.01 vs. buffer control group.

FIGS. 4A and 4B are graphs showing liver levels of triglycerides (liver TG; FIG. 4A) or total cholesterol (liver TC; FIG. 4B) at six weeks in ob/ob mice receiving subcutaneous injections of buffer, mARC1 siRNA (duplex no. D-1000), or a control siRNA (duplex no. D-1002) once every two weeks for six weeks. Mean values±SEM are shown. ***=p<0.001 vs. buffer control group.

FIGS. 5A and 5B are bar graphs showing liver expression of mARC1 mRNA (FIG. 5A) and mARC2 mRNA (FIG. 5B) in c57BL/6 mice on a standard chow diet (chow control) or a 0.2% cholesterol diet (TD190883). Mice on the 0.2% cholesterol diet received subcutaneous injections of buffer (TD190883 control), mARC1 siRNA (duplex no. D-1000), or a control siRNA (duplex no. D-1002) once every two weeks for 24 weeks. mRNA levels were assessed by qPCR at 24 weeks and are expressed relative to mRNA levels in the chow control animals.

FIGS. 6A-6F are graphs depicting serum levels of aspartate aminotransferase (AST; FIG. 6A), alanine aminotransferase (ALT; FIG. 6B), total cholesterol (FIG. 6C), LDL cholesterol (LDL-c; FIG. 6D), HDL cholesterol (HDL-c; FIG. 6E), and triglycerides (FIG. 6F) in c57BL/6 mice on a standard chow diet (chow control) or a 0.2% cholesterol diet (TD190883). Mice on the 0.2% cholesterol diet received subcutaneous injections of buffer (TD190883 control), mARC1 siRNA (duplex no. D-1000), or a control siRNA (duplex no. D-1002) once every two weeks for 24 weeks. Serum levels of the different analytes were measured using a clinical analyzer at the indicated time post dosing. Mean values±standard error of the mean (SEM) are shown. *=p<0.05;**=p<0.01, ***=p<0.001 vs. TD190883 control group.

FIGS. 7A-7D are graphs showing body weight (FIG. 7A), liver weight (FIG. 7B), liver levels of triglycerides (FIG. 7C) and liver levels of total cholesterol (FIG. 7D) at 24 weeks in c57BL/6 mice on a standard chow diet (chow control) or a 0.2% cholesterol diet (TD190883). Mice on the 0.2% cholesterol diet received subcutaneous injections of buffer (TD190883 control), mARC1 siRNA (duplex no. D-1000), or a control siRNA (duplex no. D-1002) once every two weeks for 24 weeks. Mean values±SEM are shown.

FIGS. 8A-8F are antisense strand and sense strand serum concentration-time profiles in cynomolgus macaque monkeys following a single 3 mg/kg s.c. dose of GalNAc-conjugated mARC1 siRNA molecules D-2241 (FIGS. 8A and 8B), D-2081 (FIGS. 8C and 8D), and D-2258 (FIGS. 8E and 8F). FIGS. 8A, 8C, and 8E depict the concentration-time profiles from 0.083 to 24 hours post dose, whereas FIGS. 8B, 8D, and 8F depict the concentration-time profiles from 0.083 to 1056 hours post dose.

### DETAILED DESCRIPTION

The present invention is directed to compositions and methods for regulating the expression of the MARC1 gene in a cell or mammal. In some embodiments, compositions of the invention comprise RNAi constructs that target a mRNA transcribed from the MARC1 gene, particularly the human MARC1 gene, and reduce expression of the mARC1 protein in a cell or mammal. Such RNAi constructs are useful for reducing serum lipid levels (e.g., total cholesterol and LDL-cholesterol levels), treating or preventing various forms of cardiovascular disease and fatty liver disease, such as NAFLD and NASH, and reducing liver fibrosis and the risk of progression to cirrhosis.

As used herein, the term “RNAi construct” refers to an agent comprising an RNA molecule that is capable of downregulating expression of a target gene (e.g. MARC1 gene) via an RNA interference mechanism when introduced into a cell. RNA interference is the process by which a nucleic acid molecule induces the cleavage and degradation of a target RNA molecule (e.g. messenger RNA or mRNA molecule) in a sequence-specific manner, e.g. through an RNA-induced silencing complex (RISC) pathway. In some embodiments, the RNAi construct comprises a double-stranded RNA molecule comprising two antiparallel strands of contiguous nucleotides that are sufficiently complementary to each other to hybridize to form a duplex region. “Hybridize” or “hybridization” refers to the pairing of complementary polynucleotides, typically via hydrogen bonding (e.g. Watson-Crick, Hoogsteen or reversed Hoogsteen hydrogen bonding) between complementary bases in the two polynucleotides. The strand comprising a region having a sequence that is substantially complementary to a target sequence (e.g. target mRNA) is referred to as the “antisense strand” or “guide strand.” The “sense strand” or “passenger strand” refers to the strand that includes a region that is substantially complementary to a region of the antisense strand. In some embodiments, the sense strand may comprise a region that has a sequence that is substantially identical to the target sequence.

A double-stranded RNA molecule may include chemical modifications to ribonucleotides, including modifications to the ribose sugar, base, or backbone components of the ribonucleotides, such as those described herein or known in the art. Any such modifications, as used in a double-stranded RNA molecule (e.g. siRNA, shRNA, or the like), are encompassed by the term “double-stranded RNA” for the purposes of this disclosure.

As used herein, a first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of skill in the art. A first sequence is considered to be fully complementary (100% complementary) to a second sequence if a polynucleotide comprising the first sequence base pairs with a polynucleotide comprising the second sequence over the entire length of one or both nucleotide sequences without any mismatches. A sequence is “substantially complementary” to a target sequence if the sequence is at least about 80%, 85%, 90%, 95%, 96%, 97%, 98%, or 99% complementary to a target sequence. Percent complementarity can be calculated by dividing the number of bases in a first sequence that are complementary to bases at corresponding positions in a second or target sequence by the total length of the first sequence. A sequence may also be said to be substantially complementary to another sequence if there are no more than 5, 4, 3, or 2 mismatches over a 30 base pair duplex region when the two sequences are hybridized. Generally, if any nucleotide overhangs, as defined herein, are present, the sequence of such overhangs is not considered in determining the degree of complementarity between two sequences. By way of example, a sense strand of 21 nucleotides in length and an antisense strand of 21 nucleotides in length that hybridize to form a 19 base pair duplex region with a 2-nucleotide overhang at the 3′ end of each strand would be considered to be fully complementary as the term is used herein.

In some embodiments, a region of the antisense strand comprises a sequence that is substantially or fully complementary to a region of the target RNA sequence (e.g. mARC1 mRNA sequence). In such embodiments, the sense strand may comprise a sequence that is fully complementary to the sequence of the antisense strand. In other such embodiments, the sense strand may comprise a sequence that is substantially complementary to the sequence of the antisense strand, e.g. having 1, 2, 3, 4, or 5 mismatches in the duplex region formed by the sense and antisense strands. In certain embodiments, it is preferred that any mismatches occur within the terminal regions (e.g. within 6, 5, 4, 3, or 2 nucleotides of the 5′ and/or 3′ ends of the strands). In one embodiment, any mismatches in the duplex region formed from the sense and antisense strands occur within 6, 5, 4, 3, or 2 nucleotides of the 5′ end of the antisense strand.

In certain embodiments, the sense strand and antisense strand of the double-stranded RNA may be two separate molecules that hybridize to form a duplex region but are otherwise unconnected. Such double-stranded RNA molecules formed from two separate strands are referred to as “small interfering RNAs” or “short interfering RNAs” (siRNAs). Thus, in some embodiments, the RNAi constructs of the invention comprise an siRNA.

In other embodiments, the sense strand and the antisense strand that hybridize to form a duplex region may be part of a single RNA molecule, i.e. the sense and antisense strands are part of a self-complementary region of a single RNA molecule. In such cases, a single RNA molecule comprises a duplex region (also referred to as a stem region) and a loop region. The 3′ end of the sense strand is connected to the 5′ end of the antisense strand by a contiguous sequence of unpaired nucleotides, which will form the loop region. The loop region is typically of a sufficient length to allow the RNA molecule to fold back on itself such that the antisense strand can base pair with the sense strand to form the duplex or stem region. The loop region can comprise from about 3 to about 25, from about 5 to about 15, or from about 8 to about 12 unpaired nucleotides. Such RNA molecules with at least partially self-complementary regions are referred to as “short hairpin RNAs” (shRNAs). In certain embodiments, the RNAi constructs of the invention comprise a shRNA. The length of a single, at least partially self-complementary RNA molecule can be from about 40 nucleotides to about 100 nucleotides, from about 45 nucleotides to about 85 nucleotides, or from about 50 nucleotides to about 60 nucleotides and comprise a duplex region and loop region each having the lengths recited herein.

In some embodiments, the RNAi constructs of the invention comprise a sense strand and an antisense strand, wherein the antisense strand comprises a region having a sequence that is substantially or fully complementary to a mARC1 messenger RNA (mRNA) sequence. As used herein, a “mARC1 mRNA sequence” refers to any messenger RNA sequence, including allelic variants and splice variants, encoding a mARC1 protein, including mARC1 protein variants or isoforms from any species (e.g. non-human primate, human). The MARC1 gene (also known as MTARC1 or MOSC1) encodes the mitochondrial amidoxime reducing component 1 enzyme (also known as MOCO sulphurase C-terminal domain containing 1 enzyme). In humans, the MARC1 gene is found on chromosome 1 at locus 1q41.

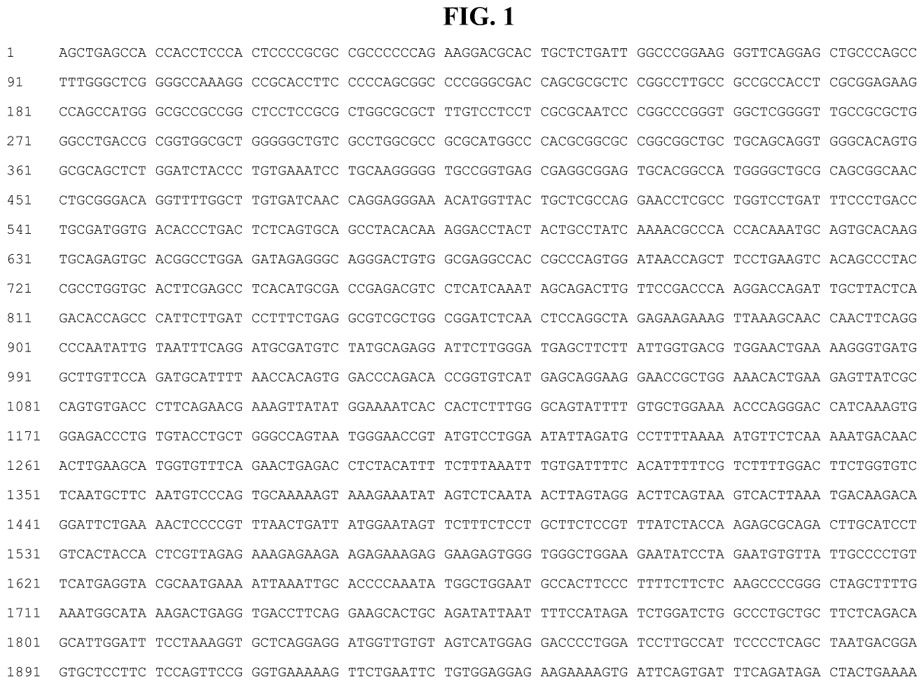

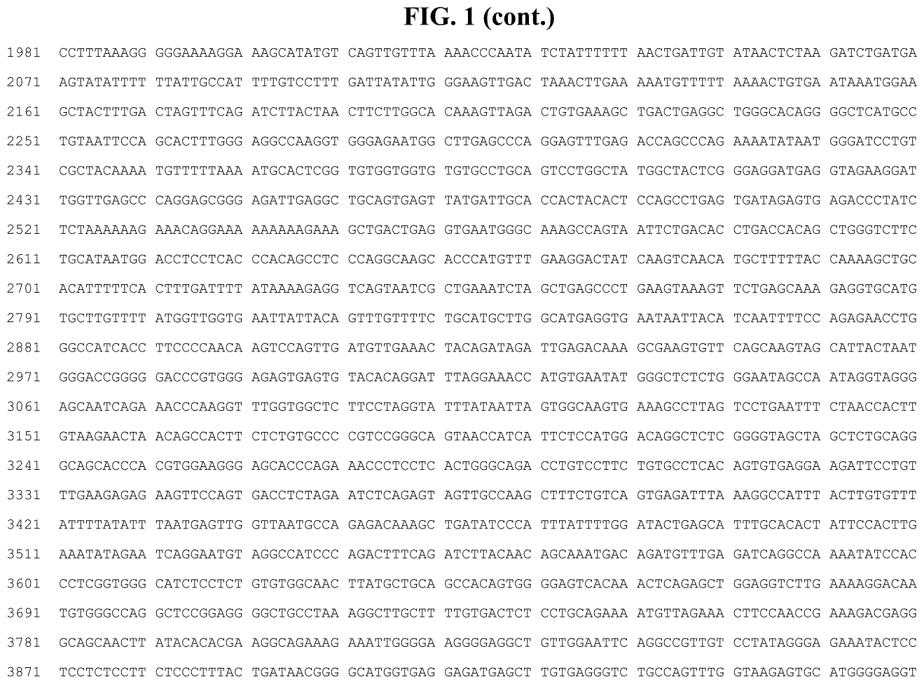

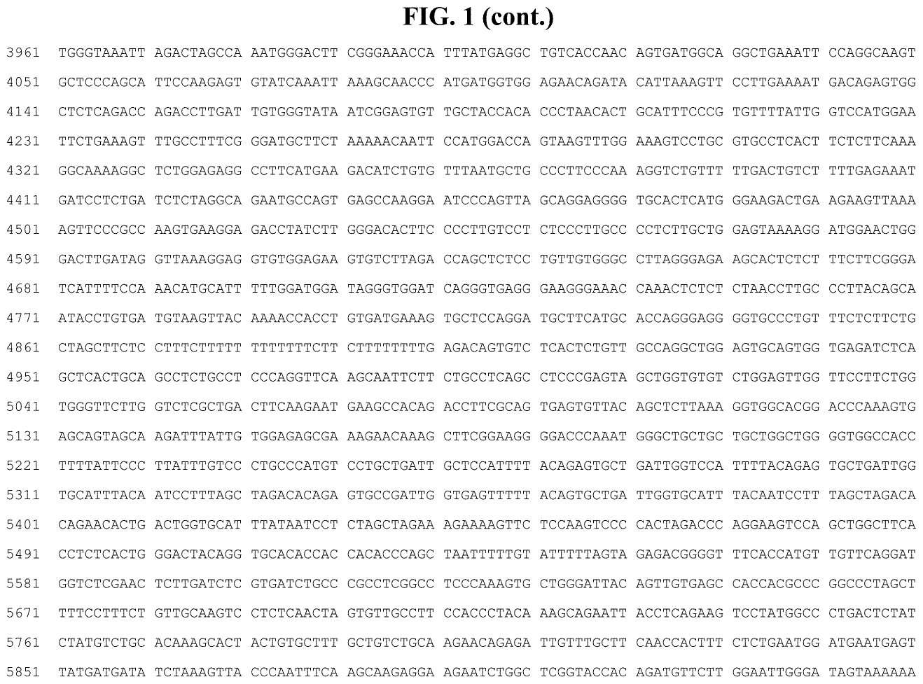

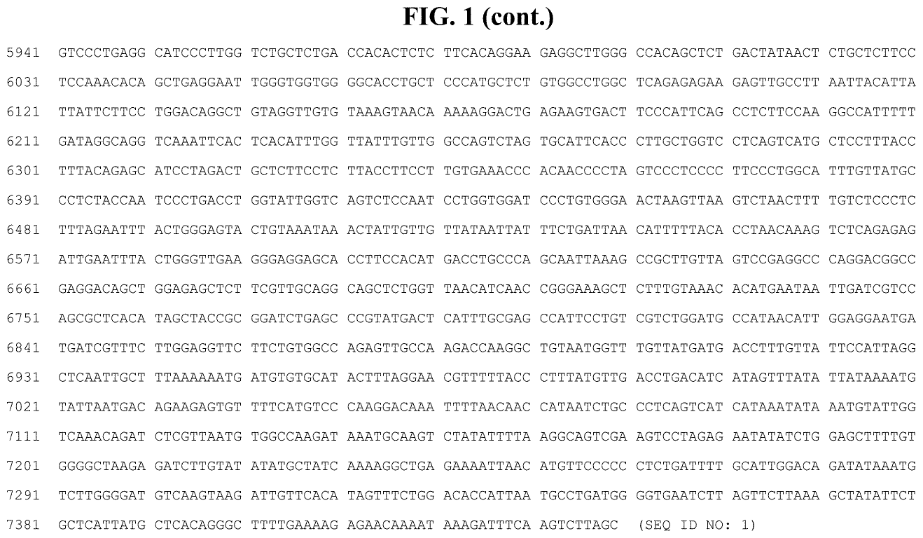

A mARC1 mRNA sequence also includes the transcript sequence expressed as its complementary DNA (cDNA) sequence. A cDNA sequence refers to the sequence of an mRNA transcript expressed as DNA bases (e.g. guanine, adenine, thymine, and cytosine) rather than RNA bases (e.g. guanine, adenine, uracil, and cytosine). Thus, the antisense strand of the RNAi constructs of the invention may comprise a region having a sequence that is substantially or fully complementary to a target mARC1 mRNA sequence or mARC1 cDNA sequence. A mARC1 mRNA or cDNA sequence can include, but is not limited to, any mARC1 mRNA or cDNA sequences in the Ensembl Genome or National Center for Biotechnology Information (NCBI) databases, such as human sequences: Ensembl transcript no. ENST00000366910.9 (FIG. 1, SEQ ID NO: 1) and NCBI Reference sequence NM_022746.4; cynomolgus monkey sequences: NCBI Reference sequences XR_001490722.1, XR_001490722.1, XR_001490723.1, XR_001490726.1, XR_273285.2, XM_005540901.2, XR_273286.2, XM_005540898.2, and XM_005540899.2; rhesus monkey sequences: NCBI Reference sequences XM_015115809.2, XM_015115815.2, XM_001102192.4, and XM_001102284.3; chimpanzee sequences: NCBI Reference sequences XM_009441519.3, XM_001172926.4, and XM_009441521.3; rat sequences: NCBI Reference sequence XM_017598938.1; and mouse sequences: NCBI Reference sequence XM_006497192.4. In certain embodiments, the mARC1 mRNA sequence is the human transcript set forth in FIG. 1 (SEQ ID NO: 1).

A region of the antisense strand can be substantially complementary or fully complementary to at least 15 consecutive nucleotides of the mARC1 mRNA sequence. In certain embodiments, the region of the antisense strand comprises a sequence that is substantially complementary to the sequence of at least 15, at least 16, at least 17, at least 18, or at least 19 contiguous nucleotides of a region of the mARC1 mRNA sequence (e.g. a human mARC1 mRNA sequence (SEQ ID NO: 1)) with no more than 1, 2, or 3 mismatches. In related embodiments, the antisense strand comprises a region having a sequence that is substantially complementary to the sequence of at least 15, at least 16, at least 17, at least 18, or at least 19 contiguous nucleotides of a region of the mARC1 mRNA sequence with no more than 1 mismatch. In embodiments in which the sequence of the antisense strand is not fully complementary to the target mARC1 mRNA sequence and contains a mismatch, the mismatch may occur between the target mARC1 mRNA sequence and the nucleotide at position 6 and/or position 8 from the 5′ end of the antisense strand. In some embodiments, the target region of the mARC1 mRNA sequence to which the antisense strand comprises a region of complementarity can range from about 15 to about 30 consecutive nucleotides, from about 16 to about 28 consecutive nucleotides, from about 18 to about 26 consecutive nucleotides, from about 17 to about 24 consecutive nucleotides, from about 19 to about 30 consecutive nucleotides, from about 19 to about 25 consecutive nucleotides, from about 19 to about 23 consecutive nucleotides, or from about 19 to about 21 consecutive nucleotides. In certain embodiments, the region of the antisense strand comprising a sequence that is substantially or fully complementary to a mARC1 mRNA sequence may comprise at least 15 contiguous nucleotides from an antisense sequence listed in Table 1 or Table 2. In other embodiments, the sequence of the antisense strand comprises at least 16, at least 17, at least 18, or at least 19 contiguous nucleotides from an antisense sequence listed in Table 1 or Table 2.

The sense strand of the RNAi construct typically comprises a sequence that is sufficiently complementary to the sequence of the antisense strand such that the two strands hybridize under physiological conditions to form a duplex region. A “duplex region” refers to the region in two complementary or substantially complementary polynucleotides that form base pairs with one another, either by Watson-Crick base pairing or other hydrogen bonding interaction, to create a duplex between the two polynucleotides. The duplex region of the RNAi construct should be of sufficient length to allow the RNAi construct to enter the RNA interference pathway, e.g. by engaging the Dicer enzyme and/or the RISC complex. For instance, in some embodiments, the duplex region is about 15 to about 30 base pairs in length. Other lengths for the duplex region within this range are also suitable, such as about 15 to about 28 base pairs, about 15 to about 26 base pairs, about 15 to about 24 base pairs, about 15 to about 22 base pairs, about 17 to about 28 base pairs, about 17 to about 26 base pairs, about 17 to about 24 base pairs, about 17 to about 23 base pairs, about 17 to about 21 base pairs, about 19 to about 25 base pairs, about 19 to about 23 base pairs, or about 19 to about 21 base pairs. In certain embodiments, the duplex region is about 17 to about 24 base pairs in length. In other embodiments, the duplex region is about 19 to about 21 base pairs in length. In one embodiment, the duplex region is about 19 base pairs in length. In another embodiment, the duplex region is about 21 base pairs in length.

For embodiments in which the sense strand and antisense strand are two separate molecules (e.g. RNAi construct comprises an siRNA), the sense strand and antisense strand need not be the same length as the length of the duplex region. For instance, one or both strands may be longer than the duplex region and have one or more unpaired nucleotides or mismatches flanking the duplex region. Thus, in some embodiments, the RNAi construct comprises at least one nucleotide overhang. As used herein, a “nucleotide overhang” refers to the unpaired nucleotide or nucleotides that extend beyond the duplex region at the terminal ends of the strands. Nucleotide overhangs are typically created when the 3′ end of one strand extends beyond the 5′ end of the other strand or when the 5′ end of one strand extends beyond the 3′ end of the other strand. The length of a nucleotide overhang is generally between 1 and 6 nucleotides, 1 and 5 nucleotides, 1 and 4 nucleotides, 1 and 3 nucleotides, 2 and 6 nucleotides, 2 and 5 nucleotides, or 2 and 4 nucleotides. In some embodiments, the nucleotide overhang comprises 1, 2, 3, 4, 5, or 6 nucleotides. In one particular embodiment, the nucleotide overhang comprises 1 to 4 nucleotides. In certain embodiments, the nucleotide overhang comprises 2 nucleotides. In certain other embodiments, the nucleotide overhang comprises a single nucleotide.

The nucleotides in the overhang can be ribonucleotides or modified nucleotides as described herein. In some embodiments, the nucleotides in the overhang are 2′-modified nucleotides (e.g. 2′-fluoro modified nucleotides, 2′-O-methyl modified nucleotides), deoxyribonucleotides, abasic nucleotides, inverted nucleotides (e.g. inverted abasic nucleotides, inverted deoxyribonucleotides), or combinations thereof. For instance, in one embodiment, the nucleotides in the overhang are deoxyribonucleotides, e.g. deoxythymidine. In another embodiment, the nucleotides in the overhang are 2′-O-methyl modified nucleotides, 2′-fluoro modified nucleotides, 2′-methoxyethyl modified nucleotides, or combinations thereof. In other embodiments, the overhang comprises a 5′-uridine-uridine-3′ (5′-UU-3′) dinucleotide. In such embodiments, the UU dinucleotide may comprise ribonucleotides or modified nucleotides, e.g. 2′-modified nucleotides. In other embodiments, the overhang comprises a 5′-deoxythymidine-deoxythymidine-3′ (5′-dTdT-3′) dinucleotide. When a nucleotide overhang is present in the antisense strand, the nucleotides in the overhang can be complementary to the target gene sequence, form a mismatch with the target gene sequence, or comprise some other sequence (e.g. polypyrimidine or polypurine sequence, such as UU, TT, AA, GG, etc.).

The nucleotide overhang can be at the 5′ end or 3′ end of one or both strands. For example, in one embodiment, the RNAi construct comprises a nucleotide overhang at the 5′ end and the 3′ end of the antisense strand. In another embodiment, the RNAi construct comprises a nucleotide overhang at the 5′ end and the 3′ end of the sense strand. In some embodiments, the RNAi construct comprises a nucleotide overhang at the 5′ end of the sense strand and the 5′ end of the antisense strand. In other embodiments, the RNAi construct comprises a nucleotide overhang at the 3′ end of the sense strand and the 3′ end of the antisense strand.

The RNAi constructs may comprise a single nucleotide overhang at one end of the double-stranded RNA molecule and a blunt end at the other. A “blunt end” means that the sense strand and antisense strand are fully base-paired at the end of the molecule and there are no unpaired nucleotides that extend beyond the duplex region. In some embodiments, the RNAi construct comprises a nucleotide overhang at the 3′ end of the sense strand and a blunt end at the 5′ end of the sense strand and 3′ end of the antisense strand. In other embodiments, the RNAi construct comprises a nucleotide overhang at the 3′ end of the antisense strand and a blunt end at the 5′ end of the antisense strand and the 3′ end of the sense strand. In certain embodiments, the RNAi construct comprises a blunt end at both ends of the double-stranded RNA molecule. In such embodiments, the sense strand and antisense strand have the same length and the duplex region is the same length as the sense and antisense strands (i.e. the molecule is double-stranded over its entire length).

The sense strand and antisense strand in the RNAi constructs of the invention can each independently be about 15 to about 30 nucleotides in length, about 19 to about 30 nucleotides in length, about 18 to about 28 nucleotides in length, about 19 to about 27 nucleotides in length, about 19 to about 25 nucleotides in length, about 19 to about 23 nucleotides in length, about 19 to about 21 nucleotides in length, about 21 to about 25 nucleotides in length, or about 21 to about 23 nucleotides in length. In certain embodiments, the sense strand and antisense strand are each independently about 18, about 19, about 20, about 21, about 22, about 23, about 24, or about 25 nucleotides in length. In some embodiments, the sense strand and antisense strand have the same length but form a duplex region that is shorter than the strands such that the RNAi construct has two nucleotide overhangs. For instance, in one embodiment, the RNAi construct comprises (i) a sense strand and an antisense strand that are each 21 nucleotides in length, (ii) a duplex region that is 19 base pairs in length, and (iii) nucleotide overhangs of 2 unpaired nucleotides at both the 3′ end of the sense strand and the 3′ end of the antisense strand. In another embodiment, the RNAi construct comprises (i) a sense strand and an antisense strand that are each 23 nucleotides in length, (ii) a duplex region that is 21 base pairs in length, and (iii) nucleotide overhangs of 2 unpaired nucleotides at both the 3′ end of the sense strand and the 3′ end of the antisense strand. In other embodiments, the sense strand and antisense strand have the same length and form a duplex region over their entire length such that there are no nucleotide overhangs on either end of the double-stranded molecule. In one such embodiment, the RNAi construct is blunt ended (e.g. has two blunt ends) and comprises (i) a sense strand and an antisense strand, each of which is 21 nucleotides in length, and (ii) a duplex region that is 21 base pairs in length. In another such embodiment, the RNAi construct is blunt ended (e.g. has two blunt ends) and comprises (i) a sense strand and an antisense strand, each of which is 23 nucleotides in length, and (ii) a duplex region that is 23 base pairs in length. In still another such embodiment, the RNAi construct is blunt ended (e.g. has two blunt ends) and comprises (i) a sense strand and an antisense strand, each of which is 19 nucleotides in length, and (ii) a duplex region that is 19 base pairs in length.

In other embodiments, the sense strand or the antisense strand is longer than the other strand and the two strands form a duplex region having a length equal to that of the shorter strand such that the RNAi construct comprises at least one nucleotide overhang. For example, in one embodiment, the RNAi construct comprises (i) a sense strand that is 19 nucleotides in length, (ii) an antisense strand that is 21 nucleotides in length, (iii) a duplex region of 19 base pairs in length, and (iv) a nucleotide overhang of 2 unpaired nucleotides at the 3′ end of the antisense strand. In another embodiment, the RNAi construct comprises (i) a sense strand that is 21 nucleotides in length, (ii) an antisense strand that is 23 nucleotides in length, (iii) a duplex region of 21 base pairs in length, and (iv) a nucleotide overhang of 2 unpaired nucleotides at the 3′ end of the antisense strand.

The antisense strand of the RNAi constructs of the invention can comprise or consist of the sequence of any one of the antisense sequences listed in Table 1 or Table 2, the sequence of nucleotides 1-19 of any of these antisense sequences, or the sequence of nucleotides 2-19 of any of these antisense sequences. Thus, in some embodiments, the antisense strand comprises or consists of a sequence selected from SEQ ID NOs: 671-1339, 2072-2803, 2906-3061, or 3321-3655. In other embodiments, the antisense strand comprises or consists of a sequence of nucleotides 1-19 of any one of SEQ ID NOs: 671-1339, 2072-2803, 2906-3061, or 3321-3655. In still other embodiments, the antisense strand comprises or consists of a sequence of nucleotides 2-19 of any one of SEQ ID NOs: 671-1339, 2072-2803, 2906-3061, or 3321-3655. In certain embodiments, the antisense strand comprises or consists of a sequence selected from SEQ ID NO: 715; SEQ ID NO: 725; SEQ ID NO: 732; SEQ ID NO: 733; SEQ ID NO: 737; SEQ ID NO: 738; SEQ ID NO: 739; SEQ ID NO: 745; SEQ ID NO: 754; SEQ ID NO: 757; SEQ ID NO: 758; SEQ ID NO: 761; SEQ ID NO: 762; SEQ ID NO: 763; SEQ ID NO: 764; SEQ ID NO: 766; SEQ ID NO: 767; SEQ ID NO: 768; SEQ ID NO: 770; SEQ ID NO: 782; SEQ ID NO: 784; SEQ ID NO: 801; SEQ ID NO: 809; SEQ ID NO: 810; SEQ ID NO: 811; SEQ ID NO: 814; SEQ ID NO: 818; SEQ ID NO: 821; SEQ ID NO: 837; SEQ ID NO: 841; SEQ ID NO: 842; SEQ ID NO: 845; SEQ ID NO: 847; SEQ ID NO: 848; SEQ ID NO: 850; SEQ ID NO: 851; SEQ ID NO: 855; SEQ ID NO: 856; SEQ ID NO: 860; SEQ ID NO: 861; SEQ ID NO: 862; SEQ ID NO: 865; SEQ ID NO: 875; SEQ ID NO: 884; SEQ ID NO: 886; SEQ ID NO: 891; SEQ ID NO: 899; SEQ ID NO: 901; SEQ ID NO: 907; SEQ ID NO: 914; SEQ ID NO: 916; SEQ ID NO: 920; SEQ ID NO: 927; SEQ ID NO: 937; SEQ ID NO: 1056; SEQ ID NO: 1057; SEQ ID NO: 1058; SEQ ID NO: 1059; SEQ ID NO: 1078; SEQ ID NO: 2917; SEQ ID NO: 2919; SEQ ID NO: 2926; SEQ ID NO: 2946; SEQ ID NO: 2949; SEQ ID NO: 2951; SEQ ID NO: 2953; and SEQ ID NO: 2956. In some embodiments, the antisense strand comprises or consists of a sequence selected from SEQ ID NO: 715; SEQ ID NO: 732; SEQ ID NO: 733; SEQ ID NO: 737; SEQ ID NO: 738; SEQ ID NO: 739; SEQ ID NO: 745; SEQ ID NO: 754; SEQ ID NO: 757; SEQ ID NO: 761; SEQ ID NO: 762; SEQ ID NO: 763; SEQ ID NO: 764; SEQ ID NO: 766; SEQ ID NO: 767; SEQ ID NO: 784; SEQ ID NO: 801; SEQ ID NO: 809; SEQ ID NO: 810; SEQ ID NO: 811; SEQ ID NO: 814; SEQ ID NO: 841; SEQ ID NO: 842; SEQ ID NO: 845; SEQ ID NO: 848; SEQ ID NO: 851; SEQ ID NO: 856; SEQ ID NO: 860; SEQ ID NO: 862; SEQ ID NO: 914; SEQ ID NO: 916; SEQ ID NO: 927; SEQ ID NO: 937; SEQ ID NO: 1056; SEQ ID NO: 1057; SEQ ID NO: 1058; SEQ ID NO: 1059; SEQ ID NO: 1078; SEQ ID NO: 2917; SEQ ID NO: 2919; SEQ ID NO: 2926; SEQ ID NO: 2946; SEQ ID NO: 2949; SEQ ID NO: 2951; SEQ ID NO: 2953; and SEQ ID NO: 2956. In other embodiments, the antisense strand comprises or consists of a sequence selected from SEQ ID NO: 715; SEQ ID NO: 732; SEQ ID NO: 733; SEQ ID NO: 738; SEQ ID NO: 754; SEQ ID NO: 761; SEQ ID NO: 763; SEQ ID NO: 764; SEQ ID NO: 766; SEQ ID NO: 809; SEQ ID NO: 810; SEQ ID NO: 814; SEQ ID NO: 841; SEQ ID NO: 848; SEQ ID NO: 851; SEQ ID NO: 862; SEQ ID NO: 916; SEQ ID NO: 1057; SEQ ID NO: 1078; SEQ ID NO: 2919; SEQ ID NO: 2926; SEQ ID NO: 2946; SEQ ID NO: 2949; SEQ ID NO: 2953; and SEQ ID NO: 2956.

In these and other embodiments, the sense strand of the RNAi constructs of the invention can comprise or consist of the sequence of any one of the sense sequences listed in Table 1 or Table 2, the sequence of nucleotides 1-19 of any of these sense sequences, or the sequence of nucleotides 2-19 of any of these sense sequences. Thus, in some embodiments, the sense strand comprises or consists of a sequence selected from SEQ ID NOs: 2-670, 1340-2071, 2804-2905, or 3062-3320. In other embodiments, the sense strand comprises or consists of a sequence of nucleotides 1-19 of any one of SEQ ID NOs: 2-670, 1340-2071, 2804-2905, or 3062-3320. In still other embodiments, the sense strand comprises or consists of a sequence of nucleotides 2-19 of any one of SEQ ID NOs: 2-670, 1340-2071, 2804-2905, or 3062-3320. In certain embodiments, the sense strand comprises or consists of a sequence selected from SEQ ID NO: 46; SEQ ID NO: 56; SEQ ID NO: 63; SEQ ID NO: 64; SEQ ID NO: 68; SEQ ID NO: 69; SEQ ID NO: 70; SEQ ID NO: 76; SEQ ID NO: 85; SEQ ID NO: 88; SEQ ID NO: 89; SEQ ID NO: 92; SEQ ID NO: 93; SEQ ID NO: 94; SEQ ID NO: 95; SEQ ID NO: 97; SEQ ID NO: 98; SEQ ID NO: 99; SEQ ID NO: 101; SEQ ID NO: 113; SEQ ID NO: 115; SEQ ID NO: 132; SEQ ID NO: 140; SEQ ID NO: 141; SEQ ID NO: 142; SEQ ID NO: 145; SEQ ID NO: 149; SEQ ID NO: 152; SEQ ID NO: 168; SEQ ID NO: 172; SEQ ID NO: 173; SEQ ID NO: 176; SEQ ID NO: 178; SEQ ID NO: 179; SEQ ID NO: 181; SEQ ID NO: 182; SEQ ID NO: 186; SEQ ID NO: 187; SEQ ID NO: 191; SEQ ID NO: 192; SEQ ID NO: 193; SEQ ID NO: 196; SEQ ID NO: 206; SEQ ID NO: 215; SEQ ID NO: 217; SEQ ID NO: 222; SEQ ID NO: 230; SEQ ID NO: 232; SEQ ID NO: 238; SEQ ID NO: 245; SEQ ID NO: 247; SEQ ID NO: 251; SEQ ID NO: 258; SEQ ID NO: 268; SEQ ID NO: 387; SEQ ID NO: 388; SEQ ID NO: 389; SEQ ID NO: 390; SEQ ID NO: 391; SEQ ID NO: 392; SEQ ID NO: 409; SEQ ID NO: 2808; and SEQ ID NO: 2820. In certain other embodiments, the sense strand comprises or consists of a sequence selected from SEQ ID NO: 46; SEQ ID NO: 63; SEQ ID NO: 64; SEQ ID NO: 68; SEQ ID NO: 69; SEQ ID NO: 70; SEQ ID NO: 76; SEQ ID NO: 85; SEQ ID NO: 88; SEQ ID NO: 92; SEQ ID NO: 93; SEQ ID NO: 94; SEQ ID NO: 95; SEQ ID NO: 97; SEQ ID NO: 98; SEQ ID NO: 115; SEQ ID NO: 132; SEQ ID NO: 140; SEQ ID NO: 141; SEQ ID NO: 142; SEQ ID NO: 145; SEQ ID NO: 172; SEQ ID NO: 173; SEQ ID NO: 176; SEQ ID NO: 179; SEQ ID NO: 182; SEQ ID NO: 187; SEQ ID NO: 191; SEQ ID NO: 193; SEQ ID NO: 245; SEQ ID NO: 247; SEQ ID NO: 258; SEQ ID NO: 268; SEQ ID NO: 387; SEQ ID NO: 388; SEQ ID NO: 389; SEQ ID NO: 390; SEQ ID NO: 391; SEQ ID NO: 392; SEQ ID NO: 409; SEQ ID NO: 2808; and SEQ ID NO: 2820. In yet other embodiments, the sense strand comprises or consists of a sequence selected from SEQ ID NO: 46; SEQ ID NO: 63; SEQ ID NO: 64; SEQ ID NO: 69; SEQ ID NO: 85; SEQ ID NO: 92; SEQ ID NO: 94; SEQ ID NO: 95; SEQ ID NO: 97; SEQ ID NO: 140; SEQ ID NO: 141; SEQ ID NO: 145; SEQ ID NO: 172; SEQ ID NO: 179; SEQ ID NO: 182; SEQ ID NO: 193; SEQ ID NO: 247; SEQ ID NO: 388; SEQ ID NO: 390; SEQ ID NO: 391; SEQ ID NO: 409; SEQ ID NO: 2808; and SEQ ID NO: 2820.

In certain embodiments of the invention, the RNAi constructs comprise (i) a sense strand comprising or consisting of a sequence selected from 2-670, 1340-2071, 2804-2905, or 3062-3320 and (ii) an antisense strand comprising or consisting of a sequence selected from SEQ ID NOs: 671-1339, 2072-2803, 2906-3061, or 3321-3655. In some embodiments, the RNAi constructs comprise (i) a sense strand comprising or consisting of a sequence selected from SEQ ID NO: 46; SEQ ID NO: 56; SEQ ID NO: 63; SEQ ID NO: 64; SEQ ID NO: 68; SEQ ID NO: 69; SEQ ID NO: 70; SEQ ID NO: 76; SEQ ID NO: 85; SEQ ID NO: 88; SEQ ID NO: 89; SEQ ID NO: 92; SEQ ID NO: 93; SEQ ID NO: 94; SEQ ID NO: 95; SEQ ID NO: 97; SEQ ID NO: 98; SEQ ID NO: 99; SEQ ID NO: 101; SEQ ID NO: 113; SEQ ID NO: 115; SEQ ID NO: 132; SEQ ID NO: 140; SEQ ID NO: 141; SEQ ID NO: 142; SEQ ID NO: 145; SEQ ID NO: 149; SEQ ID NO: 152; SEQ ID NO: 168; SEQ ID NO: 172; SEQ ID NO: 173; SEQ ID NO: 176; SEQ ID NO: 178; SEQ ID NO: 179; SEQ ID NO: 181; SEQ ID NO: 182; SEQ ID NO: 186; SEQ ID NO: 187; SEQ ID NO: 191; SEQ ID NO: 192; SEQ ID NO: 193; SEQ ID NO: 196; SEQ ID NO: 206; SEQ ID NO: 215; SEQ ID NO: 217; SEQ ID NO: 222; SEQ ID NO: 230; SEQ ID NO: 232; SEQ ID NO: 238; SEQ ID NO: 245; SEQ ID NO: 247; SEQ ID NO: 251; SEQ ID NO: 258; SEQ ID NO: 268; SEQ ID NO: 387; SEQ ID NO: 388; SEQ ID NO: 389; SEQ ID NO: 390; SEQ ID NO: 391; SEQ ID NO: 392; SEQ ID NO: 409; SEQ ID NO: 2808; and SEQ ID NO: 2820 and (ii) an antisense strand comprising or consisting of a sequence selected from SEQ ID NO: 715; SEQ ID NO: 725; SEQ ID NO: 732; SEQ ID NO: 733; SEQ ID NO: 737; SEQ ID NO: 738; SEQ ID NO: 739; SEQ ID NO: 745; SEQ ID NO: 754; SEQ ID NO: 757; SEQ ID NO: 758; SEQ ID NO: 761; SEQ ID NO: 762; SEQ ID NO: 763; SEQ ID NO: 764; SEQ ID NO: 766; SEQ ID NO: 767; SEQ ID NO: 768; SEQ ID NO: 770; SEQ ID NO: 782; SEQ ID NO: 784; SEQ ID NO: 801; SEQ ID NO: 809; SEQ ID NO: 810; SEQ ID NO: 811; SEQ ID NO: 814; SEQ ID NO: 818; SEQ ID NO: 821; SEQ ID NO: 837; SEQ ID NO: 841; SEQ ID NO: 842; SEQ ID NO: 845; SEQ ID NO: 847; SEQ ID NO: 848; SEQ ID NO: 850; SEQ ID NO: 851; SEQ ID NO: 855; SEQ ID NO: 856; SEQ ID NO: 860; SEQ ID NO: 861; SEQ ID NO: 862; SEQ ID NO: 865; SEQ ID NO: 875; SEQ ID NO: 884; SEQ ID NO: 886; SEQ ID NO: 891; SEQ ID NO: 899; SEQ ID NO: 901; SEQ ID NO: 907; SEQ ID NO: 914; SEQ ID NO: 916; SEQ ID NO: 920; SEQ ID NO: 927; SEQ ID NO: 937; SEQ ID NO: 1056; SEQ ID NO: 1057; SEQ ID NO: 1058; SEQ ID NO: 1059; SEQ ID NO: 1078; SEQ ID NO: 2917; SEQ ID NO: 2919; SEQ ID NO: 2926; SEQ ID NO: 2946; SEQ ID NO: 2949; SEQ ID NO: 2951; SEQ ID NO: 2953; and SEQ ID NO: 2956. In other embodiments, the RNAi constructs comprise (i) a sense strand comprising or consisting of a sequence selected from SEQ ID NO: 46; SEQ ID NO: 63; SEQ ID NO: 64; SEQ ID NO: 68; SEQ ID NO: 69; SEQ ID NO: 70; SEQ ID NO: 76; SEQ ID NO: 85; SEQ ID NO: 88; SEQ ID NO: 92; SEQ ID NO: 93; SEQ ID NO: 94; SEQ ID NO: 95; SEQ ID NO: 97; SEQ ID NO: 98; SEQ ID NO: 115; SEQ ID NO: 132; SEQ ID NO: 140; SEQ ID NO: 141; SEQ ID NO: 142; SEQ ID NO: 145; SEQ ID NO: 172; SEQ ID NO: 173; SEQ ID NO: 176; SEQ ID NO: 179; SEQ ID NO: 182; SEQ ID NO: 187; SEQ ID NO: 191; SEQ ID NO: 193; SEQ ID NO: 245; SEQ ID NO: 247; SEQ ID NO: 258; SEQ ID NO: 268; SEQ ID NO: 387; SEQ ID NO: 388; SEQ ID NO: 389; SEQ ID NO: 390; SEQ ID NO: 391; SEQ ID NO: 392; SEQ ID NO: 409; SEQ ID NO: 2808; and SEQ ID NO: 2820 and (ii) an antisense strand comprising or consisting of a sequence selected from SEQ ID NO: 715; SEQ ID NO: 732; SEQ ID NO: 733; SEQ ID NO: 737; SEQ ID NO: 738; SEQ ID NO: 739; SEQ ID NO: 745; SEQ ID NO: 754; SEQ ID NO: 757; SEQ ID NO: 761; SEQ ID NO: 762; SEQ ID NO: 763; SEQ ID NO: 764; SEQ ID NO: 766; SEQ ID NO: 767; SEQ ID NO: 784; SEQ ID NO: 801; SEQ ID NO: 809; SEQ ID NO: 810; SEQ ID NO: 811; SEQ ID NO: 814; SEQ ID NO: 841; SEQ ID NO: 842; SEQ ID NO: 845; SEQ ID NO: 848; SEQ ID NO: 851; SEQ ID NO: 856; SEQ ID NO: 860; SEQ ID NO: 862; SEQ ID NO: 914; SEQ ID NO: 916; SEQ ID NO: 927; SEQ ID NO: 937; SEQ ID NO: 1056; SEQ ID NO: 1057; SEQ ID NO: 1058; SEQ ID NO: 1059; SEQ ID NO: 1078; SEQ ID NO: 2917; SEQ ID NO: 2919; SEQ ID NO: 2926; SEQ ID NO: 2946; SEQ ID NO: 2949; SEQ ID NO: 2951; SEQ ID NO: 2953; and SEQ ID NO: 2956. In still other embodiments, the RNAi constructs comprise (i) a sense strand comprising or consisting of a sequence selected from SEQ ID NO: 46; SEQ ID NO: 63; SEQ ID NO: 64; SEQ ID NO: 69; SEQ ID NO: 85; SEQ ID NO: 92; SEQ ID NO: 94; SEQ ID NO: 95; SEQ ID NO: 97; SEQ ID NO: 140; SEQ ID NO: 141; SEQ ID NO: 145; SEQ ID NO: 172; SEQ ID NO: 179; SEQ ID NO: 182; SEQ ID NO: 193; SEQ ID NO: 247; SEQ ID NO: 388; SEQ ID NO: 390; SEQ ID NO: 391; SEQ ID NO: 409; SEQ ID NO: 2808; and SEQ ID NO: 2820 and (ii) an antisense strand comprising or consisting of a sequence selected from SEQ ID NO: 715; SEQ ID NO: 732; SEQ ID NO: 733; SEQ ID NO: 738; SEQ ID NO: 754; SEQ ID NO: 761; SEQ ID NO: 763; SEQ ID NO: 764; SEQ ID NO: 766; SEQ ID NO: 809; SEQ ID NO: 810; SEQ ID NO: 814; SEQ ID NO: 841; SEQ ID NO: 848; SEQ ID NO: 851; SEQ ID NO: 862; SEQ ID NO: 916; SEQ ID NO: 1057; SEQ ID NO: 1078; SEQ ID NO: 2919; SEQ ID NO: 2926; SEQ ID NO: 2946; SEQ ID NO: 2949; SEQ ID NO: 2953; and SEQ ID NO: 2956.

In certain embodiments, the RNAi constructs of the invention comprise: (i) a sense strand comprising or consisting of the sequence of SEQ ID NO: 46 and an antisense strand comprising or consisting of the sequence of SEQ ID NO: 715; (ii) a sense strand comprising or consisting of the sequence of SEQ ID NO: 63 and an antisense strand comprising or consisting of the sequence of SEQ ID NO: 732; (iii) a sense strand comprising or consisting of the sequence of SEQ ID NO: 64 and an antisense strand comprising or consisting of the sequence of SEQ ID NO: 733; (iv) a sense strand comprising or consisting of the sequence of SEQ ID NO: 69 and an antisense strand comprising or consisting of the sequence of SEQ ID NO: 738; (v) a sense strand comprising or consisting of the sequence of SEQ ID NO: 85 and an antisense strand comprising or consisting of the sequence of SEQ ID NO: 754; (vi) a sense strand comprising or consisting of the sequence of SEQ ID NO: 92 and an antisense strand comprising or consisting of the sequence of SEQ ID NO: 761; (vii) a sense strand comprising or consisting of the sequence of SEQ ID NO: 94 and an antisense strand comprising or consisting of the sequence of SEQ ID NO: 763; (viii) a sense strand comprising or consisting of the sequence of SEQ ID NO: 95 and an antisense strand comprising or consisting of the sequence of SEQ ID NO: 764; (ix) a sense strand comprising or consisting of the sequence of SEQ ID NO: 97 and an antisense strand comprising or consisting of the sequence of SEQ ID NO: 766; (x) a sense strand comprising or consisting of the sequence of SEQ ID NO: 140 and an antisense strand comprising or consisting of the sequence of SEQ ID NO: 809; (xi) a sense strand comprising or consisting of the sequence of SEQ ID NO: 141 and an antisense strand comprising or consisting of the sequence of SEQ ID NO: 810; (xii) a sense strand comprising or consisting of the sequence of SEQ ID NO: 145 and an antisense strand comprising or consisting of the sequence of SEQ ID NO: 814; (xiii) a sense strand comprising or consisting of the sequence of SEQ ID NO: 172 and an antisense strand comprising or consisting of the sequence of SEQ ID NO: 841; (xiv) a sense strand comprising or consisting of the sequence of SEQ ID NO: 179 and an antisense strand comprising or consisting of the sequence of SEQ ID NO: 848; (xv) a sense strand comprising or consisting of the sequence of SEQ ID NO: 182 and an antisense strand comprising or consisting of the sequence of SEQ ID NO: 851; (xvi) a sense strand comprising or consisting of the sequence of SEQ ID NO: 193 and an antisense strand comprising or consisting of the sequence of SEQ ID NO: 862; or (xvii) a sense strand comprising or consisting of the sequence of SEQ ID NO: 247 and an antisense strand comprising or consisting of the sequence of SEQ ID NO: 916.

In certain other embodiments, the RNAi constructs of the invention comprise: (i) a sense strand comprising or consisting of the sequence of SEQ ID NO: 409 and an antisense strand comprising or consisting of the sequence of SEQ ID NO: 1078; (ii) a sense strand comprising or consisting of the sequence of SEQ ID NO: 388 and an antisense strand comprising or consisting of the sequence of SEQ ID NO: 1057; (iii) a sense strand comprising or consisting of the sequence of SEQ ID NO: 2808 and an antisense strand comprising or consisting of the sequence of SEQ ID NO: 2926; (iv) a sense strand comprising or consisting of the sequence of SEQ ID NO: 2820 and an antisense strand comprising or consisting of the sequence of SEQ ID NO: 2946; (v) a sense strand comprising or consisting of the sequence of SEQ ID NO: 391 and an antisense strand comprising or consisting of the sequence of SEQ ID NO: 2949; (vi) a sense strand comprising or consisting of the sequence of SEQ ID NO: 390 and an antisense strand comprising or consisting of the sequence of SEQ ID NO: 2956; (vii) a sense strand comprising or consisting of the sequence of SEQ ID NO: 179 and an antisense strand comprising or consisting of the sequence of SEQ ID NO: 2919; (viii) a sense strand comprising or consisting of the sequence of SEQ ID NO: 388 and an antisense strand comprising or consisting of the sequence of SEQ ID NO: 2953; or (ix) a sense strand comprising or consisting of the sequence of SEQ ID NO: 388 and an antisense strand comprising or consisting of the sequence of SEQ ID NO: 1057.

In some embodiments, the RNAi constructs of the invention comprise: (i) a sense strand comprising or consisting of the sequence of modified nucleotides according to SEQ ID NO: 2009 and an antisense strand comprising or consisting of the sequence of modified nucleotides according to SEQ ID NO: 2741; (ii) a sense strand comprising or consisting of the sequence of modified nucleotides according to SEQ ID NO: 2011 and an antisense strand comprising or consisting of the sequence of modified nucleotides according to SEQ ID NO: 2743; (iii) a sense strand comprising or consisting of the sequence of modified nucleotides according to SEQ ID NO: 2012 and an antisense strand comprising or consisting of the sequence of modified nucleotides according to SEQ ID NO: 2744; (iv) a sense strand comprising or consisting of the sequence of modified nucleotides according to SEQ ID NO: 2013 and an antisense strand comprising or consisting of the sequence of modified nucleotides according to SEQ ID NO: 2745; (v) a sense strand comprising or consisting of the sequence of modified nucleotides according to SEQ ID NO: 2020 and an antisense strand comprising or consisting of the sequence of modified nucleotides according to SEQ ID NO: 2752; (vi) a sense strand comprising or consisting of the sequence of modified nucleotides according to SEQ ID NO: 2035 and an antisense strand comprising or consisting of the sequence of modified nucleotides according to SEQ ID NO: 2767; (vii) a sense strand comprising or consisting of the sequence of modified nucleotides according to SEQ ID NO: 2037 and an antisense strand comprising or consisting of the sequence of modified nucleotides according to SEQ ID NO: 2769; (viii) a sense strand comprising or consisting of the sequence of modified nucleotides according to SEQ ID NO: 2041 and an antisense strand comprising or consisting of the sequence of modified nucleotides according to SEQ ID NO: 2773; (ix) a sense strand comprising or consisting of the sequence of modified nucleotides according to SEQ ID NO: 2042 and an antisense strand comprising or consisting of the sequence of modified nucleotides according to SEQ ID NO: 2774; (x) a sense strand comprising or consisting of the sequence of modified nucleotides according to SEQ ID NO: 2043 and an antisense strand comprising or consisting of the sequence of modified nucleotides according to SEQ ID NO: 2775; (xi) a sense strand comprising or consisting of the sequence of modified nucleotides according to SEQ ID NO: 2044 and an antisense strand comprising or consisting of the sequence of modified nucleotides according to SEQ ID NO: 2776; (xii) a sense strand comprising or consisting of the sequence of modified nucleotides according to SEQ ID NO: 2045 and an antisense strand comprising or consisting of the sequence of modified nucleotides according to SEQ ID NO: 2777; (xiii) a sense strand comprising or consisting of the sequence of modified nucleotides according to SEQ ID NO: 2051 and an antisense strand comprising or consisting of the sequence of modified nucleotides according to SEQ ID NO: 2783; (xiv) a sense strand comprising or consisting of the sequence of modified nucleotides according to SEQ ID NO: 2053 and an antisense strand comprising or consisting of the sequence of modified nucleotides according to SEQ ID NO: 2785; (xv) a sense strand comprising or consisting of the sequence of modified nucleotides according to SEQ ID NO: 2054 and an antisense strand comprising or consisting of the sequence of modified nucleotides according to SEQ ID NO: 2786; (xvi) a sense strand comprising or consisting of the sequence of modified nucleotides according to SEQ ID NO: 2055 and an antisense strand comprising or consisting of the sequence of modified nucleotides according to SEQ ID NO: 2787; or (xvii) a sense strand comprising or consisting of the sequence of modified nucleotides according to SEQ ID NO: 2059 and an antisense strand comprising or consisting of the sequence of modified nucleotides according to SEQ ID NO: 2791.

In other embodiments, the RNAi constructs of the invention comprise: (i) a sense strand comprising or consisting of the sequence of modified nucleotides according to SEQ ID NO: 3078 and an antisense strand comprising or consisting of the sequence of modified nucleotides according to SEQ ID NO: 3337; (ii) a sense strand comprising or consisting of the sequence of modified nucleotides according to SEQ ID NO: 3080 and an antisense strand comprising or consisting of the sequence of modified nucleotides according to SEQ ID NO: 3339; (iii) a sense strand comprising or consisting of the sequence of modified nucleotides according to SEQ ID NO: 3163 and an antisense strand comprising or consisting of the sequence of modified nucleotides according to SEQ ID NO: 3441; (iv) a sense strand comprising or consisting of the sequence of modified nucleotides according to SEQ ID NO: 3183 and an antisense strand comprising or consisting of the sequence of modified nucleotides according to SEQ ID NO: 3469; (v) a sense strand comprising or consisting of the sequence of modified nucleotides according to SEQ ID NO: 3076 and an antisense strand comprising or consisting of the sequence of modified nucleotides according to SEQ ID NO: 3472; (vi) a sense strand comprising or consisting of the sequence of modified nucleotides according to SEQ ID NO: 3077 and an antisense strand comprising or consisting of the sequence of modified nucleotides according to SEQ ID NO: 3484; (vii) a sense strand comprising or consisting of the sequence of modified nucleotides according to SEQ ID NO: 2051 and an antisense strand comprising or consisting of the sequence of modified nucleotides according to SEQ ID NO: 3545; (viii) a sense strand comprising or consisting of the sequence of modified nucleotides according to SEQ ID NO: 3080 and an antisense strand comprising or consisting of the sequence of modified nucleotides according to SEQ ID NO: 3481; (ix) a sense strand comprising or consisting of the sequence of modified nucleotides according to SEQ ID NO: 3188 and an antisense strand comprising or consisting of the sequence of modified nucleotides according to SEQ ID NO: 3339; (x) a sense strand comprising or consisting of the sequence of modified nucleotides according to SEQ ID NO: 3080 and an antisense strand comprising or consisting of the sequence of modified nucleotides according to SEQ ID NO: 3476; or (xi) a sense strand comprising or consisting of the sequence of modified nucleotides according to SEQ ID NO: 3223 and an antisense strand comprising or consisting of the sequence of modified nucleotides according to SEQ ID NO: 3517.

The RNAi construct of the invention can be any of the duplex compounds listed in Tables 1 to 24 (including the unmodified nucleotide sequences and/or modified nucleotide sequences of the compounds). In some embodiments, the RNAi construct is any of the duplex compounds listed in Table 1. In other embodiments, the RNAi construct is any of the duplex compounds listed in Table 2 (including the unmodified nucleotide sequences and/or modified nucleotide sequences of the compounds). In certain embodiments, the RNAi construct is D-1044, D-1061, D-1062, D-1067, D-1083, D-1090, D-1092, D-1093, D-1095, D-1138, D-1139, D-1143, D-1170, D-1177, D-1180, D-1191, D-1245, D-2000, D-2002, D-2003, D-2004, D-2011, D-2026, D-2028, D-2032, D-2033, D-2034, D-2035, D-2036, D-2042, D-2044, D-2045, D-2046, D-2050, D-2078, D-2079, D-2081, D-2182, D-2196, D-2238, D-2241, D-2243, D-2246, D-2255, D-2258, D-2301, D-2316, D-2317, D-2329, D-2332, D-2341, D-2344, D-2356, D-2357, D-2399, or D-2510. In certain other embodiments, the RNAi construct is D-2079, D-2081, D-2196, D-2238, D-2241, D-2255, D-2258, D-2317, D-2332, D-2357, or D-2399.

In certain embodiments, the RNAi constructs of the invention may target a particular region of the human mARC1 transcript sequence. As described in Example 4 and summarized in Table 23, it was found that certain RNAi constructs with antisense strands designed to have a sequence complementary to certain regions of the human mARC1 transcript (SEQ ID NO: 1) exhibited superior in vivo knockdown activity of human mARC1 mRNA as compared to RNAi constructs with antisense strands complementary to other regions of the transcript. Thus, in some embodiments of the invention, RNAi constructs that are particularly suitable for inhibiting expression of a human MARC1 gene in a cell comprise a sense strand and an antisense strand that hybridize to form a duplex region of about 15 to about 30 base pairs in length, wherein the antisense strand comprises a region having a sequence that is substantially complementary to the sequence of at least 15 contiguous nucleotides of nucleotides 1205 to 1250 of SEQ ID NO: 1. In one embodiment, the antisense strand comprises a region having a sequence that is substantially complementary to the sequence of at least 15 contiguous nucleotides of nucleotides 1209 to 1239 of SEQ ID NO: 1. In another embodiment, the antisense strand comprises a region having a sequence that is substantially complementary to the sequence of at least 15 contiguous nucleotides of nucleotides 1211 to 1236 of SEQ ID NO: 1. In some such embodiments, the antisense strand has a sequence that is substantially complementary with no more than 1, 2, or 3 mismatches to the sequence of at least 15 contiguous nucleotides of nucleotides 1205 to 1250, nucleotides 1209 to 1239, or nucleotides 1211 to 1236 of SEQ ID NO: 1. In other embodiments, the antisense strand has a sequence that is fully complementary to the sequence of at least 15 contiguous nucleotides of nucleotides 1205 to 1250, nucleotides 1209 to 1239, or nucleotides 1211 to 1236 of SEQ ID NO: 1. RNAi constructs targeting nucleotides 1205 to 1250 of the human mARC1 transcript include, but are not limited to, D-2063, D-2066, D-2076, D-2077, D-2078, D-2080, D-2081, D-2108, D-2113, D-2142, D-2240, D-2241, D-2243, D-2245, D-2246, D-2248, D-2250, D-2251, D-2253, D-2255, D-2256, D-2258, D-2259, D-2261, D-2264, D-2265, D-2268, D-2269, D-2270, D-2271, D-2301, D-2309, D-2311, D-2312, D-2314, D-2316, D-2317, D-2319, D-2321, D-2322, D-2324, D-2326, D-2327, D-2329, D-2331, D-2332, D-2334, D-2336, D-2337, D-2339, D-2341, D-2342, D-2344, D-2346, D-2347, D-2349, D-2351, D-2352, D-2354, D-2356, D-2357, D-2376, D-2380, D-2393, D-2395, D-2396, D-2431, D-2436, D-2437, D-2440, D-2441, D-2444, D-2445, D-2447, D-2453, D-2518, D-2519, D-2520, D-2521, D-2522, D-2523, D-2524, D-2525, D-2526, D-2527, D-2528, D-2529, D-2530, D-2531, D-2532, D-2533, D-2534, and D-2535. In some embodiments, the RNAi construct targeting nucleotides 1205 to 1250 of the human mARC1 transcript is D-2063, D-2066, D-2076, D-2077, D-2078, D-2080, D-2081, D-2108, D-2113, D-2142, or D-2301. In certain embodiments, RNAi constructs targeting nucleotides 1205 to 1250, particularly nucleotides 1211 to 1236, of SEQ ID NO: 1 comprise an antisense strand comprising the sequence of 5′-CAUCUAAUAUUCCAG-3′ (SEQ ID NO: 3656).

In other embodiments, the RNAi constructs of the invention comprise a sense strand and an antisense strand that hybridize to form a duplex region of about 15 to about 30 base pairs in length, wherein the antisense strand comprises a region having a sequence that is substantially complementary to the sequence of at least 15 contiguous nucleotides of nucleotides 1345 to 1375 of SEQ ID NO: 1. In one embodiment, the antisense strand comprises a sequence that is substantially complementary with no more than 1, 2, or 3 mismatches to the sequence of at least 15 contiguous nucleotides of nucleotides 1345 to 1375 of SEQ ID NO: 1. In another embodiment, the antisense strand comprises a sequence that is fully complementary to the sequence of at least 15 contiguous nucleotides of nucleotides 1345 to 1375 of SEQ ID NO: 1. Exemplary RNAi constructs targeting nucleotides 1345 to 1375 of the human mARC1 transcript include, but are not limited to, D-2042, D-2043, D-2047, D-2052, D-2158, D-2162, D-2169, D-2182, D-2183, D-2184, D-2185, D-2186, D-2187, D-2189, D-2211, D-2213, D-2304, D-2305, D-2306, D-2307, D-2308, D-2384, D-2384, D-2385, D-2386, D-2387, D-2388, D-2389, D-2390, D-2391, D-2392, D-2399, D-2400, D-2401, D-2402, D-2403, D-2488, D-2494, D-2500, D-2506, D-2512, D-2538, D-2539, D-2540, and D-2541. In some embodiments, the RNAi construct targeting nucleotides 1345 to 1375 of the human mARC1 transcript is D-2042, D-2043, D-2047, D-2052, D-2304, D-2305, D-2306, D-2307, or D-2308. In certain embodiments, RNAi constructs targeting nucleotides 1345 to 1375, particularly nucleotides 1350 to 1375, of SEQ ID NO: 1 comprise an antisense strand comprising the sequence of 5′-UGGGACAUUGAAGCA-3′ (SEQ ID NO: 3657).

In still other embodiments, RNAi constructs of the invention comprise a sense strand and an antisense strand that hybridize to form a duplex region of about 15 to about 30 base pairs in length, wherein the antisense strand comprises a region having a sequence that is substantially complementary to the sequence of at least 15 contiguous nucleotides of nucleotides 2039 to 2078 of SEQ ID NO: 1. In one embodiment, the antisense strand comprises a region having a sequence that is substantially complementary to the sequence of at least 15 contiguous nucleotides of nucleotides 2048 to 2074 of SEQ ID NO: 1. In some such embodiments, the antisense strand has a sequence that is substantially complementary with no more than 1, 2, or 3 mismatches to the sequence of at least 15 contiguous nucleotides of nucleotides 2039 to 2078 or nucleotides 2048 to 2074 of SEQ ID NO: 1. In other embodiments, the antisense strand has a sequence that is fully complementary to the sequence of at least 15 contiguous nucleotides of nucleotides 2039 to 2078 or nucleotides 2048 to 2074 of SEQ ID NO: 1. RNAi constructs targeting nucleotides 2039 to 2078 of the human mARC1 transcript include, but are not limited to, D-2045, D-2065, D-2079, D-2082, D-2105, D-2106, D-2137, D-2143, D-2166, D-2173, D-2193, D-2242, D-2247, D-2252, D-2257, D-2260, D-2262, D-2266, D-2272, D-2273, D-2302, D-2303, D-2310, D-2313, D-2315, D-2318, D-2320, D-2323, D-2325, D-2328, D-2330, D-2333, D-2335, D-2338, D-2340, D-2343, D-2345, D-2348, D-2350, D-2353, D-2355, D-2358, D-2394, D-2397, D-2454, D-2455, D-2456, D-2457, D-2458, D-2459, D-2460, D-2463, D-2465, D-2465, D-2468, D-2470, D-2472, D-2473, D-2477, D-2487, D-2493, D-2499, D-2505, D-2511, D-2552, D-2553, D-2554, D-2555, D-2556, and D-2557. In certain embodiments, the RNAi construct targeting nucleotides 2039 to 2078 of the human mARC1 transcript is D-2045, D-2065, D-2079, D-2082, D-2105, D-2106, D-2137, D-2143, D-2302, or D-2303. In certain other embodiments, RNAi constructs targeting nucleotides 2039 to 2078, particularly nucleotides 2048 to 2074, of SEQ ID NO: 1 comprise an antisense strand comprising the sequence of 5′-AUCAGAUCUUAGAGU-3′ (SEQ ID NO: 3658).

The RNAi constructs of the invention may comprise one or more modified nucleotides. A “modified nucleotide” refers to a nucleotide that has one or more chemical modifications to the nucleoside, nucleobase, pentose ring, or phosphate group. As used herein, modified nucleotides do not encompass ribonucleotides containing adenosine monophosphate, guanosine monophosphate, uridine monophosphate, and cytidine monophosphate. However, the RNAi constructs may comprise combinations of modified nucleotides and ribonucleotides. Incorporation of modified nucleotides into one or both strands of double-stranded RNA molecules can improve the in vivo stability of the RNA molecules, e.g., by reducing the molecules' susceptibility to nucleases and other degradation processes. The potency of RNAi constructs for reducing expression of the target gene can also be enhanced by incorporation of modified nucleotides.

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than OH. Such 2′-modifications include, but are not limited to, 2′-H (e.g. deoxyribonucleotides), 2′-O-alkyl (e.g. —O—C₁-C₁₀ or —O—C₁-C₁₀ substituted alkyl), 2′-O-allyl (—O—CH₂CH═CH₂), 2′-C-allyl, 2′-deoxy-2′-fluoro (also referred to as 2′-F or 2′-fluoro), 2′-O-methyl (—OCH₃), 2′-O-methoxyethyl (—O—(CH₂)₂OCH₃), 2′-OCF₃, 2′-O(CH₂)₂SCH₃, 2′-O-aminoalkyl, 2′-amino (e.g. —NH₂), 2′-O-ethylamine, and 2′-azido. Modifications at the 5′ position of the pentose ring include, but are not limited to, 5′-methyl (R or S configuration); 5′-vinyl, and 5′-methoxy.

A “bicyclic sugar modification” refers to a modification of the pentose ring where a bridge connects two atoms of the ring to form a second ring resulting in a bicyclic sugar structure. In some embodiments the bicyclic sugar modification comprises a bridge between the 4′ and 2′ carbons of the pentose ring. Nucleotides comprising a sugar moiety with a bicyclic sugar modification are referred to herein as bicyclic nucleic acids or BNAs. Exemplary bicyclic sugar modifications include, but are not limited to, α-L-Methyleneoxy (4′-CH₂—O-2′) bicyclic nucleic acid (BNA); β-D-Methyleneoxy (4′-CH₂—O-2′) BNA (also referred to as a locked nucleic acid or LNA); Ethyleneoxy (4′-(CH₂)₂—O-2′) BNA; Aminooxy (4′-CH₂—O—N(R)-2′, wherein R is H, C₁-C₁₂ alkyl, or a protecting group) BNA; Oxyamino (4′-CH₂—N(R)—O-2′, wherein R is H, C₁-C₁₂ alkyl, or a protecting group) BNA; Methyl(methyleneoxy) (4′-CH(CH₃)—O-2′) BNA (also referred to as constrained ethyl or cEt); methylene-thio (4′-CH₂—S-2′) BNA; methylene-amino (4′-CH2-N(R)-2′, wherein R is H, C₁-C₁₂ alkyl, or a protecting group) BNA; methyl carbocyclic (4′-CH₂—CH(CH₃)-2′) BNA; propylene carbocyclic (4′-(CH₂)₃-2′) BNA; and Methoxy(ethyleneoxy) (4′-CH(CH₂OMe)-O-2′) BNA (also referred to as constrained MOE or cMOE). These and other sugar-modified nucleotides that can be incorporated into the RNAi constructs of the invention are described in U.S. Pat. No. 9,181,551, U.S. Patent Publication No. 2016/0122761, and Deleavey and Damha, Chemistry and Biology, Vol. 19: 937-954, 2012, all of which are hereby incorporated by reference in their entireties.

In some embodiments, the RNAi constructs comprise one or more 2′-fluoro modified nucleotides, 2′-O-methyl modified nucleotides, 2′-O-methoxyethyl modified nucleotides, 2′-O-alkyl modified nucleotides, 2′-O-allyl modified nucleotides, bicyclic nucleic acids (BNAs), deoxyribonucleotides, or combinations thereof. In certain embodiments, the RNAi constructs comprise one or more 2′-fluoro modified nucleotides, 2′-O-methyl modified nucleotides, 2′-O-methoxyethyl modified nucleotides, or combinations thereof. In one particular embodiment, the RNAi constructs comprise one or more 2′-fluoro modified nucleotides, 2′-O-methyl modified nucleotides or combinations thereof.

Both the sense and antisense strands of the RNAi constructs can comprise one or multiple modified nucleotides. For instance, in some embodiments, the sense strand comprises 1, 2, 3, 4, 5, 6, 7, 8, 9, 10 or more modified nucleotides. In certain embodiments, all nucleotides in the sense strand are modified nucleotides. In some embodiments, the antisense strand comprises 1, 2, 3, 4, 5, 6, 7, 8, 9, 10 or more modified nucleotides. In other embodiments, all nucleotides in the antisense strand are modified nucleotides. In certain other embodiments, all nucleotides in the sense strand and all nucleotides in the antisense strand are modified nucleotides. In these and other embodiments, the modified nucleotides can be 2′-fluoro modified nucleotides, 2′-O-methyl modified nucleotides, or combinations thereof.

In certain embodiments, the modified nucleotides incorporated into one or both of the strands of the RNAi constructs of the invention have a modification of the nucleobase (also referred to herein as “base”). A “modified nucleobase” or “modified base” refers to a base other than the naturally occurring purine bases adenine (A) and guanine (G) and pyrimidine bases thymine (T), cytosine (C), and uracil (U). Modified nucleobases can be synthetic or naturally occurring modifications and include, but are not limited to, universal bases, 5-methylcytosine (5-me-C), 5-hydroxymethyl cytosine, xanthine (X), hypoxanthine (I), 2-aminoadenine, 6-methyladenine, 6-methylguanine, and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

In some embodiments, the modified base is a universal base. A “universal base” refers to a base analog that indiscriminately forms base pairs with all of the natural bases in RNA and DNA without altering the double helical structure of the resulting duplex region. Universal bases are known to those of skill in the art and include, but are not limited to, inosine, C-phenyl, C-naphthyl and other aromatic derivatives, azole carboxamides, and nitroazole derivatives, such as 3-nitropyrrole, 4-nitroindole, 5-nitroindole, and 6-nitroindole.

Other suitable modified bases that can be incorporated into the RNAi constructs of the invention include those described in Herdewijn, Antisense Nucleic Acid Drug Dev., Vol. 10: 297-310, 2000 and Peacock et al., J. Org. Chem., Vol. 76: 7295-7300, 2011, both of which are hereby incorporated by reference in their entireties. The skilled person is well aware that guanine, cytosine, adenine, thymine, and uracil may be replaced by other nucleobases, such as the modified nucleobases described above, without substantially altering the base pairing properties of a polynucleotide comprising a nucleotide bearing such replacement nucleobase.

In some embodiments, the sense and antisense strands of the RNAi constructs may comprise one or more abasic nucleotides. An “abasic nucleotide” or “abasic nucleoside” is a nucleotide or nucleoside that lacks a nucleobase at the 1′ position of the ribose sugar. In certain embodiments, the abasic nucleotides are incorporated into the terminal ends of the sense and/or antisense strands of the RNAi constructs. In one embodiment, the sense strand comprises an abasic nucleotide as the terminal nucleotide at its 3′ end, its 5′ end, or both its 3′ and 5′ ends. In another embodiment, the antisense strand comprises an abasic nucleotide as the terminal nucleotide at its 3′ end, its 5′ end, or both its 3′ and 5′ ends. In such embodiments in which the abasic nucleotide is a terminal nucleotide, it may be an inverted nucleotide—that is, linked to the adjacent nucleotide through a 3′-3′ internucleotide linkage (when on the 3′ end of a strand) or through a 5′-5′ internucleotide linkage (when on the 5′ end of a strand) rather than the natural 3′-5′ internucleotide linkage. Abasic nucleotides may also comprise a sugar modification, such as any of the sugar modifications described above. In certain embodiments, abasic nucleotides comprise a 2′-modification, such as a 2′-fluoro modification, 2′-O-methyl modification, or a 2′-H (deoxy) modification. In one embodiment, the abasic nucleotide comprises a 2′-O-methyl modification. In another embodiment, the abasic nucleotide comprises a 2′-H modification (i.e. a deoxy abasic nucleotide).

In certain embodiments, the RNAi constructs of the invention may comprise modified nucleotides incorporated into the sense and anti sense strands according to a particular pattern, such as the patterns described in WIPO Publication No. WO 2020/123410, which is hereby incorporated by reference in its entirety. RNAi constructs having such chemical modification patterns have been shown to have improved gene silencing activity in vivo. In one embodiment, the RNAi construct of the invention comprises a sense strand and an antisense strand that comprise sequences that are sufficiently complementary to each other to form a duplex region of at least 15 base pairs, wherein:
nucleotides at positions 2, 7, and 14 in the antisense strand (counting from the 5′ end) are 2′-fluoro modified nucleotides;
nucleotides in the sense strand at positions paired with positions 8 to 11 and 13 in the antisense strand (counting from the 5′ end) are 2′-fluoro modified nucleotides; and
neither the sense strand nor the antisense strand each have more than 7 total 2′-fluoro modified nucleotides.

In other embodiments, the RNAi construct of the invention comprises a sense strand and an antisense strand that comprise sequences that are sufficiently complementary to each other to form a duplex region of at least 19 base pairs, wherein:
nucleotides at positions 2, 7, and 14 in the antisense strand (counting from the 5′ end) are 2′-fluoro modified nucleotides, nucleotides at positions 4, 6, 10, and 12 (counting from the 5′ end) are optionally 2′-fluoro modified nucleotides, and all other nucleotides in the antisense strand are modified nucleotides other than 2′-fluoro modified nucleotides; and
nucleotides in the sense strand at positions paired with positions 8 to 11 and 13 in the antisense strand (counting from the 5′ end) are 2′-fluoro modified nucleotides, nucleotides in the sense strand at positions paired with positions 3 and 5 in the antisense strand (counting from the 5′ end) are optionally 2′-fluoro modified nucleotides; and all other nucleotides in the sense strand are modified nucleotides other than 2′-fluoro modified nucleotides.

In such embodiments, the modified nucleotides other than 2′-fluoro modified nucleotides can be selected from 2′-O-methyl modified nucleotides, 2′-O-methoxyethyl modified nucleotides, 2′-O-alkyl modified nucleotides, 2′-O-allyl modified nucleotides, BNAs, and deoxyribonucleotides. In these and other embodiments, the terminal nucleotide at the 3′ end, the 5′ end, or both the 3′ end and the 5′ end of the sense strand can be an abasic nucleotide or a deoxyribonucleotide. In such embodiments, the abasic nucleotide or deoxyribonucleotide may be inverted—i.e. linked to the adjacent nucleotide through a 3′-3′ internucleotide linkage (when on the 3′ end of a strand) or through a 5′-5′ internucleotide linkage (when on the 5′ end of a strand) rather than the natural 3′-5′ internucleotide linkage.

In any of the above-described embodiments, nucleotides at positions 2, 7, 12, and 14 in the antisense strand (counting from the 5′ end) are 2′-fluoro modified nucleotides. In other embodiments, nucleotides at positions 2, 4, 7, 12, and 14 in the antisense strand (counting from the 5′ end) are 2′-fluoro modified nucleotides. In yet other embodiments, nucleotides at positions 2, 4, 6, 7, 12, and 14 in the antisense strand (counting from the 5′ end) are 2′-fluoro modified nucleotides. In still other embodiments, nucleotides at positions 2, 4, 6, 7, 10, 12, and 14 in the antisense strand (counting from the 5′ end) are 2′-fluoro modified nucleotides. In alternative embodiments, nucleotides at positions 2, 7, 10, 12, and 14 in the antisense strand (counting from the 5′ end) are 2′-fluoro modified nucleotides. In certain other embodiments, nucleotides at positions 2, 4, 7, 10, 12, and 14 in the antisense strand (counting from the 5′ end) are 2′-fluoro modified nucleotides.

In any of the above-described embodiments, nucleotides in the sense strand at positions paired with positions 3, 8 to 11, and 13 in the antisense strand (counting from the 5′ end) are 2′-fluoro modified nucleotides. In some embodiments, nucleotides in the sense strand at positions paired with positions 5, 8 to 11, and 13 in the antisense strand (counting from the 5′ end) are 2′-fluoro modified nucleotides. In other embodiments, nucleotides in the sense strand at positions paired with positions 3, 5, 8 to 11, and 13 in the antisense strand (counting from the 5′ end) are 2′-fluoro modified nucleotides.

In some embodiments, the RNAi construct of the invention comprises a structure represented by Formula (A):

5′-(N_{A})ₓN_{L}N_{L}N_{L}N_{L}N_{L}N_{L}N_{F}N_{L}N_{F}N_{F}N_{F}N_{F}N_{L}N_{L}N_{M}N_{L}N_{M}N_{L}N_{T}(n)_{y}-3′

3′-(N_{B})_{z}N_{L}N_{L}N_{L}N_{L}N_{L}N_{F}N_{L}N_{M}N_{L}N_{M}N_{L}N_{L}N_{F}N_{M}N_{L}N_{M}N_{L}N_{F}N_{L}-5′ (A)

In Formula (A), the top strand listed in the 5′ to 3′ direction is the sense strand and the bottom strand listed in the 3′ to 5′ direction is the antisense strand; each N_{F} represents a 2′-fluoro modified nucleotide; each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; and N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. X can be an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the N_{A} nucleotides can be complementary to nucleotides in the antisense strand. Y can be an integer from 0 to 4, provided that when y is 1, 2, 3, or 4, one or more n nucleotides are modified or unmodified overhang nucleotides that do not base pair with nucleotides in the antisense strand. Z can be an integer from 0 to 4, provided that when z is 1, 2, 3, or 4, one or more of the N_{B} nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the N_{B} nucleotides can be complementary to N_{A} nucleotides when present in the sense strand or can be overhang nucleotides that do not base pair with nucleotides in the sense strand.

In some embodiments in which the RNAi construct comprises a structure represented by Formula (A), there is a nucleotide overhang at the 3′ end of the sense strand—i.e. y is 1, 2, 3, or 4. In one such embodiment, y is 2. In embodiments in which there is an overhang of 2 nucleotides at the 3′ end of the sense strand (i.e. y is 2), x is 0 and z is 2 or x is 1 and z is 2. In other embodiments in which the RNAi construct comprises a structure represented by Formula (A), the RNAi construct comprises a blunt end at the 3′ end of the sense strand and the 5′ end of the antisense strand (i.e. y is 0). In such embodiments where there is no nucleotide overhang at the 3′ end of the sense strand (i.e. y is 0): (i) x is 2 and z is 4, (ii) x is 3 and z is 4, (iii) x is 0 and z is 2, (iv) x is 1 and z is 2, or (v) x is 2 and z is 2. In any of the embodiments in which x is greater than 0, the N_{A} nucleotide that is the terminal nucleotide at the 5′ end of the sense strand can be an inverted nucleotide, such as an inverted abasic nucleotide or an inverted deoxyribonucleotide.

In certain embodiments in which the RNAi construct comprises a structure represented by Formula (A), the N_{M} at positions 4 and 12 in the antisense strand counting from the 5′ end are each a 2′-fluoro modified nucleotide. In other embodiments, the N_{M} at positions 4, 6, and 12 in the antisense strand counting from the 5′ end are each a 2′-fluoro modified nucleotide. In yet other embodiments, the N_{M} at positions 4, 6, 10, and 12 in the antisense strand counting from the 5′ end are each a 2′-fluoro modified nucleotide. In alternative embodiments in which the RNAi construct comprises a structure represented by Formula (A), the N_{M} at positions 10 and 12 in the antisense strand counting from the 5′ end are each a 2′-fluoro modified nucleotide. In related embodiments, the N_{M} at positions 4, 10, and 12 in the antisense strand counting from the 5′ end are each a 2′-fluoro modified nucleotide. In other alternative embodiments in which the RNAi construct comprises a structure represented by Formula (A), the N_{M} at positions 4, 6, and 10 in the antisense strand counting from the 5′ end are each a 2′-O-methyl modified nucleotide, and the N_{M} at position 12 in the antisense strand counting from the 5′ end is a 2′-fluoro modified nucleotide. In some embodiments in which the RNAi construct comprises a structure represented by Formula (A), each N_{M} in the sense strand is a 2′-O-methyl modified nucleotide. In other embodiments, each N_{M} in the sense strand is a 2′-fluoro modified nucleotide. In still other embodiments in which the RNAi construct comprises a structure represented by Formula (A), each N_{M} in both the sense and antisense strands is a 2′-O-methyl modified nucleotide.

In any of the above-described embodiments in which the RNAi construct comprises a structure represented by Formula (A), each N_{L} in both the sense and antisense strands can be a 2′-O-methyl modified nucleotide. In these embodiments and any of the embodiments described above, N_{T} in Formula (A) can be an inverted abasic nucleotide, an inverted deoxyribonucleotide, or a 2′-O-methyl modified nucleotide.

In other embodiments of the invention, the RNAi construct of the invention comprises a structure represented by Formula (B):

5′-(N_{A})ₓN_{L}N_{L}N_{L}N_{L}N_{M}N_{L}N_{F}N_{F}N_{F}N_{F}N_{L}N_{L}N_{L}N_{L}N_{L}N_{L}N_{L}N_{L}N_{T}(n)_{y}-3′

3′-(N_{B})_{z}N_{L}N_{L}N_{L}N_{M}N_{L}N_{F}N_{L}N_{M}N_{L}N_{L}N_{M}N_{M}N_{M}N_{M}N_{L}N_{M}N_{L}N_{F}N_{L}-5′ (B)

In Formula (B), the top strand listed in the 5′ to 3′ direction is the sense strand and the bottom strand listed in the 3′ to 5′ direction is the antisense strand; each N_{F} represents a 2′-fluoro modified nucleotide; each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; and N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. X can be an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the N_{A} nucleotides can be complementary to nucleotides in the antisense strand. Y can be an integer from 0 to 4, provided that when y is 1, 2, 3, or 4, one or more n nucleotides are modified or unmodified overhang nucleotides that do not base pair with nucleotides in the antisense strand. Z can be an integer from 0 to 4, provided that when z is 1, 2, 3, or 4, one or more of the N_{B} nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the N_{B} nucleotides can be complementary to N_{A} nucleotides when present in the sense strand or can be overhang nucleotides that do not base pair with nucleotides in the sense strand.

In some embodiments in which the RNAi construct comprises a structure represented by Formula (B), there is a nucleotide overhang at the 3′ end of the sense strand—i.e. y is 1, 2, 3, or 4. In one such embodiment, y is 2. In embodiments in which there is an overhang of 2 nucleotides at the 3′ end of the sense strand (i.e. y is 2), x is 0 and z is 2 or x is 1 and z is 2. In other embodiments in which the RNAi construct comprises a structure represented by Formula (B), the RNAi construct comprises a blunt end at the 3′ end of the sense strand and the 5′ end of the antisense strand (i.e. y is 0). In such embodiments where there is no nucleotide overhang at the 3′ end of the sense strand (i.e. y is 0): (i) x is 2 and z is 4, (ii) x is 3 and z is 4, (iii) x is 0 and z is 2, (iv) x is 1 and z is 2, or (v) x is 2 and z is 2. In any of the embodiments in which x is greater than 0, the N_{A} nucleotide that is the terminal nucleotide at the 5′ end of the sense strand can be an inverted nucleotide, such as an inverted abasic nucleotide or an inverted deoxyribonucleotide.

In certain embodiments in which the RNAi construct comprises a structure represented by Formula (B), the N_{M} at positions 4, 6, 8, 9, and 16 in the antisense strand counting from the 5′ end are each a 2′-fluoro modified nucleotide and the N_{M} at positions 7 and 12 in the antisense strand counting from the 5′ end are each a 2′-O-methyl modified nucleotide. In other embodiments, the N_{M} at positions 4 and 6 in the antisense strand counting from the 5′ end are each a 2′-fluoro modified nucleotide and the N_{M} at positions 7 to 9 in the antisense strand counting from the 5′ end are each a 2′-O-methyl modified nucleotide. In still other embodiments, the N_{M} at positions 4, 6, 8, 9, and 16 in the antisense strand counting from the 5′ end are each a 2′-O-methyl modified nucleotide and the N_{M} at positions 7 and 12 in the antisense strand counting from the 5′ end are each a 2′-fluoro modified nucleotide. In alternative embodiments in which the RNAi construct comprises a structure represented by Formula (B), the N_{M} at positions 4, 6, 8, 9, and 12 in the antisense strand counting from the 5′ end are each a 2′-O-methyl modified nucleotide and the N_{M} at positions 7 and 16 in the antisense strand counting from the 5′ end are each a 2′-fluoro modified nucleotide. In certain other embodiments in which the RNAi construct comprises a structure represented by Formula (B), the N_{M} at positions 7, 8, 9, and 12 in the antisense strand counting from the 5′ end are each a 2′-O-methyl modified nucleotide and the N_{M} at positions 4, 6, and 16 in the antisense strand counting from the 5′ end are each a 2′-fluoro modified nucleotide. In these and other embodiments in which the RNAi construct comprises a structure represented by Formula (B), the N_{M} in the sense strand is a 2′-fluoro modified nucleotide. In alternative embodiments, the N_{M} in the sense strand is a 2′-O-methyl modified nucleotide.

In any of the above-described embodiments in which the RNAi construct comprises a structure represented by Formula (B), each N_{L} in both the sense and antisense strands can be a 2′-O-methyl modified nucleotide. In these embodiments and any of the embodiments described above, N_{T} in Formula (B) can be an inverted abasic nucleotide, an inverted deoxyribonucleotide, or a 2′-O-methyl modified nucleotide.

The RNAi constructs of the invention may also comprise one or more modified internucleotide linkages. As used herein, the term “modified internucleotide linkage” refers to an internucleotide linkage other than the natural 3′ to 5′ phosphodiester linkage. In some embodiments, the modified internucleotide linkage is a phosphorous-containing internucleotide linkage, such as a phosphotriester, aminoalkylphosphotriester, an alkylphosphonate (e.g. methylphosphonate, 3′-alkylene phosphonate), a phosphinate, a phosphoramidate (e.g. 3′-amino phosphoramidate and aminoalkylphosphoramidate), a phosphorothioate, a chiral phosphorothioate, a phosphorodithioate, a thionophosphoramidate, a thionoalkylphosphonate, a thionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (—O—Si(H)₂—O—); sulfide, sulfoxide and sulfone linkages; formacetyl and thioformacetyl linkages; alkene containing backbones; sulfamate backbones; methylenemethylimino (—CH₂—N(CH₃)—O—CH₂—) and methylenehydrazino linkages; sulfonate and sulfonamide linkages; amide linkages; and others having mixed N, O, S and CH₂ component parts. In one embodiment, the modified internucleoside linkage is a peptide-based linkage (e.g. aminoethylglycine) to create a peptide nucleic acid or PNA, such as those described in U.S. Pat. Nos. 5,539,082; 5,714,331; and 5,719,262. Other suitable modified internucleotide and internucleoside linkages that may be employed in the RNAi constructs of the invention are described in U.S. Pat. Nos. 6,693,187, 9,181,551, U.S. Patent Publication No. 2016/0122761, and Deleavey and Damha, Chemistry and Biology, Vol. 19: 937-954, 2012, all of which are hereby incorporated by reference in their entireties.

In certain embodiments, the RNAi constructs of the invention comprise one or more phosphorothioate internucleotide linkages. The phosphorothioate internucleotide linkages may be present in the sense strand, antisense strand, or both strands of the RNAi constructs. For instance, in some embodiments, the sense strand comprises 1, 2, 3, 4, 5, 6, 7, 8, or more phosphorothioate internucleotide linkages. In other embodiments, the antisense strand comprises 1, 2, 3, 4, 5, 6, 7, 8, or more phosphorothioate internucleotide linkages. In still other embodiments, both strands comprise 1, 2, 3, 4, 5, 6, 7, 8, or more phosphorothioate internucleotide linkages. The RNAi constructs can comprise one or more phosphorothioate internucleotide linkages at the 3′-end, the 5′-end, or both the 3′- and 5′-ends of the sense strand, the antisense strand, or both strands. For instance, in certain embodiments, the RNAi construct comprises about 1 to about 6 or more (e.g., about 1, 2, 3, 4, 5, 6 or more) consecutive phosphorothioate internucleotide linkages at the 3′-end of the sense strand, the antisense strand, or both strands. In other embodiments, the RNAi construct comprises about 1 to about 6 or more (e.g., about 1, 2, 3, 4, 5, 6 or more) consecutive phosphorothioate internucleotide linkages at the 5′-end of the sense strand, the antisense strand, or both strands. In one particular embodiment, the antisense strand comprises at least 1 but no more than 6 phosphorothioate internucleotide linkages and the sense strand comprises at least 1 but no more than 4 phosphorothioate internucleotide linkages. In another particular embodiment, the antisense strand comprises at least 1 but no more than 4 phosphorothioate internucleotide linkages and the sense strand comprises at least 1 but no more than 2 phosphorothioate internucleotide linkages.

In some embodiments, the RNAi construct comprises a single phosphorothioate internucleotide linkage between the terminal nucleotides at the 3′ end of the sense strand. In other embodiments, the RNAi construct comprises two consecutive phosphorothioate internucleotide linkages between the terminal nucleotides at the 3′ end of the sense strand. In one embodiment, the RNAi construct comprises a single phosphorothioate internucleotide linkage between the terminal nucleotides at the 3′ end of the sense strand and a single phosphorothioate internucleotide linkage between the terminal nucleotides at the 3′ end of the antisense strand. In another embodiment, the RNAi construct comprises two consecutive phosphorothioate internucleotide linkages between the terminal nucleotides at the 3′ end of the antisense strand (i.e. a phosphorothioate internucleotide linkage at the first and second internucleotide linkages at the 3′ end of the antisense strand). In another embodiment, the RNAi construct comprises two consecutive phosphorothioate internucleotide linkages between the terminal nucleotides at both the 3′ and 5′ ends of the antisense strand. In yet another embodiment, the RNAi construct comprises two consecutive phosphorothioate internucleotide linkages between the terminal nucleotides at both the 3′ and 5′ ends of the antisense strand and two consecutive phosphorothioate internucleotide linkages at the 5′ end of the sense strand. In still another embodiment, the RNAi construct comprises two consecutive phosphorothioate internucleotide linkages between the terminal nucleotides at both the 3′ and 5′ ends of the antisense strand and two consecutive phosphorothioate internucleotide linkages between the terminal nucleotides at the 3′ end of the sense strand. In another embodiment, the RNAi construct comprises two consecutive phosphorothioate internucleotide linkages between the terminal nucleotides at both the 3′ and 5′ ends of the antisense strand and two consecutive phosphorothioate internucleotide linkages between the terminal nucleotides at both the 3′ and 5′ ends of the sense strand (i.e. a phosphorothioate internucleotide linkage at the first and second internucleotide linkages at both the 5′ and 3′ ends of the antisense strand and a phosphorothioate internucleotide linkage at the first and second internucleotide linkages at both the 5′ and 3′ ends of the sense strand). In yet another embodiment, the RNAi construct comprises two consecutive phosphorothioate internucleotide linkages between the terminal nucleotides at both the 3′ and 5′ ends of the antisense strand and a single phosphorothioate internucleotide linkage between the terminal nucleotides at the 3′ end of the sense strand. In any of the embodiments in which one or both strands comprise one or more phosphorothioate internucleotide linkages, the remaining internucleotide linkages within the strands can be the natural 3′ to 5′ phosphodiester linkages. For instance, in some embodiments, each internucleotide linkage of the sense and antisense strands is selected from phosphodiester and phosphorothioate, wherein at least one internucleotide linkage is a phosphorothioate.

In embodiments in which the RNAi construct comprises a nucleotide overhang, two or more of the unpaired nucleotides in the overhang can be connected by a phosphorothioate internucleotide linkage. In certain embodiments, all the unpaired nucleotides in a nucleotide overhang at the 3′ end of the antisense strand and/or the sense strand are connected by phosphorothioate internucleotide linkages. In other embodiments, all the unpaired nucleotides in a nucleotide overhang at the 5′ end of the antisense strand and/or the sense strand are connected by phosphorothioate internucleotide linkages. In still other embodiments, all the unpaired nucleotides in any nucleotide overhang are connected by phosphorothioate internucleotide linkages.

Incorporation of a phosphorothioate internucleotide linkage introduces an additional chiral center at the phosphorous atom in the oligonucleotide and therefore creates a diastereomer pair (Rp and Sp) at each phosphorothioate internucleotide linkage. Diastereomers or diastereoisomers are different configurations of a compound that have the same molecular formula and sequence of bonded atoms but differ in the three-dimensional orientations of their atoms in space. Unlike enantiomers, diastereomers are not mirror-images of each other. Each chiral phosphate atom can be in the “R” configuration (Rp) or the “S” configuration (Sp). In certain embodiments, the RNAi constructs of the invention may comprise one or more phosphorothioate internucleotide linkages where the chiral phosphates are selected to be primarily in either the Rp or Sp configuration. For instance, in some embodiments in which the RNAi constructs have one or more phosphorothioate internucleotide linkages, at least about 70%, at least about 75%, at least about 80%, at least about 85%, at least about 90%, or at least about 95% of the chiral phosphates are in the Sp configuration. In other embodiments in which the RNAi constructs have one or more phosphorothioate internucleotide linkages, at least about 70%, at least about 75%, at least about 80%, at least about 85%, at least about 90%, or at least about 95% of the chiral phosphates are in the Rp configuration. All the chiral phosphates in the RNAi construct can be either in the Sp configuration or the Rp configuration (i.e. the RNAi construct is stereopure). In one particular embodiment, all the chiral phosphates in the RNAi construct are in the Sp configuration. In another particular embodiment, all the chiral phosphates in the RNAi construct are in the Rp configuration.

In certain embodiments, the chiral phosphates in the RNAi construct may have different configurations at different positions in the sense strand or antisense strand. In one such embodiment in which the RNAi construct comprises one or two phosphorothioate internucleotide linkages at the 5′ end of the antisense strand, the chiral phosphates at the 5′ end of the antisense strand may be in the Rp configuration. In another such embodiment in which the RNAi construct comprises one or two phosphorothioate internucleotide linkages at the 3′ end of the antisense strand, the chiral phosphates at the 3′ end of the antisense strand may be in the Sp configuration. In certain embodiments, the RNAi construct comprises two consecutive phosphorothioate internucleotide linkages between the terminal nucleotides at both the 3′ and 5′ ends of the antisense strand and two consecutive phosphorothioate internucleotide linkages between the terminal nucleotides at the 3′ end of the sense strand, wherein the chiral phosphates at the 5′ end of the antisense strand are in the Rp configuration, the chiral phosphates at the 3′ end of the antisense strand are in the Sp configuration, and the chiral phosphates at the 3′ end of the sense strand can be either in the Rp or Sp configuration. In certain other embodiments, the RNAi construct comprises two consecutive phosphorothioate internucleotide linkages between the terminal nucleotides at both the 3′ and 5′ ends of the antisense strand and a single phosphorothioate internucleotide linkage between the terminal nucleotides at the 3′ end of the sense strand, wherein the chiral phosphates at the 5′ end of the antisense strand are in the Rp configuration, the chiral phosphates at the 3′ end of the antisense strand are in the Sp configuration, and the chiral phosphate at the 3′ end of the sense strand can be either in the Rp or Sp configuration. Methods of controlling the stereochemistry of phosphorothioate linkages during oligonucleotide synthesis are known to those skilled in the art and can include methods described in Nawrot and Rebowska, Curr Protoc Nucleic Acid Chem. 2009, Chapter 4: doi:10.1002/0471142700.nc0434s362009; Jahns et al., Nat. Commun, Vol. 6: 6317, 2015; Knouse et al., Science, Vol. 361: 1234-1238, 2018; and Sakamuri et al., Chembiochem, Vol. 21(9): 1304-1308, 2020.

In some embodiments of the RNAi constructs of the invention, the 5′ end of the sense strand, antisense strand, or both the antisense and sense strands comprises a phosphate moiety. As used herein, the term “phosphate moiety” refers to a terminal phosphate group that includes unmodified phosphates (—O—P═O)(OH)OH) as well as modified phosphates. Modified phosphates include phosphates in which one or more of the O and OH groups are replaced with H, O, S, N(R) or alkyl (e.g. C₁ to C₁₂) where R is H, an amino protecting group or unsubstituted or substituted alkyl (e.g. C₁ to C₁₂). Exemplary phosphate moieties include, but are not limited to, 5′-monophosphate; 5′-diphosphate; 5′-triphosphate; 5′-guanosine cap (7-methylated or non-methylated); 5′-adenosine cap or any other modified or unmodified nucleotide cap structure; 5′-monothiophosphate (phosphorothioate); 5′-monodithiophosphate (phosphorodithioate); 5′-alpha-thiotriphosphate; 5′-gamma-thiotriphosphate, 5′-phosphoramidates; 5′-vinylphosphates; 5′-alkylphosphonates (e.g., alkyl=methyl, ethyl, isopropyl, propyl, etc.); and 5′-alkyletherphosphonates (e.g., alkylether=methoxymethyl, ethoxymethyl, etc.).

The modified nucleotides that can be incorporated into the RNAi constructs of the invention may have more than one chemical modification described herein. For instance, the modified nucleotide may have a modification to the ribose sugar as well as a modification to the nucleobase. By way of example, a modified nucleotide may comprise a 2′ sugar modification (e.g. 2′-fluoro or 2′-O-methyl) and comprise a modified base (e.g. 5-methyl cytosine or pseudouracil). In other embodiments, the modified nucleotide may comprise a sugar modification in combination with a modification to the 5′ phosphate that would create a modified internucleotide or internucleoside linkage when the modified nucleotide was incorporated into a polynucleotide. For instance, in some embodiments, the modified nucleotide may comprise a sugar modification, such as a 2′-fluoro modification, a 2′-O-methyl modification, or a bicyclic sugar modification, as well as a 5′ phosphorothioate group. Accordingly, in some embodiments, one or both strands of the RNAi constructs of the invention comprise a combination of 2′ modified nucleotides or BNAs and phosphorothioate internucleotide linkages. In certain embodiments, both the sense and antisense strands of the RNAi constructs of the invention comprise a combination of 2′-fluoro modified nucleotides, 2′-O-methyl modified nucleotides, and phosphorothioate internucleotide linkages. Exemplary RNAi constructs comprising modified nucleotides and internucleotide linkages are shown in Table 2.

The RNAi constructs of the invention can readily be made using techniques known in the art, for example, using conventional nucleic acid solid phase synthesis. The polynucleotides of the RNAi constructs can be assembled on a suitable nucleic acid synthesizer utilizing standard nucleotide or nucleoside precursors (e.g. phosphoramidites). Automated nucleic acid synthesizers are sold commercially by several vendors, including DNA/RNA synthesizers from Applied Biosystems (Foster City, Calif.), MerMade synthesizers from BioAutomation (Irving, Tex.), and OligoPilot synthesizers from GE Healthcare Life Sciences (Pittsburgh, Pa.). An exemplary method for synthesizing the RNAi constructs of the invention is described in Example 2.

A 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides.

The 2′-O-silyl group can be removed via exposure to fluoride ions, which can include any source of fluoride ion, e.g., those salts containing fluoride ion paired with inorganic counterions e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Exemplary fluoride ion sources are tetrabutylammonium fluoride or aminohydrofluorides (e.g., combining aqueous HF with triethylamine in a dipolar aprotic solvent, e.g., dimethylformamide).

The choice of protecting groups for use on the phosphite triesters and phosphotriesters can alter the stability of the triesters towards fluoride. Methyl protection of the phosphotriester or phosphite triester can stabilize the linkage against fluoride ions and improve process yields.

Since ribonucleosides have a reactive 2′ hydroxyl substituent, it can be desirable to protect the reactive 2′ position in RNA with a protecting group that is orthogonal to a 5′-O-dimethoxytrityl protecting group, e.g., one stable to treatment with acid. Silyl protecting groups meet this criterion and can be readily removed in a final fluoride deprotection step that can result in minimal RNA degradation.

Tetrazole catalysts can be used in the standard phosphoramidite coupling reaction. Exemplary catalysts include, e.g., tetrazole, S-ethyl-tetrazole, benzylthiotetrazole, p-nitrophenyltetrazole.

As can be appreciated by the skilled artisan, further methods of synthesizing the RNAi constructs described herein will be evident to those of ordinary skill in the art. Additionally, the various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing the RNAi constructs described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Custom synthesis of RNAi constructs is also available from several commercial vendors, including Dharmacon, Inc. (Lafayette, Colo.), AxoLabs GmbH (Kulmbach, Germany), and Ambion, Inc. (Foster City, Calif.).

The RNAi constructs of the invention may comprise a ligand. As used herein, a “ligand” refers to any compound or molecule that is capable of interacting with another compound or molecule, directly or indirectly. The interaction of a ligand with another compound or molecule may elicit a biological response (e.g. initiate a signal transduction cascade, induce receptor-mediated endocytosis) or may just be a physical association. The ligand can modify one or more properties of the double-stranded RNA molecule to which is attached, such as the pharmacodynamic, pharmacokinetic, binding, absorption, cellular distribution, cellular uptake, charge and/or clearance properties of the RNA molecule.

The ligand may comprise a serum protein (e.g., human serum albumin, low-density lipoprotein, globulin), a cholesterol moiety, a vitamin (biotin, vitamin E, vitamin B 12), a folate moiety, a steroid, a bile acid (e.g. cholic acid), a fatty acid (e.g., palmitic acid, myristic acid), a carbohydrate (e.g., a dextran, pullulan, chitin, chitosan, inulin, cyclodextrin or hyaluronic acid), a glycoside, a phospholipid, or antibody or binding fragment thereof (e.g. antibody or binding fragment that targets the RNAi construct to a specific cell type, such as liver). Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, O3-(oleoyl)lithocholic acid, O3-(oleoyl)cholenic acid, dimethoxytrityl, or phenoxazine), peptides (e.g., antennapedia peptide, Tat peptide, RGD peptides), alkylating agents, polymers, such as polyethylene glycol (PEG)(e.g., PEG-40K), polyamino acids, and polyamines (e.g. spermine, spermidine).

In certain embodiments, the ligands have endosomolytic properties. The endosomolytic ligands promote the lysis of the endosome and/or transport of the RNAi construct of the invention, or its components, from the endosome to the cytoplasm of the cell. The endosomolytic ligand may be a polycationic peptide or peptidomimetic, which shows pH-dependent membrane activity and fusogenicity. In one embodiment, the endosomolytic ligand assumes its active conformation at endosomal pH. The “active” conformation is that conformation in which the endosomolytic ligand promotes lysis of the endosome and/or transport of the RNAi construct of the invention, or its components, from the endosome to the cytoplasm of the cell. Exemplary endosomolytic ligands include the GALA peptide (Subbarao et al., Biochemistry, Vol. 26: 2964-2972, 1987), the EALA peptide (Vogel et al., J. Am. Chem. Soc., Vol. 118: 1581-1586, 1996), and their derivatives (Turk et al., Biochem. Biophys. Acta, Vol. 1559: 56-68, 2002). In one embodiment, the endosomolytic component may contain a chemical group (e.g., an amino acid) which will undergo a change in charge or protonation in response to a change in pH. The endosomolytic component may be linear or branched.

In some embodiments, the ligand comprises a lipid or other hydrophobic molecule. In one embodiment, the ligand comprises a cholesterol moiety or other steroid. Cholesterol-conjugated oligonucleotides have been reported to be more active than their unconjugated counterparts (Manoharan, Antisense Nucleic Acid Drug Development, Vol. 12: 103-228, 2002). Ligands comprising cholesterol moieties and other lipids for conjugation to nucleic acid molecules have also been described in U.S. Pat. Nos. 7,851,615; 7,745,608; and 7,833,992, all of which are hereby incorporated by reference in their entireties. In another embodiment, the ligand comprises a folate moiety. Polynucleotides conjugated to folate moieties can be taken up by cells via a receptor-mediated endocytosis pathway. Such folate-polynucleotide conjugates are described in U.S. Pat. No. 8,188,247, which is hereby incorporated by reference in its entirety.

In certain embodiments, it is desirable to specifically deliver the RNAi constructs of the invention to liver cells to reduce expression of mARC1 protein specifically in the liver. Accordingly, in certain embodiments, the ligand targets delivery of the RNAi construct specifically to liver cells (e.g. hepatocytes) using various approaches as described in more detail below. In certain embodiments, the RNAi constructs are targeted to liver cells with a ligand that binds to the surface-expressed asialoglycoprotein receptor (ASGR) or component thereof (e.g. ASGR1, ASGR2).

In some embodiments, RNAi constructs can be specifically targeted to the liver by employing ligands that bind to or interact with proteins expressed on the surface of liver cells. For example, in certain embodiments, the ligands may comprise antigen binding proteins (e.g. antibodies or binding fragments thereof (e.g. Fab, scFv)) that specifically bind to a receptor expressed on hepatocytes, such as the asialoglycoprotein receptor and the LDL receptor. In one particular embodiment, the ligand comprises an antibody or binding fragment thereof that specifically binds to ASGR1 and/or ASGR2. In another embodiment, the ligand comprises a Fab fragment of an antibody that specifically binds to ASGR1 and/or ASGR2. A “Fab fragment” is comprised of one immunoglobulin light chain (i.e. light chain variable region (VL) and constant region (CL)) and the CH1 region and variable region (VH) of one immunoglobulin heavy chain. In another embodiment, the ligand comprises a single-chain variable antibody fragment (scFv fragment) of an antibody that specifically binds to ASGR1 and/or ASGR2. An “scFv fragment” comprises the VH and VL regions of an antibody, wherein these regions are present in a single polypeptide chain, and optionally comprising a peptide linker between the VH and VL regions that enables the Fv to form the desired structure for antigen binding. Exemplary antibodies and binding fragments thereof that specifically bind to ASGR1 that can be used as ligands for targeting the RNAi constructs of the invention to the liver are described in WIPO Publication No. WO 2017/058944, which is hereby incorporated by reference in its entirety. Other antibodies or binding fragments thereof that specifically bind to ASGR1, LDL receptor, or other liver surface-expressed proteins suitable for use as ligands in the RNAi constructs of the invention are commercially available.

In certain embodiments, the ligand comprises a carbohydrate. A “carbohydrate” refers to a compound made up of one or more monosaccharide units having at least 6 carbon atoms (which can be linear, branched or cyclic) with an oxygen, nitrogen or sulfur atom bonded to each carbon atom. Carbohydrates include, but are not limited to, the sugars (e.g., monosaccharides, disaccharides, trisaccharides, tetrasaccharides, and oligosaccharides containing from about 4, 5, 6, 7, 8, or 9 monosaccharide units), and polysaccharides, such as starches, glycogen, cellulose and polysaccharide gums. In some embodiments, the carbohydrate incorporated into the ligand is a monosaccharide selected from a pentose, hexose, or heptose and di- and tri-saccharides including such monosaccharide units. In other embodiments, the carbohydrate incorporated into the ligand is an amino sugar, such as galactosamine, glucosamine, N-acetylgalactosamine, and N-acetylglucosamine.

In some embodiments, the ligand comprises a hexose or hexosamine. The hexose may be selected from glucose, galactose, mannose, fucose, or fructose. The hexosamine may be selected from fructosamine, galactosamine, glucosamine, or mannosamine. In certain embodiments, the ligand comprises glucose, galactose, galactosamine, or glucosamine. In one embodiment, the ligand comprises glucose, glucosamine, or N-acetylglucosamine. In another embodiment, the ligand comprises galactose, galactosamine, or N-acetyl-galactosamine. In particular embodiments, the ligand comprises N-acetyl-galactosamine. Ligands comprising glucose, galactose, and N-acetyl-galactosamine (GalNAc) are particularly effective in targeting compounds to liver cells because such ligands bind to the ASGR expressed on the surface of hepatocytes. See, e.g., D'Souza and Devarajan, J. Control Release, Vol. 203: 126-139, 2015. Examples of GalNAc- or galactose-containing ligands that can be incorporated into the RNAi constructs of the invention are described in U.S. Pat. Nos. 7,491,805; 8,106,022; and 8,877,917; U.S. Patent Publication No. 20030130186; and WIPO Publication No. WO 2013166155, all of which are hereby incorporated by reference in their entireties.

In certain embodiments, the ligand comprises a multivalent carbohydrate moiety. As used herein, a “multivalent carbohydrate moiety” refers to a moiety comprising two or more carbohydrate units capable of independently binding or interacting with other molecules. For example, a multivalent carbohydrate moiety comprises two or more binding domains comprised of carbohydrates that can bind to two or more different molecules or two or more different sites on the same molecule. The valency of the carbohydrate moiety denotes the number of individual binding domains within the carbohydrate moiety. For instance, the terms “monovalent,” “bivalent,” “trivalent,” and “tetravalent” with reference to the carbohydrate moiety refer to carbohydrate moieties with one, two, three, and four binding domains, respectively. The multivalent carbohydrate moiety may comprise a multivalent lactose moiety, a multivalent galactose moiety, a multivalent glucose moiety, a multivalent N-acetyl-galactosamine moiety, a multivalent N-acetyl-glucosamine moiety, a multivalent mannose moiety, or a multivalent fucose moiety. In some embodiments, the ligand comprises a multivalent galactose moiety. In other embodiments, the ligand comprises a multivalent N-acetyl-galactosamine moiety. In these and other embodiments, the multivalent carbohydrate moiety can be bivalent, trivalent, or tetravalent. In such embodiments, the multivalent carbohydrate moiety can be bi-antennary or tri-antennary. In one particular embodiment, the multivalent N-acetyl-galactosamine moiety is trivalent or tetravalent. In another particular embodiment, the multivalent galactose moiety is trivalent or tetravalent. Exemplary trivalent and tetravalent GalNAc-containing ligands for incorporation into the RNAi constructs of the invention are described in detail below.

The ligand can be attached or conjugated to the RNA molecule of the RNAi construct directly or indirectly. For instance, in some embodiments, the ligand is covalently attached directly to the sense or antisense strand of the RNAi construct. In other embodiments, the ligand is covalently attached via a linker to the sense or antisense strand of the RNAi construct. The ligand can be attached to nucleobases, sugar moieties, or internucleotide linkages of polynucleotides (e.g. sense strand or antisense strand) of the RNAi constructs of the invention. Conjugation or attachment to purine nucleobases or derivatives thereof can occur at any position including, endocyclic and exocyclic atoms. In certain embodiments, the 2-, 6-, 7-, or 8-positions of a purine nucleobase are attached to a ligand. Conjugation or attachment to pyrimidine nucleobases or derivatives thereof can also occur at any position. In some embodiments, the 2-, 5-, and 6-positions of a pyrimidine nucleobase can be attached to a ligand. Conjugation or attachment to sugar moieties of nucleotides can occur at any carbon atom. Exemplary carbon atoms of a sugar moiety that can be attached to a ligand include the 2′, 3′, and 5′ carbon atoms. The 1′ position can also be attached to a ligand, such as in an abasic nucleotide. Internucleotide linkages can also support ligand attachments. For phosphorus-containing linkages (e.g., phosphodiester, phosphorothioate, phosphorodithiotate, phosphoroamidate, and the like), the ligand can be attached directly to the phosphorus atom or to an O, N, or S atom bound to the phosphorus atom. For amine- or amide-containing internucleoside linkages (e.g., PNA), the ligand can be attached to the nitrogen atom of the amine or amide or to an adjacent carbon atom.

In some embodiments, the ligand may be attached to the 3′ or 5′ end of either the sense or antisense strand. In certain embodiments, the ligand is covalently attached to the 5′ end of the sense strand. In such embodiments, the ligand is attached to the 5′-terminal nucleotide of the sense strand. In these and other embodiments, the ligand is attached at the 5′-position of the 5′-terminal nucleotide of the sense strand. In embodiments in which an inverted abasic nucleotide is the 5′-terminal nucleotide of the sense strand and linked to the adjacent nucleotide via a 5′-5′ internucleotide linkage, the ligand can be attached at the 3′-position of the inverted abasic nucleotide. In other embodiments, the ligand is covalently attached to the 3′ end of the sense strand. For example, in some embodiments, the ligand is attached to the 3′-terminal nucleotide of the sense strand. In certain such embodiments, the ligand is attached at the 3′-position of the 3′-terminal nucleotide of the sense strand. In embodiments in which an inverted abasic nucleotide is the 3′-terminal nucleotide of the sense strand and linked to the adjacent nucleotide via a 3′-3′ internucleotide linkage, the ligand can be attached at the 5′-position of the inverted abasic nucleotide. In alternative embodiments, the ligand is attached near the 3′ end of the sense strand, but before one or more terminal nucleotides (i.e. before 1, 2, 3, or 4 terminal nucleotides). In some embodiments, the ligand is attached at the 2′-position of the sugar of the 3′-terminal nucleotide of the sense strand. In other embodiments, the ligand is attached at the 2′-position of the sugar of the 5′-terminal nucleotide of the sense strand.

In certain embodiments, the ligand is attached to the sense or antisense strand via a linker. A “linker” is an atom or group of atoms that covalently joins a ligand to a polynucleotide component of the RNAi construct. The linker may be from about 1 to about 30 atoms in length, from about 2 to about 28 atoms in length, from about 3 to about 26 atoms in length, from about 4 to about 24 atoms in length, from about 6 to about 20 atoms in length, from about 7 to about 20 atoms in length, from about 8 to about 20 atoms in length, from about 8 to about 18 atoms in length, from about 10 to about 18 atoms in length, and from about 12 to about 18 atoms in length. In some embodiments, the linker may comprise a bifunctional linking moiety, which generally comprises an alkyl moiety with two functional groups. One of the functional groups is selected to bind to the compound of interest (e.g. sense or antisense strand of the RNAi construct) and the other is selected to bind essentially any selected group, such as a ligand as described herein. In certain embodiments, the linker comprises a chain structure or an oligomer of repeating units, such as ethylene glycol or amino acid units. Examples of functional groups that are typically employed in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Linkers that may be used to attach a ligand to the sense or antisense strand in the RNAi constructs of the invention include, but are not limited to, pyrrolidine, 8-amino-3,6-dioxaoctanoic acid, succinimidyl 4-(N-maleimidomethyl)cyclohexane-1-carboxylate, 6-aminohexanoic acid, substituted C₁-C₁₀ alkyl, substituted or unsubstituted C₂-C₁₀ alkenyl or substituted or unsubstituted C₂-C₁₀ alkynyl. Suitable substituent groups for such linkers include, but are not limited to, hydroxyl, amino, alkoxy, carboxy, benzyl, phenyl, nitro, thiol, thioalkoxy, halogen, alkyl, aryl, alkenyl and alkynyl.

In certain embodiments, the linkers are cleavable. A cleavable linker is one which is sufficiently stable outside the cell, but which upon entry into a target cell is cleaved to release the two parts the linker is holding together. In some embodiments, the cleavable linker is cleaved at least 10 times, 20 times, 30 times, 40 times, 50 times, 60 times, 70 times, 80 times, 90 times, or more, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linkers are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linker by reduction; esterases; endosomes or agents that can create an acidic environment, e.g., those that result in a pH of five or lower; enzymes that can hydrolyze or degrade an acid cleavable linker by acting as a general acid, peptidases (which can be substrate specific), and phosphatases.

A cleavable linker may comprise a moiety that is susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable group that is cleaved at a preferred pH, thereby releasing the RNA molecule from the ligand inside the cell, or into the desired compartment of the cell.

A linker can include a cleavable group that is cleavable by a particular enzyme. The type of cleavable group incorporated into a linker can depend on the cell to be targeted. For example, liver-targeting ligands can be linked to RNA molecules through a linker that includes an ester group. Liver cells are rich in esterases, and therefore the linker will be cleaved more efficiently in liver cells than in cell types that are not esterase-rich. Other types of cells rich in esterases include cells of the lung, renal cortex, and testis. Linkers that contain peptide bonds can be used when targeting cells rich in peptidases, such as liver cells and synoviocytes.

In general, the suitability of a candidate cleavable linker can be evaluated by testing the ability of a degradative agent (or condition) to cleave the candidate linker. It will also be desirable to also test the candidate cleavable linker for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In some embodiments, useful candidate linkers are cleaved at least 2, 4, 10, 20, 50, 70, or 100 times faster in the cell (or under in vitro conditions selected to mimic intracellular conditions) as compared to blood or serum (or under in vitro conditions selected to mimic extracellular conditions).

In other embodiments, redox cleavable linkers are utilized. Redox cleavable linkers are cleaved upon reduction or oxidation. An example of a reductively cleavable group is a disulfide linking group (—S—S—). To determine if a candidate cleavable linker is a suitable “reductively cleavable linker,” or for example is suitable for use with a particular RNAi construct and particular ligand, one can use one or more methods described herein. For example, a candidate linker can be evaluated by incubation with dithiothreitol (DTT), or other reducing agent known in the art, which mimics the rate of cleavage that would be observed in a cell, e.g., a target cell. The candidate linkers can also be evaluated under conditions which are selected to mimic blood or serum conditions. In a specific embodiment, candidate linkers are cleaved by at most 10% in the blood. In other embodiments, useful candidate linkers are degraded at least 2, 4, 10, 20, 50, 70, or 100 times faster in the cell (or under in vitro conditions selected to mimic intracellular conditions) as compared to blood (or under in vitro conditions selected to mimic extracellular conditions).

In yet other embodiments, phosphate-based cleavable linkers, which are cleaved by agents that degrade or hydrolyze the phosphate group, are employed to covalently attach a ligand to the sense or antisense strand of the RNAi construct. An example of an agent that hydrolyzes phosphate groups in cells are enzymes, such as phosphatases in cells. Examples of phosphate-based cleavable groups are —O—P(O)(ORk)-O—, —O—P(S)(ORk)-O—, —O—P(S)(SRk)-O—, —S—P(O) (ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, and —O—P(S)(Rk)-S—, where Rk can be hydrogen or C₁-C₁₀ alkyl. Specific embodiments include —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—. Another specific embodiment is —O—P(O)(OH)—O—. These candidate linkers can be evaluated using methods analogous to those described above.

In other embodiments, the linkers may comprise acid cleavable groups, which are groups that are cleaved under acidic conditions. In some embodiments, acid cleavable groups are cleaved in an acidic environment with a pH of about 6.5 or lower (e.g., about 6.0, 5.5, 5.0, or lower), or by agents, such as enzymes that can act as a general acid. In a cell, specific low pH organelles, such as endosomes and lysosomes, can provide a cleaving environment for acid cleavable groups. Examples of acid cleavable linking groups include, but are not limited to, hydrazones, esters, and esters of amino acids. Acid cleavable groups can have the general formula —C═NN—, C(O)O, or —OC(O). A specific embodiment is when the carbon attached to the oxygen of the ester (the alkoxy group) is an aryl group, substituted alkyl group, or tertiary alkyl group such as dimethyl, pentyl or t-butyl. These candidates can be evaluated using methods analogous to those described above.

In other embodiments, the linkers may comprise ester-based cleavable groups, which are cleaved by enzymes, such as esterases and amidases in cells. Examples of ester-based cleavable groups include, but are not limited to, esters of alkylene, alkenylene and alkynylene groups. Ester cleavable groups have the general formula —C(O)O—, or —OC(O)—. These candidate linkers can be evaluated using methods analogous to those described above.

In further embodiments, the linkers may comprise peptide-based cleavable groups, which are cleaved by enzymes, such as peptidases and proteases in cells. Peptide-based cleavable groups are peptide bonds formed between amino acids to yield oligopeptides (e.g., dipeptides, tripeptides etc.) and polypeptides. Peptide-based cleavable groups include the amide group (—C(O)NH—). The amide group can be formed between any alkylene, alkenylene or alkynylene. A peptide bond is a special type of amide bond formed between amino acids to yield peptides and proteins. The peptide-based cleavage group is generally limited to the peptide bond (i.e., the amide bond) formed between amino acids yielding peptides and proteins. Peptide-based cleavable linking groups have the general formula —NHCHR^{A}C(O)NHCH^{B}C(O)—, where R^{A} and R^{B} are the side chains of the two adjacent amino acids. These candidates can be evaluated using methods analogous to those described above.

Other types of linkers suitable for attaching ligands to the sense or antisense strands in the RNAi constructs of the invention are known in the art and can include the linkers described in U.S. Pat. Nos. 7,723,509; 8,017,762; 8,828,956; 8,877,917; and 9,181,551, all of which are hereby incorporated by reference in their entireties.

In certain embodiments, the ligand covalently attached to the sense or antisense strand of the RNAi constructs of the invention comprises a GalNAc moiety, e.g, a multivalent GalNAc moiety. In some embodiments, the multivalent GalNAc moiety is a trivalent GalNAc moiety and is attached to the 3′ end of the sense strand. In other embodiments, the multivalent GalNAc moiety is a trivalent GalNAc moiety and is attached to the 5′ end of the sense strand. In yet other embodiments, the multivalent GalNAc moiety is a tetravalent GalNAc moiety and is attached to the 3′ end of the sense strand. In still other embodiments, the multivalent GalNAc moiety is a tetravalent GalNAc moiety and is attached to the 5′ end of the sense strand.

In certain embodiments, the RNAi constructs of the invention comprise a ligand having the following structure ([Structure 1]):

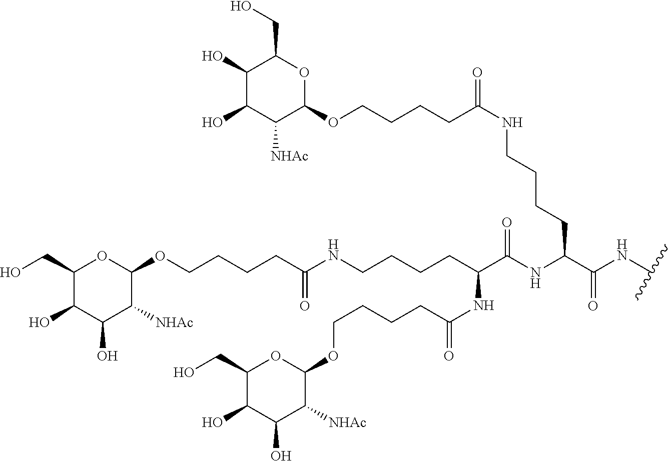 [SMILES: *NC(=O)[C@H](CCCCNC(=O)CCCCO[C@@H]1O[C@H](CO)[C@H](O)[C@H](O)[C@H]1NC(C)=O)NC(=O)[C@H](CCCCNC(=O)CCCCO[C@@H]1O[C@H](CO)[C@H](O)[C@H](O)[C@H]1NC(C)=O)NC(=O)CCCCO[C@@H]1O[C@H](CO)[C@H](O)[C@H](O)[C@H]1NC(C)=O]

In preferred embodiments, the ligand having this structure is covalently attached to the 5′ end of the sense strand (e.g. to the 5′ terminal nucleotide of the sense strand) via a linker, such as the linkers described herein. In one embodiment, the linker is an aminohexyl linker.

Exemplary trivalent and tetravalent GalNAc moieties and linkers that can be attached to the double-stranded RNA molecules in the RNAi constructs of the invention are provided in the structural formulas I-IX below. “Ac” in the formulas listed herein represents an acetyl group.

In one embodiment, the RNAi construct comprises a ligand and linker having the following structure of Formula I, wherein each n is independently 1 to 3, k is 1 to 3, m is 1 or 2, j is 1 or 2, and the ligand is attached to the 3′ end of the sense strand of the double-stranded RNA molecule (represented by the solid wavy line):

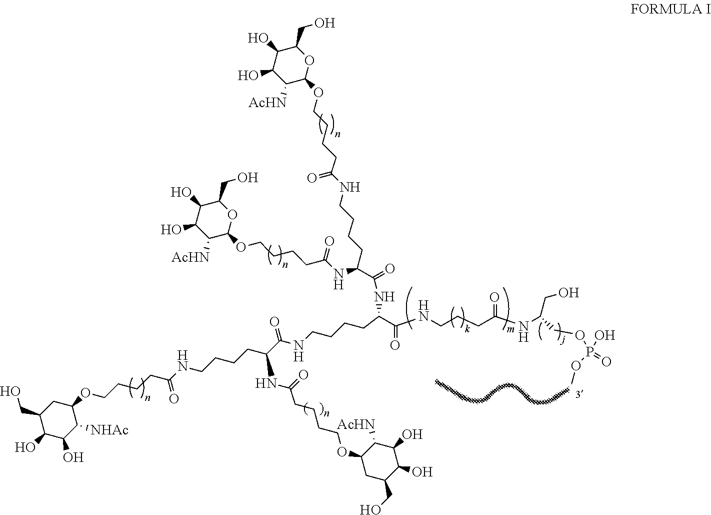

In another embodiment, the RNAi construct comprises a ligand and linker having the following structure of Formula II, wherein each n is independently 1 to 3, k is 1 to 3, m is 1 or 2, j is 1 or 2, and the ligand is attached to the 3′ end of the sense strand of the double-stranded RNA molecule (represented by the solid wavy line):

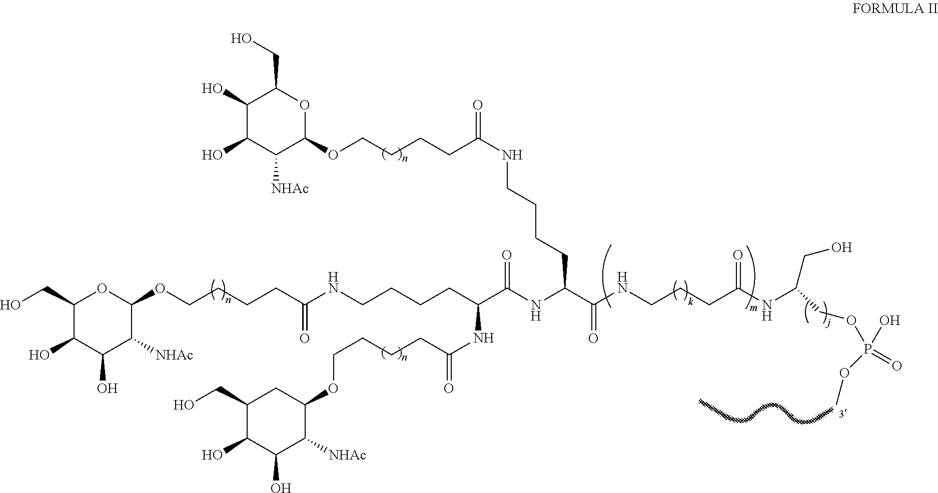

In yet another embodiment, the RNAi construct comprises a ligand and linker having the following structure of Formula III, wherein the ligand is attached to the 3′ end of the sense strand of the double-stranded RNA molecule (represented by the solid wavy line):

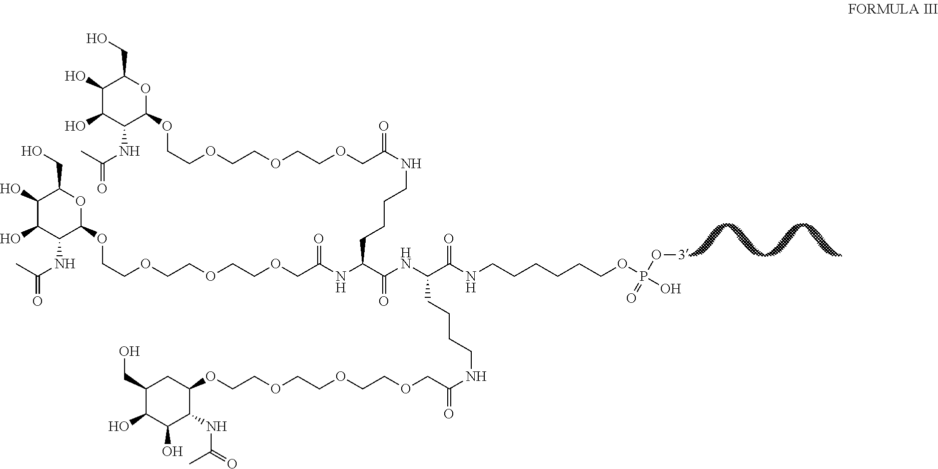

In still another embodiment, the RNAi construct comprises a ligand and linker having the following structure of Formula IV, wherein the ligand is attached to the 3′ end of the sense strand of the double-stranded RNA molecule (represented by the solid wavy line):

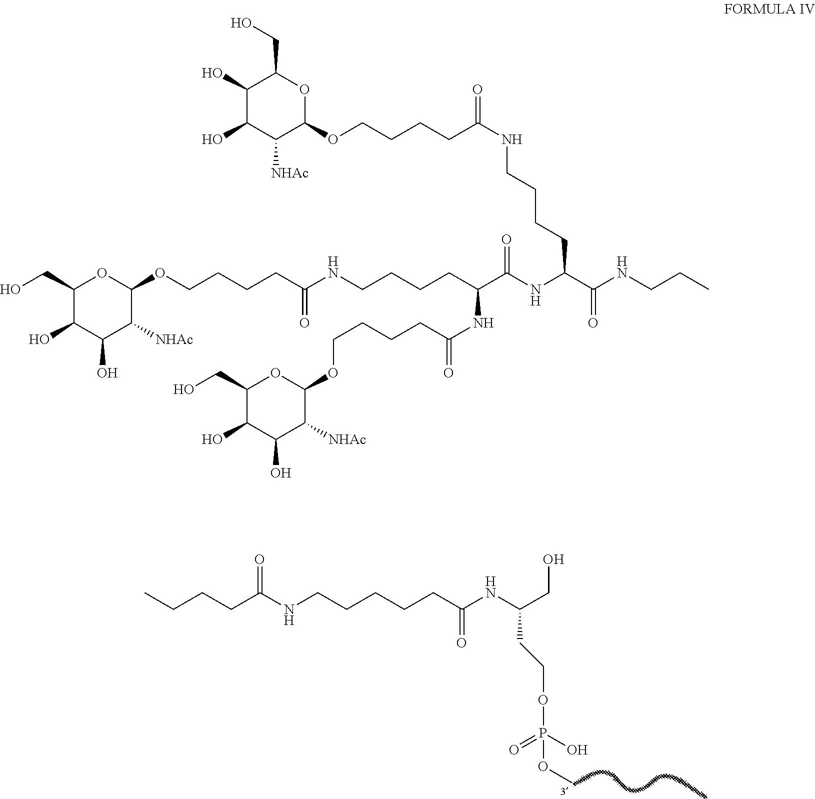

In certain embodiments, the RNAi construct comprises a ligand and linker having the following structure of Formula V, wherein each n is independently 1 to 3, k is 1 to 3, and the ligand is attached to the 5′ end of the sense strand of the double-stranded RNA molecule (represented by the solid wavy line):

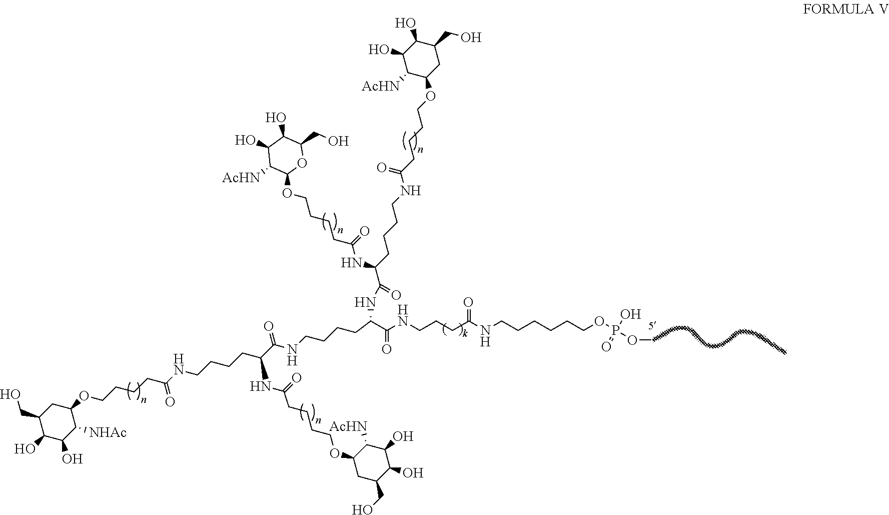

In other embodiments, the RNAi construct comprises a ligand and linker having the following structure of Formula VI, wherein each n is independently 1 to 3, k is 1 to 3, and the ligand is attached to the 5′ end of the sense strand of the double-stranded RNA molecule (represented by the solid wavy line):

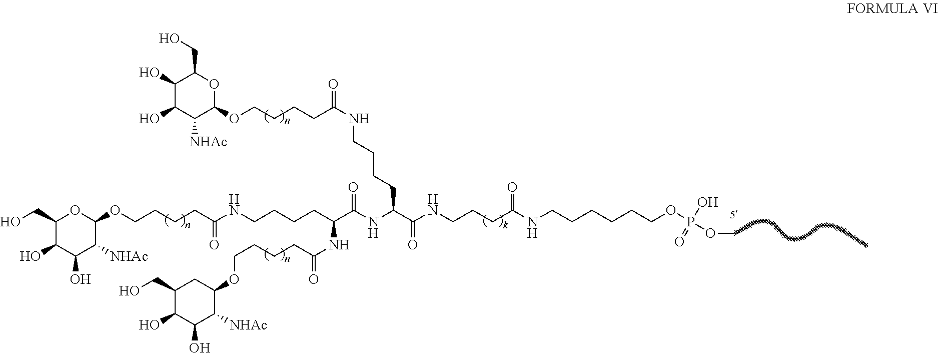

In one particular embodiment, the RNAi construct comprises a ligand and linker having the following structure of Formula VII, wherein X=O or S and wherein the ligand is attached to the 5′ end of the sense strand of the double-stranded RNA molecule (represented by the squiggly line):

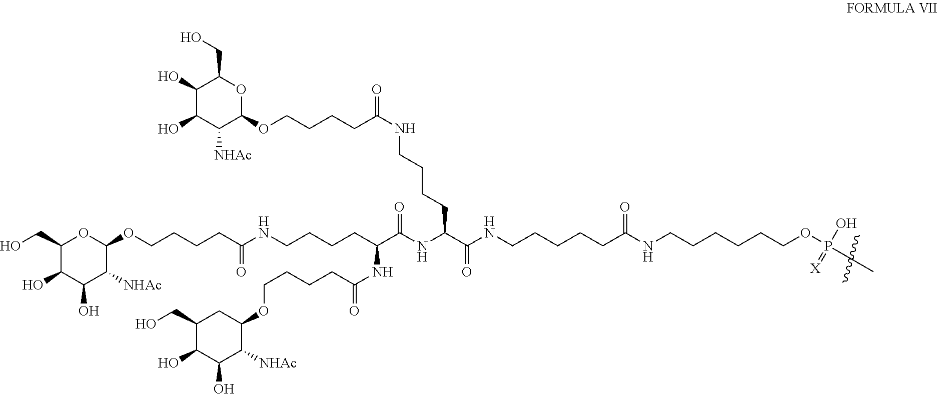

In some embodiments, the RNAi construct comprises a ligand and linker having the following structure of Formula VIII, wherein each n is independently 1 to 3 and the ligand is attached to the 5′ end of the sense strand of the double-stranded RNA molecule (represented by the solid wavy line):

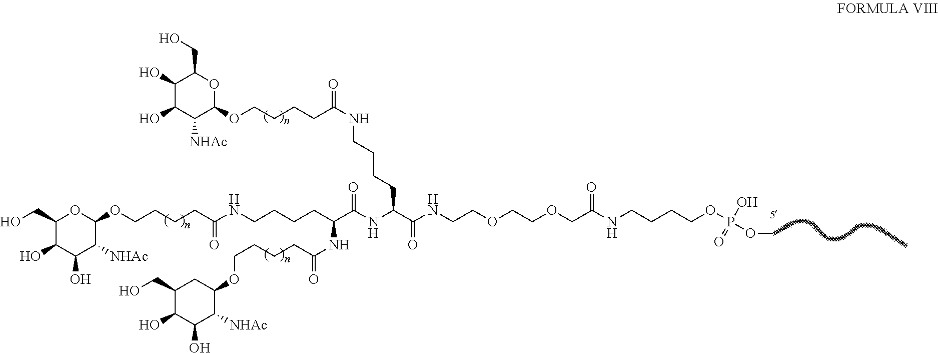

In certain embodiments, the RNAi construct comprises a ligand and linker having the following structure of Formula IX, wherein the ligand is attached to the 5′ end of the sense strand of the double-stranded RNA molecule (represented by the solid wavy line):

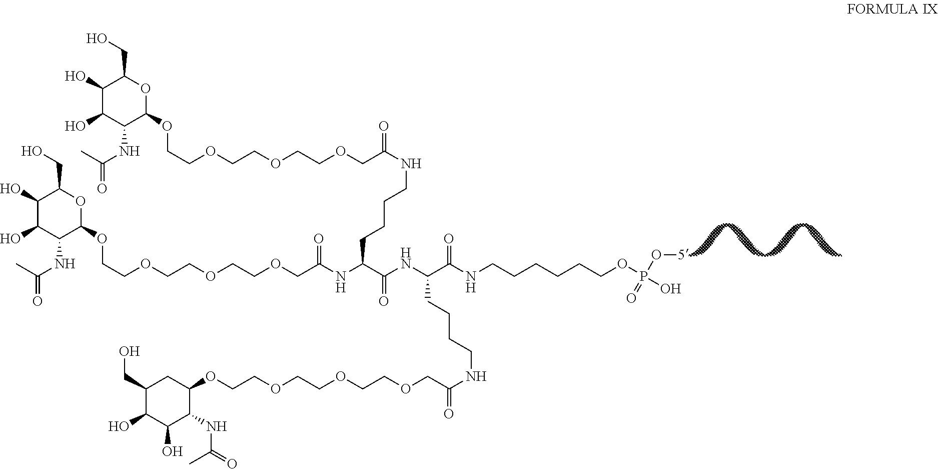

A phosphorothioate bond can be substituted for the phosphodiester bond shown in any one of Formulas I-IX to covalently attach the ligand and linker to the nucleic acid strand.

The present invention also includes pharmaceutical compositions and formulations comprising the RNAi constructs described herein and pharmaceutically acceptable carriers, excipients, or diluents. Such compositions and formulations are useful for reducing expression of the MARC1 gene in a patient in need thereof. Where clinical applications are contemplated, pharmaceutical compositions and formulations will be prepared in a form appropriate for the intended application. Generally, this will entail preparing compositions that are essentially free of pyrogens, as well as other impurities that could be harmful to humans or animals.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier, excipient, or diluent” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the RNAi constructs of the present invention, its use in therapeutic compositions is contemplated. Supplementary active ingredients also can be incorporated into the compositions, provided they do not inactivate the RNAi constructs of the compositions.

Compositions and methods for the formulation of pharmaceutical compositions depend on a number of criteria, including, but not limited to, route of administration, type and extent of disease or disorder to be treated, or dose to be administered. In some embodiments, the pharmaceutical compositions are formulated based on the intended route of delivery. For instance, in certain embodiments, the pharmaceutical compositions are formulated for parenteral delivery. Parenteral forms of delivery include intravenous, intraarterial, subcutaneous, intrathecal, intraperitoneal or intramuscular injection or infusion. In one embodiment, the pharmaceutical composition is formulated for intravenous delivery. In such an embodiment, the pharmaceutical composition may include a lipid-based delivery vehicle. In another embodiment, the pharmaceutical composition is formulated for subcutaneous delivery. In such an embodiment, the pharmaceutical composition may include a targeting ligand (e.g. GalNAc-containing or antibody-containing ligands described herein).

In some embodiments, the pharmaceutical compositions comprise an effective amount of an RNAi construct described herein. An “effective amount” is an amount sufficient to produce a beneficial or desired clinical result. In some embodiments, an effective amount is an amount sufficient to reduce MARC1 gene expression in a particular tissue or cell-type (e.g. liver or hepatocytes) of a patient. An effective amount of an RNAi construct of the invention may be from about 0.01 mg/kg body weight to about 100 mg/kg body weight, and may be administered daily, weekly, monthly, or at longer intervals. The precise determination of what would be considered an effective amount and frequency of administration may be based on several factors, including a patient's size, age, and general condition, type of disorder to be treated (e.g. fatty liver disease, liver fibrosis, or cardiovascular disease), particular RNAi construct employed, and route of administration.

Administration of the pharmaceutical compositions of the present invention may be via any common route so long as the target tissue is available via that route. Such routes include, but are not limited to, parenteral (e.g., subcutaneous, intramuscular, intraperitoneal or intravenous), oral, nasal, buccal, intradermal, transdermal, and sublingual routes, or by direct injection into liver tissue or delivery through the hepatic portal vein. In some embodiments, the pharmaceutical composition is administered parenterally. For instance, in certain embodiments, the pharmaceutical composition is administered intravenously. In other embodiments, the pharmaceutical composition is administered subcutaneously.

Colloidal dispersion systems, such as macromolecule complexes, nanocapsules, microspheres, beads, and lipid-based systems, including oil-in-water emulsions, micelles, mixed micelles, and liposomes, may be used as delivery vehicles for the RNAi constructs of the invention. Commercially available fat emulsions that are suitable for delivering the nucleic acids of the invention include Intralipid® (Baxter International Inc.), Liposyn® (Abbott Pharmaceuticals), Liposyn®II (Hospira), Liposyn®III (Hospira), Nutrilipid (B. Braun Medical Inc.), and other similar lipid emulsions. An exemplary colloidal system for use as a delivery vehicle in vivo is a liposome (i.e., an artificial membrane vesicle). The RNAi constructs of the invention may be encapsulated within liposomes or may form complexes thereto, in particular to cationic liposomes. Alternatively, RNAi constructs of the invention may be complexed to lipids, in particular to cationic lipids. Suitable lipids and liposomes include neutral (e.g., dioleoylphosphatidyl ethanolamine (DOPE), dimyristoylphosphatidyl choline (DMPC), and dipalmitoyl phosphatidylcholine (DPPC)), distearolyphosphatidyl choline), negative (e.g., dimyristoylphosphatidyl glycerol (DMPG)), and cationic (e.g., dioleoyltetramethylaminopropyl (DOTAP) and dioleoylphosphatidyl ethanolamine (DOTMA)). The preparation and use of such colloidal dispersion systems are well known in the art. Exemplary formulations are also disclosed in U.S. Pat. Nos. 5,981,505; 6,217,900; 6,383,512; 5,783,565; 7,202,227; 6,379,965; 6,127,170; 5,837,533; 6,747,014; and WIPO Publication No. WO 03/093449.

In some embodiments, the RNAi constructs of the invention are fully encapsulated in a lipid formulation, e.g., to form a SNALP or other nucleic acid-lipid particle. As used herein, the term “SNALP” refers to a stable nucleic acid-lipid particle. SNALPs typically contain a cationic lipid, a non-cationic lipid, and a lipid that prevents aggregation of the particle (e.g., a PEG-lipid conjugate). SNALPs are exceptionally useful for systemic applications, as they exhibit extended circulation lifetimes following intravenous injection and accumulate at distal sites (e.g., sites physically separated from the administration site). The nucleic acid-lipid particles typically have a mean diameter of about 50 nm to about 150 nm, about 60 nm to about 130 nm, about 70 nm to about 110 nm, or about 70 nm to about 90 nm, and are substantially nontoxic. In addition, the nucleic acids when present in the nucleic acid-lipid particles are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and WIPO Publication No. WO 96/40964.

The pharmaceutical compositions suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically acceptable salts include, for example, acid addition salts (formed with free amino groups) derived from inorganic acids (e.g., hydrochloric or phosphoric acids), or from organic acids (e.g., acetic, oxalic, tartaric, mandelic, and the like). Salts formed with the free carboxyl groups can also be derived from inorganic bases (e.g., sodium, potassium, ammonium, calcium, or ferric hydroxides) or from organic bases (e.g., isopropylamine, trimethylamine, histidine, procaine and the like). Pharmaceutically acceptable salts are described in detail in Berge et al., J. Pharmaceutical Sciences, Vol. 66: 1-19, 1977.

For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). For human administration, preparations should meet sterility, pyrogenicity, general safety and purity standards as required by FDA standards. In certain embodiments, a pharmaceutical composition of the invention comprises or consists of a sterile saline solution and an RNAi construct described herein. In other embodiments, a pharmaceutical composition of the invention comprises or consists of an RNAi construct described herein and sterile water (e.g. water for injection, WFI). In still other embodiments, a pharmaceutical composition of the invention comprises or consists of an RNAi construct described herein and phosphate-buffered saline (PBS).

In some embodiments, the pharmaceutical compositions of the invention are packaged with or stored within a device for administration. Devices for injectable formulations include, but are not limited to, injection ports, pre-filled syringes, autoinjectors, injection pumps, on-body injectors, and injection pens. Devices for aerosolized or powder formulations include, but are not limited to, inhalers, insufflators, aspirators, and the like. Thus, the present invention includes administration devices comprising a pharmaceutical composition of the invention for treating or preventing one or more of the diseases or disorders described herein.

The present invention provides a method for reducing or inhibiting expression of the MARC1 gene, and thus the production of mARC1 protein, in a cell (e.g. liver cell) by contacting the cell with any one of the RNAi constructs described herein. The cell may be in vitro or in vivo. mARC1 expression can be assessed by measuring the amount or level of mARC1 mRNA, mARC1 protein, or another biomarker linked to mARC1 expression, such as serum levels of cholesterol, LDL-cholesterol, or liver enzymes, such as alanine aminotransferase (ALT). The reduction of mARC1 expression in cells or animals treated with an RNAi construct of the invention can be determined relative to the mARC1 expression in cells or animals not treated with the RNAi construct or treated with a control RNAi construct. For instance, in some embodiments, reduction of mARC1 expression is assessed by (a) measuring the amount or level of mARC1 mRNA in liver cells treated with an RNAi construct of the invention, (b) measuring the amount or level of mARC1 mRNA in liver cells treated with a control RNAi construct (e.g. RNAi construct directed to an RNA molecule not expressed in liver cells or a RNAi construct having a nonsense or scrambled sequence) or no construct, and (c) comparing the measured mARC1 mRNA levels from treated cells in (a) to the measured mARC1 mRNA levels from control cells in (b). The mARC1 mRNA levels in the treated cells and controls cells can be normalized to RNA levels for a control gene (e.g. 18S ribosomal RNA or housekeeping gene) prior to comparison. mARC1 mRNA levels can be measured by a variety of methods, including Northern blot analysis, nuclease protection assays, fluorescence in situ hybridization (FISH), reverse-transcriptase (RT)-PCR, real-time RT-PCR, quantitative PCR, droplet digital PCR, and the like.

In other embodiments, reduction of mARC1 expression is assessed by (a) measuring the amount or level of mARC1 protein in liver cells treated with an RNAi construct of the invention, (b) measuring the amount or level of mARC1 protein in liver cells treated with a control RNAi construct (e.g. RNAi construct directed to an RNA molecule not expressed in liver cells or a RNAi construct having a nonsense or scrambled sequence) or no construct, and (c) comparing the measured mARC1 protein levels from treated cells in (a) to the measured mARC1 protein levels from control cells in (b). Methods of measuring mARC1 protein levels are known to those of skill in the art, and include Western Blots, immunoassays (e.g. ELISA), and flow cytometry. Any method capable of measuring mARC1 mRNA or mARC1 protein can be used to assess the efficacy of the RNAi constructs of the invention.

In some embodiments, the methods to assess mARC1 expression levels are performed in vitro in cells that natively express mARC1 (e.g. liver cells) or cells that have been engineered to express mARC1. In certain embodiments, the methods are performed in vitro in liver cells. Suitable liver cells include, but are not limited to, primary hepatocytes (e.g. human or non-human primate hepatocytes), HepAD38 cells, HuH-6 cells, HuH-7 cells, HuH-5-2 cells, BNLCL2 cells, Hep3B cells, or HepG2 cells. In one embodiment, the liver cells are HuH-7 cells. In another embodiment, the liver cells are human primary hepatocytes. In yet another embodiment, the liver cells are Hep3B cells.

In other embodiments, the methods to assess mARC1 expression levels are performed in vivo. The RNAi constructs and any control RNAi constructs can be administered to an animal and mARC1 mRNA or mARC1 protein levels assessed in liver tissue harvested from the animal following treatment. Alternatively or additionally, a biomarker or functional phenotype associated with mARC1 expression can be assessed in the treated animals. For instance, MARC1 loss of function variants have been associated with reduced serum total cholesterol, LDL-cholesterol, and liver enzyme levels (see Emdin et al., PLoS Genet, Vol. 16(4): e1008629, 2020). Thus, serum or plasma levels of cholesterol, LDL-cholesterol, or liver enzymes (e.g. ALT) can be measured in animals treated with RNAi constructs of the invention to assess the functional efficacy of reducing mARC1 expression. Exemplary methods for measuring serum or plasma cholesterol or enzyme levels are described in Examples 1, 4, and 5.

In certain embodiments, expression of mARC1 mRNA or protein is reduced in liver cells by at least 40%, at least 45%, or at least 50% by an RNAi construct of the invention. In some embodiments, expression of mARC1 mRNA or protein is reduced in liver cells by at least 60%, at least 65%, at least 70%, at least 75%, at least 80%, or at least 85% by an RNAi construct of the invention. In other embodiments, the expression of mARC1 mRNA or protein is reduced in liver cells by about 90% or more, e.g., 91%, 92%, 93%, 94%, 95%, 96%, 97%, 98%, 99%, or more by an RNAi construct of the invention. The percent reduction of mARC1 expression can be measured by any of the methods described herein as well as others known in the art.

The present invention provides methods for reducing or inhibiting expression of the MARC1 gene, and thus the production of mARC1 protein, in a patient in need thereof as well as methods of treating or preventing conditions, diseases, or disorders associated with mARC1 expression or activity. A “condition, disease, or disorder associated with mARC1 expression” refers to conditions, diseases, or disorders in which mARC1 expression levels are altered or where elevated expression levels of mARC1 are associated with an increased risk of developing the condition, disease or disorder. A condition, disease, or disorder associated with mARC1 expression can also include conditions, diseases, or disorders resulting from aberrant changes in lipoprotein metabolism, such as changes resulting in abnormal or elevated levels of cholesterol, lipids, triglycerides, etc. or impaired clearance of these molecules. Recent genetic studies have reported an association between loss-of-function variants in the MARC1 gene and decreased blood levels of cholesterol and liver enzymes, reduced liver fat, and protection from cirrhosis (Spracklen et al., Hum Mol Genet., Vol. 26(9):1770-178, 2017; Emdin et al., bioRxiv 594523; //doi.org/10.1101/594523, 2019; and Emdin et al., PLoS Genet, Vol. 16(4): e1008629, 2020)). See Emdin et al., bioRxiv 594523; //doi.org/10.1101/594523, 2019; and Emdin et al., PLoS Genet, Vol. 16(4): e1008629, 2020). Thus, in certain embodiments, the RNAi constructs of the invention are particularly useful for treating or preventing fatty liver disease (e.g. NAFLD and NASH) and cardiovascular disease (e.g. coronary artery disease and myocardial infarction) as well as reducing liver fibrosis and serum cholesterol levels.

Conditions, diseases, and disorders associated with mARC1 expression that can be treated or prevented according to the methods of the invention include, but are not limited to, fatty liver disease, such as alcoholic fatty liver disease, alcoholic steatohepatitis, NAFLD and NASH; chronic liver disease; cirrhosis; cardiovascular disease, such as myocardial infarction, heart failure, stroke (ischemic and hemorrhagic), atherosclerosis, coronary artery disease, peripheral vascular disease (e.g. peripheral artery disease), cerebrovascular disease, vulnerable plaque, and aortic valve stenosis; familial hypercholesterolemia; venous thrombosis; hypercholesterolemia; hyperlipidemia; and dyslipidemia (manifesting, e.g., as elevated total cholesterol, elevated low-density lipoprotein (LDL), elevated very low-density lipoprotein (VLDL), elevated triglycerides, and/or low levels of high-density lipoprotein (HDL)).

In certain embodiments, the present invention provides a method for reducing the expression of mARC1 protein in a patient in need thereof comprising administering to the patient any of the RNAi constructs described herein. The term “patient,” as used herein, refers to a mammal, including humans, and can be used interchangeably with the term “subject.” Preferably, the expression level of mARC1 in hepatocytes in the patient is reduced following administration of the RNAi construct as compared to the mARC1 expression level in a patient not receiving the RNAi construct or as compared to the mARC1 expression level in the patient prior to administration of the RNAi construct. In some embodiments, following administration of an RNAi construct of the invention, expression of mARC1 is reduced in the patient by at least 30%, at least 35%, at least 40%, at least 45%, at least 50%, at least 55%, at least 60%, at least 65%, at least 70%, at least 75%, at least 80%, at least 85%, or at least 90%, e.g., 91%, 92%, 93%, 94%, 95%, 96%, 97%, 98%, or 99%. The percent reduction of mARC1 expression can be measured by any of the methods described herein as well as others known in the art. In certain embodiments, the percent reduction of mARC1 expression is determined by assessing levels of a serum or plasma biomarker, such as total cholesterol, LDL-cholesterol, or liver enzyme (e.g. ALT) levels, in the patient according to methods described herein.

In some embodiments, a patient in need of reduction of mARC1 expression is a patient who is at risk of having a myocardial infarction. A patient who is at risk of having a myocardial infarction may be a patient who has a history of myocardial infarction (e.g. has had a previous myocardial infarction). A patient at risk of having a myocardial infarction may also be a patient who has a familial history of myocardial infarction or who has one or more risk factors of myocardial infarction. Such risk factors include, but are not limited to, hypertension, elevated levels of non-HDL cholesterol, elevated levels of triglycerides, diabetes, obesity, or history of autoimmune diseases (e.g. rheumatoid arthritis, lupus). In one embodiment, a patient who is at risk of having a myocardial infarction is a patient who has or is diagnosed with coronary artery disease. The risk of myocardial infarction in these and other patients can be reduced by administering to the patients any of the RNAi constructs described herein. Accordingly, the present invention provides a method for reducing the risk of myocardial infarction in a patient in need thereof comprising administering to the patient an RNAi construct described herein. In some embodiments, the present invention includes use of any of the RNAi constructs described herein in the preparation of a medicament for reducing the risk of myocardial infarction in a patient in need thereof. In other embodiments, the present invention provides a mARC1-targeting RNAi construct for use in a method for reducing the risk of myocardial infarction in a patient in need thereof.

In certain embodiments, a patient in need of reduction of mARC1 expression is a patient who is diagnosed with or at risk of cardiovascular disease. Thus, the present invention includes a method for treating or preventing cardiovascular disease in a patient in need thereof by administering any of the RNAi constructs of the invention. In some embodiments, the present invention includes use of any of the RNAi constructs described herein in the preparation of a medicament for treating or preventing cardiovascular disease in a patient in need thereof. In other embodiments, the present invention provides a mARC1-targeting RNAi construct for use in a method for treating or preventing cardiovascular disease in a patient in need thereof. Cardiovascular disease includes, but is not limited to, myocardial infarction, heart failure, stroke (ischemic and hemorrhagic), atherosclerosis, coronary artery disease, peripheral vascular disease (e.g. peripheral artery disease), cerebrovascular disease, vulnerable plaque, and aortic valve stenosis. In some embodiments, the cardiovascular disease to be treated or prevented according to the methods of the invention is coronary artery disease. In other embodiments, the cardiovascular disease to be treated or prevented according to the methods of the invention is myocardial infarction. In yet other embodiments, the cardiovascular disease to be treated or prevented according to the methods of the invention is stroke. In still other embodiments, the cardiovascular disease to be treated or prevented according to the methods of the invention is peripheral artery disease. In certain embodiments, administration of the RNAi constructs described herein reduces the risk of non-fatal myocardial infarctions, fatal and non-fatal strokes, certain types of heart surgery (e.g. angioplasty, bypass), hospitalization for heart failure, chest pain in patients with heart disease, and/or cardiovascular events in patients with established heart disease (e.g. prior myocardial infarction, prior heart surgery, and/or chest pain with evidence of blocked arteries). In some embodiments, administration of the RNAi constructs described herein according to the methods of the invention can be used to reduce the risk of recurrent cardiovascular events.

In some embodiments, a patient to be treated according to the methods of the invention is a patient who has a vulnerable plaque (also referred to as unstable plaque). Vulnerable plaques are a build-up of macrophages and lipids containing predominantly cholesterol that lie underneath the endothelial lining of the arterial wall. These vulnerable plaques can rupture resulting in the formation of a blood clot, which can potentially block blood flow through the artery and cause a myocardial infarction or stroke. Vulnerable plaques can be identified by methods known in the art, including, but not limited to, intravascular ultrasound and computed tomography (see Sahara et al., European Heart Journal, Vol. 25: 2026-2033, 2004; Budhoff, J. Am. Coll. Cardiol., Vol. 48: 319-321, 2006; Hausleiter et al., J. Am. Coll. Cardiol., Vol. 48: 312-318, 2006).

In other embodiments, a patient in need of reduction of mARC1 expression is a patient who has elevated blood levels of cholesterol (e.g. total cholesterol, non-HDL cholesterol, or LDL cholesterol). Accordingly, in some embodiments, the present invention provides a method for reducing blood levels (e.g. serum or plasma) of cholesterol in a patient in need thereof comprising administering to the patient any of the RNAi constructs described herein. In some embodiments, the present invention includes use of any of the RNAi constructs described herein in the preparation of a medicament for reducing blood levels (e.g. serum or plasma) of cholesterol in a patient in need thereof. In other embodiments, the present invention provides a mARC1-targeting RNAi construct for use in a method for reducing blood levels (e.g. serum or plasma) of cholesterol in a patient in need thereof. In certain embodiments, the cholesterol reduced according to the methods of the invention is LDL cholesterol. In other embodiments, the cholesterol reduced according to the methods of the invention is non-HDL cholesterol. Non-HDL cholesterol is a measure of all cholesterol-containing proatherogenic lipoproteins, including LDL cholesterol, very low-density lipoprotein, intermediate-density lipoprotein, lipoprotein(a), chylomicron, and chylomicron remnants. Non-HDL cholesterol has been reported to be a good predictor of cardiovascular risk (Rana et al., Curr. Atheroscler. Rep., Vol. 14:130-134, 2012). Non-HDL cholesterol levels can be calculated by subtracting HDL cholesterol levels from total cholesterol levels.

In some embodiments, a patient to be treated according to the methods of the invention is a patient who has elevated levels of non-HDL cholesterol (e.g. elevated serum or plasma levels of non-HDL cholesterol). Ideally, levels of non-HDL cholesterol should be about 30 mg/dL above the target for LDL cholesterol levels for any given patient. In particular embodiments, a patient is administered an RNAi construct of the invention if the patient has a non-HDL cholesterol level of about 130 mg/dL or greater. In one embodiment, a patient is administered an RNAi construct of the invention if the patient has a non-HDL cholesterol level of about 160 mg/dL or greater. In another embodiment, a patient is administered an RNAi construct of the invention if the patient has a non-HDL cholesterol level of about 190 mg/dL or greater. In still another embodiment, a patient is administered an RNAi construct of the invention if the patient has a non-HDL cholesterol level of about 220 mg/dL or greater. In certain embodiments, a patient is administered an RNAi construct of the invention if the patient is at a high or very high risk of cardiovascular disease according to the 2013 ACC/AHA Guideline on the Assessment of Cardiovascular Risk (Goff et al., ACC/AHA guideline on the assessment of cardiovascular risk: a report of the American College of Cardiology/American Heart Association Task Force on Practice Guidelines. J Am Coll Cardiol, Vol. 63:2935-2959, 2014) and has a non-HDL cholesterol level of about 100 mg/dL or greater.

In certain embodiments of the methods of the invention, a patient is administered an RNAi construct described herein if they are at a moderate risk or higher for cardiovascular disease according to the 2013 ACC/AHA Guideline on the Assessment of Cardiovascular Risk (referred to herein as the “2013 Guidelines”). In certain embodiments, an RNAi construct of the invention is administered to a patient if the patient's LDL cholesterol level is greater than about 160 mg/dL. In other embodiments, an RNAi construct of the invention is administered to a patient if the patient's LDL cholesterol level is greater than about 130 mg/dL and the patient has a moderate risk of cardiovascular disease according to the 2013 Guidelines. In still other embodiments, an RNAi construct of the invention is administered to a patient if the patient's LDL cholesterol level is greater than 100 mg/dL and the patient has a high or very high risk of cardiovascular disease according to the 2013 Guidelines.

In other embodiments, a patient in need of reduction of mARC1 expression is a patient who is diagnosed with or at risk of fatty liver disease. Thus, the present invention includes a method for treating, preventing, or reducing the risk of developing fatty liver disease in a patient in need thereof comprising administering to the patient any of the RNAi constructs of the invention. In some embodiments, the present invention includes use of any of the RNAi constructs described herein in the preparation of a medicament for treating, preventing, or reducing the risk of developing fatty liver disease in a patient in need thereof. In other embodiments, the present invention provides a mARC1-targeting RNAi construct for use in a method for treating, preventing, or reducing the risk of developing fatty liver disease in a patient in need thereof. Fatty liver disease is a condition in which fat accumulates in the liver. There are two primary types of fatty liver disease: a first type that is associated with heavy alcohol use (alcoholic steatohepatitis) and a second type that is not related to use of alcohol (nonalcoholic fatty liver disease (NAFLD)). NAFLD is typically characterized by the presence of fat accumulation in the liver but little or no inflammation or liver cell damage. NAFLD can progress to nonalcoholic steatohepatitis (NASH), which is characterized by liver inflammation and cell damage, both of which in turn can lead to liver fibrosis and eventually cirrhosis or hepatic cancer. In certain embodiments, the fatty liver disease to be treated, prevented, or reduce the risk of developing according to the methods of the invention is NAFLD. In other embodiments, the fatty liver disease to be treated, prevented, or reduce the risk of developing according to the methods of the invention is NASH. In still other embodiments, the fatty liver disease to be treated, prevented, or reduce the risk of developing according to the methods of the invention is alcoholic steatohepatitis. In some embodiments, a patient in need of treatment or prevention for fatty liver disease according to the methods of the invention or is at risk of developing fatty liver disease has been diagnosed with type 2 diabetes, a metabolic disorder, or is obese (e.g. body mass index of ≥30.0). In other embodiments, a patient in need of treatment or prevention for fatty liver disease according to the methods of the invention or is at risk of developing fatty liver disease has elevated levels of non-HDL cholesterol or triglycerides. Depending on the particular patient and other risk factors that patient may have, elevated levels of non-HDL cholesterol may be about 130 mg/dL or greater, about 160 mg/dL or greater, about 190 mg/dL or greater, or about 220 mg/dL or greater. Elevated triglyceride levels may be about 150 mg/dL or greater, about 175 mg/dL or greater, about 200 mg/dL or greater, or about 250 mg/dL or greater.

In certain embodiments, a patient in need of reduction of mARC1 expression is a patient who is diagnosed with or at risk of developing hepatic fibrosis or cirrhosis. Accordingly, the present invention encompasses a method for treating, preventing, or reducing liver fibrosis in a patient in need thereof comprising administering to the patient any of the RNAi constructs of the invention. In some embodiments, the present invention includes use of any of the RNAi constructs described herein in the preparation of a medicament for treating, preventing, or reducing liver fibrosis in a patient in need thereof. In other embodiments, the present invention provides a mARC1-targeting RNAi construct for use in a method for treating, preventing, or reducing liver fibrosis in a patient in need thereof. In some embodiments, a patient at risk for developing hepatic fibrosis or cirrhosis is diagnosed with NAFLD. In other embodiments, a patient at risk for developing hepatic fibrosis or cirrhosis is diagnosed with NASH. In yet other embodiments, a patient at risk for developing hepatic fibrosis or cirrhosis is diagnosed with alcoholic steatohepatitis. In still other embodiments, a patient at risk for developing hepatic fibrosis or cirrhosis is diagnosed with hepatitis. In certain embodiments, administration of an RNAi construct of the invention prevents or delays the development of cirrhosis in the patient.

The following examples, including the experiments conducted and the results achieved, are provided for illustrative purposes only and are not to be construed as limiting the scope of the appended claims.

### EXAMPLES

### Example 1. Inhibition of mARC1 Expression in Ob/Ob Animals Regulates Lipid Levels

Genetic studies have reported an association between the A165T missense mutation in the MARC1 gene and reduced serum low-density lipoprotein (LDL)-cholesterol and total cholesterol levels (Spracklen et al., Hum Mol Genet., Vol. 26(9):1770-178, 2017; Emdin et al., bioRxiv 594523; //doi.org/10.1101/594523, 2019; and Emdin et al., PLoS Genet, Vol. 16(4): e1008629, 2020)). This mutation as well as other loss of function variants of the MARC1 gene have also been recently associated with lower levels of hepatic fat, reduced liver enzyme levels, and reduced risk of cirrhosis (Emdin et al., 2019 and Emdin et al, 2020). To evaluate whether inhibition of mARC1 expression could reduce serum cholesterol levels as observed in human carriers of the MARC1 A165T variant allele, aged obese mice (ob/ob) were administered an siRNA molecule targeting the mouse Marc1 gene or a control siRNA molecule. Ob/ob mice are obese and have elevated lipid levels, and therefore these mice are often used as a model of type II diabetes and other metabolic disorders.

18-20-week-old male ob/ob animals (The Jackson Laboratory) were fed standard chow (Harlan, 2020× Teklad global soy protein-free extruded rodent diet). Mice received, by subcutaneous injection, buffer (phosphate-buffered saline) alone (n=8), mARC1-targeted siRNA (duplex no. D-1000; n=8), or a control siRNA (duplex no. D-1002; n=8) at 3 mg/kg body weight in 0.2 ml buffer once every two weeks for six weeks. The siRNA molecules were synthesized and conjugated to a trivalent GalNAc moiety (structure shown in Formula VII) as described in Example 2 below. The structure of each of the siRNA molecules is provided in Tables 1 and 2 below. Animals were fasted and harvested on week 6 for further analysis. Liver total RNA from harvested animals was processed for qPCR analysis and serum parameters were measured by clinical analyzer (AU400 Chemistry Analyzer, Olympus). mRNA levels were first normalized to 18S ribosomal RNA levels in each liver sample, and then compared to the expression levels in the buffer alone group. Data were presented as relative fold over expression in the buffer alone group. Liver tissues were homogenized and extracted by isopropanol for total cholesterol and total triglyceride measurement (ThermoFisher, Infinity cholesterol and Infinity triglyceride reagents). All animal housing conditions and research protocols were approved by the Amgen Institutional Animal Care and Use Committee (IACUC). Mice were housed in a specified-pathogen free, AAALAC, Intl-accredited facility in ventilated microisolators. Procedures and housing rooms were positively pressured and regulated on a 12:12 dark:light cycle. All animals received reverse-osmosis purified water ad libitum via an automatic watering system.

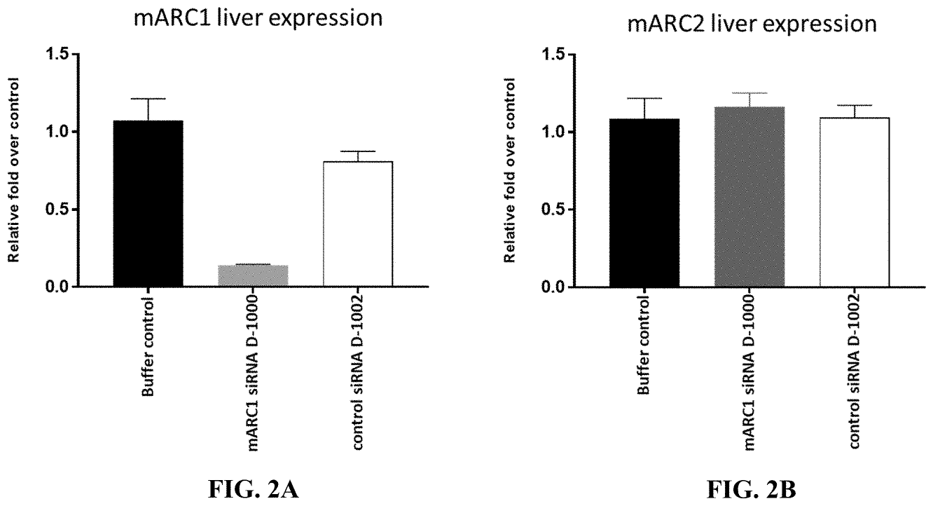

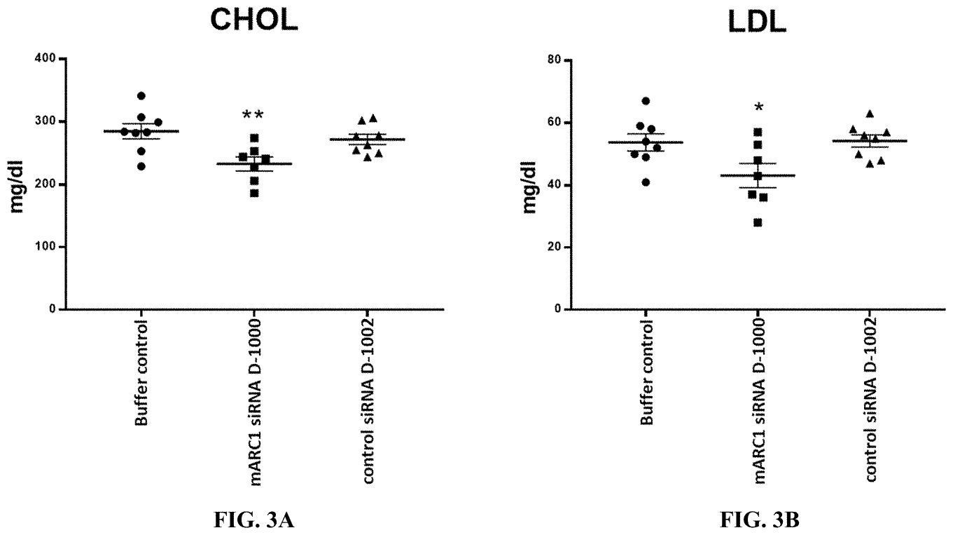

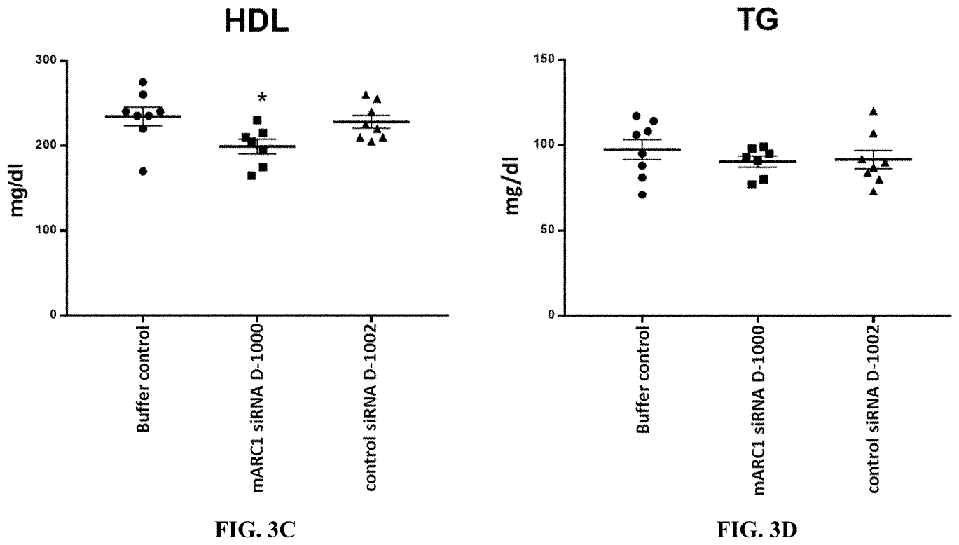

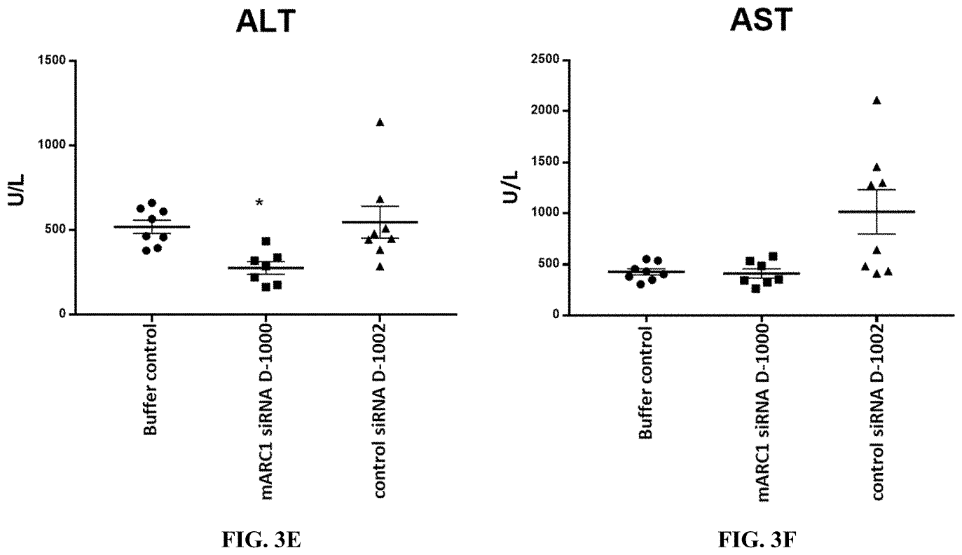

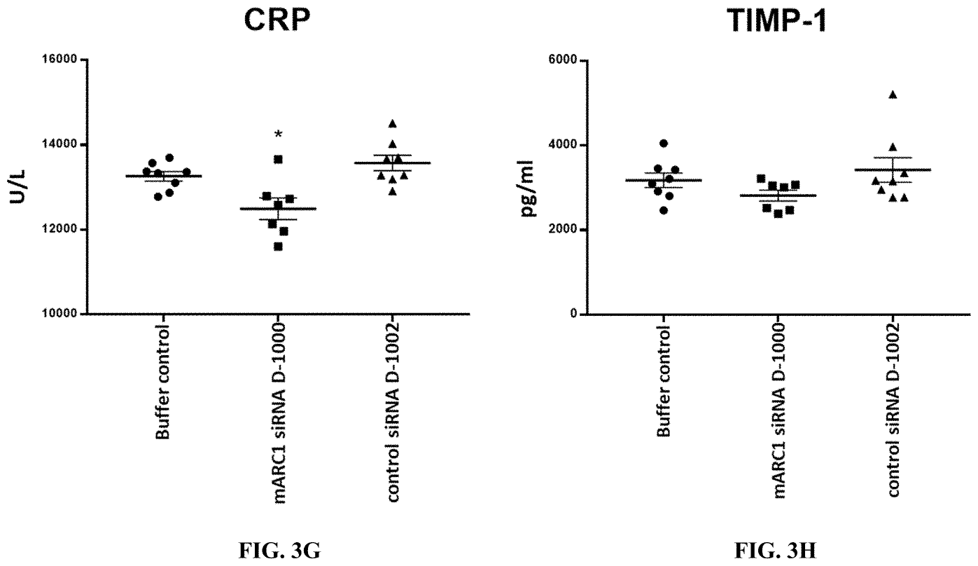

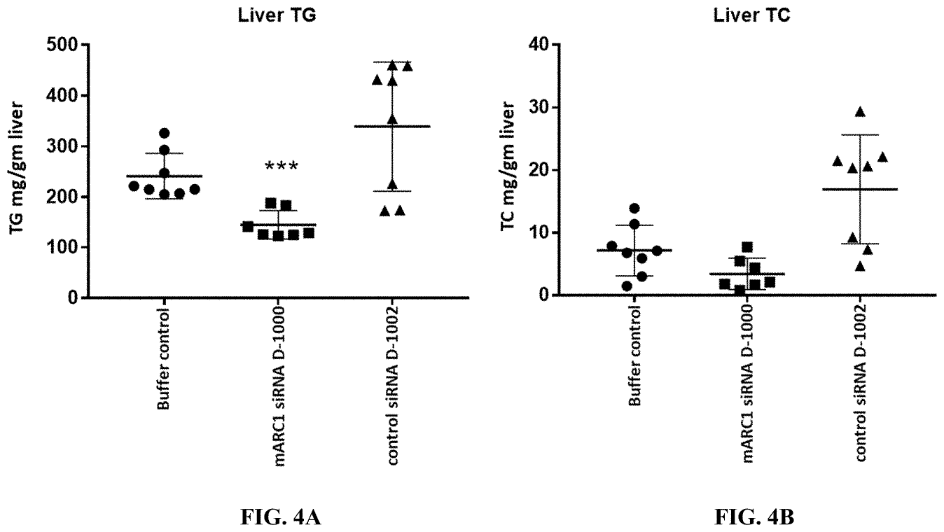

Animals treated with the mARC1-targeted siRNA exhibited approximately an 80% reduction of mARC1 expression in the liver as compared to animals receiving buffer only injections (FIG. 2A). The reduction in mARC1 expression by the siRNA molecule was specific as liver expression of mARC2 mRNA was not affected (FIG. 2B). Treatment with the mARC1-targeted siRNA reduced serum high-density lipoprotein (HDL), LDL, and total cholesterol levels as well as serum levels of alanine aminotransferase (ALT) and C-reactive protein (CRP) (FIGS. 3A-3H). Triglyceride levels in the liver were also reduced in ob/ob animals receiving the mARC1-targeted siRNA (FIGS. 4A and 4B). Liver expression of fibrosis genes in animals receiving the mARC1-targeted siRNA were not significantly altered as compared to buffer-injected animals in this animal model (data not shown).

The results of this series of experiments show that specific inhibition of mARC1 expression in the liver with a mARC1-targeted siRNA molecule reduces serum cholesterol, LDL-cholesterol, ALT levels, and liver triglycerides, demonstrating a causal effect of mARC1 in lipid regulation in hepatocytes. The observed reductions in serum cholesterol, LDL-cholesterol, and ALT levels in the ob/ob animals treated with the mARC1-targeted siRNA are consistent with the reduced levels of these analytes observed in human carriers of the of the MARC1 A165T variant allele. Thus, inhibition of mARC1 expression with siRNA molecules, such as those described herein, may be useful to reduce cholesterol and triglyceride levels in patients with hypercholesterolemia or hyperlipidemic disorders and may be therapeutic for other liver disorders, such as nonalcoholic fatty liver disease, nonalcoholic steatohepatitis, alcoholic fatty liver disease, alcoholic steatohepatitis, liver fibrosis, and cirrhosis.

### Example 2. Design and Synthesis of mARC1 siRNA Molecules

Candidate sequences for the design of therapeutic siRNA molecules targeting the human MARC1 gene were identified using a bioinformatics analysis of the human MARC1 transcript, the sequence of which is provided herein as SEQ ID NO: 1 (Ensembl transcript no. ENST00000366910.9; see FIG. 1). Sequences were analyzed using an in-house siRNA design algorithm and selected if certain criteria were met. The bioinformatics analysis was conducted in two phases. In the first phase, sequences were evaluated for various features, including cross-reactivity with MARC1 transcripts from cynomolgus monkeys (Macaca fascicularis; NCBI Reference Sequence Nos.: XR_001490722.1, XR_001490722.1, XR_001490723.1, XR_001490726.1, XR_273285.2, XM_005540901.2, XR_273286.2, XM_005540898.2, and XM_005540899.2), sequence identity to other human, cynomolgus monkey, and rodent gene sequences, and for overlap with known human single nucleotide polymorphisms. In the second phase, selection criteria were adjusted to include sequences with specificity for only the human MARC1 transcript and to evaluate sequences for seed region matches to human microRNA (miRNA) sequences to predict off-target effects. Based on the results of the bioinformatics analysis, 665 sequences were selected for initial synthesis and in vitro testing.

RNAi constructs were synthesized using solid phase phosphoramidite chemistry. Synthesis was performed on a MerMade12 or MerMade192X (Bioautomation) instrument. Various chemical modifications, including 2′-fluoro modified nucleotides, 2′-O-methyl modified nucleotides, inverted abasic nucleotides, and phosphorothioate internucleotide linkages, were incorporated into the molecules. The RNAi constructs were generally formatted to be duplexes of 19-21 base pairs when annealed with either no overhangs (double bluntmer) or one or two overhangs of 2 nucleotides at the 3′ end of the antisense strand and/or the sense strand. For in vivo studies, the sense strands of the RNAi constructs were conjugated to a trivalent N-acetyl-galactosamine (GalNAc) moiety as described further below.

### Materials

Acetonitrile (DNA Synthesis Grade, AX0152-2505, EMD)

Capping Reagent A (80:10:10 (v/v/v) tetrahydrofuran/lutidine/acetic anhydride, BI0221/4000, EMD)

Capping Reagent B (16% 1-methylimidazole/tetrahydrofuran, BI0345/4000, EMD)

Activator Solution (0.25 M 5-(ethylthio)-1H-tetrazole (ETT) in acetonitrile, BI0152/0960, EMD)

Detritylation Reagent (3% dichloroacetic acid in dichloromethane, BI0830/4000, EMD)

Oxidation Reagent (0.02 M iodine in 70:20:10 (v/v/v) tetrahydrofuran/pyridine/water, BI0420/4000, EMD)

Diethylamine solution (20% DEA in acetonitrile, NC0017-0505, EMD)

Thiolation Reagent (0.05 M 5-N-[(dimethylamino)methylene]amino-3H-1,2,4-dithiazole-3-thione (BIOSULII/160K) in pyridine)

5′-Aminohexyl linker phosphoramidite and 2′-methoxy and 2′-fluoro phosphoramidites of adenosine, guanosine, and cytosine (Thermo Fisher Scientific), 0.10 M in acetonitrile over Molecular Trap Packs (0.5 g per 30 mL, Bioautomation)

2′-methoxy-uridine phosphoramidite (Thermo Fisher Scientific), 0.10 M in 90:10 (v/v) acetonitrile/DMF over Molecular Trap Packs (0.5 g per 30 mL, Bioautomation)

2′-deoxy-reverse absaic phosphoramidite (ChemGenes), 0.10 M in acetonitrile over Molecular Trap Packs (0.5 g per 30 mL, Bioautomation)

CPG Support (Hi-Load Universal Support, 500A (BH5-3500-G1), 79.6 μmol/g, 0.126 g (10 μmol)) or 1 μmol Universal Synthesis Column, 500A, Pipette Style Body (MM5-3500-1, Bioautomation)

Ammonium hydroxide (concentrated, J. T. Baker)

### Synthesis

Reagent solutions, phosphoramidite solutions, and solvents were attached to the MerMade12 or MerMade192X instrument. Solid support was added to each column (4 mL SPE tube with top and bottom frit for 10 μmol), and the columns were affixed to the instrument. The columns were washed twice with acetonitrile. The phosphoramidite and reagent solution lines were purged. The synthesis was initiated using the Poseidon software. The synthesis was accomplished by repetition of the deprotection/coupling/oxidation/capping synthesis cycle. Specifically, to the solid support was added detritylation reagent to remove the 5′-dimethoxytrityl (DMT) protecting group. The solid support was washed with acetonitrile. To the support was added phosphoramidite and activator solution followed by incubation to couple the incoming nucleotide to the free 5′-hydroxyl group. The support was washed with acetonitrile. To the support was added oxidation or thiolation reagent to convert the phosphite triester to the phosphate triester or phosphorothioate. To the support was added capping reagents A and B to terminate any unreacted oligonucleotide chains. The support was washed with acetonitrile. After the final reaction cycle, the resin was washed with diethylamine solution to remove the 2-cyanoethyl protecting groups. The support was washed with acetonitrile and dried under vacuum.

### GalNAc Conjugation

Sense strands for conjugation to a trivalent GalNAc moiety (structure shown in Formula VII below) were prepared with a 5′-aminohexyl linker. After automated synthesis, the column was removed from the instrument and transferred to a vacuum manifold in a hood. The 5′-monomethoxytrityl (MMT) protecting group was removed from the solid support by successive treatments with 2 mL aliquots of 1% trifluoroacetic acid (TFA) in dichloromethane (DCM) with vacuum filtration. When the orange/yellow color was no longer observable in the eluent, the resin was washed with dichloromethane. The resin was washed with 5 mL of 10% diisopropylethylamine in N,N-dimethylformamide (DMF). In a separate vial a solution of GalNAc3-Lys2-Ahx (67 mg, 40 μmol) in DMF (0.5 mL), the structure and synthesis of which is described below, was prepared with 1,1,3,3-tetramethyluronium tetrafluoroborate (TATU, 12.83 mg, 40 μmol) and diisopropylethylamine (DIEA, 13.9 μL, 80 μmol). The activated coupling solution was added to the resin, and the column was capped and incubated at room temperature overnight. The resin was washed with DMF, DCM, and dried under vacuum.

### Cleavage

The synthesis columns were removed from the synthesizer or vacuum manifold and transferred to a cleavage apparatus. To the solid support was added 4×1 mL (for 10 μmol) or 4×250 μL (for 1 μmol) of concentrated ammonium hydroxide. The eluent was collected by gravity or light vacuum filtration into a 24- or 96-well deep well plate, respectively. The plate was sealed, bolted into a cleavage chuck (Bioautomation), and the mixture was heated at 55° C. for 4 h. The plate was moved to the freezer and cooled for 20 minutes before opening the cleavage chuck in the hood.

### Analysis and Purification

A portion of the cleavage solution was analyzed and purified by anion exchange chromatography. The pooled fractions were desalted by size exclusion chromatography and analyzed by ion pair-reversed phase high-performance liquid chromatograph-mass spectrometry (HPLC-MS). The pooled fractions were lyophilized to obtain a white amorphous powder.

### Analytical Anion Exchange Chromatography (AEX):

Column: Thermo DNAPac PA200RS (4.6×50 mm, 4 μm)

Instrument: Agilent 1100 HPLC

Buffer A: 20 mM sodium phosphate, 10% acetonitrile, pH 8.5

Buffer B: 20 mM sodium phosphate, 10% acetonitrile, pH 8.5, 1 M sodium bromide

Flow rate: 1 mL/min at 40° C.

Gradient: 20-65% B in 6.2 min

### Preparative Anion Exchange Chromatography (AEX):

Column: Tosoh TSK Gel SuperQ-SPW, 21×150 mm, 13 μm

Instrument: Agilent 1200 HPLC

Buffer A: 20 mM sodium phosphate, 10% acetonitrile, pH 8.5

Buffer B: 20 mM sodium phosphate, 10% acetonitrile, pH 8.5, 1 M sodium bromide

Flow rate: 8 mL/min

Injection volume: 5 mL

Gradient: 35-55% B over 40 min for sense strands and 50-100% B over 40 min for antisense strands

### Preparative Size Exclusion Chromatography (SEC):

Column: 3×GE Hi-Prep 26/10 in series

Instrument: GE AKTA Pure

Buffer: 20% ethanol in water

Flow Rate: 10 mL/min

Injection volume: 45 mL using sample loading pump

### Ion Pair-Reversed Phase (IP-RP) HPLC:

Column: Water Xbridge BEH OST C18, 2.5 μm, 2.1×50 mm

Instrument: Agilent 1100 HPLC

Buffer A: 15.7 mM DIEA, 50 mM hexafluoroisopropanol (HFIP) in water

Buffer B: 15.7 mM DIEA, 50 mM HFIP in 50:50 water/acetonitrile

Flow rate: 0.5 mL/min

Gradient: 10-30% B over 6 min

### Annealing

A small amount of the sense strand and the antisense strand were weighed into individual vials. To the vials was added phosphate buffered saline (PBS, Gibco) to an approximate concentration of 2 mM based on the dry weight. The actual sample concentration was measured on the NanoDrop One (ssDNA, extinction coefficient=33 μg/OD260). The two strands were then mixed in an equimolar ratio, and the sample was heated for 5 minutes in a 90° C. incubator and allowed to cool slowly to room temperature. The sample was analyzed by AEX. The duplex was registered and submitted for in vitro and in vivo testing as described in more detail in Examples 3 and 4 below.

### Preparation of GalNAc3-Lys2-Ahx

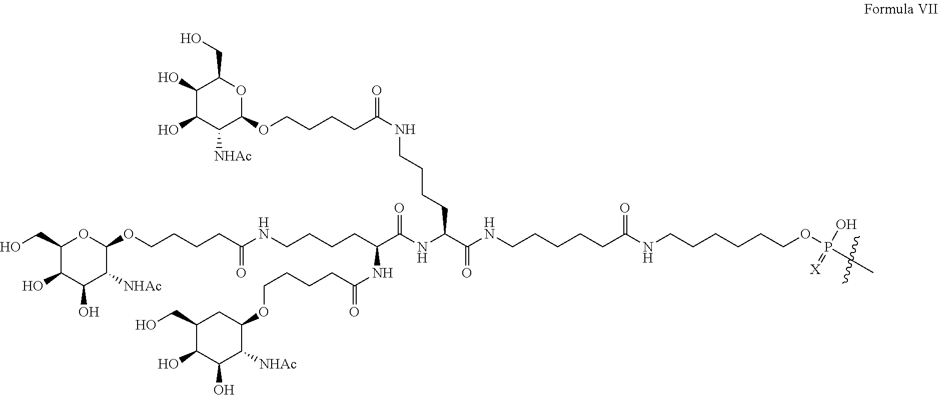

wherein X=O or S. The squiggly line represents the point of attachment to the 5′ terminal nucleotide of the sense strand of the RNAi construct. The GalNAc moiety was attached to the 5′ carbon of the 5′ terminal nucleotide of the sense strand except where an inverted abasic (invAb) deoxynucleotide was the 5′ terminal nucleotide and linked to the adjacent nucleotide via a 5′-5′ internucleotide linkage, in which case the GalNAc moiety was attached to the 3′ carbon of the inverted abasic deoxynucleotide.

To a 50 mL falcon tube was added Fmoc-Ahx-OH (1.13 g, 3.19 mmol) in DCM (30 mL) followed by DIEA (2.23 mL, 12.78 mmol). The solution was added to 2-Cl Trityl chloride resin (3.03 g, 4.79 mmol) in a 50 mL centrifuge tube and loaded onto a shaker for 2 h. The solvent was drained and the resin was washed with 17:2:1 DCM/MeOH/DIEA (30 ml×2), DCM (30 mL×4) and dried. The loading was determined to be 0.76 mmol/g with UV spectrophotometric detection at 290 nm.

3 g of the loaded 2-Cl Trityl resin was suspended in 20% 4-methylpiperidine in DMF (20 mL), and after 30 min the solvent was drained. The process was repeated one more time, and the resin was washed with DMF (30 mL×3) and DCM (30 mL×3).

To a solution of Fmoc-Lys(ivDde)-OH (3.45 g, 6 mmol) in DMF (20 mL) was added TATU (1.94 g, 6 mmol) followed by DIEA (1.83 mL, 10.5 mmol). The solution was then added to the above deprotected resin, and the suspension was set on a shaker overnight. The solvent was drained and the resin was washed with DMF (30 mL×3) and DCM (30 mL×3).

The resin was treated with 20% 4-methylpiperidine in DMF (15 mL) and after 10 min the solvent was drained. The process was repeated one more time and the resin was washed with DMF (15 mL×4) and DCM (15 mL×4).

To a solution of Fmoc-Lys(Fmoc)-OH (3.54 g, 6 mmol) in DMF (20 mL) was added TATU (1.94 g, 6 mmol) followed by DIEA (1.83 mL, 10.5 mmol). The solution was then added to the above deprotected resin and the suspension was set on a shaker overnight. The solvent was drained and the resin was washed with DMF (30 mL×3) and DCM (30 mL×3).

The resin was treated with 5% hydrazine in DMF (20 mL) and after 5 min, the solvent was drained. The process was repeated four more times and the resin was washed with DMF (30 mL×4) and DCM (30 mL×4).

To a solution of 5-(((2R,3R,4R,5R,6R)-3-acetamido-4,5-diacetoxy-6-(acetoxymethyl)tetrahydro-2H-pyran-2-yl)oxy)pentanoic acid (4.47 g, 10 mmol) in DMF (40 mL) was added TATU (3.22 g, 10 mmol), and the solution was stirred for 5 min. DIEA (2.96 mL, 17 mmol) was added to the solution, and the mixture was then added to the resin above. The suspension was kept at room temperature overnight and the solvent was drained. The resin was washed with DMF (3×30 mL) and DCM (3×30 mL).

The resin was treated with 1% TFA in DCM (30 mL with 3% triisopropylsilane) and after 5 min, the solvent was drained. The process was repeated three more times, and the combined filtrate was concentrated in vacuo. The residue was triturated with diethyl ether (50 mL) and the suspension was filtered and dried to give the crude product. The crude product was purified with reverse phase chromatography and eluted with 0-20% of MeCN in water. The fractions were combined and lyophilized to give the product as a white solid.

Table 1 below lists the unmodified sense and antisense sequences for molecules prioritized from the bioinformatics analysis. The range of nucleotides targeted by siRNA molecules in each sequence family within the human MARC1 transcript (SEQ ID NO: 1) is also shown in Table 1. Duplex nos. D-1000 to D-1003 were designed to target the Marc1 mouse transcript and do not cross-react with the human MARC1 transcript. Table 2 provides the sequences of the sense and antisense strands with chemical modifications. Based on activity in in vitro cell-based assays and in vivo mouse studies as described in Examples 3 and 4, respectively, sequences targeting specific regions of the human MARC1 transcript were selected for structure-activity relationship (SAR) studies. The nucleotide sequences are listed according to the following notations: a, u, g, and c=corresponding 2′-O-methyl ribonucleotide; Af, Uf, Gf, and Cf=corresponding 2′-deoxy-2′-fluoro (“2′-fluoro”) ribonucleotide; and invAb=inverted abasic deoxynucleotide (i.e. abasic deoxynucleotide linked to adjacent nucleotide via a substituent at its 3′ position (a 3′-3′ linkage) when on the 3′ end of a strand or linked to adjacent nucleotide via a substituent at its 5′ position (a 5′-5′ internucleotide linkage) when on the 5′ end of a strand. Insertion of an “s” in the sequence indicates that the two adjacent nucleotides are connected by a phosphorothiodiester group (e.g. a phosphorothioate internucleotide linkage). Unless indicated otherwise, all other nucleotides are connected by 3′-5′ phosphodiester groups. [GalNAc3] represents the GalNAc moiety shown in Formula VII, which was covalently attached to the 5′ terminal nucleotide at the 5′ end of the sense strand via a phophodiester bond or a phoshorothioate bond when an “s” follows the [GalNAc3] notation. When an invAb nucleotide was the 5′ terminal nucleotide at the 5′ end of the sense strand, it was linked to the adjacent nucleotide via a 5′-5′ linkage and the GalNAc moiety was covalently attached to the 3′ carbon of the invAb nucleotide. Otherwise, the GalNAc moiety was covalently attached to the 5′ carbon of the 5′ terminal nucleotide of the sense strand.

TABLE 1
Unmodified mARC1 siRNA sequences

| | Target site | | | | |
|---|---|---|---|---|---|
| | within human | | | | |
| | MARC1 | | SEQ | | SEQ |
| Duplex | transcript | | ID | | ID |
| No. | (SEQ ID NO: 1) | Sense Sequence (5′-3′) | NO: | Antisense Sequence (5′-3′) | NO: |
| D-1000 | — | GAGCAAGCACUAUAUGGAAU | 2 | UUCCAUAUAGUGCUUGCUCGG | 671 |
| D-1001 | — | AGAAGUUCUCGGCAAAUGAU | 3 | UCAUUUGCCGAGAACUUCUGG | 672 |
| D-1002 | — | GAGCAAGCUGAAUUUGGAAU | 4 | UUCCAAAUUCAGCUUGCUCGG | 673 |
| D-1003 | — | AGAAGUUCAGCGCUAAUGAU | 5 | UCAUUAGCGCUGAACUUCUGG | 674 |
| D-1004 | 40-60 | GAAGGACGCACUGCUCUGAU | 6 | AAUCAGAGCAGUGCGUCCUUCUU | 675 |
| D-1005 | 42-62 | AGGACGCACUGCUCUGAUUG | 7 | ACAAUCAGAGCAGUGCGUCCUUU | 676 |
| D-1006 | 43-63 | GGACGCACUGCUCUGAUUGG | 8 | ACCAAUCAGAGCAGUGCGUCCUU | 677 |
| D-1007 | 45-65 | ACGCACUGCUCUGAUUGGCC | 9 | AGGCCAAUCAGAGCAGUGCGUUU | 678 |
| D-1008 | 50-70 | CUGCUCUGAUUGGCCCGGAA | 10 | AUUCCGGGCCAAUCAGAGCAGUU | 679 |
| D-1009 | 51-71 | UGCUCUGAUUGGCCCGGAAG | 11 | ACUUCCGGGCCAAUCAGAGCAUU | 680 |
| D-1010 | 52-72 | GCUCUGAUUGGCCCGGAAGG | 12 | ACCUUCCGGGCCAAUCAGAGCUU | 681 |
| D-1011 | 99-119 | CGGGGCCAAAGGCCGCACCU | 13 | AAGGUGCGGCCUUUGGCCCCGUU | 682 |
| D-1012 | 100-120 | GGGGCCAAAGGCCGCACCUU | 14 | AAAGGUGCGGCCUUUGGCCCCUU | 683 |
| D-1013 | 103-123 | GCCAAAGGCCGCACCUUCCC | 15 | AGGGAAGGUGCGGCCUUUGGCUU | 684 |
| D-1014 | 104-124 | CCAAAGGCCGCACCUUCCCC | 16 | AGGGGAAGGUGCGGCCUUUGGUU | 685 |
| D-1015 | 163-183 | CGCCACCUCGCGGAGAAGCC | 17 | UGGCUUCUCCGCGAGGUGGCGUU | 686 |
| D-1016 | 164-184 | GCCACCUCGCGGAGAAGCCA | 18 | AUGGCUUCUCCGCGAGGUGGCUU | 687 |
| D-1017 | 165-185 | CCACCUCGCGGAGAAGCCAG | 19 | ACUGGCUUCUCCGCGAGGUGGUU | 688 |
| D-1018 | 167-187 | ACCUCGCGGAGAAGCCAGCC | 20 | UGGCUGGCUUCUCCGCGAGGUUU | 689 |
| D-1019 | 473-493 | UGAUCAACCAGGAGGGAAAC | 21 | UGUUUCCCUCCUGGUUGAUCAUU | 690 |
| D-1020 | 475-495 | AUCAACCAGGAGGGAAACAU | 22 | AAUGUUUCCCUCCUGGUUGAUUU | 691 |
| D-1021 | 476-496 | UCAACCAGGAGGGAAACAUG | 23 | ACAUGUUUCCCUCCUGGUUGAUU | 692 |
| D-1022 | 477-497 | CAACCAGGAGGGAAACAUGG | 24 | ACCAUGUUUCCCUCCUGGUUGUU | 693 |
| D-1023 | 478-498 | AACCAGGAGGGAAACAUGGU | 25 | AACCAUGUUUCCCUCCUGGUUUU | 694 |
| D-1024 | 479-499 | ACCAGGAGGGAAACAUGGUU | 26 | UAACCAUGUUUCCCUCCUGGUUU | 695 |
| D-1025 | 501-521 | UGCUCGCCAGGAACCUCGCC | 27 | AGGCGAGGUUCCUGGCGAGCAUU | 696 |
| D-1026 | 503-523 | CUCGCCAGGAACCUCGCCUG | 28 | ACAGGCGAGGUUCCUGGCGAGUU | 697 |
| D-1027 | 510-530 | GGAACCUCGCCUGGUCCUGA | 29 | AUCAGGACCAGGCGAGGUUCCUU | 698 |
| D-1028 | 512-532 | AACCUCGCCUGGUCCUGAUU | 30 | AAAUCAGGACCAGGCGAGGUUUU | 699 |
| D-1029 | 513-533 | ACCUCGCCUGGUCCUGAUUU | 31 | AAAAUCAGGACCAGGCGAGGUUU | 700 |
| D-1030 | 514-534 | CCUCGCCUGGUCCUGAUUUC | 32 | AGAAAUCAGGACCAGGCGAGGUU | 701 |
| D-1031 | 515-535 | CUCGCCUGGUCCUGAUUUCC | 33 | AGGAAAUCAGGACCAGGCGAGUU | 702 |
| D-1032 | 519-539 | CCUGGUCCUGAUUUCCCUGA | 34 | AUCAGGGAAAUCAGGACCAGGUU | 703 |
| D-1033 | 558-578 | GACUCUCAGUGCAGCCUACA | 35 | AUGUAGGCUGCACUGAGAGUCUU | 704 |
| D-1034 | 560-580 | CUCUCAGUGCAGCCUACACA | 36 | UUGUGUAGGCUGCACUGAGAGUU | 705 |
| D-1035 | 561-581 | UCUCAGUGCAGCCUACACAA | 37 | UUUGUGUAGGCUGCACUGAGAUU | 706 |
| D-1036 | 562-582 | CUCAGUGCAGCCUACACAAA | 38 | AUUUGUGUAGGCUGCACUGAGUU | 707 |
| D-1037 | 563-583 | UCAGUGCAGCCUACACAAAG | 39 | ACUUUGUGUAGGCUGCACUGAUU | 708 |
| D-1038 | 596-616 | CUAUCAAAACGCCCACCACA | 40 | UUGUGGUGGGCGUUUUGAUAGUU | 709 |
| D-1039 | 597-617 | UAUCAAAACGCCCACCACAA | 41 | UUUGUGGUGGGCGUUUUGAUAUU | 710 |
| D-1040 | 598-618 | AUCAAAACGCCCACCACAAA | 42 | AUUUGUGGUGGGCGUUUUGAUUU | 711 |
| D-1041 | 599-619 | UCAAAACGCCCACCACAAAU | 43 | AAUUUGUGGUGGGCGUUUUGAUU | 712 |
| D-1042 | 602-622 | AAACGCCCACCACAAAUGCA | 44 | AUGCAUUUGUGGUGGGCGUUUUU | 713 |
| D-1043 | 603-623 | AACGCCCACCACAAAUGCAG | 45 | ACUGCAUUUGUGGUGGGCGUUUU | 714 |
| D-1044; | 684-704 | CCAGUGGAUAACCAGCUUCC | 46 | AGGAAGCUGGUUAUCCACUGGUU | 715 |
| D-2004; | | | | | |
| D-2165; | | | | | |
| D-2172 | | | | | |
| D-1045 | 685-705 | CAGUGGAUAACCAGCUUCCU | 47 | AAGGAAGCUGGUUAUCCACUGUU | 716 |
| D-1046 | 687-707 | GUGGAUAACCAGCUUCCUGA | 48 | UUCAGGAAGCUGGUUAUCCACUU | 717 |
| D-1047 | 690-710 | GAUAACCAGCUUCCUGAAGU | 49 | AACUUCAGGAAGCUGGUUAUCUU | 718 |
| D-1048 | 764-784 | AUCAAAUAGCAGACUUGUUC | 50 | AGAACAAGUCUGCUAUUUGAUUU | 719 |
| D-1049 | 766-786 | CAAAUAGCAGACUUGUUCCG | 51 | UCGGAACAAGUCUGCUAUUUGUU | 720 |
| D-1050 | 767-787 | AAAUAGCAGACUUGUUCCGA | 52 | AUCGGAACAAGUCUGCUAUUUUU | 721 |
| D-1051 | 951-971 | UGAGCUUCUUAUUGGUGACG | 53 | ACGUCACCAAUAAGAAGCUCAUU | 722 |
| D-1052 | 953-973 | AGCUUCUUAUUGGUGACGUG | 54 | ACACGUCACCAAUAAGAAGCUUU | 723 |
| D-1053 | 954-974 | GCUUCUUAUUGGUGACGUGG | 55 | UCCACGUCACCAAUAAGAAGCUU | 724 |
| D-1054; | 956-976 | UUCUUAUUGGUGACGUGGAA | 56 | AUUCCACGUCACCAAUAAGAAUU | 725 |
| D-2029 | | | | | |
| D-1055 | 962-982 | UUGGUGACGUGGAACUGAAA | 57 | UUUUCAGUUCCACGUCACCAAUU | 726 |
| D-1056 | 963-983 | UGGUGACGUGGAACUGAAAA | 58 | AUUUUCAGUUCCACGUCACCAUU | 727 |
| D-1057 | 964-984 | GGUGACGUGGAACUGAAAAG | 59 | ACUUUUCAGUUCCACGUCACCUU | 728 |
| D-1058 | 965-985 | GUGACGUGGAACUGAAAAGG | 60 | ACCUUUUCAGUUCCACGUCACUU | 729 |
| D-1059 | 991-1011 | GCUUGUUCCAGAUGCAUUUU | 61 | UAAAAUGCAUCUGGAACAAGCUU | 730 |
| D-1060 | 995-1015 | GUUCCAGAUGCAUUUUAACC | 62 | UGGUUAAAAUGCAUCUGGAACUU | 731 |
| D-1061; | 996-1016 | UUCCAGAUGCAUUUUAACCA | 63 | AUGGUUAAAAUGCAUCUGGAAUU | 732 |
| D-2002; | | | | | |
| D-2228 | | | | | |
| D-1062; | 1003-1023 | UGCAUUUUAACCACAGUGGA | 64 | AUCCACUGUGGUUAAAAUGCAUU | 733 |
| D-2003 | | | | | |
| D-1063 | 1004-1024 | GCAUUUUAACCACAGUGGAC | 65 | AGUCCACUGUGGUUAAAAUGCUU | 734 |
| D-1064 | 1033-1053 | GGUGUCAUGAGCAGGAAGGA | 66 | UUCCUUCCUGCUCAUGACACCUU | 735 |
| D-1065 | 1051-1071 | GAACCGCUGGAAACACUGAA | 67 | AUUCAGUGUUUCCAGCGGUUCUU | 736 |
| D-1066; | 1056-1076 | GCUGGAAACACUGAAGAGUU | 68 | UAACUCUUCAGUGUUUCCAGCUU | 737 |
| D-2005 | | | | | |
| D-1067; | 1059-1079 | GGAAACACUGAAGAGUUAUC | 69 | AGAUAACUCUUCAGUGUUUCCUU | 738 |
| D-2035 | | | | | |
| D-1068; | 1060-1080 | GAAACACUGAAGAGUUAUCG | 70 | ACGAUAACUCUUCAGUGUUUCUU | 739 |
| D-2006 | | | | | |
| D-1069 | 1061-1081 | AAACACUGAAGAGUUAUCGC | 71 | AGCGAUAACUCUUCAGUGUUUUU | 740 |
| D-1070; | 1062-1082 | AACACUGAAGAGUUAUCGCC | 72 | UGGCGAUAACUCUUCAGUGUUUU | 741 |
| D-2007 | | | | | |
| D-1071 | 1063-1083 | ACACUGAAGAGUUAUCGCCA | 73 | AUGGCGAUAACUCUUCAGUGUUU | 742 |
| D-1072 | 1064-1084 | CACUGAAGAGUUAUCGCCAG | 74 | ACUGGCGAUAACUCUUCAGUGUU | 743 |
| D-1073 | 1065-1085 | ACUGAAGAGUUAUCGCCAGU | 75 | AACUGGCGAUAACUCUUCAGUUU | 744 |
| D-1074; | 1066-1086 | CUGAAGAGUUAUCGCCAGUG | 76 | ACACUGGCGAUAACUCUUCAGUU | 745 |
| D-2025 | | | | | |
| D-1075 | 1067-1087 | UGAAGAGUUAUCGCCAGUGU | 77 | AACACUGGCGAUAACUCUUCAUU | 746 |
| D-1076 | 1068-1088 | GAAGAGUUAUCGCCAGUGUG | 78 | UCACACUGGCGAUAACUCUUCUU | 747 |
| D-1077 | 1071-1091 | GAGUUAUCGCCAGUGUGACC | 79 | AGGUCACACUGGCGAUAACUCUU | 748 |
| D-1078 | 1072-1092 | AGUUAUCGCCAGUGUGACCC | 80 | AGGGUCACACUGGCGAUAACUUU | 749 |
| D-1079 | 1073-1093 | GUUAUCGCCAGUGUGACCCU | 81 | AAGGGUCACACUGGCGAUAACUU | 750 |
| D-1080 | 1074-1094 | UUAUCGCCAGUGUGACCCUU | 82 | AAAGGGUCACACUGGCGAUAAUU | 751 |
| D-1081 | 1078-1098 | CGCCAGUGUGACCCUUCAGA | 83 | UUCUGAAGGGUCACACUGGCGUU | 752 |
| D-1082 | 1079-1099 | GCCAGUGUGACCCUUCAGAA | 84 | AUUCUGAAGGGUCACACUGGCUU | 753 |
| D-1083; | 1081-1101 | CAGUGUGACCCUUCAGAACG | 85 | UCGUUCUGAAGGGUCACACUGUU | 754 |
| D-2050 | | | | | |
| D-1084 | 1082-1102 | AGUGUGACCCUUCAGAACGA | 86 | UUCGUUCUGAAGGGUCACACUUU | 755 |
| D-1085 | 1083-1103 | GUGUGACCCUUCAGAACGAA | 87 | UUUCGUUCUGAAGGGUCACACUU | 756 |
| D-1086; | 1084-1104 | UGUGACCCUUCAGAACGAAA | 88 | AUUUCGUUCUGAAGGGUCACAUU | 757 |
| D-2049 | | | | | |
| D-1087; | 1085-1105 | GUGACCCUUCAGAACGAAAG | 89 | ACUUUCGUUCUGAAGGGUCACUU | 758 |
| D-2027 | | | | | |
| D-1088 | 1086-1106 | UGACCCUUCAGAACGAAAGU | 90 | AACUUUCGUUCUGAAGGGUCAUU | 759 |
| D-1089 | 1087-1107 | GACCCUUCAGAACGAAAGUU | 91 | UAACUUUCGUUCUGAAGGGUCUU | 760 |
| D-1090; | 1088-1108 | ACCCUUCAGAACGAAAGUUA | 92 | AUAACUUUCGUUCUGAAGGGUUU | 761 |
| D-2026 | | | | | |
| D-1091; | 1089-1109 | CCCUUCAGAACGAAAGUUAU | 93 | UAUAACUUUCGUUCUGAAGGGUU | 762 |
| D-2031 | | | | | |
| D-1092; | 1090-1110 | CCUUCAGAACGAAAGUUAUA | 94 | AUAUAACUUUCGUUCUGAAGGUU | 763 |
| D-2032; | | | | | |
| D-2229 | | | | | |
| D-1093; | 1091-1111 | CUUCAGAACGAAAGUUAUAU | 95 | AAUAUAACUUUCGUUCUGAAGUU | 764 |
| D-2033; | | | | | |
| D-2230 | | | | | |
| D-1094 | 1092-1112 | UUCAGAACGAAAGUUAUAUG | 96 | ACAUAUAACUUUCGUUCUGAAUU | 765 |
| D-1095; | 1093-1113 | UCAGAACGAAAGUUAUAUGG | 97 | UCCAUAUAACUUUCGUUCUGAUU | 766 |
| D-2028; | | | | | |
| D-2227 | | | | | |
| D-1096; | 1094-1114 | CAGAACGAAAGUUAUAUGGA | 98 | UUCCAUAUAACUUUCGUUCUGUU | 767 |
| D-2001; | | | | | |
| D-2163; | | | | | |
| D-2170 | | | | | |
| D-1097; | 1103-1123 | AGUUAUAUGGAAAAUCACCA | 99 | AUGGUGAUUUUCCAUAUAACUUU | 768 |
| D-2030 | | | | | |
| D-1098 | 1105-1125 | UUAUAUGGAAAAUCACCACU | 100 | AAGUGGUGAUUUUCCAUAUAAUU | 769 |
| D-1099; | 768-788 | AAUAGCAGACUUGUUCCGAC | 101 | AGUCGGAACAAGUCUGCUAUUUU | 770 |
| D-2057; | | | | | |
| D-2489; | | | | | |
| D-2501; | | | | | |
| D-2507 | | | | | |
| D-1100 | 769-789 | AUAGCAGACUUGUUCCGACC | 102 | AGGUCGGAACAAGUCUGCUAUUU | 771 |
| D-1101 | 770-790 | UAGCAGACUUGUUCCGACCC | 103 | UGGGUCGGAACAAGUCUGCUAUU | 772 |
| D-1102 | 771-791 | AGCAGACUUGUUCCGACCCA | 104 | UUGGGUCGGAACAAGUCUGCUUU | 773 |
| D-1103 | 772-792 | GCAGACUUGUUCCGACCCAA | 105 | AUUGGGUCGGAACAAGUCUGCUU | 774 |
| D-1104 | 773-793 | CAGACUUGUUCCGACCCAAG | 106 | ACUUGGGUCGGAACAAGUCUGUU | 775 |
| D-1105 | 774-794 | AGACUUGUUCCGACCCAAGG | 107 | UCCUUGGGUCGGAACAAGUCUUU | 776 |
| D-1106 | 775-795 | GACUUGUUCCGACCCAAGGA | 108 | AUCCUUGGGUCGGAACAAGUCUU | 777 |
| D-1107 | 776-796 | ACUUGUUCCGACCCAAGGAC | 109 | AGUCCUUGGGUCGGAACAAGUUU | 778 |
| D-1108 | 777-797 | CUUGUUCCGACCCAAGGACC | 110 | UGGUCCUUGGGUCGGAACAAGUU | 779 |
| D-1109 | 781-801 | UUCCGACCCAAGGACCAGAU | 111 | AAUCUGGUCCUUGGGUCGGAAUU | 780 |
| D-1110 | 790-810 | AAGGACCAGAUUGCUUACUC | 112 | UGAGUAAGCAAUCUGGUCCUUUU | 781 |
| D-1111; | 793-813 | GACCAGAUUGCUUACUCAGA | 113 | AUCUGAGUAAGCAAUCUGGUCUU | 782 |
| D-2039 | | | | | |
| D-1112 | 795-815 | CCAGAUUGCUUACUCAGACA | 114 | AUGUCUGAGUAAGCAAUCUGGUU | 783 |
| D-1113; | 796-816 | CAGAUUGCUUACUCAGACAC | 115 | AGUGUCUGAGUAAGCAAUCUGUU | 784 |
| D-2024; | | | | | |
| D-2167; | | | | | |
| D-2174 | | | | | |
| D-1114 | 797-817 | AGAUUGCUUACUCAGACACC | 116 | UGGUGUCUGAGUAAGCAAUCUUU | 785 |
| D-1115 | 798-818 | GAUUGCUUACUCAGACACCA | 117 | AUGGUGUCUGAGUAAGCAAUCUU | 786 |
| D-1116 | 799-819 | AUUGCUUACUCAGACACCAG | 118 | ACUGGUGUCUGAGUAAGCAAUUU | 787 |
| D-1117 | 802-822 | GCUUACUCAGACACCAGCCC | 119 | UGGGCUGGUGUCUGAGUAAGCUU | 788 |
| D-1118 | 804-824 | UUACUCAGACACCAGCCCAU | 120 | AAUGGGCUGGUGUCUGAGUAAUU | 789 |
| D-1119 | 851-871 | CGGAUCUCAACUCCAGGCUA | 121 | AUAGCCUGGAGUUGAGAUCCGUU | 790 |
| D-1120 | 852-872 | GGAUCUCAACUCCAGGCUAG | 122 | UCUAGCCUGGAGUUGAGAUCCUU | 791 |
| D-1121 | 853-873 | GAUCUCAACUCCAGGCUAGA | 123 | AUCUAGCCUGGAGUUGAGAUCUU | 792 |
| D-1122 | 854-874 | AUCUCAACUCCAGGCUAGAG | 124 | UCUCUAGCCUGGAGUUGAGAUUU | 793 |
| D-1123 | 859-879 | AACUCCAGGCUAGAGAAGAA | 125 | UUUCUUCUCUAGCCUGGAGUUUU | 794 |
| D-1124 | 872-892 | AGAAGAAAGUUAAAGCAACC | 126 | UGGUUGCUUUAACUUUCUUCUUU | 795 |
| D-1125 | 873-893 | GAAGAAAGUUAAAGCAACCA | 127 | UUGGUUGCUUUAACUUUCUUCUU | 796 |
| D-1126 | 874-894 | AAGAAAGUUAAAGCAACCAA | 128 | AUUGGUUGCUUUAACUUUCUUUU | 797 |
| D-1127 | 875-895 | AGAAAGUUAAAGCAACCAAC | 129 | AGUUGGUUGCUUUAACUUUCUUU | 798 |
| D-1128 | 876-896 | GAAAGUUAAAGCAACCAACU | 130 | AAGUUGGUUGCUUUAACUUUCUU | 799 |
| D-1129 | 877-897 | AAAGUUAAAGCAACCAACUU | 131 | AAAGUUGGUUGCUUUAACUUUUU | 800 |
| D-1130; | 878-898 | AAGUUAAAGCAACCAACUUC | 132 | UGAAGUUGGUUGCUUUAACUUUU | 801 |
| D-2037 | | | | | |
| D-1131 | 879-899 | AGUUAAAGCAACCAACUUCA | 133 | AUGAAGUUGGUUGCUUUAACUUU | 802 |
| D-1132 | 880-900 | GUUAAAGCAACCAACUUCAG | 134 | ACUGAAGUUGGUUGCUUUAACUU | 803 |
| D-1133 | 883-903 | AAAGCAACCAACUUCAGGCC | 135 | AGGCCUGAAGUUGGUUGCUUUUU | 804 |
| D-1134 | 885-905 | AGCAACCAACUUCAGGCCCA | 136 | UUGGGCCUGAAGUUGGUUGCUUU | 805 |
| D-1135 | 887-907 | CAACCAACUUCAGGCCCAAU | 137 | UAUUGGGCCUGAAGUUGGUUGUU | 806 |
| D-1136 | 888-908 | AACCAACUUCAGGCCCAAUA | 138 | AUAUUGGGCCUGAAGUUGGUUUU | 807 |
| D-1137 | 890-910 | CCAACUUCAGGCCCAAUAUU | 139 | AAAUAUUGGGCCUGAAGUUGGUU | 808 |
| D-1138; | 891-911 | CAACUUCAGGCCCAAUAUUG | 140 | ACAAUAUUGGGCCUGAAGUUGUU | 809 |
| D-2034; | | | | | |
| D-2231 | | | | | |
| D-1139; | 893-913 | ACUUCAGGCCCAAUAUUGUA | 141 | UUACAAUAUUGGGCCUGAAGUUU | 810 |
| D-2036; | | | | | |
| D-2232 | | | | | |
| D-1140; | 894-914 | CUUCAGGCCCAAUAUUGUAA | 142 | AUUACAAUAUUGGGCCUGAAGUU | 811 |
| D-2038; | | | | | |
| D-2161; | | | | | |
| D-2168 | | | | | |
| D-1141 | 895-915 | UUCAGGCCCAAUAUUGUAAU | 143 | AAUUACAAUAUUGGGCCUGAAUU | 812 |
| D-1142 | 896-916 | UCAGGCCCAAUAUUGUAAUU | 144 | AAAUUACAAUAUUGGGCCUGAUU | 813 |
| D-1143; | 898-918 | AGGCCCAAUAUUGUAAUUUC | 145 | UGAAAUUACAAUAUUGGGCCUUU | 814 |
| D-2000 | | | | | |
| D-1144 | 899-919 | GGCCCAAUAUUGUAAUUUCA | 146 | AUGAAAUUACAAUAUUGGGCCUU | 815 |
| D-1145 | 949-969 | GAUGAGCUUCUUAUUGGUGA | 147 | AUCACCAAUAAGAAGCUCAUCUU | 816 |
| D-1146 | 950-970 | AUGAGCUUCUUAUUGGUGAC | 148 | AGUCACCAAUAAGAAGCUCAUUU | 817 |
| D-1147; | 1107-1127 | AUAUGGAAAAUCACCACUCU | 149 | AAGAGUGGUGAUUUUCCAUAUUU | 818 |
| D-2041 | | | | | |
| D-1148 | 1108-1128 | UAUGGAAAAUCACCACUCUU | 150 | AAAGAGUGGUGAUUUUCCAUAUU | 819 |
| D-1149 | 1109-1129 | AUGGAAAAUCACCACUCUUU | 151 | AAAAGAGUGGUGAUUUUCCAUUU | 820 |
| D-1150; | 1110-1130 | UGGAAAAUCACCACUCUUUG | 152 | ACAAAGAGUGGUGAUUUUCCAUU | 821 |
| D-2060; | | | | | |
| D-2207; | | | | | |
| D-2215 | | | | | |
| D-1151 | 1144-1164 | CUGGAAAACCCAGGGACCAU | 153 | AAUGGUCCCUGGGUUUUCCAGUU | 822 |
| D-1152 | 1146-1166 | GGAAAACCCAGGGACCAUCA | 154 | UUGAUGGUCCCUGGGUUUUCCUU | 823 |
| D-1153 | 1147-1167 | GAAAACCCAGGGACCAUCAA | 155 | UUUGAUGGUCCCUGGGUUUUCUU | 824 |
| D-1154 | 1152-1172 | CCCAGGGACCAUCAAAGUGG | 156 | ACCACUUUGAUGGUCCCUGGGUU | 825 |
| D-1155 | 1153-1173 | CCAGGGACCAUCAAAGUGGG | 157 | UCCCACUUUGAUGGUCCCUGGUU | 826 |
| D-1156 | 1156-1176 | GGGACCAUCAAAGUGGGAGA | 158 | AUCUCCCACUUUGAUGGUCCCUU | 827 |
| D-1157 | 1170-1190 | GGGAGACCCUGUGUACCUGC | 159 | AGCAGGUACACAGGGUCUCCCUU | 828 |
| D-1158 | 1182-1202 | GUACCUGCUGGGCCAGUAAU | 160 | AAUUACUGGCCCAGCAGGUACUU | 829 |
| D-1159 | 1187-1207 | UGCUGGGCCAGUAAUGGGAA | 161 | AUUCCCAUUACUGGCCCAGCAUU | 830 |
| D-1160 | 1239-1259 | AAAUGUUCUCAAAAAUGACA | 162 | UUGUCAUUUUUGAGAACAUUUUU | 831 |
| D-1161 | 1240-1260 | AAUGUUCUCAAAAAUGACAA | 163 | AUUGUCAUUUUUGAGAACAUUUU | 832 |
| D-1162 | 1250-1270 | AAAAUGACAACACUUGAAGC | 164 | UGCUUCAAGUGUUGUCAUUUUUU | 833 |
| D-1163; | 1251-1271 | AAAUGACAACACUUGAAGCA | 165 | AUGCUUCAAGUGUUGUCAUUUUU | 834 |
| D-2009 | | | | | |
| D-1164 | 1252-1272 | AAUGACAACACUUGAAGCAU | 166 | AAUGCUUCAAGUGUUGUCAUUUU | 835 |
| D-1165 | 1254-1274 | UGACAACACUUGAAGCAUGG | 167 | ACCAUGCUUCAAGUGUUGUCAUU | 836 |
| D-1166; | 1255-1275 | GACAACACUUGAAGCAUGGU | 168 | AACCAUGCUUCAAGUGUUGUCUU | 837 |
| D-2058; | | | | | |
| D-2210; | | | | | |
| D-2218 | | | | | |
| D-1167 | 1256-1276 | ACAACACUUGAAGCAUGGUG | 169 | ACACCAUGCUUCAAGUGUUGUUU | 838 |
| D-1168; | 1260-1280 | CACUUGAAGCAUGGUGUUUC | 170 | UGAAACACCAUGCUUCAAGUGUU | 839 |
| D-2010 | | | | | |
| D-1169 | 1262-1282 | CUUGAAGCAUGGUGUUUCAG | 171 | UCUGAAACACCAUGCUUCAAGUU | 840 |
| D-1170; | 1343-1363 | CUGGUGUCUCAAUGCUUCAA | 172 | AUUGAAGCAUUGAGACACCAGUU | 841 |
| D-2046 | | | | | |
| D-1171; | 1344-1364 | UGGUGUCUCAAUGCUUCAAU | 173 | AAUUGAAGCAUUGAGACACCAUU | 842 |
| D-2013 | | | | | |
| D-1172; | 1345-1365 | GGUGUCUCAAUGCUUCAAUG | 174 | ACAUUGAAGCAUUGAGACACCUU | 843 |
| D-2304 | | | | | |
| D-1173; | 1346-1366 | GUGUCUCAAUGCUUCAAUGU | 175 | AACAUUGAAGCAUUGAGACACUU | 844 |
| D-2305; | | | | | |
| D-2494; | | | | | |
| D-2506; | | | | | |
| D-2512 | | | | | |
| D-1174; | 1347-1367 | UGUCUCAAUGCUUCAAUGUC | 176 | AGACAUUGAAGCAUUGAGACAUU | 845 |
| D-2047 | | | | | |
| D-1175; | 1349-1369 | UCUCAAUGCUUCAAUGUCCC | 177 | UGGGACAUUGAAGCAUUGAGAUU | 846 |
| D-2306 | | | | | |
| D-1176; | 1350-1370 | CUCAAUGCUUCAAUGUCCCA | 178 | AUGGGACAUUGAAGCAUUGAGUU | 847 |
| D-2052; | | | | | |
| D-2203; | | | | | |
| D-2211 | | | | | |
| D-1177; | 1352-1372 | CAAUGCUUCAAUGUCCCAGU | 179 | AACUGGGACAUUGAAGCAUUGUU | 848 |
| D-2042; | | | | | |
| D-2162; | | | | | |
| D-2169; | | | | | |
| D-2183; | | | | | |
| D-2184; | | | | | |
| D-2185; | | | | | |
| D-2186; | | | | | |
| D-2187; | | | | | |
| D-2291; | | | | | |
| D-2292; | | | | | |
| D-2293; | | | | | |
| D-2294; | | | | | |
| D-2295; | | | | | |
| D-2296; | | | | | |
| D-2297; | | | | | |
| D-2298; | | | | | |
| D-2299; | | | | | |
| D-2388 | | | | | |
| D-1178; | 1354-1374 | AUGCUUCAAUGUCCCAGUGC | 180 | UGCACUGGGACAUUGAAGCAUUU | 849 |
| D-2308 | | | | | |
| D-1179; | 1355-1375 | UGCUUCAAUGUCCCAGUGCA | 181 | UUGCACUGGGACAUUGAAGCAUU | 850 |
| D-2043; | | | | | |
| D-2205; | | | | | |
| D-2213 | | | | | |
| D-1180; | 1429-1449 | AAUGACAAGACAGGAUUCUG | 182 | UCAGAAUCCUGUCUUGUCAUUUU | 851 |
| D-2044 | | | | | |
| D-1181 | 1430-1450 | AUGACAAGACAGGAUUCUGA | 183 | UUCAGAAUCCUGUCUUGUCAUUU | 852 |
| D-1182; | 1432-1452 | GACAAGACAGGAUUCUGAAA | 184 | UUUUCAGAAUCCUGUCUUGUCUU | 853 |
| D-2014 | | | | | |
| D-1183 | 1435-1455 | AAGACAGGAUUCUGAAAACU | 185 | AAGUUUUCAGAAUCCUGUCUUUU | 854 |
| D-1184; | 1438-1458 | ACAGGAUUCUGAAAACUCCC | 186 | AGGGAGUUUUCAGAAUCCUGUUU | 855 |
| D-2053; | | | | | |
| D-2209; | | | | | |
| D-2217 | | | | | |
| D-1185; | 1456-1476 | CCCGUUUAACUGAUUAUGGA | 187 | UUCCAUAAUCAGUUAAACGGGUU | 856 |
| D-2008 | | | | | |
| D-1186 | 1460-1480 | UUUAACUGAUUAUGGAAUAG | 188 | ACUAUUCCAUAAUCAGUUAAAUU | 857 |
| D-1187 | 1461-1481 | UUAACUGAUUAUGGAAUAGU | 189 | AACUAUUCCAUAAUCAGUUAAUU | 858 |
| D-1188 | 1463-1483 | AACUGAUUAUGGAAUAGUUC | 190 | AGAACUAUUCCAUAAUCAGUUUU | 859 |
| D-1189; | 1464-1484 | ACUGAUUAUGGAAUAGUUCU | 191 | AAGAACUAUUCCAUAAUCAGUUU | 860 |
| D-2040 | | | | | |
| D-1190; | 1465-1485 | CUGAUUAUGGAAUAGUUCUU | 192 | AAAGAACUAUUCCAUAAUCAGUU | 861 |
| D-2062 | | | | | |
| D-1191; | 1467-1487 | GAUUAUGGAAUAGUUCUUUC | 193 | AGAAAGAACUAUUCCAUAAUCUU | 862 |
| D-2011 | | | | | |
| D-1192; | 1468-1488 | AUUAUGGAAUAGUUCUUUCU | 194 | AAGAAAGAACUAUUCCAUAAUUU | 863 |
| D-2012 | | | | | |
| D-1193 | 1522-1542 | UUGCAUCCUGUCACUACCAC | 195 | AGUGGUAGUGACAGGAUGCAAUU | 864 |
| D-1194; | 1650-1670 | CACCCCAAAUAUGGCUGGAA | 196 | AUUCCAGCCAUAUUUGGGGUGUU | 865 |
| D-2051 | | | | | |
| D-1195 | 1652-1672 | CCCCAAAUAUGGCUGGAAUG | 197 | ACAUUCCAGCCAUAUUUGGGGUU | 866 |
| D-1196 | 1688-1708 | CUCAAGCCCCGGGCUAGCUU | 198 | AAAGCUAGCCCGGGGCUUGAGUU | 867 |
| D-1197 | 1689-1709 | UCAAGCCCCGGGCUAGCUUU | 199 | AAAAGCUAGCCCGGGGCUUGAUU | 868 |
| D-1198 | 1691-1711 | AAGCCCCGGGCUAGCUUUUG | 200 | UCAAAAGCUAGCCCGGGGCUUUU | 869 |
| D-1199 | 1692-1712 | AGCCCCGGGCUAGCUUUUGA | 201 | UUCAAAAGCUAGCCCGGGGCUUU | 870 |
| D-1200 | 1693-1713 | GCCCCGGGCUAGCUUUUGAA | 202 | UUUCAAAAGCUAGCCCGGGGCUU | 871 |
| D-1201 | 1695-1715 | CCCGGGCUAGCUUUUGAAAU | 203 | AAUUUCAAAAGCUAGCCCGGGUU | 872 |
| D-1202 | 1699-1719 | GGCUAGCUUUUGAAAUGGCA | 204 | AUGCCAUUUCAAAAGCUAGCCUU | 873 |
| D-1203 | 1718-1738 | AUAAAGACUGAGGUGACCUU | 205 | AAAGGUCACCUCAGUCUUUAUUU | 874 |
| D-1204; | 1747-1767 | CUGCAGAUAUUAAUUUUCCA | 206 | AUGGAAAAUUAAUAUCUGCAGUU | 875 |
| D-2055 | | | | | |
| D-1205 | 1752-1772 | GAUAUUAAUUUUCCAUAGAU | 207 | AAUCUAUGGAAAAUUAAUAUCUU | 876 |
| D-1206 | 1753-1773 | AUAUUAAUUUUCCAUAGAUC | 208 | AGAUCUAUGGAAAAUUAAUAUUU | 877 |
| D-1207 | 1757-1777 | UAAUUUUCCAUAGAUCUGGA | 209 | AUCCAGAUCUAUGGAAAAUUAUU | 878 |
| D-1208 | 1758-1778 | AAUUUUCCAUAGAUCUGGAU | 210 | AAUCCAGAUCUAUGGAAAAUUUU | 879 |
| D-1209 | 1759-1779 | AUUUUCCAUAGAUCUGGAUC | 211 | AGAUCCAGAUCUAUGGAAAAUUU | 880 |
| D-1210 | 1761-1781 | UUUCCAUAGAUCUGGAUCUG | 212 | ACAGAUCCAGAUCUAUGGAAAUU | 881 |
| D-1211 | 1788-1808 | UGCUUCUCAGACAGCAUUGG | 213 | UCCAAUGCUGUCUGAGAAGCAUU | 882 |
| D-1212 | 1789-1809 | GCUUCUCAGACAGCAUUGGA | 214 | AUCCAAUGCUGUCUGAGAAGCUU | 883 |
| D-1213; | 1794-1814 | UCAGACAGCAUUGGAUUUCC | 215 | AGGAAAUCCAAUGCUGUCUGAUU | 884 |
| D-2059; | | | | | |
| D-2206; | | | | | |
| D-2214 | | | | | |
| D-1214 | 1795-1815 | CAGACAGCAUUGGAUUUCCU | 216 | UAGGAAAUCCAAUGCUGUCUGUU | 885 |
| D-1215; | 1796-1816 | AGACAGCAUUGGAUUUCCUA | 217 | UUAGGAAAUCCAAUGCUGUCUUU | 886 |
| D-2061; | | | | | |
| D-2208; | | | | | |
| D-2216; | | | | | |
| D-2267 | | | | | |
| D-1216 | 1810-1830 | UUCCUAAAGGUGCUCAGGAG | 218 | ACUCCUGAGCACCUUUAGGAAUU | 887 |
| D-1217 | 1849-1869 | AGGACCCCUGGAUCCUUGCC | 219 | UGGCAAGGAUCCAGGGGUCCUUU | 888 |
| D-1218 | 1854-1874 | CCCUGGAUCCUUGCCAUUCC | 220 | AGGAAUGGCAAGGAUCCAGGGUU | 889 |
| D-1219 | 1856-1876 | CUGGAUCCUUGCCAUUCCCC | 221 | AGGGGAAUGGCAAGGAUCCAGUU | 890 |
| D-1220; | 1858-1878 | GGAUCCUUGCCAUUCCCCUC | 222 | UGAGGGGAAUGGCAAGGAUCCUU | 891 |
| D-2054 | | | | | |
| D-1221 | 1859-1879 | GAUCCUUGCCAUUCCCCUCA | 223 | AUGAGGGGAAUGGCAAGGAUCUU | 892 |
| D-1222 | 1862-1882 | CCUUGCCAUUCCCCUCAGCU | 224 | UAGCUGAGGGGAAUGGCAAGGUU | 893 |
| D-1223 | 1863-1883 | CUUGCCAUUCCCCUCAGCUA | 225 | UUAGCUGAGGGGAAUGGCAAGUU | 894 |
| D-1224 | 1866-1886 | GCCAUUCCCCUCAGCUAAUG | 226 | UCAUUAGCUGAGGGGAAUGGCUU | 895 |
| D-1225 | 1868-1888 | CAUUCCCCUCAGCUAAUGAC | 227 | AGUCAUUAGCUGAGGGGAAUGUU | 896 |
| D-1226 | 1886-1906 | ACGGAGUGCUCCUUCUCCAG | 228 | ACUGGAGAAGGAGCACUCCGUUU | 897 |
| D-1227 | 1976-1996 | GAAAACCUUUAAAGGGGGAA | 229 | UUUCCCCCUUUAAAGGUUUUCUU | 898 |
| D-1228; | 2004-2024 | CAUAUGUCAGUUGUUUAAAA | 230 | AUUUUAAACAACUGACAUAUGUU | 899 |
| D-2015 | | | | | |
| D-1229 | 2010-2030 | UCAGUUGUUUAAAACCCAAU | 231 | UAUUGGGUUUUAAACAACUGAUU | 900 |
| D-1230; | 2012-2032 | AGUUGUUUAAAACCCAAUAU | 232 | AAUAUUGGGUUUUAAACAACUUU | 901 |
| D-2016 | | | | | |
| D-1231 | 41-61 | AAGGACGCACUGCUCUGAUU | 233 | AAAUCAGAGCAGUGCGUCCUUUU | 902 |
| D-1232 | 1690-1710 | CAAGCCCCGGGCUAGCUUUU | 234 | AAAAAGCUAGCCCGGGGCUUGUU | 903 |
| D-1233 | 1694-1714 | CCCCGGGCUAGCUUUUGAAA | 235 | AUUUCAAAAGCUAGCCCGGGGUU | 904 |
| D-1234 | 1723-1743 | GACUGAGGUGACCUUCAGGA | 236 | UUCCUGAAGGUCACCUCAGUCUU | 905 |
| D-1235 | 1754-1774 | UAUUAAUUUUCCAUAGAUCU | 237 | AAGAUCUAUGGAAAAUUAAUAUU | 906 |
| D-1236; | 1760-1780 | UUUUCCAUAGAUCUGGAUCU | 238 | AAGAUCCAGAUCUAUGGAAAAUU | 907 |
| D-2048 | | | | | |
| D-1237 | 1791-1811 | UUCUCAGACAGCAUUGGAUU | 239 | AAAUCCAAUGCUGUCUGAGAAUU | 908 |
| D-1238 | 1809-1829 | UUUCCUAAAGGUGCUCAGGA | 240 | AUCCUGAGCACCUUUAGGAAAUU | 909 |
| D-1239 | 1855-1875 | CCUGGAUCCUUGCCAUUCCC | 241 | AGGGAAUGGCAAGGAUCCAGGUU | 910 |
| D-1240 | 1861-1881 | UCCUUGCCAUUCCCCUCAGC | 242 | AGCUGAGGGGAAUGGCAAGGAUU | 911 |
| D-1241 | 1867-1887 | CCAUUCCCCUCAGCUAAUGA | 243 | AUCAUUAGCUGAGGGGAAUGGUU | 912 |
| D-1242 | 1977-1997 | AAAACCUUUAAAGGGGGAAA | 244 | UUUUCCCCCUUUAAAGGUUUUUU | 913 |
| D-1243; | 2014-2034 | UUGUUUAAAACCCAAUAUCU | 245 | UAGAUAUUGGGUUUUAAACAAUU | 914 |
| D-2017; | | | | | |
| D-2204; | | | | | |
| D-2212 | | | | | |
| D-1244 | 2055-2075 | CUCUAAGAUCUGAUGAAGUA | 246 | AUACUUCAUCAGAUCUUAGAGUU | 915 |
| D-1245; | 2057-2077 | CUAAGAUCUGAUGAAGUAUA | 247 | AUAUACUUCAUCAGAUCUUAGUU | 916 |
| D-2045; | | | | | |
| D-2166; | | | | | |
| D-2173 | | | | | |
| D-1246; | 2058-2078 | UAAGAUCUGAUGAAGUAUAU | 248 | AAUAUACUUCAUCAGAUCUUAUU | 917 |
| D-2303 | | | | | |
| D-1247; | 2059-2079 | AAGAUCUGAUGAAGUAUAUU | 249 | AAAUAUACUUCAUCAGAUCUUUU | 918 |
| D-2056 | | | | | |
| D-1248; | 2066-2086 | GAUGAAGUAUAUUUUUUAUU | 250 | AAAUAAAAAAUAUACUUCAUCUU | 919 |
| D-2018 | | | | | |
| D-1249; | 2079-2099 | UUUUAUUGCCAUUUUGUCCU | 251 | AAGGACAAAAUGGCAAUAAAAUU | 920 |
| D-2019 | | | | | |
| D-1250 | 2080-2100 | UUUAUUGCCAUUUUGUCCUU | 252 | AAAGGACAAAAUGGCAAUAAAUU | 921 |
| D-1251 | 2081-2101 | UUAUUGCCAUUUUGUCCUUU | 253 | AAAAGGACAAAAUGGCAAUAAUU | 922 |
| D-1252; | 2083-2103 | AUUGCCAUUUUGUCCUUUGA | 254 | AUCAAAGGACAAAAUGGCAAUUU | 923 |
| D-2020 | | | | | |
| D-1253; | 2105-2125 | AUAUUGGGAAGUUGACUAAA | 255 | AUUUAGUCAACUUCCCAAUAUUU | 924 |
| D-2021 | | | | | |
| D-1254 | 2109-2129 | UGGGAAGUUGACUAAACUUG | 256 | UCAAGUUUAGUCAACUUCCCAUU | 925 |
| D-1255 | 2110-2130 | GGGAAGUUGACUAAACUUGA | 257 | UUCAAGUUUAGUCAACUUCCCUU | 926 |
| D-1256; | 2111-2131 | GGAAGUUGACUAAACUUGAA | 258 | UUUCAAGUUUAGUCAACUUCCUU | 927 |
| D-2022; | | | | | |
| D-2164; | | | | | |
| D-2171 | | | | | |
| D-1257; | 2144-2164 | ACUGUGAAUAAAUGGAAGCU | 259 | UAGCUUCCAUUUAUUCACAGUUU | 928 |
| D-2023 | | | | | |
| D-1258 | 2148-2168 | UGAAUAAAUGGAAGCUACUU | 260 | AAAGUAGCUUCCAUUUAUUCAUU | 929 |
| D-1259 | 2152-2172 | UAAAUGGAAGCUACUUUGAC | 261 | AGUCAAAGUAGCUUCCAUUUAUU | 930 |
| D-1260 | 2153-2173 | AAAUGGAAGCUACUUUGACU | 262 | UAGUCAAAGUAGCUUCCAUUUUU | 931 |
| D-1261 | 2159-2179 | AAGCUACUUUGACUAGUUUC | 263 | UGAAACUAGUCAAAGUAGCUUUU | 932 |
| D-1262 | 2160-2180 | AGCUACUUUGACUAGUUUCA | 264 | AUGAAACUAGUCAAAGUAGCUUU | 933 |
| D-1263 | 1888-1908 | GGAGUGCUCCUUCUCCAGUU | 265 | AAACUGGAGAAGGAGCACUCCUU | 934 |
| D-1264 | 1979-1999 | AACCUUUAAAGGGGGAAAAG | 266 | ACUUUUCCCCCUUUAAAGGUUUU | 935 |
| D-1265 | 1980-2000 | ACCUUUAAAGGGGGAAAAGG | 267 | UCCUUUUCCCCCUUUAAAGGUUU | 936 |
| D-1266; | 2082-2102 | UAUUGCCAUUUUGUCCUUUG | 268 | UCAAAGGACAAAAUGGCAAUAUU | 937 |
| D-2145; | | | | | |
| D-2492; | | | | | |
| D-2504; | | | | | |
| D-2510 | | | | | |
| D-1267 | 2112-2132 | GAAGUUGACUAAACUUGAAA | 269 | UAUUCAAGUUUAGUCAACUUCUU | 938 |
| D-1268 | 2113-2133 | AAGUUGACUAAACUUGAAAA | 270 | UAUUUCAAGUUUAGUCAACUUUU | 939 |
| D-1269 | 2161-2181 | GCUACUUUGACUAGUUUCAG | 271 | UCUGAAACUAGUCAAAGUAGCUU | 940 |
| D-1270 | 557-577 | UGACUCUCAGUGCAGCCUAC | 272 | UGUAGGCUGCACUGAGAGUCAUU | 941 |
| D-1271 | 604-624 | ACGCCCACCACAAAUGCAGU | 273 | AACUGCAUUUGUGGUGGGCGUUU | 942 |
| D-1272 | 683-703 | CCCAGUGGAUAACCAGCUUC | 274 | AGAAGCUGGUUAUCCACUGGGUU | 943 |
| D-1273 | 763-783 | CAUCAAAUAGCAGACUUGUU | 275 | AAACAAGUCUGCUAUUUGAUGUU | 944 |
| D-1274 | 765-785 | UCAAAUAGCAGACUUGUUCC | 276 | AGGAACAAGUCUGCUAUUUGAUU | 945 |
| D-1275 | 864-884 | CAGGCUAGAGAAGAAAGUUA | 277 | UUAACUUUCUUCUCUAGCCUGUU | 946 |
| D-1276; | 865-885 | AGGCUAGAGAAGAAAGUUAA | 278 | UUUAACUUUCUUCUCUAGCCUUU | 947 |
| D-2064 | | | | | |
| D-1277 | 889-909 | ACCAACUUCAGGCCCAAUAU | 279 | AAUAUUGGGCCUGAAGUUGGUUU | 948 |
| D-1278 | 952-972 | GAGCUUCUUAUUGGUGACGU | 280 | AACGUCACCAAUAAGAAGCUCUU | 949 |
| D-1279 | 955-975 | CUUCUUAUUGGUGACGUGGA | 281 | UUCCACGUCACCAAUAAGAAGUU | 950 |
| D-1280 | 957-977 | UCUUAUUGGUGACGUGGAAC | 282 | AGUUCCACGUCACCAAUAAGAUU | 951 |
| D-1281 | 961-981 | AUUGGUGACGUGGAACUGAA | 283 | UUUCAGUUCCACGUCACCAAUUU | 952 |
| D-1282; | 992-1012 | CUUGUUCCAGAUGCAUUUUA | 284 | UUAAAAUGCAUCUGGAACAAGUU | 953 |
| D-2144 | | | | | |
| D-1283 | 994-1014 | UGUUCCAGAUGCAUUUUAAC | 285 | AGUUAAAAUGCAUCUGGAACAUU | 954 |
| D-1284; | 1057-1077 | CUGGAAACACUGAAGAGUUA | 286 | AUAACUCUUCAGUGUUUCCAGUU | 955 |
| D-2074 | | | | | |
| D-1285; | 1058-1078 | UGGAAACACUGAAGAGUUAU | 287 | AAUAACUCUUCAGUGUUUCCAUU | 956 |
| D-2125 | | | | | |
| D-1286; | 1069-1089 | AAGAGUUAUCGCCAGUGUGA | 288 | AUCACACUGGCGAUAACUCUUUU | 957 |
| D-2138 | | | | | |
| D-1287 | 1070-1090 | AGAGUUAUCGCCAGUGUGAC | 289 | AGUCACACUGGCGAUAACUCUUU | 958 |
| D-1288; | 1104-1124 | GUUAUAUGGAAAAUCACCAC | 290 | AGUGGUGAUUUUCCAUAUAACUU | 959 |
| D-2140 | | | | | |
| D-1289 | 1141-1161 | GUGCUGGAAAACCCAGGGAC | 291 | AGUCCCUGGGUUUUCCAGCACUU | 960 |
| D-1290 | 1142-1162 | UGCUGGAAAACCCAGGGACC | 292 | UGGUCCCUGGGUUUUCCAGCAUU | 961 |
| D-1291 | 1143-1163 | GCUGGAAAACCCAGGGACCA | 293 | AUGGUCCCUGGGUUUUCCAGCUU | 962 |
| D-1292 | 1148-1168 | AAAACCCAGGGACCAUCAAA | 294 | AUUUGAUGGUCCCUGGGUUUUUU | 963 |
| D-1293 | 1149-1169 | AAACCCAGGGACCAUCAAAG | 295 | ACUUUGAUGGUCCCUGGGUUUUU | 964 |
| D-1294 | 1150-1170 | AACCCAGGGACCAUCAAAGU | 296 | AACUUUGAUGGUCCCUGGGUUUU | 965 |
| D-1295 | 1151-1171 | ACCCAGGGACCAUCAAAGUG | 297 | ACACUUUGAUGGUCCCUGGGUUU | 966 |
| D-1296 | 1168-1188 | GUGGGAGACCCUGUGUACCU | 298 | AAGGUACACAGGGUCUCCCACUU | 967 |
| D-1297 | 1188-1208 | GCUGGGCCAGUAAUGGGAAC | 299 | AGUUCCCAUUACUGGCCCAGCUU | 968 |
| D-1298; | 1248-1268 | CAAAAAUGACAACACUUGAA | 300 | AUUCAAGUGUUGUCAUUUUUGUU | 969 |
| D-2067 | | | | | |
| D-1299; | 1253-1273 | AUGACAACACUUGAAGCAUG | 301 | ACAUGCUUCAAGUGUUGUCAUUU | 970 |
| D-2119; | | | | | |
| D-2491; | | | | | |
| D-2503; | | | | | |
| D-2509 | | | | | |
| D-1300 | 1261-1281 | ACUUGAAGCAUGGUGUUUCA | 302 | AUGAAACACCAUGCUUCAAGUUU | 971 |
| D-1301 | 1306-1326 | AAAUUUGUGAUUUUCACAUU | 303 | AAAUGUGAAAAUCACAAAUUUUU | 972 |
| D-1302; | 1353-1373 | AAUGCUUCAAUGUCCCAGUG | 304 | ACACUGGGACAUUGAAGCAUUUU | 973 |
| D-2307 | | | | | |
| D-1303 | 1428-1448 | AAAUGACAAGACAGGAUUCU | 305 | AAGAAUCCUGUCUUGUCAUUUUU | 974 |
| D-1304; | 1469-1489 | UUAUGGAAUAGUUCUUUCUC | 306 | AGAGAAAGAACUAUUCCAUAAUU | 975 |
| D-2149 | | | | | |
| D-1305 | 1470-1490 | UAUGGAAUAGUUCUUUCUCC | 307 | AGGAGAAAGAACUAUUCCAUAUU | 976 |
| D-1306 | 1474-1494 | GAAUAGUUCUUUCUCCUGCU | 308 | AAGCAGGAGAAAGAACUAUUCUU | 977 |
| D-1307 | 1475-1495 | AAUAGUUCUUUCUCCUGCUU | 309 | AAAGCAGGAGAAAGAACUAUUUU | 978 |
| D-1308 | 1523-1543 | UGCAUCCUGUCACUACCACU | 310 | AAGUGGUAGUGACAGGAUGCAUU | 979 |
| D-1309 | 1524-1544 | GCAUCCUGUCACUACCACUC | 311 | AGAGUGGUAGUGACAGGAUGCUU | 980 |
| D-1310; | 1696-1716 | CCGGGCUAGCUUUUGAAAUG | 312 | ACAUUUCAAAAGCUAGCCCGGUU | 981 |
| D-2139 | | | | | |
| D-1311; | 1697-1717 | CGGGCUAGCUUUUGAAAUGG | 313 | ACCAUUUCAAAAGCUAGCCCGUU | 982 |
| D-2073 | | | | | |
| D-1312 | 1721-1741 | AAGACUGAGGUGACCUUCAG | 314 | ACUGAAGGUCACCUCAGUCUUUU | 983 |
| D-1313 | 1728-1748 | AGGUGACCUUCAGGAAGCAC | 315 | AGUGCUUCCUGAAGGUCACCUUU | 984 |
| D-1314 | 1764-1784 | CCAUAGAUCUGGAUCUGGCC | 316 | AGGCCAGAUCCAGAUCUAUGGUU | 985 |
| D-1315; | 1805-1825 | UGGAUUUCCUAAAGGUGCUC | 317 | UGAGCACCUUUAGGAAAUCCAUU | 986 |
| D-2130 | | | | | |
| D-1316 | 1807-1827 | GAUUUCCUAAAGGUGCUCAG | 318 | ACUGAGCACCUUUAGGAAAUCUU | 987 |
| D-1317 | 1811-1831 | UCCUAAAGGUGCUCAGGAGG | 319 | UCCUCCUGAGCACCUUUAGGAUU | 988 |
| D-1318 | 1846-1866 | UGGAGGACCCCUGGAUCCUU | 320 | AAAGGAUCCAGGGGUCCUCCAUU | 989 |
| D-1319 | 1847-1867 | GGAGGACCCCUGGAUCCUUG | 321 | ACAAGGAUCCAGGGGUCCUCCUU | 990 |
| D-1320 | 1848-1868 | GAGGACCCCUGGAUCCUUGC | 322 | AGCAAGGAUCCAGGGGUCCUCUU | 991 |
| D-1321 | 1887-1907 | CGGAGUGCUCCUUCUCCAGU | 323 | AACUGGAGAAGGAGCACUCCGUU | 992 |
| D-1322 | 39-59 | AGAAGGACGCACUGCUCUGA | 324 | AUCAGAGCAGUGCGUCCUUCUUU | 993 |
| D-1323 | 53-73 | CUCUGAUUGGCCCGGAAGGG | 325 | ACCCUUCCGGGCCAAUCAGAGUU | 994 |
| D-1324 | 54-74 | UCUGAUUGGCCCGGAAGGGU | 326 | AACCCUUCCGGGCCAAUCAGAUU | 995 |
| D-1325 | 55-75 | CUGAUUGGCCCGGAAGGGUU | 327 | AAACCCUUCCGGGCCAAUCAGUU | 996 |
| D-1326 | 102-122 | GGCCAAAGGCCGCACCUUCC | 328 | AGGAAGGUGCGGCCUUUGGCCUU | 997 |
| D-1327 | 168-188 | CCUCGCGGAGAAGCCAGCCA | 329 | AUGGCUGGCUUCUCCGCGAGGUU | 998 |
| D-1328 | 174-194 | GGAGAAGCCAGCCAUGGGCG | 330 | ACGCCCAUGGCUGGCUUCUCCUU | 999 |
| D-1329 | 175-195 | GAGAAGCCAGCCAUGGGCGC | 331 | AGCGCCCAUGGCUGGCUUCUCUU | 1000 |
| D-1330 | 474-494 | GAUCAACCAGGAGGGAAACA | 332 | AUGUUUCCCUCCUGGUUGAUCUU | 1001 |
| D-1331 | 499-519 | ACUGCUCGCCAGGAACCUCG | 333 | ACGAGGUUCCUGGCGAGCAGUUU | 1002 |
| D-1332 | 504-524 | UCGCCAGGAACCUCGCCUGG | 334 | ACCAGGCGAGGUUCCUGGCGAUU | 1003 |
| D-1333 | 506-526 | GCCAGGAACCUCGCCUGGUC | 335 | AGACCAGGCGAGGUUCCUGGCUU | 1004 |
| D-1334 | 511-531 | GAACCUCGCCUGGUCCUGAU | 336 | AAUCAGGACCAGGCGAGGUUCUU | 1005 |
| D-1335 | 545-565 | AUGGUGACACCCUGACUCUC | 337 | UGAGAGUCAGGGUGUCACCAUUU | 1006 |
| D-1336 | 546-566 | UGGUGACACCCUGACUCUCA | 338 | AUGAGAGUCAGGGUGUCACCAUU | 1007 |
| D-1337 | 550-570 | GACACCCUGACUCUCAGUGC | 339 | UGCACUGAGAGUCAGGGUGUCUU | 1008 |
| D-1338; | 553-573 | ACCCUGACUCUCAGUGCAGC | 340 | AGCUGCACUGAGAGUCAGGGUUU | 1009 |
| D-2085 | | | | | |
| D-1339 | 680-700 | CCGCCCAGUGGAUAACCAGC | 341 | AGCUGGUUAUCCACUGGGCGGUU | 1010 |
| D-1340 | 720-740 | CCGCCUGGUGCACUUCGAGC | 342 | AGCUCGAAGUGCACCAGGCGGUU | 1011 |
| D-1341 | 721-741 | CGCCUGGUGCACUUCGAGCC | 343 | AGGCUCGAAGUGCACCAGGCGUU | 1012 |
| D-1342 | 722-742 | GCCUGGUGCACUUCGAGCCU | 344 | AAGGCUCGAAGUGCACCAGGCUU | 1013 |
| D-1343 | 723-743 | CCUGGUGCACUUCGAGCCUC | 345 | UGAGGCUCGAAGUGCACCAGGUU | 1014 |
| D-1344 | 724-744 | CUGGUGCACUUCGAGCCUCA | 346 | AUGAGGCUCGAAGUGCACCAGUU | 1015 |
| D-1345 | 725-745 | UGGUGCACUUCGAGCCUCAC | 347 | UGUGAGGCUCGAAGUGCACCAUU | 1016 |
| D-1346 | 726-746 | GGUGCACUUCGAGCCUCACA | 348 | AUGUGAGGCUCGAAGUGCACCUU | 1017 |
| D-1347 | 727-747 | GUGCACUUCGAGCCUCACAU | 349 | AAUGUGAGGCUCGAAGUGCACUU | 1018 |
| D-1348 | 728-748 | UGCACUUCGAGCCUCACAUG | 350 | ACAUGUGAGGCUCGAAGUGCAUU | 1019 |
| D-1349 | 729-749 | GCACUUCGAGCCUCACAUGC | 351 | AGCAUGUGAGGCUCGAAGUGCUU | 1020 |
| D-1350 | 730-750 | CACUUCGAGCCUCACAUGCG | 352 | UCGCAUGUGAGGCUCGAAGUGUU | 1021 |
| D-1351 | 731-751 | ACUUCGAGCCUCACAUGCGA | 353 | AUCGCAUGUGAGGCUCGAAGUUU | 1022 |
| D-1352 | 732-752 | CUUCGAGCCUCACAUGCGAC | 354 | AGUCGCAUGUGAGGCUCGAAGUU | 1023 |
| D-1353 | 733-753 | UUCGAGCCUCACAUGCGACC | 355 | AGGUCGCAUGUGAGGCUCGAAUU | 1024 |
| D-1354 | 734-754 | UCGAGCCUCACAUGCGACCG | 356 | UCGGUCGCAUGUGAGGCUCGAUU | 1025 |
| D-1355 | 735-755 | CGAGCCUCACAUGCGACCGA | 357 | AUCGGUCGCAUGUGAGGCUCGUU | 1026 |
| D-1356 | 738-758 | GCCUCACAUGCGACCGAGAC | 358 | AGUCUCGGUCGCAUGUGAGGCUU | 1027 |
| D-1357 | 825-845 | CUUGAUCCUUUCUGAGGCGU | 359 | AACGCCUCAGAAAGGAUCAAGUU | 1028 |
| D-1358 | 847-867 | CUGGCGGAUCUCAACUCCAG | 360 | ACUGGAGUUGAGAUCCGCCAGUU | 1029 |
| D-1359 | 848-868 | UGGCGGAUCUCAACUCCAGG | 361 | ACCUGGAGUUGAGAUCCGCCAUU | 1030 |
| D-1360 | 923-943 | GCGAUGUCUAUGCAGAGGAU | 362 | AAUCCUCUGCAUAGACAUCGCUU | 1031 |
| D-1361 | 925-945 | GAUGUCUAUGCAGAGGAUUC | 363 | AGAAUCCUCUGCAUAGACAUCUU | 1032 |
| D-1362 | 927-947 | UGUCUAUGCAGAGGAUUCUU | 364 | AAAGAAUCCUCUGCAUAGACAUU | 1033 |
| D-1363; | 928-948 | GUCUAUGCAGAGGAUUCUUG | 365 | ACAAGAAUCCUCUGCAUAGACUU | 1034 |
| D-2084 | | | | | |
| D-1364 | 929-949 | UCUAUGCAGAGGAUUCUUGG | 366 | ACCAAGAAUCCUCUGCAUAGAUU | 1035 |
| D-1365 | 930-950 | CUAUGCAGAGGAUUCUUGGG | 367 | UCCCAAGAAUCCUCUGCAUAGUU | 1036 |
| D-1366 | 984-1004 | GGUGAUGGCUUGUUCCAGAU | 368 | AAUCUGGAACAAGCCAUCACCUU | 1037 |
| D-1367; | 985-1005 | GUGAUGGCUUGUUCCAGAUG | 369 | ACAUCUGGAACAAGCCAUCACUU | 1038 |
| D-2083; | | | | | |
| D-2244; | | | | | |
| D-2249 | | | | | |
| D-1368 | 989-1009 | UGGCUUGUUCCAGAUGCAUU | 370 | AAAUGCAUCUGGAACAAGCCAUU | 1039 |
| D-1369 | 1005-1025 | CAUUUUAACCACAGUGGACC | 371 | AGGUCCACUGUGGUUAAAAUGUU | 1040 |
| D-1370 | 1007-1027 | UUUUAACCACAGUGGACCCA | 372 | AUGGGUCCACUGUGGUUAAAAUU | 1041 |
| D-1371 | 1008-1028 | UUUAACCACAGUGGACCCAG | 373 | UCUGGGUCCACUGUGGUUAAAUU | 1042 |
| D-1372 | 1118-1138 | CACCACUCUUUGGGCAGUAU | 374 | AAUACUGCCCAAAGAGUGGUGUU | 1043 |
| D-1373 | 1119-1139 | ACCACUCUUUGGGCAGUAUU | 375 | AAAUACUGCCCAAAGAGUGGUUU | 1044 |
| D-1374 | 1125-1145 | CUUUGGGCAGUAUUUUGUGC | 376 | AGCACAAAAUACUGCCCAAAGUU | 1045 |
| D-1375; | 1126-1146 | UUUGGGCAGUAUUUUGUGCU | 377 | AAGCACAAAAUACUGCCCAAAUU | 1046 |
| D-2071 | | | | | |
| D-1376 | 1127-1147 | UUGGGCAGUAUUUUGUGCUG | 378 | ACAGCACAAAAUACUGCCCAAUU | 1047 |
| D-1377 | 1128-1148 | UGGGCAGUAUUUUGUGCUGG | 379 | UCCAGCACAAAAUACUGCCCAUU | 1048 |
| D-1378 | 1130-1150 | GGCAGUAUUUUGUGCUGGAA | 380 | UUUCCAGCACAAAAUACUGCCUU | 1049 |
| D-1379 | 1135-1155 | UAUUUUGUGCUGGAAAACCC | 381 | UGGGUUUUCCAGCACAAAAUAUU | 1050 |
| D-1380 | 1136-1156 | AUUUUGUGCUGGAAAACCCA | 382 | AUGGGUUUUCCAGCACAAAAUUU | 1051 |
| D-1381; | 1206-1226 | ACCGUAUGUCCUGGAAUAUU | 383 | UAAUAUUCCAGGACAUACGGUUU | 1052 |
| D-2154 | | | | | |
| D-1382; | 1207-1227 | CCGUAUGUCCUGGAAUAUUA | 384 | AUAAUAUUCCAGGACAUACGGUU | 1053 |
| D-2066 | | | | | |
| D-1383; | 1209-1229 | GUAUGUCCUGGAAUAUUAGA | 385 | AUCUAAUAUUCCAGGACAUACUU | 1054 |
| D-2063 | | | | | |
| D-1384; | 1210-1230 | UAUGUCCUGGAAUAUUAGAU | 386 | AAUCUAAUAUUCCAGGACAUAUU | 1055 |
| D-2142 | | | | | |
| D-1385; | 1211-1231 | AUGUCCUGGAAUAUUAGAUG | 387 | ACAUCUAAUAUUCCAGGACAUUU | 1056 |
| D-2301; | | | | | |
| D-2441; | | | | | |
| D-2445 | | | | | |
| D-1386; | 1212-1232 | UGUCCUGGAAUAUUAGAUGC | 388 | AGCAUCUAAUAUUCCAGGACAUU | 1057 |
| D-2081; | | | | | |
| D-2245; | | | | | |
| D-2250; | | | | | |
| D-2312; | | | | | |
| D-2317; | | | | | |
| D-2322; | | | | | |
| D-2327; | | | | | |
| D-2332; | | | | | |
| D-2337; | | | | | |
| D-2342; | | | | | |
| D-2347; | | | | | |
| D-2352; | | | | | |
| D-2357; | | | | | |
| D-2396 | | | | | |
| D-1387; | 1213-1233 | GUCCUGGAAUAUUAGAUGCC | 389 | AGGCAUCUAAUAUUCCAGGACUU | 1058 |
| D-2080; | | | | | |
| D-2246; | | | | | |
| D-2251; | | | | | |
| D-2264; | | | | | |
| D-2276; | | | | | |
| D-2277; | | | | | |
| D-2278; | | | | | |
| D-2279; | | | | | |
| D-2280; | | | | | |
| D-2281; | | | | | |
| D-2282; | | | | | |
| D-2283; | | | | | |
| D-2284; | | | | | |
| D-2285; | | | | | |
| D-2286; | | | | | |
| D-2287; | | | | | |
| D-2288; | | | | | |
| D-2289; | | | | | |
| D-2311; | | | | | |
| D-2316; | | | | | |
| D-2321; | | | | | |
| D-2326; | | | | | |
| D-2331; | | | | | |
| D-2336; | | | | | |
| D-2341; | | | | | |
| D-2346; | | | | | |
| D-2351; | | | | | |
| D-2356; | | | | | |
| D-2395 | | | | | |
| D-1388; | 1214-1234 | UCCUGGAAUAUUAGAUGCCU | 390 | AAGGCAUCUAAUAUUCCAGGAUU | 1059 |
| D-2078; | | | | | |
| D-2248; | | | | | |
| D-2253; | | | | | |
| D-2265; | | | | | |
| D-2309; | | | | | |
| D-2314; | | | | | |
| D-2319; | | | | | |
| D-2324; | | | | | |
| D-2329; | | | | | |
| D-2334; | | | | | |
| D-2339; | | | | | |
| D-2344; | | | | | |
| D-2349; | | | | | |
| D-2354; | | | | | |
| D-2393 | | | | | |
| D-1389; | 1215-1235 | CCUGGAAUAUUAGAUGCCUU | 391 | AAAGGCAUCUAAUAUUCCAGGUU | 1060 |
| D-2077 | | | | | |
| D-1390; | 1216-1236 | CUGGAAUAUUAGAUGCCUUU | 392 | AAAAGGCAUCUAAUAUUCCAGUU | 1061 |
| D-2076 | | | | | |
| D-1391; | 1269-1289 | CAUGGUGUUUCAGAACUGAG | 393 | UCUCAGUUCUGAAACACCAUGUU | 1062 |
| D-2150 | | | | | |
| D-1392 | 1270-1290 | AUGGUGUUUCAGAACUGAGA | 394 | AUCUCAGUUCUGAAACACCAUUU | 1063 |
| D-1393 | 1271-1291 | UGGUGUUUCAGAACUGAGAC | 395 | AGUCUCAGUUCUGAAACACCAUU | 1064 |
| D-1394 | 1272-1292 | GGUGUUUCAGAACUGAGACC | 396 | AGGUCUCAGUUCUGAAACACCUU | 1065 |
| D-1395; | 1935-1955 | GAGGAGAAGAAAAGUGAUUC | 397 | UGAAUCACUUUUCUUCUCCUCUU | 1066 |
| D-2131 | | | | | |
| D-1396; | 1939-1959 | AGAAGAAAAGUGAUUCAGUG | 398 | UCACUGAAUCACUUUUCUUCUUU | 1067 |
| D-2070; | | | | | |
| D-2471 | | | | | |
| D-1397; | 1940-1960 | GAAGAAAAGUGAUUCAGUGA | 399 | AUCACUGAAUCACUUUUCUUCUU | 1068 |
| D-2151 | | | | | |
| D-1398 | 1943-1963 | GAAAAGUGAUUCAGUGAUUU | 400 | AAAAUCACUGAAUCACUUUUCUU | 1069 |
| D-1399; | 1945-1965 | AAAGUGAUUCAGUGAUUUCA | 401 | AUGAAAUCACUGAAUCACUUUUU | 1070 |
| D-2141 | | | | | |
| D-1400; | 1970-1990 | ACUACUGAAAACCUUUAAAG | 402 | ACUUUAAAGGUUUUCAGUAGUUU | 1071 |
| D-2069 | | | | | |
| D-1401; | 1972-1992 | UACUGAAAACCUUUAAAGGG | 403 | ACCCUUUAAAGGUUUUCAGUAUU | 1072 |
| D-2068 | | | | | |
| D-1402; | 2048-2068 | UGUAUAACUCUAAGAUCUGA | 404 | AUCAGAUCUUAGAGUUAUACAUU | 1073 |
| D-2065 | | | | | |
| D-1403; | 2050-2070 | UAUAACUCUAAGAUCUGAUG | 405 | UCAUCAGAUCUUAGAGUUAUAUU | 1074 |
| D-2302; | | | | | |
| D-2493; | | | | | |
| D-2505; | | | | | |
| D-2511 | | | | | |
| D-1404; | 2051-2071 | AUAACUCUAAGAUCUGAUGA | 406 | UUCAUCAGAUCUUAGAGUUAUUU | 1075 |
| D-2143 | | | | | |
| D-1405; | 2052-2072 | UAACUCUAAGAUCUGAUGAA | 407 | AUUCAUCAGAUCUUAGAGUUAUU | 1076 |
| D-2082; | | | | | |
| D-2313; | | | | | |
| D-2318; | | | | | |
| D-2323; | | | | | |
| D-2328; | | | | | |
| D-2333; | | | | | |
| D-2338; | | | | | |
| D-2343; | | | | | |
| D-2348; | | | | | |
| D-2353; | | | | | |
| D-2358; | | | | | |
| D-2397; | | | | | |
| D-2470 | | | | | |
| D-1406; | 2053-2073 | AACUCUAAGAUCUGAUGAAG | 408 | ACUUCAUCAGAUCUUAGAGUUUU | 1077 |
| D-2137 | | | | | |
| D-1407; | 2054-2074 | ACUCUAAGAUCUGAUGAAGU | 409 | UACUUCAUCAGAUCUUAGAGUUU | 1078 |
| D-2079; | | | | | |
| D-2247; | | | | | |
| D-2252; | | | | | |
| D-2266; | | | | | |
| D-2310; | | | | | |
| D-2315; | | | | | |
| D-2320; | | | | | |
| D-2325; | | | | | |
| D-2330; | | | | | |
| D-2335; | | | | | |
| D-2340; | | | | | |
| D-2345; | | | | | |
| D-2350; | | | | | |
| D-2355; | | | | | |
| D-2394 | | | | | |
| D-1408 | 57-77 | GAUUGGCCCGGAAGGGUUCA | 410 | AUGAACCCUUCCGGGCCAAUCUU | 1079 |
| D-1409 | 89-109 | CCUUUGGGCUCGGGGCCAAA | 411 | AUUUGGCCCCGAGCCCAAAGGUU | 1080 |
| D-1410 | 92-112 | UUGGGCUCGGGGCCAAAGGC | 412 | AGCCUUUGGCCCCGAGCCCAAUU | 1081 |
| D-1411 | 112-132 | CGCACCUUCCCCCAGCGGCC | 413 | AGGCCGCUGGGGGAAGGUGCGUU | 1082 |
| D-1412 | 159-179 | CCGCCGCCACCUCGCGGAGA | 414 | UUCUCCGCGAGGUGGCGGCGGUU | 1083 |
| D-1413 | 205-225 | UCCGCGCUGGCGCGCUUUGU | 415 | AACAAAGCGCGCCAGCGCGGAUU | 1084 |
| D-1414 | 206-226 | CCGCGCUGGCGCGCUUUGUC | 416 | AGACAAAGCGCGCCAGCGCGGUU | 1085 |
| D-1415 | 207-227 | CGCGCUGGCGCGCUUUGUCC | 417 | AGGACAAAGCGCGCCAGCGCGUU | 1086 |
| D-1416; | 215-235 | CGCGCUUUGUCCUCCUCGCG | 418 | ACGCGAGGAGGACAAAGCGCGUU | 1087 |
| D-2091 | | | | | |
| D-1417 | 216-236 | GCGCUUUGUCCUCCUCGCGC | 419 | UGCGCGAGGAGGACAAAGCGCUU | 1088 |
| D-1418 | 217-237 | CGCUUUGUCCUCCUCGCGCA | 420 | UUGCGCGAGGAGGACAAAGCGUU | 1089 |
| D-1419 | 218-238 | GCUUUGUCCUCCUCGCGCAA | 421 | AUUGCGCGAGGAGGACAAAGCUU | 1090 |
| D-1420; | 219-239 | CUUUGUCCUCCUCGCGCAAU | 422 | AAUUGCGCGAGGAGGACAAAGUU | 1091 |
| D-2093 | | | | | |
| D-1421; | 220-240 | UUUGUCCUCCUCGCGCAAUC | 423 | AGAUUGCGCGAGGAGGACAAAUU | 1092 |
| D-2095 | | | | | |
| D-1422 | 222-242 | UGUCCUCCUCGCGCAAUCCC | 424 | AGGGAUUGCGCGAGGAGGACAUU | 1093 |
| D-1423 | 223-243 | GUCCUCCUCGCGCAAUCCCG | 425 | ACGGGAUUGCGCGAGGAGGACUU | 1094 |
| D-1424 | 224-244 | UCCUCCUCGCGCAAUCCCGG | 426 | ACCGGGAUUGCGCGAGGAGGAUU | 1095 |
| D-1425 | 225-245 | CCUCCUCGCGCAAUCCCGGC | 427 | AGCCGGGAUUGCGCGAGGAGGUU | 1096 |
| D-1426 | 229-249 | CUCGCGCAAUCCCGGCCCGG | 428 | ACCGGGCCGGGAUUGCGCGAGUU | 1097 |
| D-1427 | 232-252 | GCGCAAUCCCGGCCCGGGUG | 429 | ACACCCGGGCCGGGAUUGCGCUU | 1098 |
| D-1428 | 233-253 | CGCAAUCCCGGCCCGGGUGG | 430 | ACCACCCGGGCCGGGAUUGCGUU | 1099 |
| D-1429 | 234-254 | GCAAUCCCGGCCCGGGUGGC | 431 | AGCCACCCGGGCCGGGAUUGCUU | 1100 |
| D-1430 | 242-262 | GGCCCGGGUGGCUCGGGGUU | 432 | AAACCCCGAGCCACCCGGGCCUU | 1101 |
| D-1431 | 243-263 | GCCCGGGUGGCUCGGGGUUG | 433 | ACAACCCCGAGCCACCCGGGCUU | 1102 |
| D-1432 | 244-264 | CCCGGGUGGCUCGGGGUUGC | 434 | AGCAACCCCGAGCCACCCGGGUU | 1103 |
| D-1433 | 254-274 | UCGGGGUUGCCGCGCUGGGC | 435 | AGCCCAGCGCGGCAACCCCGAUU | 1104 |
| D-1434 | 258-278 | GGUUGCCGCGCUGGGCCUGA | 436 | AUCAGGCCCAGCGCGGCAACCUU | 1105 |
| D-1435 | 262-282 | GCCGCGCUGGGCCUGACCGC | 437 | AGCGGUCAGGCCCAGCGCGGCUU | 1106 |
| D-1436 | 265-285 | GCGCUGGGCCUGACCGCGGU | 438 | AACCGCGGUCAGGCCCAGCGCUU | 1107 |
| D-1437 | 269-289 | UGGGCCUGACCGCGGUGGCG | 439 | ACGCCACCGCGGUCAGGCCCAUU | 1108 |
| D-1438 | 270-290 | GGGCCUGACCGCGGUGGCGC | 440 | AGCGCCACCGCGGUCAGGCCCUU | 1109 |
| D-1439; | 484-504 | GAGGGAAACAUGGUUACUGC | 441 | AGCAGUAACCAUGUUUCCCUCUU | 1110 |
| D-2122 | | | | | |
| D-1440 | 487-507 | GGAAACAUGGUUACUGCUCG | 442 | ACGAGCAGUAACCAUGUUUCCUU | 1111 |
| D-1441; | 488-508 | GAAACAUGGUUACUGCUCGC | 443 | AGCGAGCAGUAACCAUGUUUCUU | 1112 |
| D-2092 | | | | | |
| D-1442 | 489-509 | AAACAUGGUUACUGCUCGCC | 444 | UGGCGAGCAGUAACCAUGUUUUU | 1113 |
| D-1443 | 490-510 | AACAUGGUUACUGCUCGCCA | 445 | AUGGCGAGCAGUAACCAUGUUUU | 1114 |
| D-1444 | 491-511 | ACAUGGUUACUGCUCGCCAG | 446 | ACUGGCGAGCAGUAACCAUGUUU | 1115 |
| D-1445 | 496-516 | GUUACUGCUCGCCAGGAACC | 447 | AGGUUCCUGGCGAGCAGUAACUU | 1116 |
| D-1446 | 531-551 | UUCCCUGACCUGCGAUGGUG | 448 | UCACCAUCGCAGGUCAGGGAAUU | 1117 |
| D-1447 | 538-558 | ACCUGCGAUGGUGACACCCU | 449 | AAGGGUGUCACCAUCGCAGGUUU | 1118 |
| D-1448 | 566-586 | GUGCAGCCUACACAAAGGAC | 450 | AGUCCUUUGUGUAGGCUGCACUU | 1119 |
| D-1449 | 567-587 | UGCAGCCUACACAAAGGACC | 451 | AGGUCCUUUGUGUAGGCUGCAUU | 1120 |
| D-1450 | 569-589 | CAGCCUACACAAAGGACCUA | 452 | AUAGGUCCUUUGUGUAGGCUGUU | 1121 |
| D-1451; | 570-590 | AGCCUACACAAAGGACCUAC | 453 | AGUAGGUCCUUUGUGUAGGCUUU | 1122 |
| D-2086 | | | | | |
| D-1452 | 571-591 | GCCUACACAAAGGACCUACU | 454 | UAGUAGGUCCUUUGUGUAGGCUU | 1123 |
| D-1453 | 572-592 | CCUACACAAAGGACCUACUA | 455 | AUAGUAGGUCCUUUGUGUAGGUU | 1124 |
| D-1454 | 573-593 | CUACACAAAGGACCUACUAC | 456 | AGUAGUAGGUCCUUUGUGUAGUU | 1125 |
| D-1455; | 576-596 | CACAAAGGACCUACUACUGC | 457 | AGCAGUAGUAGGUCCUUUGUGUU | 1126 |
| D-2110 | | | | | |
| D-1456 | 581-601 | AGGACCUACUACUGCCUAUC | 458 | UGAUAGGCAGUAGUAGGUCCUUU | 1127 |
| D-1457 | 582-602 | GGACCUACUACUGCCUAUCA | 459 | UUGAUAGGCAGUAGUAGGUCCUU | 1128 |
| D-1458; | 584-604 | ACCUACUACUGCCUAUCAAA | 460 | UUUUGAUAGGCAGUAGUAGGUUU | 1129 |
| D-2134 | | | | | |
| D-1459; | 587-607 | UACUACUGCCUAUCAAAACG | 461 | ACGUUUUGAUAGGCAGUAGUAUU | 1130 |
| D-2135 | | | | | |
| D-1460; | 589-609 | CUACUGCCUAUCAAAACGCC | 462 | AGGCGUUUUGAUAGGCAGUAGUU | 1131 |
| D-2098 | | | | | |
| D-1461 | 594-614 | GCCUAUCAAAACGCCCACCA | 463 | AUGGUGGGCGUUUUGAUAGGCUU | 1132 |
| D-1462 | 610-630 | ACCACAAAUGCAGUGCACAA | 464 | AUUGUGCACUGCAUUUGUGGUUU | 1133 |
| D-1463 | 612-632 | CACAAAUGCAGUGCACAAGU | 465 | AACUUGUGCACUGCAUUUGUGUU | 1134 |
| D-1464 | 614-634 | CAAAUGCAGUGCACAAGUGC | 466 | UGCACUUGUGCACUGCAUUUGUU | 1135 |
| D-1465 | 617-637 | AUGCAGUGCACAAGUGCAGA | 467 | AUCUGCACUUGUGCACUGCAUUU | 1136 |
| D-1466 | 621-641 | AGUGCACAAGUGCAGAGUGC | 468 | UGCACUCUGCACUUGUGCACUUU | 1137 |
| D-1467 | 626-646 | ACAAGUGCAGAGUGCACGGC | 469 | AGCCGUGCACUCUGCACUUGUUU | 1138 |
| D-1468 | 627-647 | CAAGUGCAGAGUGCACGGCC | 470 | AGGCCGUGCACUCUGCACUUGUU | 1139 |
| D-1469 | 631-651 | UGCAGAGUGCACGGCCUGGA | 471 | AUCCAGGCCGUGCACUCUGCAUU | 1140 |
| D-1470 | 634-654 | AGAGUGCACGGCCUGGAGAU | 472 | UAUCUCCAGGCCGUGCACUCUUU | 1141 |
| D-1471 | 635-655 | GAGUGCACGGCCUGGAGAUA | 473 | AUAUCUCCAGGCCGUGCACUCUU | 1142 |
| D-1472 | 647-667 | UGGAGAUAGAGGGCAGGGAC | 474 | AGUCCCUGCCCUCUAUCUCCAUU | 1143 |
| D-1473 | 701-721 | UCCUGAAGUCACAGCCCUAC | 475 | AGUAGGGCUGUGACUUCAGGAUU | 1144 |
| D-1474 | 703-723 | CUGAAGUCACAGCCCUACCG | 476 | ACGGUAGGGCUGUGACUUCAGUU | 1145 |
| D-1475 | 705-725 | GAAGUCACAGCCCUACCGCC | 477 | AGGCGGUAGGGCUGUGACUUCUU | 1146 |
| D-1476 | 739-759 | CCUCACAUGCGACCGAGACG | 478 | ACGUCUCGGUCGCAUGUGAGGUU | 1147 |
| D-1477 | 740-760 | CUCACAUGCGACCGAGACGU | 479 | AACGUCUCGGUCGCAUGUGAGUU | 1148 |
| D-1478 | 741-761 | UCACAUGCGACCGAGACGUC | 480 | AGACGUCUCGGUCGCAUGUGAUU | 1149 |
| D-1479 | 742-762 | CACAUGCGACCGAGACGUCC | 481 | AGGACGUCUCGGUCGCAUGUGUU | 1150 |
| D-1480 | 743-763 | ACAUGCGACCGAGACGUCCU | 482 | AAGGACGUCUCGGUCGCAUGUUU | 1151 |
| D-1481 | 746-766 | UGCGACCGAGACGUCCUCAU | 483 | AAUGAGGACGUCUCGGUCGCAUU | 1152 |
| D-1482 | 747-767 | GCGACCGAGACGUCCUCAUC | 484 | UGAUGAGGACGUCUCGGUCGCUU | 1153 |
| D-1483 | 751-771 | CCGAGACGUCCUCAUCAAAU | 485 | UAUUUGAUGAGGACGUCUCGGUU | 1154 |
| D-1484 | 752-772 | CGAGACGUCCUCAUCAAAUA | 486 | AUAUUUGAUGAGGACGUCUCGUU | 1155 |
| D-1485 | 754-774 | AGACGUCCUCAUCAAAUAGC | 487 | UGCUAUUUGAUGAGGACGUCUUU | 1156 |
| D-1486 | 759-779 | UCCUCAUCAAAUAGCAGACU | 488 | AAGUCUGCUAUUUGAUGAGGAUU | 1157 |
| D-1487; | 761-781 | CUCAUCAAAUAGCAGACUUG | 489 | ACAAGUCUGCUAUUUGAUGAGUU | 1158 |
| D-2099 | | | | | |
| D-1488 | 809-829 | CAGACACCAGCCCAUUCUUG | 490 | UCAAGAAUGGGCUGGUGUCUGUU | 1159 |
| D-1489; | 810-830 | AGACACCAGCCCAUUCUUGA | 491 | AUCAAGAAUGGGCUGGUGUCUUU | 1160 |
| D-2100 | | | | | |
| D-1490 | 811-831 | GACACCAGCCCAUUCUUGAU | 492 | AAUCAAGAAUGGGCUGGUGUCUU | 1161 |
| D-1491; | 816-836 | CAGCCCAUUCUUGAUCCUUU | 493 | AAAAGGAUCAAGAAUGGGCUGUU | 1162 |
| D-2101 | | | | | |
| D-1492; | 820-840 | CCAUUCUUGAUCCUUUCUGA | 494 | AUCAGAAAGGAUCAAGAAUGGUU | 1163 |
| D-2112 | | | | | |
| D-1493; | 934-954 | GCAGAGGAUUCUUGGGAUGA | 495 | AUCAUCCCAAGAAUCCUCUGCUU | 1164 |
| D-2102 | | | | | |
| D-1494 | 941-961 | AUUCUUGGGAUGAGCUUCUU | 496 | UAAGAAGCUCAUCCCAAGAAUUU | 1165 |
| D-1495 | 944-964 | CUUGGGAUGAGCUUCUUAUU | 497 | AAAUAAGAAGCUCAUCCCAAGUU | 1166 |
| D-1496 | 947-967 | GGGAUGAGCUUCUUAUUGGU | 498 | AACCAAUAAGAAGCUCAUCCCUU | 1167 |
| D-1497 | 948-968 | GGAUGAGCUUCUUAUUGGUG | 499 | UCACCAAUAAGAAGCUCAUCCUU | 1168 |
| D-1498 | 970-990 | GUGGAACUGAAAAGGGUGAU | 500 | AAUCACCCUUUUCAGUUCCACUU | 1169 |
| D-1499 | 971-991 | UGGAACUGAAAAGGGUGAUG | 501 | ACAUCACCCUUUUCAGUUCCAUU | 1170 |
| D-1500 | 973-993 | GAACUGAAAAGGGUGAUGGC | 502 | AGCCAUCACCCUUUUCAGUUCUU | 1171 |
| D-1501 | 976-996 | CUGAAAAGGGUGAUGGCUUG | 503 | ACAAGCCAUCACCCUUUUCAGUU | 1172 |
| D-1502 | 977-997 | UGAAAAGGGUGAUGGCUUGU | 504 | AACAAGCCAUCACCCUUUUCAUU | 1173 |
| D-1503; | 978-998 | GAAAAGGGUGAUGGCUUGUU | 505 | AAACAAGCCAUCACCCUUUUCUU | 1174 |
| D-2096 | | | | | |
| D-1504; | 979-999 | AAAAGGGUGAUGGCUUGUUC | 506 | AGAACAAGCCAUCACCCUUUUUU | 1175 |
| D-2097 | | | | | |
| D-1505 | 1018-1038 | GUGGACCCAGACACCGGUGU | 507 | AACACCGGUGUCUGGGUCCACUU | 1176 |
| D-1506 | 1019-1039 | UGGACCCAGACACCGGUGUC | 508 | UGACACCGGUGUCUGGGUCCAUU | 1177 |
| D-1507 | 1020-1040 | GGACCCAGACACCGGUGUCA | 509 | AUGACACCGGUGUCUGGGUCCUU | 1178 |
| D-1508 | 1022-1042 | ACCCAGACACCGGUGUCAUG | 510 | UCAUGACACCGGUGUCUGGGUUU | 1179 |
| D-1509 | 1024-1044 | CCAGACACCGGUGUCAUGAG | 511 | ACUCAUGACACCGGUGUCUGGUU | 1180 |
| D-1510 | 1029-1049 | CACCGGUGUCAUGAGCAGGA | 512 | UUCCUGCUCAUGACACCGGUGUU | 1181 |
| D-1511 | 1036-1056 | GUCAUGAGCAGGAAGGAACC | 513 | AGGUUCCUUCCUGCUCAUGACUU | 1182 |
| D-1512 | 1039-1059 | AUGAGCAGGAAGGAACCGCU | 514 | AAGCGGUUCCUUCCUGCUCAUUU | 1183 |
| D-1513 | 1045-1065 | AGGAAGGAACCGCUGGAAAC | 515 | UGUUUCCAGCGGUUCCUUCCUUU | 1184 |
| D-1514 | 1046-1066 | GGAAGGAACCGCUGGAAACA | 516 | AUGUUUCCAGCGGUUCCUUCCUU | 1185 |
| D-1515; | 1047-1067 | GAAGGAACCGCUGGAAACAC | 517 | AGUGUUUCCAGCGGUUCCUUCUU | 1186 |
| D-2088 | | | | | |
| D-1516 | 1189-1209 | CUGGGCCAGUAAUGGGAACC | 518 | AGGUUCCCAUUACUGGCCCAGUU | 1187 |
| D-1517 | 1191-1211 | GGGCCAGUAAUGGGAACCGU | 519 | UACGGUUCCCAUUACUGGCCCUU | 1188 |
| D-1518 | 1192-1212 | GGCCAGUAAUGGGAACCGUA | 520 | AUACGGUUCCCAUUACUGGCCUU | 1189 |
| D-1519 | 1193-1213 | GCCAGUAAUGGGAACCGUAU | 521 | AAUACGGUUCCCAUUACUGGCUU | 1190 |
| D-1520 | 1194-1214 | CCAGUAAUGGGAACCGUAUG | 522 | ACAUACGGUUCCCAUUACUGGUU | 1191 |
| D-1521 | 1195-1215 | CAGUAAUGGGAACCGUAUGU | 523 | AACAUACGGUUCCCAUUACUGUU | 1192 |
| D-1522 | 1196-1216 | AGUAAUGGGAACCGUAUGUC | 524 | AGACAUACGGUUCCCAUUACUUU | 1193 |
| D-1523 | 1201-1221 | UGGGAACCGUAUGUCCUGGA | 525 | UUCCAGGACAUACGGUUCCCAUU | 1194 |
| D-1524 | 1203-1223 | GGAACCGUAUGUCCUGGAAU | 526 | UAUUCCAGGACAUACGGUUCCUU | 1195 |
| D-1525; | 1219-1239 | GAAUAUUAGAUGCCUUUUAA | 527 | UUUAAAAGGCAUCUAAUAUUCUU | 1196 |
| D-2113; | | | | | |
| D-2376; | | | | | |
| D-2380 | | | | | |
| D-1526; | 1227-1247 | GAUGCCUUUUAAAAAUGUUC | 528 | AGAACAUUUUUAAAAGGCAUCUU | 1197 |
| D-2108; | | | | | |
| D-2440; | | | | | |
| D-2444 | | | | | |
| D-1527 | 1275-1295 | GUUUCAGAACUGAGACCUCU | 529 | UAGAGGUCUCAGUUCUGAAACUU | 1198 |
| D-1528 | 1278-1298 | UCAGAACUGAGACCUCUACA | 530 | AUGUAGAGGUCUCAGUUCUGAUU | 1199 |
| D-1529 | 1283-1303 | ACUGAGACCUCUACAUUUUC | 531 | AGAAAAUGUAGAGGUCUCAGUUU | 1200 |
| D-1530 | 1284-1304 | CUGAGACCUCUACAUUUUCU | 532 | AAGAAAAUGUAGAGGUCUCAGUU | 1201 |
| D-1531 | 1285-1305 | UGAGACCUCUACAUUUUCUU | 533 | AAAGAAAAUGUAGAGGUCUCAUU | 1202 |
| D-1532; | 1313-1333 | UGAUUUUCACAUUUUUCGUC | 534 | AGACGAAAAAUGUGAAAAUCAUU | 1203 |
| D-2146 | | | | | |
| D-1533; | 1314-1334 | GAUUUUCACAUUUUUCGUCU | 535 | AAGACGAAAAAUGUGAAAAUCUU | 1204 |
| D-2111; | | | | | |
| D-2374; | | | | | |
| D-2375; | | | | | |
| D-2379; | | | | | |
| D-2383 | | | | | |
| D-1534 | 1315-1335 | AUUUUCACAUUUUUCGUCUU | 536 | AAAGACGAAAAAUGUGAAAAUUU | 1205 |
| D-1535 | 1317-1337 | UUUCACAUUUUUCGUCUUUU | 537 | AAAAAGACGAAAAAUGUGAAAUU | 1206 |
| D-1536 | 1318-1338 | UUCACAUUUUUCGUCUUUUG | 538 | ACAAAAGACGAAAAAUGUGAAUU | 1207 |
| D-1537 | 1319-1339 | UCACAUUUUUCGUCUUUUGG | 539 | UCCAAAAGACGAAAAAUGUGAUU | 1208 |
| D-1538 | 1321-1341 | ACAUUUUUCGUCUUUUGGAC | 540 | AGUCCAAAAGACGAAAAAUGUUU | 1209 |
| D-1539 | 1325-1345 | UUUUCGUCUUUUGGACUUCU | 541 | AAGAAGUCCAAAAGACGAAAAUU | 1210 |
| D-1540 | 1326-1346 | UUUCGUCUUUUGGACUUCUG | 542 | ACAGAAGUCCAAAAGACGAAAUU | 1211 |
| D-1541 | 1327-1347 | UUCGUCUUUUGGACUUCUGG | 543 | ACCAGAAGUCCAAAAGACGAAUU | 1212 |
| D-1542 | 1328-1348 | UCGUCUUUUGGACUUCUGGU | 544 | AACCAGAAGUCCAAAAGACGAUU | 1213 |
| D-1543 | 1332-1352 | CUUUUGGACUUCUGGUGUCU | 545 | AAGACACCAGAAGUCCAAAAGUU | 1214 |
| D-1544 | 1335-1355 | UUGGACUUCUGGUGUCUCAA | 546 | AUUGAGACACCAGAAGUCCAAUU | 1215 |
| D-1545 | 1337-1357 | GGACUUCUGGUGUCUCAAUG | 547 | ACAUUGAGACACCAGAAGUCCUU | 1216 |
| D-1546 | 1338-1358 | GACUUCUGGUGUCUCAAUGC | 548 | AGCAUUGAGACACCAGAAGUCUU | 1217 |
| D-1547 | 1341-1361 | UUCUGGUGUCUCAAUGCUUC | 549 | UGAAGCAUUGAGACACCAGAAUU | 1218 |
| D-1548 | 1356-1376 | GCUUCAAUGUCCCAGUGCAA | 550 | UUUGCACUGGGACAUUGAAGCUU | 1219 |
| D-1549; | 1362-1382 | AUGUCCCAGUGCAAAAAGUA | 551 | UUACUUUUUGCACUGGGACAUUU | 1220 |
| D-2075 | | | | | |
| D-1550 | 1363-1383 | UGUCCCAGUGCAAAAAGUAA | 552 | UUUACUUUUUGCACUGGGACAUU | 1221 |
| D-1551; | 1364-1384 | GUCCCAGUGCAAAAAGUAAA | 553 | AUUUACUUUUUGCACUGGGACUU | 1222 |
| D-2152 | | | | | |
| D-1552; | 1365-1385 | UCCCAGUGCAAAAAGUAAAG | 554 | UCUUUACUUUUUGCACUGGGAUU | 1223 |
| D-2157 | | | | | |
| D-1553 | 1369-1389 | AGUGCAAAAAGUAAAGAAAU | 555 | UAUUUCUUUACUUUUUGCACUUU | 1224 |
| D-1554 | 1382-1402 | AAGAAAUAUAGUCUCAAUAA | 556 | AUUAUUGAGACUAUAUUUCUUUU | 1225 |
| D-1555; | 1385-1405 | AAAUAUAGUCUCAAUAACUU | 557 | UAAGUUAUUGAGACUAUAUUUUU | 1226 |
| D-2136; | | | | | |
| D-2438; | | | | | |
| D-2442 | | | | | |
| D-1556; | 1387-1407 | AUAUAGUCUCAAUAACUUAG | 558 | ACUAAGUUAUUGAGACUAUAUUU | 1227 |
| D-2156 | | | | | |
| D-1557 | 1388-1408 | UAUAGUCUCAAUAACUUAGU | 559 | UACUAAGUUAUUGAGACUAUAUU | 1228 |
| D-1558 | 1389-1409 | AUAGUCUCAAUAACUUAGUA | 560 | AUACUAAGUUAUUGAGACUAUUU | 1229 |
| D-1559 | 1390-1410 | UAGUCUCAAUAACUUAGUAG | 561 | ACUACUAAGUUAUUGAGACUAUU | 1230 |
| D-1560 | 1391-1411 | AGUCUCAAUAACUUAGUAGG | 562 | UCCUACUAAGUUAUUGAGACUUU | 1231 |
| D-1561 | 1395-1415 | UCAAUAACUUAGUAGGACUU | 563 | AAAGUCCUACUAAGUUAUUGAUU | 1232 |
| D-1562 | 1396-1416 | CAAUAACUUAGUAGGACUUC | 564 | UGAAGUCCUACUAAGUUAUUGUU | 1233 |
| D-1563 | 1398-1418 | AUAACUUAGUAGGACUUCAG | 565 | ACUGAAGUCCUACUAAGUUAUUU | 1234 |
| D-1564 | 1400-1420 | AACUUAGUAGGACUUCAGUA | 566 | UUACUGAAGUCCUACUAAGUUUU | 1235 |
| D-1565 | 1401-1421 | ACUUAGUAGGACUUCAGUAA | 567 | AUUACUGAAGUCCUACUAAGUUU | 1236 |
| D-1566 | 1403-1423 | UUAGUAGGACUUCAGUAAGU | 568 | AACUUACUGAAGUCCUACUAAUU | 1237 |
| D-1567 | 1404-1424 | UAGUAGGACUUCAGUAAGUC | 569 | UGACUUACUGAAGUCCUACUAUU | 1238 |
| D-1568 | 1405-1425 | AGUAGGACUUCAGUAAGUCA | 570 | AUGACUUACUGAAGUCCUACUUU | 1239 |
| D-1569 | 1407-1427 | UAGGACUUCAGUAAGUCACU | 571 | AAGUGACUUACUGAAGUCCUAUU | 1240 |
| D-1570 | 1427-1447 | UAAAUGACAAGACAGGAUUC | 572 | AGAAUCCUGUCUUGUCAUUUAUU | 1241 |
| D-1571 | 1441-1461 | GGAUUCUGAAAACUCCCCGU | 573 | AACGGGGAGUUUUCAGAAUCCUU | 1242 |
| D-1572 | 1442-1462 | GAUUCUGAAAACUCCCCGUU | 574 | AAACGGGGAGUUUUCAGAAUCUU | 1243 |
| D-1573 | 1444-1464 | UUCUGAAAACUCCCCGUUUA | 575 | UUAAACGGGGAGUUUUCAGAAUU | 1244 |
| D-1574 | 1445-1465 | UCUGAAAACUCCCCGUUUAA | 576 | AUUAAACGGGGAGUUUUCAGAUU | 1245 |
| D-1575 | 1446-1466 | CUGAAAACUCCCCGUUUAAC | 577 | AGUUAAACGGGGAGUUUUCAGUU | 1246 |
| D-1576; | 1447-1467 | UGAAAACUCCCCGUUUAACU | 578 | AAGUUAAACGGGGAGUUUUCAUU | 1247 |
| D-2107 | | | | | |
| D-1577 | 1449-1469 | AAAACUCCCCGUUUAACUGA | 579 | AUCAGUUAAACGGGGAGUUUUUU | 1248 |
| D-1578 | 1452-1472 | ACUCCCCGUUUAACUGAUUA | 580 | AUAAUCAGUUAAACGGGGAGUUU | 1249 |
| D-1579 | 1453-1473 | CUCCCCGUUUAACUGAUUAU | 581 | AAUAAUCAGUUAAACGGGGAGUU | 1250 |
| D-1580; | 1454-1474 | UCCCCGUUUAACUGAUUAUG | 582 | ACAUAAUCAGUUAAACGGGGAUU | 1251 |
| D-2155 | | | | | |
| D-1581; | 1455-1475 | CCCCGUUUAACUGAUUAUGG | 583 | UCCAUAAUCAGUUAAACGGGGUU | 1252 |
| D-2116; | | | | | |
| D-2490; | | | | | |
| D-2502; | | | | | |
| D-2508 | | | | | |
| D-1582 | 1484-1504 | UUCUCCUGCUUCUCCGUUUA | 584 | AUAAACGGAGAAGCAGGAGAAUU | 1253 |
| D-1583 | 1485-1505 | UCUCCUGCUUCUCCGUUUAU | 585 | AAUAAACGGAGAAGCAGGAGAUU | 1254 |
| D-1584; | 1486-1506 | CUCCUGCUUCUCCGUUUAUC | 586 | AGAUAAACGGAGAAGCAGGAGUU | 1255 |
| D-2120 | | | | | |
| D-1585 | 1488-1508 | CCUGCUUCUCCGUUUAUCUA | 587 | AUAGAUAAACGGAGAAGCAGGUU | 1256 |
| D-1586 | 1491-1511 | GCUUCUCCGUUUAUCUACCA | 588 | UUGGUAGAUAAACGGAGAAGCUU | 1257 |
| D-1587 | 1492-1512 | CUUCUCCGUUUAUCUACCAA | 589 | AUUGGUAGAUAAACGGAGAAGUU | 1258 |
| D-1588 | 1493-1513 | UUCUCCGUUUAUCUACCAAG | 590 | UCUUGGUAGAUAAACGGAGAAUU | 1259 |
| D-1589 | 1494-1514 | UCUCCGUUUAUCUACCAAGA | 591 | AUCUUGGUAGAUAAACGGAGAUU | 1260 |
| D-1590 | 1495-1515 | CUCCGUUUAUCUACCAAGAG | 592 | ACUCUUGGUAGAUAAACGGAGUU | 1261 |
| D-1591 | 1496-1516 | UCCGUUUAUCUACCAAGAGC | 593 | AGCUCUUGGUAGAUAAACGGAUU | 1262 |
| D-1592 | 1498-1518 | CGUUUAUCUACCAAGAGCGC | 594 | UGCGCUCUUGGUAGAUAAACGUU | 1263 |
| D-1593 | 1501-1521 | UUAUCUACCAAGAGCGCAGA | 595 | AUCUGCGCUCUUGGUAGAUAAUU | 1264 |
| D-1594 | 1503-1523 | AUCUACCAAGAGCGCAGACU | 596 | AAGUCUGCGCUCUUGGUAGAUUU | 1265 |
| D-1595; | 1506-1526 | UACCAAGAGCGCAGACUUGC | 597 | UGCAAGUCUGCGCUCUUGGUAUU | 1266 |
| D-2072; | | | | | |
| D-2439; | | | | | |
| D-2443 | | | | | |
| D-1596; | 1507-1527 | ACCAAGAGCGCAGACUUGCA | 598 | AUGCAAGUCUGCGCUCUUGGUUU | 1267 |
| D-2087 | | | | | |
| D-1597; | 1509-1529 | CAAGAGCGCAGACUUGCAUC | 599 | AGAUGCAAGUCUGCGCUCUUGUU | 1268 |
| D-2147 | | | | | |
| D-1598 | 1510-1530 | AAGAGCGCAGACUUGCAUCC | 600 | AGGAUGCAAGUCUGCGCUCUUUU | 1269 |
| D-1599 | 1512-1532 | GAGCGCAGACUUGCAUCCUG | 601 | ACAGGAUGCAAGUCUGCGCUCUU | 1270 |
| D-1600 | 1514-1534 | GCGCAGACUUGCAUCCUGUC | 602 | UGACAGGAUGCAAGUCUGCGCUU | 1271 |
| D-1601 | 1515-1535 | CGCAGACUUGCAUCCUGUCA | 603 | AUGACAGGAUGCAAGUCUGCGUU | 1272 |
| D-1602; | 1517-1537 | CAGACUUGCAUCCUGUCACU | 604 | UAGUGACAGGAUGCAAGUCUGUU | 1273 |
| D-2121 | | | | | |
| D-1603 | 1521-1541 | CUUGCAUCCUGUCACUACCA | 605 | AUGGUAGUGACAGGAUGCAAGUU | 1274 |
| D-1604 | 1525-1545 | CAUCCUGUCACUACCACUCG | 606 | ACGAGUGGUAGUGACAGGAUGUU | 1275 |
| D-1605 | 1527-1547 | UCCUGUCACUACCACUCGUU | 607 | UAACGAGUGGUAGUGACAGGAUU | 1276 |
| D-1606; | 1528-1548 | CCUGUCACUACCACUCGUUA | 608 | AUAACGAGUGGUAGUGACAGGUU | 1277 |
| D-2090 | | | | | |
| D-1607 | 1529-1549 | CUGUCACUACCACUCGUUAG | 609 | UCUAACGAGUGGUAGUGACAGUU | 1278 |
| D-1608 | 1530-1550 | UGUCACUACCACUCGUUAGA | 610 | AUCUAACGAGUGGUAGUGACAUU | 1279 |
| D-1609 | 1534-1554 | ACUACCACUCGUUAGAGAAA | 611 | AUUUCUCUAACGAGUGGUAGUUU | 1280 |
| D-1610 | 1572-1592 | AAGAGUGGGUGGGCUGGAAG | 612 | UCUUCCAGCCCACCCACUCUUUU | 1281 |
| D-1611; | 1596-1616 | UCCUAGAAUGUGUUAUUGCC | 613 | AGGCAAUAACACAUUCUAGGAUU | 1282 |
| D-2124 | | | | | |
| D-1612 | 1597-1617 | CCUAGAAUGUGUUAUUGCCC | 614 | AGGGCAAUAACACAUUCUAGGUU | 1283 |
| D-1613 | 1602-1622 | AAUGUGUUAUUGCCCCUGUU | 615 | AAACAGGGGCAAUAACACAUUUU | 1284 |
| D-1614 | 1605-1625 | GUGUUAUUGCCCCUGUUCAU | 616 | AAUGAACAGGGGCAAUAACACUU | 1285 |
| D-1615 | 1608-1628 | UUAUUGCCCCUGUUCAUGAG | 617 | ACUCAUGAACAGGGGCAAUAAUU | 1286 |
| D-1616 | 1610-1630 | AUUGCCCCUGUUCAUGAGGU | 618 | UACCUCAUGAACAGGGGCAAUUU | 1287 |
| D-1617 | 1634-1654 | AAUGAAAAUUAAAUUGCACC | 619 | AGGUGCAAUUUAAUUUUCAUUUU | 1288 |
| D-1618 | 1635-1655 | AUGAAAAUUAAAUUGCACCC | 620 | AGGGUGCAAUUUAAUUUUCAUUU | 1289 |
| D-1619 | 1639-1659 | AAAUUAAAUUGCACCCCAAA | 621 | AUUUGGGGUGCAAUUUAAUUUUU | 1290 |
| D-1620 | 1641-1661 | AUUAAAUUGCACCCCAAAUA | 622 | AUAUUUGGGGUGCAAUUUAAUUU | 1291 |
| D-1621 | 1642-1662 | UUAAAUUGCACCCCAAAUAU | 623 | AAUAUUUGGGGUGCAAUUUAAUU | 1292 |
| D-1622 | 1645-1665 | AAUUGCACCCCAAAUAUGGC | 624 | AGCCAUAUUUGGGGUGCAAUUUU | 1293 |
| D-1623 | 1646-1666 | AUUGCACCCCAAAUAUGGCU | 625 | AAGCCAUAUUUGGGGUGCAAUUU | 1294 |
| D-1624 | 1658-1678 | AUAUGGCUGGAAUGCCACUU | 626 | AAAGUGGCAUUCCAGCCAUAUUU | 1295 |
| D-1625 | 1665-1685 | UGGAAUGCCACUUCCCUUUU | 627 | AAAAAGGGAAGUGGCAUUCCAUU | 1296 |
| D-1626; | 1676-1696 | UUCCCUUUUCUUCUCAAGCC | 628 | AGGCUUGAGAAGAAAAGGGAAUU | 1297 |
| D-2089 | | | | | |
| D-1627 | 1684-1704 | UCUUCUCAAGCCCCGGGCUA | 629 | AUAGCCCGGGGCUUGAGAAGAUU | 1298 |
| D-1628 | 1687-1707 | UCUCAAGCCCCGGGCUAGCU | 630 | AAGCUAGCCCGGGGCUUGAGAUU | 1299 |
| D-1629 | 1704-1724 | GCUUUUGAAAUGGCAUAAAG | 631 | UCUUUAUGCCAUUUCAAAAGCUU | 1300 |
| D-1630 | 1707-1727 | UUUGAAAUGGCAUAAAGACU | 632 | AAGUCUUUAUGCCAUUUCAAAUU | 1301 |
| D-1631 | 1712-1732 | AAUGGCAUAAAGACUGAGGU | 633 | AACCUCAGUCUUUAUGCCAUUUU | 1302 |
| D-1632; | 1714-1734 | UGGCAUAAAGACUGAGGUGA | 634 | AUCACCUCAGUCUUUAUGCCAUU | 1303 |
| D-2123 | | | | | |
| D-1633; | 1716-1736 | GCAUAAAGACUGAGGUGACC | 635 | AGGUCACCUCAGUCUUUAUGCUU | 1304 |
| D-2094 | | | | | |
| D-1634 | 1741-1761 | GAAGCACUGCAGAUAUUAAU | 636 | AAUUAAUAUCUGCAGUGCUUCUU | 1305 |
| D-1635; | 1813-1833 | CUAAAGGUGCUCAGGAGGAU | 637 | AAUCCUCCUGAGCACCUUUAGUU | 1306 |
| D-2103 | | | | | |
| D-1636 | 1817-1837 | AGGUGCUCAGGAGGAUGGUU | 638 | AAACCAUCCUCCUGAGCACCUUU | 1307 |
| D-1637 | 1819-1839 | GUGCUCAGGAGGAUGGUUGU | 639 | AACAACCAUCCUCCUGAGCACUU | 1308 |
| D-1638 | 1827-1847 | GAGGAUGGUUGUGUAGUCAU | 640 | AAUGACUACACAACCAUCCUCUU | 1309 |
| D-1639 | 1828-1848 | AGGAUGGUUGUGUAGUCAUG | 641 | ACAUGACUACACAACCAUCCUUU | 1310 |
| D-1640 | 1829-1849 | GGAUGGUUGUGUAGUCAUGG | 642 | UCCAUGACUACACAACCAUCCUU | 1311 |
| D-1641 | 1835-1855 | UUGUGUAGUCAUGGAGGACC | 643 | AGGUCCUCCAUGACUACACAAUU | 1312 |
| D-1642 | 1843-1863 | UCAUGGAGGACCCCUGGAUC | 644 | AGAUCCAGGGGUCCUCCAUGAUU | 1313 |
| D-1643 | 1869-1889 | AUUCCCCUCAGCUAAUGACG | 645 | ACGUCAUUAGCUGAGGGGAAUUU | 1314 |
| D-1644 | 1870-1890 | UUCCCCUCAGCUAAUGACGG | 646 | UCCGUCAUUAGCUGAGGGGAAUU | 1315 |
| D-1645 | 1871-1891 | UCCCCUCAGCUAAUGACGGA | 647 | AUCCGUCAUUAGCUGAGGGGAUU | 1316 |
| D-1646 | 1876-1896 | UCAGCUAAUGACGGAGUGCU | 648 | AAGCACUCCGUCAUUAGCUGAUU | 1317 |
| D-1647 | 1914-1934 | GAAAAAGUUCUGAAUUCUGU | 649 | AACAGAAUUCAGAACUUUUUCUU | 1318 |
| D-1648 | 1919-1939 | AGUUCUGAAUUCUGUGGAGG | 650 | UCCUCCACAGAAUUCAGAACUUU | 1319 |
| D-1649 | 1955-1975 | AGUGAUUUCAGAUAGACUAC | 651 | AGUAGUCUAUCUGAAAUCACUUU | 1320 |
| D-1650 | 1959-1979 | AUUUCAGAUAGACUACUGAA | 652 | UUUCAGUAGUCUAUCUGAAAUUU | 1321 |
| D-1651; | 1963-1983 | CAGAUAGACUACUGAAAACC | 653 | AGGUUUUCAGUAGUCUAUCUGUU | 1322 |
| D-2148 | | | | | |
| D-1652 | 1967-1987 | UAGACUACUGAAAACCUUUA | 654 | UUAAAGGUUUUCAGUAGUCUAUU | 1323 |
| D-1653 | 1968-1988 | AGACUACUGAAAACCUUUAA | 655 | UUUAAAGGUUUUCAGUAGUCUUU | 1324 |
| D-1654; | 1996-2016 | AAGGAAAGCAUAUGUCAGUU | 656 | AAACUGACAUAUGCUUUCCUUUU | 1325 |
| D-2114 | | | | | |
| D-1655; | 1997-2017 | AGGAAAGCAUAUGUCAGUUG | 657 | ACAACUGACAUAUGCUUUCCUUU | 1326 |
| D-2115; | | | | | |
| D-2377; | | | | | |
| D-2381 | | | | | |
| D-1656; | 1998-2018 | GGAAAGCAUAUGUCAGUUGU | 658 | AACAACUGACAUAUGCUUUCCUU | 1327 |
| D-2117 | | | | | |
| D-1657; | 2000-2020 | AAAGCAUAUGUCAGUUGUUU | 659 | UAAACAACUGACAUAUGCUUUUU | 1328 |
| D-2104 | | | | | |
| D-1658 | 2001-2021 | AAGCAUAUGUCAGUUGUUUA | 660 | UUAAACAACUGACAUAUGCUUUU | 1329 |
| D-1659 | 2002-2022 | AGCAUAUGUCAGUUGUUUAA | 661 | UUUAAACAACUGACAUAUGCUUU | 1330 |
| D-1660 | 2019-2039 | UAAAACCCAAUAUCUAUUUU | 662 | AAAAAUAGAUAUUGGGUUUUAUU | 1331 |
| D-1661 | 2022-2042 | AACCCAAUAUCUAUUUUUUA | 663 | UUAAAAAAUAGAUAUUGGGUUUU | 1332 |
| D-1662; | 2039-2059 | UUAACUGAUUGUAUAACUCU | 664 | UAGAGUUAUACAAUCAGUUAAUU | 1333 |
| D-2105 | | | | | |
| D-1663; | 2040-2060 | UAACUGAUUGUAUAACUCUA | 665 | UUAGAGUUAUACAAUCAGUUAUU | 1334 |
| D-2106 | | | | | |
| D-1664; | 2042-2062 | ACUGAUUGUAUAACUCUAAG | 666 | UCUUAGAGUUAUACAAUCAGUUU | 1335 |
| D-2153 | | | | | |
| D-1665 | 2043-2063 | CUGAUUGUAUAACUCUAAGA | 667 | AUCUUAGAGUUAUACAAUCAGUU | 1336 |
| D-1666 | 2045-2065 | GAUUGUAUAACUCUAAGAUC | 668 | AGAUCUUAGAGUUAUACAAUCUU | 1337 |
| D-1667; | 2086-2106 | GCCAUUUUGUCCUUUGAUUA | 669 | AUAAUCAAAGGACAAAAUGGCUU | 1338 |
| D-2118; | | | | | |
| D-2378; | | | | | |
| D-2382 | | | | | |
| D-1668 | 2093-2113 | UGUCCUUUGAUUAUAUUGGG | 670 | UCCCAAUAUAAUCAAAGGACAUU | 1339 |
| D-2179 | 682-704 | CCAGUGGAUAACCAGCUUCC | 46 | AGGAAGCUGGUUAUCCACUGGUG | 2914 |
| D-2192 | 684-704 | CCAGUGGAUAACCAGCUUCC | 46 | AGGAAGCUGGUUAUCCACUGG | 2922 |
| D-2177 | 1092-1114 | CAGAACGAAAGUUAUAUGGA | 98 | UUCCAUAUAACUUUCGUUCUGUG | 2912 |
| D-2190 | 1094-1114 | CAGAACGAAAGUUAUAUGGA | 98 | UUCCAUAUAACUUUCGUUCUG | 2920 |
| D-2462 | 1092-1114 | CAGAACGAAAGUUAUAUGGA | 98 | UUCCAUAUAACUUUCGUUCUGAA | 2992 |
| D-2483 | 768-788 | AAUAGCAGACUUGUUCCGAC | 101 | AGUCGGAACAAGUCUGCUAUU | 3003 |
| D-2181 | 794-816 | CAGAUUGCUUACUCAGACAC | 115 | AGUGUCUGAGUAAGCAAUCUGUG | 2916 |
| D-2194 | 796-816 | CAGAUUGCUUACUCAGACAC | 115 | AGUGUCUGAGUAAGCAAUCUG | 2924 |
| D-2175 | 892-914 | CUUCAGGCCCAAUAUUGUAA | 142 | AUUACAAUAUUGGGCCUGAAGUG | 2910 |
| D-2188 | 894-914 | CUUCAGGCCCAAUAUUGUAA | 142 | AUUACAAUAUUGGGCCUGAAG | 2918 |
| D-2223 | 1110-1130 | UGGAAAAUCACCACUCUUUG | 152 | ACAAAGAGUGGUGAUUUUCCA | 2937 |
| D-2464 | 1108-1130 | UGGAAAAUCACCACUCUUUG | 152 | ACAAAGAGUGGUGAUUUUCCAUA | 2994 |
| D-2226 | 1255-1275 | GACAACACUUGAAGCAUGGU | 168 | AACCAUGCUUCAAGUGUUGUC | 2940 |
| D-2488 | 1346-1366 | GUGUCUCAAUGCUUCAAUGU | 175 | AACAUUGAAGCAUUGAGACAC | 3008 |
| D-2219 | 1350-1370 | CUCAAUGCUUCAAUGUCCCA | 178 | AUGGGACAUUGAAGCAUUGAG | 2933 |
| D-2176 | 1350-1372 | CAAUGCUUCAAUGUCCCAGU | 179 | AACUGGGACAUUGAAGCAUUGUG | 2911 |
| D-2182; | 1350-1372 | CAAUGCUUCAAUGUCCCAGU | 179 | AACUGGGACAUUGAAGCAUUGAG | 2917 |
| D-2389; | | | | | |
| D-2391; | | | | | |
| D-2401; | | | | | |
| D-2402; | | | | | |
| D-2403 | | | | | |
| D-2189; | 1352-1372 | CAAUGCUUCAAUGUCCCAGU | 179 | AACUGGGACAUUGAAGCAUUG | 2919 |
| D-2384; | | | | | |
| D-2385; | | | | | |
| D-2399 | | | | | |
| D-2221 | 1355-1375 | UGCUUCAAUGUCCCAGUGCA | 181 | UUGCACUGGGACAUUGAAGCA | 2935 |
| D-2225 | 1438-1458 | ACAGGAUUCUGAAAACUCCC | 186 | AGGGAGUUUUCAGAAUCCUGU | 2939 |
| D-2222 | 1794-1814 | UCAGACAGCAUUGGAUUUCC | 215 | AGGAAAUCCAAUGCUGUCUGA | 2936 |
| D-2224 | 1796-1816 | AGACAGCAUUGGAUUUCCUA | 217 | UUAGGAAAUCCAAUGCUGUCU | 2938 |
| D-2220 | 2014-2034 | UUGUUUAAAACCCAAUAUCU | 245 | UAGAUAUUGGGUUUUAAACAA | 2934 |
| D-2461 | 2012-2034 | UUGUUUAAAACCCAAUAUCU | 245 | UAGAUAUUGGGUUUUAAACAACU | 2991 |
| D-2180 | 2055-2077 | CUAAGAUCUGAUGAAGUAUA | 247 | AUAUACUUCAUCAGAUCUUAGUG | 2915 |
| D-2193 | 2057-2077 | CUAAGAUCUGAUGAAGUAUA | 247 | AUAUACUUCAUCAGAUCUUAG | 2923 |
| D-2463 | 2055-2077 | CUAAGAUCUGAUGAAGUAUA | 247 | AUAUACUUCAUCAGAUCUUAGAG | 2993 |
| D-2472 | 2057-2077 | AAGAUCUGAUGAAGUAUAUU | 249 | AUAUACUUCAUCAGAUCUUAG | 2923 |
| D-2178 | 2109-2131 | GGAAGUUGACUAAACUUGAA | 258 | UUUCAAGUUUAGUCAACUUCCUG | 2913 |
| D-2191 | 2111-2131 | GGAAGUUGACUAAACUUGAA | 258 | UUUCAAGUUUAGUCAACUUCC | 2921 |
| D-2486 | 2082-2102 | UAUUGCCAUUUUGUCCUUUG | 268 | UCAAAGGACAAAAUGGCAAUA | 3006 |
| D-2109 | 2113-2133 | AAGUUGACUAAACUUGAAAA | 270 | UUUUUCAAGUUUAGUCAACUUUU | 2906 |
| D-2485 | 1253-1273 | AUGACAACACUUGAAGCAUG | 301 | ACAUGCUUCAAGUGUUGUCAU | 3005 |
| D-2254 | 985-1005 | GUGAUGGCUUGUUCCAGAUG | 369 | ACAUCUGGAACAAGCCAUCAC | 2952 |
| D-2437 | 1211-1231 | AUGUCCUGGAAUAUUAGAUG | 387 | ACAUCUAAUAUUCCAGGACAU | 2982 |
| D-2255 | 1212-1232 | UGUCCUGGAAUAUUAGAUGC | 388 | AGCAUCUAAUAUUCCAGGACA | 2953 |
| D-2256 | 1213-1233 | GUCCUGGAAUAUUAGAUGCC | 389 | AGGCAUCUAAUAUUCCAGGAC | 2954 |
| D-2258 | 1214-1234 | UCCUGGAAUAUUAGAUGCCU | 390 | AAGGCAUCUAAUAUUCCAGGA | 2956 |
| D-2241; | 1215-1233 | CCUGGAAUAUUAGAUGCCUU | 391 | AGGCAUCUAAUAUUCCAGGUU | 2949 |
| D-2482 | | | | | |
| D-2243 | 1216-1234 | CUGGAAUAUUAGAUGCCUUU | 392 | AAGGCAUCUAAUAUUCCAGUU | 2951 |
| D-2466 | 1939-1959 | AGAAGAAAAGUGAUUCAGUG | 398 | UCACUGAAUCACUUUUCUUCU | 2996 |
| D-2469 | 1937-1959 | AGAAGAAAAGUGAUUCAGUG | 398 | UCACUGAAUCACUUUUCUUCUCC | 2999 |
| D-2487 | 2050-2070 | UAUAACUCUAAGAUCUGAUG | 405 | UCAUCAGAUCUUAGAGUUAUA | 3007 |
| D-2465 | 2052-2072 | UAACUCUAAGAUCUGAUGAA | 407 | AUUCAUCAGAUCUUAGAGUUA | 2995 |
| D-2468 | 2050-2072 | UAACUCUAAGAUCUGAUGAA | 407 | AUUCAUCAGAUCUUAGAGUUAUA | 2998 |
| D-2257 | 2054-2074 | ACUCUAAGAUCUGAUGAAGU | 409 | UACUUCAUCAGAUCUUAGAGU | 2955 |
| D-2431 | 1219-1239 | GAAUAUUAGAUGCCUUUUAA | 527 | UUUAAAAGGCAUCUAAUAUUC | 2976 |
| D-2436 | 1227-1247 | GAUGCCUUUUAAAAAUGUUC | 528 | AGAACAUUUUUAAAAGGCAUC | 2981 |
| D-2430 | 1314-1334 | GAUUUUCACAUUUUUCGUCU | 535 | AAGACGAAAAAUGUGAAAAUC | 2975 |
| D-2467 | 1312-1334 | GAUUUUCACAUUUUUCGUCU | 535 | AAGACGAAAAAUGUGAAAAUCAC | 2997 |
| D-2434 | 1385-1405 | AAAUAUAGUCUCAAUAACUU | 557 | UAAGUUAUUGAGACUAUAUUU | 2979 |
| D-2484 | 1455-1475 | CCCCGUUUAACUGAUUAUGG | 583 | UCCAUAAUCAGUUAAACGGGG | 3004 |
| D-2435 | 1506-1526 | UACCAAGAGCGCAGACUUGC | 597 | UGCAAGUCUGCGCUCUUGGUA | 2980 |
| D-2480 | 1504-1526 | UACCAAGAGCGCAGACUUGC | 597 | UGCAAGUCUGCGCUCUUGGUAGA | 3002 |
| D-2432 | 1997-2017 | AGGAAAGCAUAUGUCAGUUG | 657 | ACAACUGACAUAUGCUUUCCU | 2977 |
| D-2433 | 2086-2106 | GCCAUUUUGUCCUUUGAUUA | 669 | AUAAUCAAAGGACAAAAUGGC | 2978 |
| D-2158; | 1354-1372 | AUGCUUCAAUGUCCCAGUUU | 2804 | AACUGGGACAUUGAAGCAUUU | 2907 |
| D-2387; | | | | | |
| D-2390; | | | | | |
| D-2400 | | | | | |
| D-2386; | 1352-1372 | AUGCUUCAAUGUCCCAGUUU | 2804 | AACUGGGACAUUGAAGCAUUG | 2919 |
| D-2392 | | | | | |
| D-2159 | 1096-1114 | GAACGAAAGUUAUAUGGAAU | 2805 | UUCCAUAUAACUUUCGUUCUU | 2908 |
| D-2479 | 1094-1114 | GAACGAAAGUUAUAUGGAAU | 2805 | UUCCAUAUAACUUUCGUUCUG | 2920 |
| D-2160 | 798-816 | GAUUGCUUACUCAGACACUU | 2806 | AGUGUCUGAGUAAGCAAUCUU | 2909 |
| D-2195 | 1352-1370 | CAAUGCUUCAAUGUCCCAUU | 2807 | AUGGGACAUUGAAGCAUUGUU | 2925 |
| D-2196 | 2016-2034 | GUUUAAAACCCAAUAUCUAU | 2808 | UAGAUAUUGGGUUUUAAACUU | 2926 |
| D-2197 | 1357-1375 | CUUCAAUGUCCCAGUGCAAU | 2809 | UUGCACUGGGACAUUGAAGUU | 2927 |
| D-2198 | 1796-1814 | AGACAGCAUUGGAUUUCCUU | 2810 | AGGAAAUCCAAUGCUGUCUUU | 2928 |
| D-2199 | 1112-1130 | GAAAAUCACCACUCUUUGUU | 2811 | ACAAAGAGUGGUGAUUUUCUU | 2929 |
| D-2475 | 1110-1130 | GAAAAUCACCACUCUUUGUU | 2811 | ACAAAGAGUGGUGAUUUUCCA | 2937 |
| D-2200 | 1798-1816 | ACAGCAUUGGAUUUCCUAAU | 2812 | UUAGGAAAUCCAAUGCUGUUU | 2930 |
| D-2201 | 1440-1458 | AGGAUUCUGAAAACUCCCUU | 2813 | AGGGAGUUUUCAGAAUCCUUU | 2931 |
| D-2202 | 1257-1275 | CAACACUUGAAGCAUGGUUU | 2814 | AACCAUGCUUCAAGUGUUGUU | 2932 |
| D-2233 | 2014-2034 | CUGUUUAAAACCCAAUAUCU | 2815 | UAGAUAUUGGGUUUUAAACAGUU | 2941 |
| D-2234 | 1355-1375 | CGCUUCAAUGUCCCAGUGCA | 2816 | UUGCACUGGGACAUUGAAGCGUU | 2942 |
| D-2235 | 1794-1814 | CCAGACAGCAUUGGAUUUCC | 2817 | AGGAAAUCCAAUGCUGUCUGGUU | 2943 |
| D-2236 | 1110-1130 | CGGAAAAUCACCACUCUUUG | 2818 | ACAAAGAGUGGUGAUUUUCCGUU | 2944 |
| D-2237 | 1796-1816 | GGACAGCAUUGGAUUUCCUA | 2819 | UUAGGAAAUCCAAUGCUGUCCUU | 2945 |
| D-2238 | 1438-1458 | GCAGGAUUCUGAAAACUCCC | 2820 | AGGGAGUUUUCAGAAUCCUGCUU | 2946 |
| D-2239 | 987-1005 | GAUGGCUUGUUCCAGAUGUU | 2821 | ACAUCUGGAACAAGCCAUCUU | 2947 |
| D-2240 | 1214-1232 | UCCUGGAAUAUUAGAUGCUU | 2822 | AGCAUCUAAUAUUCCAGGAUU | 2948 |
| D-2242 | 2056-2074 | UCUAAGAUCUGAUGAAGUAU | 2823 | UACUUCAUCAGAUCUUAGAUU | 2950 |
| D-2259 | 1214-1234 | CCCUGGAAUAUUAGAUGCCU | 2824 | AAGGCAUCUAAUAUUCCAGGGUU | 2957 |
| D-2260; | 2054-2074 | GCUCUAAGAUCUGAUGAAGU | 2825 | UACUUCAUCAGAUCUUAGAGCUU | 2958 |
| D-2454; | | | | | |
| D-2455; | | | | | |
| D-2456 | | | | | |
| D-2261 | 1212-1232 | CGUCCUGGAAUAUUAGAUGC | 2826 | AGCAUCUAAUAUUCCAGGACGUU | 2959 |
| D-2262 | 2052-2072 | CAACUCUAAGAUCUGAUGAA | 2827 | AUUCAUCAGAUCUUAGAGUUGUU | 2960 |
| D-2263 | 1939-1959 | GGAAGAAAAGUGAUUCAGUG | 2828 | UCACUGAAUCACUUUUCUUCCUU | 2961 |
| D-2268 | 1213-1233 | GUCCAGGAAUAUUAGAUGCC | 2829 | AGGCAUCUAAUAUUCCUGGACUU | 2962 |
| D-2269 | 1213-1233 | GUGCUGGAAUAUUAGAUGCC | 2830 | AGGCAUCUAAUAUUCCAGCACUU | 2963 |
| D-2270 | 1214-1234 | UCCUCGAAUAUUAGAUGCCU | 2831 | AAGGCAUCUAAUAUUCGAGGAUU | 2964 |
| D-2271 | 1214-1234 | UCGUGGAAUAUUAGAUGCCU | 2832 | AAGGCAUCUAAUAUUCCACGAUU | 2965 |
| D-2272 | 2054-2074 | ACACUAAGAUCUGAUGAAGU | 2833 | UACUUCAUCAGAUCUUAGUGUUU | 2966 |
| D-2273 | 2054-2074 | AGUCUAAGAUCUGAUGAAGU | 2834 | UACUUCAUCAGAUCUUAGACUUU | 2967 |
| D-2274 | 1796-1816 | AGUCAGCAUUGGAUUUCCUA | 2835 | UUAGGAAAUCCAAUGCUGACUUU | 2968 |
| D-2275 | 1796-1816 | ACACAGCAUUGGAUUUCCUA | 2836 | UUAGGAAAUCCAAUGCUGUGUUU | 2969 |
| D-2359; | 986-1006 | UGAUGGCUUGUUCCAGAUGC | 2837 | UGCAUCUGGAACAAGCCAUCAUU | 2970 |
| D-2364; | | | | | |
| D-2369 | | | | | |
| D-2360; | 987-1007 | GAUGGCUUGUUCCAGAUGCA | 2838 | AUGCAUCUGGAACAAGCCAUCUU | 2971 |
| D-2365; | | | | | |
| D-2370 | | | | | |
| D-2361; | 1793-1813 | CUCAGACAGCAUUGGAUUUC | 2839 | AGAAAUCCAAUGCUGUCUGAGUU | 2972 |
| D-2366; | | | | | |
| D-2371 | | | | | |
| D-2362; | 2085-2105 | UGCCAUUUUGUCCUUUGAUU | 2840 | UAAUCAAAGGACAAAAUGGCAUU | 2973 |
| D-2367; | | | | | |
| D-2372 | | | | | |
| D-2363 | 2087-2107 | CCAUUUUGUCCUUUGAUUAU | 2841 | UAUAAUCAAAGGACAAAAUGGUU | 2974 |
| D-2368 | | | | | |
| D-2373 | | | | | |
| D-2446 | 1316-1334 | UUUUCACAUUUUUCGUCUUU | 2842 | AAGACGAAAAAUGUGAAAAUU | 2983 |
| D-2476 | 1314-1334 | UUUUCACAUUUUUCGUCUUU | 2842 | AAGACGAAAAAUGUGAAAAUC | 2975 |
| D-2473 | 2054-2072 | ACUCUAAGAUCUGAUGAAUU | 2843 | AUUCAUCAGAUCUUAGAGUUU | 3000 |
| D-2477 | 2052-2072 | ACUCUAAGAUCUGAUGAAUU | 2843 | AUUCAUCAGAUCUUAGAGUUA | 2995 |
| D-2474 | 1941-1959 | AAGAAAAGUGAUUCAGUGAU | 2844 | UCACUGAAUCACUUUUCUUUU | 3001 |
| D-2478 | 1939-1959 | AAGAAAAGUGAUUCAGUGAU | 2844 | UCACUGAAUCACUUUUCUUCU | 2996 |
| D-2447 | 1221-1239 | AUAUUAGAUGCCUUUUAAAU | 2845 | UUUAAAAGGCAUCUAAUAUUU | 2984 |
| D-2448 | 1999-2017 | GAAAGCAUAUGUCAGUUGUU | 2846 | ACAACUGACAUAUGCUUUCUU | 2985 |
| D-2449 | 2088-2106 | CAUUUUGUCCUUUGAUUAUU | 2847 | AUAAUCAAAGGACAAAAUGUU | 2986 |
| D-2450 | 1387-1405 | AUAUAGUCUCAAUAACUUAU | 2848 | UAAGUUAUUGAGACUAUAUUU | 2979 |
| D-2451 | 1508-1526 | CCAAGAGCGCAGACUUGCAU | 2849 | UGCAAGUCUGCGCUCUUGGUU | 2987 |
| D-2481 | 1506-1526 | CCAAGAGCGCAGACUUGCAU | 2849 | UGCAAGUCUGCGCUCUUGGUA | 2980 |
| D-2452 | 1229-1247 | UGCCUUUUAAAAAUGUUCUU | 2850 | AGAACAUUUUUAAAAGGCAUU | 2988 |
| D-2453 | 1213-1231 | GUCCUGGAAUAUUAGAUGUU | 2851 | ACAUCUAAUAUUCCAGGACUU | 2989 |
| D-2457 | 2056-2074 | CCUAAGAUCUGAUGAAGUAU | 2852 | UACUUCAUCAGAUCUUAGGUU | 2990 |
| D-2458; | 2056-2074 | CCUAAGAUCUGAUGAAGUAU | 2852 | UACUUCAUCAGAUCUUAGGUU | 2990 |
| D-2459; | | | | | |
| D-2460 | | | | | |
| D-2495 | 768-788 | UAGCAGACUUGUUCCGACUU | 2853 | AGUCGGAACAAGUCUGCUAUU | 3003 |
| D-2496 | 1455-1475 | CCGUUUAACUGAUUAUGGAU | 2854 | UCCAUAAUCAGUUAAACGGUU | 3009 |
| D-2497 | 1253-1273 | GACAACACUUGAAGCAUGUU | 2855 | ACAUGCUUCAAGUGUUGUCUU | 3010 |
| D-2498 | 2082-2102 | UUGCCAUUUUGUCCUUUGAU | 2856 | UCAAAGGACAAAAUGGCAAUU | 3011 |
| D-2499 | 2050-2070 | UAACUCUAAGAUCUGAUGAU | 2857 | UCAUCAGAUCUUAGAGUUAUU | 3012 |
| D-2500 | 1346-1366 | GUCUCAAUGCUUCAAUGUUU | 2858 | AACAUUGAAGCAUUGAGACUU | 3013 |
| D-2514 | 985-1005 | GUGAUGGCUUGUUCCGGAUG | 2859 | ACAUCCGGAACAAGCCAUCAC | 3014 |
| D-2515 | 985-1005 | GUGAUGGCUUGUUGCAGAUG | 2860 | ACAUCUGCAACAAGCCAUCAC | 3015 |
| D-2516 | 1092-1114 | CAGAACGAAAGUUAUGUGGA | 2861 | UUCCACAUAACUUUCGUUCUGAA | 3016 |
| D-2517 | 1092-1114 | CAGAACGAAAGUUGUAUGGA | 2862 | UUCCAUACAACUUUCGUUCUGAA | 3017 |
| D-2518 | 1210-1230 | UAUGUCCUGGAAUAUAAGAU | 2863 | AAUCUUAUAUUCCAGGACAUAUU | 3018 |
| D-2519 | 1210-1230 | UAUGUCCUGGAAUGUUAGAU | 2864 | AAUCUAACAUUCCAGGACAUAUU | 3019 |
| D-2520 | 1211-1231 | AUGUCCUGGAAUAUUGGAUG | 2865 | ACAUCCAAUAUUCCAGGACAUUU | 3020 |
| D-2521 | 1211-1231 | AUGUCCUGGAAUAAUAGAUG | 2866 | ACAUCUAUUAUUCCAGGACAUUU | 3021 |
| D-2522 | 1212-1232 | UGUCCUGGAAUAUUAAAUGC | 2867 | AGCAUUUAAUAUUCCAGGACAUU | 3022 |
| D-2523 | 1212-1232 | UGUCCUGGAAUAUAAGAUGC | 2868 | AGCAUCUUAUAUUCCAGGACAUU | 3023 |
| D-2524 | 1215-1233 | CCUGGAAUAUUAGGUGCCUU | 2869 | AGGCACCUAAUAUUCCAGGUU | 3024 |
| D-2529 | 1215-1235 | CCUGGAAUAUUAGGUGCCUU | 2869 | AAAGGCACCUAAUAUUCCAGGUU | 3029 |
| D-2525 | 1215-1233 | CCUGGAAUAUUGGAUGCCUU | 2870 | AGGCAUCCAAUAUUCCAGGUU | 3025 |
| D-2526 | 1214-1234 | UCCUGGAAUAUUAGAAGCCU | 2871 | AAGGCUUCUAAUAUUCCAGGA | 3026 |
| D-2527 | 1214-1234 | UCCUGGAAUAUUAAAUGCCU | 2872 | AAGGCAUUUAAUAUUCCAGGA | 3027 |
| D-2528 | 1215-1235 | CCUGGAAUAUUAGAUACCUU | 2873 | AAAGGUAUCUAAUAUUCCAGGUU | 3028 |
| D-2530 | 1216-1236 | CUGGAAUAUUAGAUGGCUUU | 2874 | AAAAGCCAUCUAAUAUUCCAGUU | 3030 |
| D-2531 | 1216-1236 | CUGGAAUAUUAGAAGCCUUU | 2875 | AAAAGGCUUCUAAUAUUCCAGUU | 3031 |
| D-2532 | 1219-1239 | GAAUAUUAGAUGCCUAUUAA | 2876 | UUUAAUAGGCAUCUAAUAUUCUU | 3032 |
| D-2533 | 1219-1239 | GAAUAUUAGAUGCGUUUUAA | 2877 | UUUAAAACGCAUCUAAUAUUCUU | 3033 |
| D-2534 | 1227-1247 | GAUGCCUUUUAAAAAAGUUC | 2878 | AGAACUUUUUUAAAAGGCAUCUU | 3034 |
| D-2535 | 1227-1247 | GAUGCCUUUUAAAGAUGUUC | 2879 | AGAACAUCUUUAAAAGGCAUCUU | 3035 |
| D-2536 | 1314-1334 | GAUUUUCACAUUUUUGGUCU | 2880 | AAGACCAAAAAUGUGAAAAUCUU | 3036 |
| D-2537 | 1314-1334 | GAUUUUCACAUUUAUCGUCU | 2881 | AAGACGAUAAAUGUGAAAAUCUU | 3037 |
| D-2538 | 1350-1370 | CUCAAUGCUUCAAUGACCCA | 2882 | AUGGGUCAUUGAAGCAUUGAGUU | 3038 |
| D-2539 | 1350-1370 | CUCAAUGCUUCAAAGUCCCA | 2883 | AUGGGACUUUGAAGCAUUGAGUU | 3039 |
| D-2540 | 1352-1372 | CAAUGCUUCAAUGUCGCAGU | 2884 | AACUGCGACAUUGAAGCAUUG | 3040 |
| D-2541 | 1352-1372 | CAAUGCUUCAAUGACCCAGU | 2885 | AACUGGGUCAUUGAAGCAUUG | 3041 |
| D-2542 | 1385-1405 | AAAUAUAGUCUCAAUGACUU | 2886 | UAAGUCAUUGAGACUAUAUUUUU | 3042 |
| D-2543 | 1385-1405 | AAAUAUAGUCUCAGUAACUU | 2887 | UAAGUUACUGAGACUAUAUUUUU | 3043 |
| D-2544 | 1438-1458 | GCAGGAUUCUGAAAAGUCCC | 2888 | AGGGACUUUUCAGAAUCCUGCUU | 3044 |
| D-2545 | 1438-1458 | GCAGGAUUCUGAAGACUCCC | 2889 | AGGGAGUCUUCAGAAUCCUGCUU | 3045 |
| D-2546 | 1506-1526 | UACCAAGAGCGCAGAGUUGC | 2890 | UGCAACUCUGCGCUCUUGGUAUU | 3046 |
| D-2547 | 1506-1526 | UACCAAGAGCGCAAACUUGC | 2891 | UGCAAGUUUGCGCUCUUGGUAUU | 3047 |
| D-2548 | 1997-2017 | AGGAAAGCAUAUGUCGGUUG | 2892 | ACAACCGACAUAUGCUUUCCUUU | 3048 |
| D-2549 | 1997-2017 | AGGAAAGCAUAUGACAGUUG | 2893 | ACAACUGUCAUAUGCUUUCCUUU | 3049 |
| D-2550 | 2016-2034 | GUUUAAAACCCAAAAUCUAU | 2894 | UAGAUUUUGGGUUUUAAACUU | 3050 |
| D-2551 | 2016-2034 | GUUUAAAACCCGAUAUCUAU | 2895 | UAGAUAUCGGGUUUUAAACUU | 3051 |
| D-2552 | 2039-2059 | UUAACUGAUUGUAUAGCUCU | 2896 | UAGAGCUAUACAAUCAGUUAAUU | 3052 |
| D-2553 | 2039-2059 | UUAACUGAUUGUAAAACUCU | 2897 | UAGAGUUUUACAAUCAGUUAAUU | 3053 |
| D-2554 | 2052-2072 | UAACUCUAAGAUCUGGUGAA | 2898 | AUUCACCAGAUCUUAGAGUUA | 3054 |
| D-2555 | 2052-2072 | UAACUCUAAGAUCAGAUGAA | 2899 | AUUCAUCUGAUCUUAGAGUUA | 3055 |
| D-2556 | 2054-2074 | ACUCUAAGAUCUGAUAAAGU | 2900 | UACUUUAUCAGAUCUUAGAGUUU | 3056 |
| D-2557 | 2054-2074 | ACUCUAAGAUCUGGUGAAGU | 2901 | UACUUCACCAGAUCUUAGAGUUU | 3057 |
| D-2558 | 2082-2102 | UAUUGCCAUUUUGUCGUUUG | 2902 | UCAAACGACAAAAUGGCAAUAUU | 3058 |
| D-2559 | 2082-2102 | UAUUGCCAUUUUGACCUUUG | 2903 | UCAAAGGUCAAAAUGGCAAUAUU | 3059 |
| D-2560 | 2086-2106 | GCCAUUUUGUCCUUUAAUUA | 2904 | AUAAUUAAAGGACAAAAUGGCUU | 3060 |
| D-2561 | 2086-2106 | GCCAUUUUGUCCUAUGAUUA | 2905 | AUAAUCAUAGGACAAAAUGGCUU | 3061 |

TABLE 2
Modified mARC1 siRNA sequences

| Duplex | | SEQ ID | | SEQ ID |
|---|---|---|---|---|
| No. | Sense Sequence (5′-3′) | NO: | Antisense Sequence (5′-3′) | NO: |
| D-1000 | [GalNAc3]sgagcaaGfcAfCfUfAfuauggaaus{invAb} | 1340 | usUfsccauAfuaguGfcUfugcucsgsu | 2072 |
| D-1001 | [GalNAc3]sagaaguUfcUfCfGfGfcaaaugaus{invAb} | 1341 | usCfsauuuGfccgaGfaAfcuucusgsu | 2073 |
| D-1002 | [GalNAc3]sgagcaaGfcUfGfAfAfuuuggaaus{invAb} | 1342 | usUfsccaaAfuucaGfcUfugcucsgsu | 2074 |
| D-1003 | [GalNAc3]sagaaguUfcAfGfCfGfcuaaugaus{invAb} | 1343 | usCfsauuaGfcgcuGfaAfcuucusgsu | 2075 |
| D-1004 | gsasaggaCfgCfAfCfUfgcucugaus{invAb} | 1344 | asAfsuCfaGfagcagugCfgUfccuucsusu | 2076 |
| D-1005 | asgsgacgCfaCfUfGfCfucugauugs{invAb} | 1345 | asCfsaAfuCfagagcagUfgCfguccususu | 2077 |
| D-1006 | gsgsacgcAfcUfGfCfUfcugauuggs{invAb} | 1346 | asCfscAfaUfcagagcaGfuGfcguccsusu | 2078 |
| D-1007 | ascsgcacUfgCfUfCfUfgauuggccs{invAb} | 1347 | asGfsgCfcAfaucagagCfaGfugcgususu | 2079 |
| D-1008 | csusgcucUfgAfUfUfGfgcccggaas{invAb} | 1348 | asUfsuCfcGfggccaauCfaGfagcagsusu | 2080 |
| D-1009 | usgscucuGfaUfUfGfGfcccggaags{invAb} | 1349 | asCfsuUfcCfgggccaaUfcAfgagcasusu | 2081 |
| D-1010 | gscsucugAfuUfGfGfCfccggaaggs{invAb} | 1350 | asCfscUfuCfcgggccaAfuCfagagcsusu | 2082 |
| D-1011 | csgsgggcCfaAfAfGfGfccgcaccus{invAb} | 1351 | asAfsgGfuGfcggccuuUfgGfccccgsusu | 2083 |
| D-1012 | gsgsggccAfaAfGfGfCfcgcaccuus{invAb} | 1352 | asAfsaGfgUfgcggccuUfuGfgccccsusu | 2084 |
| D-1013 | gscscaaaGfgCfCfGfCfaccuucccs{invAb} | 1353 | asGfsgGfaAfggugcggCfcUfuuggcsusu | 2085 |
| D-1014 | cscsaaagGfcCfGfCfAfccuuccccs{invAb} | 1354 | asGfsgGfgAfaggugcgGfcCfuuuggsusu | 2086 |
| D-1015 | csgsccacCfuCfGfCfGfgagaagccs{invAb} | 1355 | usGfsgCfuUfcuccgcgAfgGfuggcgsusu | 2087 |
| D-1016 | gscscaccUfcGfCfGfGfagaagccas{invAb} | 1356 | asUfsgGfcUfucuccgcGfaGfguggcsusu | 2088 |
| D-1017 | cscsaccuCfgCfGfGfAfgaagccags{invAb} | 1357 | asCfsuGfgCfuucuccgCfgAfgguggsusu | 2089 |
| D-1018 | ascscucgCfgGfAfGfAfagccagccs{invAb} | 1358 | usGfsgCfuGfgcuucucCfgCfgaggususu | 2090 |
| D-1019 | usgsaucaAfcCfAfGfGfagggaaacs{invAb} | 1359 | usGfsuUfuCfccuccugGfuUfgaucasusu | 2091 |
| D-1020 | asuscaacCfaGfGfAfGfggaaacaus{invAb} | 1360 | asAfsuGfuUfucccuccUfgGfuugaususu | 2092 |
| D-1021 | uscsaaccAfgGfAfGfGfgaaacaugs{invAb} | 1361 | asCfsaUfgUfuucccucCfuGfguugasusu | 2093 |
| D-1022 | csasaccaGfgAfGfGfGfaaacauggs{invAb} | 1362 | asCfscAfuGfuuucccuCfcUfgguugsusu | 2094 |
| D-1023 | asasccagGfaGfGfGfAfaacauggus{invAb} | 1363 | asAfscCfaUfguuucccUfcCfugguususu | 2095 |
| D-1024 | ascscaggAfgGfGfAfAfacaugguus{invAb} | 1364 | usAfsaCfcAfuguuuccCfuCfcuggususu | 2096 |
| D-1025 | usgscucgCfcAfGfGfAfaccucgccs{invAb} | 1365 | asGfsgCfgAfgguuccuGfgCfgagcasusu | 2097 |
| D-1026 | csuscgccAfgGfAfAfCfcucgccugs{invAb} | 1366 | asCfsaGfgCfgagguucCfuGfgcgagsusu | 2098 |
| D-1027 | gsgsaaccUfcGfCfCfUfgguccugas{invAb} | 1367 | asUfscAfgGfaccaggcGfaGfguuccsusu | 2099 |
| D-1028 | asasccucGfcCfUfGfGfuccugauus{invAb} | 1368 | asAfsaUfcAfggaccagGfcGfagguususu | 2100 |
| D-1029 | ascscucgCfcUfGfGfUfccugauuus{invAb} | 1369 | asAfsaAfuCfaggaccaGfgCfgaggususu | 2101 |
| D-1030 | cscsucgcCfuGfGfUfCfcugauuucs{invAb} | 1370 | asGfsaAfaUfcaggaccAfgGfcgaggsusu | 2102 |
| D-1031 | csuscgccUfgGfUfCfCfugauuuccs{invAb} | 1371 | asGfsgAfaAfucaggacCfaGfgcgagsusu | 2103 |
| D-1032 | cscsugguCfcUfGfAfUfuucccugas{invAb} | 1372 | asUfscAfgGfgaaaucaGfgAfccaggsusu | 2104 |
| D-1033 | gsascucuCfaGfUfGfCfagccuacas{invAb} | 1373 | asUfsgUfaGfgcugcacUfgAfgagucsusu | 2105 |
| D-1034 | csuscucaGfuGfCfAfGfccuacacas{invAb} | 1374 | usUfsgUfgUfaggcugcAfcUfgagagsusu | 2106 |
| D-1035 | uscsucagUfgCfAfGfCfcuacacaas{invAb} | 1375 | usUfsuGfuGfuaggcugCfaCfugagasusu | 2107 |
| D-1036 | csuscaguGfcAfGfCfCfuacacaaas{invAb} | 1376 | asUfsuUfgUfguaggcuGfcAfcugagsusu | 2108 |
| D-1037 | uscsagugCfaGfCfCfUfacacaaags{invAb} | 1377 | asCfsuUfuGfuguaggcUfgCfacugasusu | 2109 |
| D-1038 | csusaucaAfaAfCfGfCfccaccacas{invAb} | 1378 | usUfsgUfgGfugggcguUfuUfgauagsusu | 2110 |
| D-1039 | usasucaaAfaCfGfCfCfcaccacaas{invAb} | 1379 | usUfsuGfuGfgugggcgUfuUfugauasusu | 2111 |
| D-1040 | asuscaaaAfcGfCfCfCfaccacaaas{invAb} | 1380 | asUfsuUfgUfggugggcGfuUfuugaususu | 2112 |
| D-1041 | uscsaaaaCfgCfCfCfAfccacaaaus{invAb} | 1381 | asAfsuUfuGfuggugggCfgUfuuugasusu | 2113 |
| D-1042 | asasacgcCfcAfCfCfAfcaaaugcas{invAb} | 1382 | asUfsgCfaUfuugugguGfgGfcguuususu | 2114 |
| D-1043 | asascgccCfaCfCfAfCfaaaugcags{invAb} | 1383 | asCfsuGfcAfuuuguggUfgGfgcguususu | 2115 |
| D-1044 | cscsagugGfaUfAfAfCfcagcuuccs{invAb} | 1384 | asGfsgAfaGfcugguuaUfcCfacuggsusu | 2116 |
| D-1045 | csasguggAfuAfAfCfCfagcuuccus{invAb} | 1385 | asAfsgGfaAfgcugguuAfuCfcacugsusu | 2117 |
| D-1046 | gsusggauAfaCfCfAfGfcuuccugas{invAb} | 1386 | usUfscAfgGfaagcuggUfuAfuccacsusu | 2118 |
| D-1047 | gsasuaacCfaGfCfUfUfccugaagus{invAb} | 1387 | asAfscUfuCfaggaagcUfgGfuuaucsusu | 2119 |
| D-1048 | asuscaaaUfaGfCfAfGfacuuguucs{invAb} | 1388 | asGfsaAfcAfagucugcUfaUfuugaususu | 2120 |
| D-1049 | csasaauaGfcAfGfAfCfuuguuccgs{invAb} | 1389 | usCfsgGfaAfcaagucuGfcUfauuugsusu | 2121 |
| D-1050 | asasauagCfaGfAfCfUfuguuccgas{invAb} | 1390 | asUfscGfgAfacaagucUfgCfuauuususu | 2122 |
| D-1051 | usgsagcuUfcUfUfAfUfuggugacgs{invAb} | 1391 | asCfsgUfcAfccaauaaGfaAfgcucasusu | 2123 |
| D-1052 | asgscuucUfuAfUfUfGfgugacgugs{invAb} | 1392 | asCfsaCfgUfcaccaauAfaGfaagcususu | 2124 |
| D-1053 | gscsuucuUfaUfUfGfGfugacguggs{invAb} | 1393 | usCfscAfcGfucaccaaUfaAfgaagcsusu | 2125 |
| D-1054 | ususcuuaUfuGfGfUfGfacguggaas{invAb} | 1394 | asUfsuCfcAfcgucaccAfaUfaagaasusu | 2126 |
| D-1055 | ususggugAfcGfUfGfGfaacugaaas{invAb} | 1395 | usUfsuUfcAfguuccacGfuCfaccaasusu | 2127 |
| D-1056 | usgsgugaCfgUfGfGfAfacugaaaas{invAb} | 1396 | asUfsuUfuCfaguuccaCfgUfcaccasusu | 2128 |
| D-1057 | gsgsugacGfuGfGfAfAfcugaaaags{invAb} | 1397 | asCfsuUfuUfcaguuccAfcGfucaccsusu | 2129 |
| D-1058 | gsusgacgUfgGfAfAfCfugaaaaggs{invAb} | 1398 | asCfscUfuUfucaguucCfaCfgucacsusu | 2130 |
| D-1059 | gscsuuguUfcCfAfGfAfugcauuuus{invAb} | 1399 | usAfsaAfaUfgcaucugGfaAfcaagcsusu | 2131 |
| D-1060 | gsusuccaGfaUfGfCfAfuuuuaaccs{invAb} | 1400 | usGfsgUfuAfaaaugcaUfcUfggaacsusu | 2132 |
| D-1061 | ususccagAfuGfCfAfUfuuuaaccas{invAb} | 1401 | asUfsgGfuUfaaaaugcAfuCfuggaasusu | 2133 |
| D-1062 | usgscauuUfuAfAfCfCfacaguggas{invAb} | 1402 | asUfscCfaCfugugguuAfaAfaugcasusu | 2134 |
| D-1063 | gscsauuuUfaAfCfCfAfcaguggacs{invAb} | 1403 | asGfsuCfcAfcugugguUfaAfaaugcsusu | 2135 |
| D-1064 | gsgsugucAfuGfAfGfCfaggaaggas{invAb} | 1404 | usUfscCfuUfccugcucAfuGfacaccsusu | 2136 |
| D-1065 | gsasaccgCfuGfGfAfAfacacugaas{invAb} | 1405 | asUfsuCfaGfuguuuccAfgCfgguucsusu | 2137 |
| D-1066 | gscsuggaAfaCfAfCfUfgaagaguus{invAb} | 1406 | usAfsaCfuCfuucagugUfuUfccagcsusu | 2138 |
| D-1067 | gsgsaaacAfcUfGfAfAfgaguuaucs{invAb} | 1407 | asGfsaUfaAfcucuucaGfuGfuuuccsusu | 2139 |
| D-1068 | gsasaacaCfuGfAfAfGfaguuaucgs{invAb} | 1408 | asCfsgAfuAfacucuucAfgUfguuucsusu | 2140 |
| D-1069 | asasacacUfgAfAfGfAfguuaucgcs{invAb} | 1409 | asGfscGfaUfaacucuuCfaGfuguuususu | 2141 |
| D-1070 | asascacuGfaAfGfAfGfuuaucgccs{invAb} | 1410 | usGfsgCfgAfuaacucuUfcAfguguususu | 2142 |
| D-1071 | ascsacugAfaGfAfGfUfuaucgccas{invAb} | 1411 | asUfsgGfcGfauaacucUfuCfagugususu | 2143 |
| D-1072 | csascugaAfgAfGfUfUfaucgccags{invAb} | 1412 | asCfsuGfgCfgauaacuCfuUfcagugsusu | 2144 |
| D-1073 | ascsugaaGfaGfUfUfAfucgccagus{invAb} | 1413 | asAfscUfgGfcgauaacUfcUfucagususu | 2145 |
| D-1074 | csusgaagAfgUfUfAfUfcgccagugs{invAb} | 1414 | asCfsaCfuGfgcgauaaCfuCfuucagsusu | 2146 |
| D-1075 | usgsaagaGfuUfAfUfCfgccagugus{invAb} | 1415 | asAfscAfcUfggcgauaAfcUfcuucasusu | 2147 |
| D-1076 | gsasagagUfuAfUfCfGfccagugugs{invAb} | 1416 | usCfsaCfaCfuggcgauAfaCfucuucsusu | 2148 |
| D-1077 | gsasguuaUfcGfCfCfAfgugugaccs{invAb} | 1417 | asGfsgUfcAfcacuggcGfaUfaacucsusu | 2149 |
| D-1078 | asgsuuauCfgCfCfAfGfugugacccs{invAb} | 1418 | asGfsgGfuCfacacuggCfgAfuaacususu | 2150 |
| D-1079 | gsusuaucGfcCfAfGfUfgugacccus{invAb} | 1419 | asAfsgGfgUfcacacugGfcGfauaacsusu | 2151 |
| D-1080 | ususaucgCfcAfGfUfGfugacccuus{invAb} | 1420 | asAfsaGfgGfucacacuGfgCfgauaasusu | 2152 |
| D-1081 | csgsccagUfgUfGfAfCfccuucagas{invAb} | 1421 | usUfscUfgAfagggucaCfaCfuggcgsusu | 2153 |
| D-1082 | gscscaguGfuGfAfCfCfcuucagaas{invAb} | 1422 | asUfsuCfuGfaagggucAfcAfcuggcsusu | 2154 |
| D-1083 | csasguguGfaCfCfCfUfucagaacgs{invAb} | 1423 | usCfsgUfuCfugaagggUfcAfcacugsusu | 2155 |
| D-1084 | asgsugugAfcCfCfUfUfcagaacgas{invAb} | 1424 | usUfscGfuUfcugaaggGfuCfacacususu | 2156 |
| D-1085 | gsusgugaCfcCfUfUfCfagaacgaas{invAb} | 1425 | usUfsuCfgUfucugaagGfgUfcacacsusu | 2157 |
| D-1086 | usgsugacCfcUfUfCfAfgaacgaaas{invAb} | 1426 | asUfsuUfcGfuucugaaGfgGfucacasusu | 2158 |
| D-1087 | gsusgaccCfuUfCfAfGfaacgaaags{invAb} | 1427 | asCfsuUfuCfguucugaAfgGfgucacsusu | 2159 |
| D-1088 | usgsacccUfuCfAfGfAfacgaaagus{invAb} | 1428 | asAfscUfuUfcguucugAfaGfggucasusu | 2160 |
| D-1089 | gsascccuUfcAfGfAfAfcgaaaguus{invAb} | 1429 | usAfsaCfuUfucguucuGfaAfgggucsusu | 2161 |
| D-1090 | ascsccuuCfaGfAfAfCfgaaaguuas{invAb} | 1430 | asUfsaAfcUfuucguucUfgAfagggususu | 2162 |
| D-1091 | cscscuucAfgAfAfCfGfaaaguuaus{invAb} | 1431 | usAfsuAfaCfuuucguuCfuGfaagggsusu | 2163 |
| D-1092 | cscsuucaGfaAfCfGfAfaaguuauas{invAb} | 1432 | asUfsaUfaAfcuuucguUfcUfgaaggsusu | 2164 |
| D-1093 | csusucagAfaCfGfAfAfaguuauaus{invAb} | 1433 | asAfsuAfuAfacuuucgUfuCfugaagsusu | 2165 |
| D-1094 | ususcagaAfcGfAfAfAfguuauaugs{invAb} | 1434 | asCfsaUfaUfaacuuucGfuUfcugaasusu | 2166 |
| D-1095 | uscsagaaCfgAfAfAfGfuuauauggs{invAb} | 1435 | usCfscAfuAfuaacuuuCfgUfucugasusu | 2167 |
| D-1096 | csasgaacGfaAfAfGfUfuauauggas{invAb} | 1436 | usUfscCfaUfauaacuuUfcGfuucugsusu | 2168 |
| D-1097 | asgsuuauAfuGfGfAfAfaaucaccas{invAb} | 1437 | asUfsgGfuGfauuuuccAfuAfuaacususu | 2169 |
| D-1098 | ususauauGfgAfAfAfAfucaccacus{invAb} | 1438 | asAfsgUfgGfugauuuuCfcAfuauaasusu | 2170 |
| D-1099 | asasuagcAfgAfCfUfUfguuccgacs{invAb} | 1439 | asGfsuCfgGfaacaaguCfuGfcuauususu | 2171 |
| D-1100 | asusagcaGfaCfUfUfGfuuccgaccs{invAb} | 1440 | asGfsgUfcGfgaacaagUfcUfgcuaususu | 2172 |
| D-1101 | usasgcagAfcUfUfGfUfuccgacccs{invAb} | 1441 | usGfsgGfuCfggaacaaGfuCfugcuasusu | 2173 |
| D-1102 | asgscagaCfuUfGfUfUfccgacccas{invAb} | 1442 | usUfsgGfgUfcggaacaAfgUfcugcususu | 2174 |
| D-1103 | gscsagacUfuGfUfUfCfcgacccaas{invAb} | 1443 | asUfsuGfgGfucggaacAfaGfucugcsusu | 2175 |
| D-1104 | csasgacuUfgUfUfCfCfgacccaags{invAb} | 1444 | asCfsuUfgGfgucggaaCfaAfgucugsusu | 2176 |
| D-1105 | asgsacuuGfuUfCfCfGfacccaaggs{invAb} | 1445 | usCfscUfuGfggucggaAfcAfagucususu | 2177 |
| D-1106 | gsascuugUfuCfCfGfAfcccaaggas{invAb} | 1446 | asUfscCfuUfgggucggAfaCfaagucsusu | 2178 |
| D-1107 | ascsuuguUfcCfGfAfCfccaaggacs{invAb} | 1447 | asGfsuCfcUfugggucgGfaAfcaagususu | 2179 |
| D-1108 | csusuguuCfcGfAfCfCfcaaggaccs{invAb} | 1448 | usGfsgUfcCfuugggucGfgAfacaagsusu | 2180 |
| D-1109 | ususccgaCfcCfAfAfGfgaccagaus{invAb} | 1449 | asAfsuCfuGfguccuugGfgUfcggaasusu | 2181 |
| D-1110 | asasggacCfaGfAfUfUfgcuuacucs{invAb} | 1450 | usGfsaGfuAfagcaaucUfgGfuccuususu | 2182 |
| D-1111 | gsasccagAfuUfGfCfUfuacucagas{invAb} | 1451 | asUfscUfgAfguaagcaAfuCfuggucsusu | 2183 |
| D-1112 | cscsagauUfgCfUfUfAfcucagacas{invAb} | 1452 | asUfsgUfcUfgaguaagCfaAfucuggsusu | 2184 |
| D-1113 | csasgauuGfcUfUfAfCfucagacacs{invAb} | 1453 | asGfsuGfuCfugaguaaGfcAfaucugsusu | 2185 |
| D-1114 | asgsauugCfuUfAfCfUfcagacaccs{invAb} | 1454 | usGfsgUfgUfcugaguaAfgCfaaucususu | 2186 |
| D-1115 | gsasuugcUfuAfCfUfCfagacaccas{invAb} | 1455 | asUfsgGfuGfucugaguAfaGfcaaucsusu | 2187 |
| D-1116 | asusugcuUfaCfUfCfAfgacaccags{invAb} | 1456 | asCfsuGfgUfgucugagUfaAfgcaaususu | 2188 |
| D-1117 | gscsuuacUfcAfGfAfCfaccagcccs{invAb} | 1457 | usGfsgGfcUfggugucuGfaGfuaagcsusu | 2189 |
| D-1118 | ususacucAfgAfCfAfCfcagcccaus{invAb} | 1458 | asAfsuGfgGfcugguguCfuGfaguaasusu | 2190 |
| D-1119 | csgsgaucUfcAfAfCfUfccaggcuas{invAb} | 1459 | asUfsaGfcCfuggaguuGfaGfauccgsusu | 2191 |
| D-1120 | gsgsaucuCfaAfCfUfCfcaggcuags{invAb} | 1460 | usCfsuAfgCfcuggaguUfgAfgauccsusu | 2192 |
| D-1121 | gsasucucAfaCfUfCfCfaggcuagas{invAb} | 1461 | asUfscUfaGfccuggagUfuGfagaucsusu | 2193 |
| D-1122 | asuscucaAfcUfCfCfAfggcuagags{invAb} | 1462 | usCfsuCfuAfgccuggaGfuUfgagaususu | 2194 |
| D-1123 | asascuccAfgGfCfUfAfgagaagaas{invAb} | 1463 | usUfsuCfuUfcucuagcCfuGfgaguususu | 2195 |
| D-1124 | asgsaagaAfaGfUfUfAfaagcaaccs{invAb} | 1464 | usGfsgUfuGfcuuuaacUfuUfcuucususu | 2196 |
| D-1125 | gsasagaaAfgUfUfAfAfagcaaccas{invAb} | 1465 | usUfsgGfuUfgcuuuaaCfuUfucuucsusu | 2197 |
| D-1126 | asasgaaaGfuUfAfAfAfgcaaccaas{invAb} | 1466 | asUfsuGfgUfugcuuuaAfcUfuucuususu | 2198 |
| D-1127 | asgsaaagUfuAfAfAfGfcaaccaacs{invAb} | 1467 | asGfsuUfgGfuugcuuuAfaCfuuucususu | 2199 |
| D-1128 | gsasaaguUfaAfAfGfCfaaccaacus{invAb} | 1468 | asAfsgUfuGfguugcuuUfaAfcuuucsusu | 2200 |
| D-1129 | asasaguuAfaAfGfCfAfaccaacuus{invAb} | 1469 | asAfsaGfuUfgguugcuUfuAfacuuususu | 2201 |
| D-1130 | asasguuaAfaGfCfAfAfccaacuucs{invAb} | 1470 | usGfsaAfgUfugguugcUfuUfaacuususu | 2202 |
| D-1131 | asgsuuaaAfgCfAfAfCfcaacuucas{invAb} | 1471 | asUfsgAfaGfuugguugCfuUfuaacususu | 2203 |
| D-1132 | gsusuaaaGfcAfAfCfCfaacuucags{invAb} | 1472 | asCfsuGfaAfguugguuGfcUfuuaacsusu | 2204 |
| D-1133 | asasagcaAfcCfAfAfCfuucaggccs{invAb} | 1473 | asGfsgCfcUfgaaguugGfuUfgcuuususu | 2205 |
| D-1134 | asgscaacCfaAfCfUfUfcaggcccas{invAb} | 1474 | usUfsgGfgCfcugaaguUfgGfuugcususu | 2206 |
| D-1135 | csasaccaAfcUfUfCfAfggcccaaus{invAb} | 1475 | usAfsuUfgGfgccugaaGfuUfgguugsusu | 2207 |
| D-1136 | asasccaaCfuUfCfAfGfgcccaauas{invAb} | 1476 | asUfsaUfuGfggccugaAfgUfugguususu | 2208 |
| D-1137 | cscsaacuUfcAfGfGfCfccaauauus{invAb} | 1477 | asAfsaUfaUfugggccuGfaAfguuggsusu | 2209 |
| D-1138 | csasacuuCfaGfGfCfCfcaauauugs{invAb} | 1478 | asCfsaAfuAfuugggccUfgAfaguugsusu | 2210 |
| D-1139 | ascsuucaGfgCfCfCfAfauauuguas{invAb} | 1479 | usUfsaCfaAfuauugggCfcUfgaagususu | 2211 |
| D-1140 | csusucagGfcCfCfAfAfuauuguaas{invAb} | 1480 | asUfsuAfcAfauauuggGfcCfugaagsusu | 2212 |
| D-1141 | ususcaggCfcCfAfAfUfauuguaaus{invAb} | 1481 | asAfsuUfaCfaauauugGfgCfcugaasusu | 2213 |
| D-1142 | uscsaggcCfcAfAfUfAfuuguaauus{invAb} | 1482 | asAfsaUfuAfcaauauuGfgGfccugasusu | 2214 |
| D-1143 | asgsgcccAfaUfAfUfUfguaauuucs{invAb} | 1483 | usGfsaAfaUfuacaauaUfuGfggccususu | 2215 |
| D-1144 | gsgscccaAfuAfUfUfGfuaauuucas{invAb} | 1484 | asUfsgAfaAfuuacaauAfuUfgggccsusu | 2216 |
| D-1145 | gsasugagCfuUfCfUfUfauuggugas{invAb} | 1485 | asUfscAfcCfaauaagaAfgCfucaucsusu | 2217 |
| D-1146 | asusgagcUfuCfUfUfAfuuggugacs{invAb} | 1486 | asGfsuCfaCfcaauaagAfaGfcucaususu | 2218 |
| D-1147 | asusauggAfaAfAfUfCfaccacucus{invAb} | 1487 | asAfsgAfgUfggugauuUfuCfcauaususu | 2219 |
| D-1148 | usasuggaAfaAfUfCfAfccacucuus{invAb} | 1488 | asAfsaGfaGfuggugauUfuUfccauasusu | 2220 |
| D-1149 | asusggaaAfaUfCfAfCfcacucuuus{invAb} | 1489 | asAfsaAfgAfguggugaUfuUfuccaususu | 2221 |
| D-1150 | usgsgaaaAfuCfAfCfCfacucuuugs{invAb} | 1490 | asCfsaAfaGfaguggugAfuUfuuccasusu | 2222 |
| D-1151 | csusggaaAfaCfCfCfAfgggaccaus{invAb} | 1491 | asAfsuGfgUfcccugggUfuUfuccagsusu | 2223 |
| D-1152 | gsgsaaaaCfcCfAfGfGfgaccaucas{invAb} | 1492 | usUfsgAfuGfgucccugGfgUfuuuccsusu | 2224 |
| D-1153 | gsasaaacCfcAfGfGfGfaccaucaas{invAb} | 1493 | usUfsuGfaUfggucccuGfgGfuuuucsusu | 2225 |
| D-1154 | cscscaggGfaCfCfAfUfcaaaguggs{invAb} | 1494 | asCfscAfcUfuugauggUfcCfcugggsusu | 2226 |
| D-1155 | cscsagggAfcCfAfUfCfaaagugggs{invAb} | 1495 | usCfscCfaCfuuugaugGfuCfccuggsusu | 2227 |
| D-1156 | gsgsgaccAfuCfAfAfAfgugggagas{invAb} | 1496 | asUfscUfcCfcacuuugAfuGfgucccsusu | 2228 |
| D-1157 | gsgsgagaCfcCfUfGfUfguaccugcs{invAb} | 1497 | asGfscAfgGfuacacagGfgUfcucccsusu | 2229 |
| D-1158 | gsusaccuGfcUfGfGfGfccaguaaus{invAb} | 1498 | asAfsuUfaCfuggcccaGfcAfgguacsusu | 2230 |
| D-1159 | usgscuggGfcCfAfGfUfaaugggaas{invAb} | 1499 | asUfsuCfcCfauuacugGfcCfcagcasusu | 2231 |
| D-1160 | asasauguUfcUfCfAfAfaaaugacas{invAb} | 1500 | usUfsgUfcAfuuuuugaGfaAfcauuususu | 2232 |
| D-1161 | asasuguuCfuCfAfAfAfaaugacaas{invAb} | 1501 | asUfsuGfuCfauuuuugAfgAfacauususu | 2233 |
| D-1162 | asasaaugAfcAfAfCfAfcuugaagcs{invAb} | 1502 | usGfscUfuCfaaguguuGfuCfauuuususu | 2234 |
| D-1163 | asasaugaCfaAfCfAfCfuugaagcas{invAb} | 1503 | asUfsgCfuUfcaaguguUfgUfcauuususu | 2235 |
| D-1164 | asasugacAfaCfAfCfUfugaagcaus{invAb} | 1504 | asAfsuGfcUfucaagugUfuGfucauususu | 2236 |
| D-1165 | usgsacaaCfaCfUfUfGfaagcauggs{invAb} | 1505 | asCfscAfuGfcuucaagUfgUfugucasusu | 2237 |
| D-1166 | gsascaacAfcUfUfGfAfagcauggus{invAb} | 1506 | asAfscCfaUfgcuucaaGfuGfuugucsusu | 2238 |
| D-1167 | ascsaacaCfuUfGfAfAfgcauggugs{invAb} | 1507 | asCfsaCfcAfugcuucaAfgUfguugususu | 2239 |
| D-1168 | csascuugAfaGfCfAfUfgguguuucs{invAb} | 1508 | usGfsaAfaCfaccaugcUfuCfaagugsusu | 2240 |
| D-1169 | csusugaaGfcAfUfGfGfuguuucags{invAb} | 1509 | usCfsuGfaAfacaccauGfcUfucaagsusu | 2241 |
| D-1170 | csusggugUfcUfCfAfAfugcuucaas{invAb} | 1510 | asUfsuGfaAfgcauugaGfaCfaccagsusu | 2242 |
| D-1171 | usgsguguCfuCfAfAfUfgcuucaaus{invAb} | 1511 | asAfsuUfgAfagcauugAfgAfcaccasusu | 2243 |
| D-1172 | gsgsugucUfcAfAfUfGfcuucaaugs{invAb} | 1512 | asCfsaUfuGfaagcauuGfaGfacaccsusu | 2244 |
| D-1173 | gsusgucuCfaAfUfGfCfuucaaugus{invAb} | 1513 | asAfscAfuUfgaagcauUfgAfgacacsusu | 2245 |
| D-1174 | usgsucucAfaUfGfCfUfucaaugucs{invAb} | 1514 | asGfsaCfaUfugaagcaUfuGfagacasusu | 2246 |
| D-1175 | uscsucaaUfgCfUfUfCfaaugucccs{invAb} | 1515 | usGfsgGfaCfauugaagCfaUfugagasusu | 2247 |
| D-1176 | csuscaauGfcUfUfCfAfaugucccas{invAb} | 1516 | asUfsgGfgAfcauugaaGfcAfuugagsusu | 2248 |
| D-1177 | csasaugcUfuCfAfAfUfgucccagus{invAb} | 1517 | asAfscUfgGfgacauugAfaGfcauugsusu | 2249 |
| D-1178 | asusgcuuCfaAfUfGfUfcccagugcs{invAb} | 1518 | usGfscAfcUfgggacauUfgAfagcaususu | 2250 |
| D-1179 | usgscuucAfaUfGfUfCfccagugcas{invAb} | 1519 | usUfsgCfaCfugggacaUfuGfaagcasusu | 2251 |
| D-1180 | asasugacAfaGfAfCfAfggauucugs{invAb} | 1520 | usCfsaGfaAfuccugucUfuGfucauususu | 2252 |
| D-1181 | asusgacaAfgAfCfAfGfgauucugas{invAb} | 1521 | usUfscAfgAfauccuguCfuUfgucaususu | 2253 |
| D-1182 | gsascaagAfcAfGfGfAfuucugaaas{invAb} | 1522 | usUfsuUfcAfgaauccuGfuCfuugucsusu | 2254 |
| D-1183 | asasgacaGfgAfUfUfCfugaaaacus{invAb} | 1523 | asAfsgUfuUfucagaauCfcUfgucuususu | 2255 |
| D-1184 | ascsaggaUfuCfUfGfAfaaacucccs{invAb} | 1524 | asGfsgGfaGfuuuucagAfaUfccugususu | 2256 |
| D-1185 | cscscguuUfaAfCfUfGfauuauggas{invAb} | 1525 | usUfscCfaUfaaucaguUfaAfacgggsusu | 2257 |
| D-1186 | ususuaacUfgAfUfUfAfuggaauags{invAb} | 1526 | asCfsuAfuUfccauaauCfaGfuuaaasusu | 2258 |
| D-1187 | ususaacuGfaUfUfAfUfggaauagus{invAb} | 1527 | asAfscUfaUfuccauaaUfcAfguuaasusu | 2259 |
| D-1188 | asascugaUfuAfUfGfGfaauaguucs{invAb} | 1528 | asGfsaAfcUfauuccauAfaUfcaguususu | 2260 |
| D-1189 | ascsugauUfaUfGfGfAfauaguucus{invAb} | 1529 | asAfsgAfaCfuauuccaUfaAfucagususu | 2261 |
| D-1190 | csusgauuAfuGfGfAfAfuaguucuus{invAb} | 1530 | asAfsaGfaAfcuauuccAfuAfaucagsusu | 2262 |
| D-1191 | gsasuuauGfgAfAfUfAfguucuuucs{invAb} | 1531 | asGfsaAfaGfaacuauuCfcAfuaaucsusu | 2263 |
| D-1192 | asusuaugGfaAfUfAfGfuucuuucus{invAb} | 1532 | asAfsgAfaAfgaacuauUfcCfauaaususu | 2264 |
| D-1193 | ususgcauCfcUfGfUfCfacuaccacs{invAb} | 1533 | asGfsuGfgUfagugacaGfgAfugcaasusu | 2265 |
| D-1194 | csasccccAfaAfUfAfUfggcuggaas{invAb} | 1534 | asUfsuCfcAfgccauauUfuGfgggugsusu | 2266 |
| D-1195 | cscsccaaAfuAfUfGfGfcuggaaugs{invAb} | 1535 | asCfsaUfuCfcagccauAfuUfuggggsusu | 2267 |
| D-1196 | csuscaagCfcCfCfGfGfgcuagcuus{invAb} | 1536 | asAfsaGfcUfagcccggGfgCfuugagsusu | 2268 |
| D-1197 | uscsaagcCfcCfGfGfGfcuagcuuus{invAb} | 1537 | asAfsaAfgCfuagcccgGfgGfcuugasusu | 2269 |
| D-1198 | asasgcccCfgGfGfCfUfagcuuuugs{invAb} | 1538 | usCfsaAfaAfgcuagccCfgGfggcuususu | 2270 |
| D-1199 | asgsccccGfgGfCfUfAfgcuuuugas{invAb} | 1539 | usUfscAfaAfagcuagcCfcGfgggcususu | 2271 |
| D-1200 | gscscccgGfgCfUfAfGfcuuuugaas{invAb} | 1540 | usUfsuCfaAfaagcuagCfcCfggggcsusu | 2272 |
| D-1201 | cscscgggCfuAfGfCfUfuuugaaaus{invAb} | 1541 | asAfsuUfuCfaaaagcuAfgCfccgggsusu | 2273 |
| D-1202 | gsgscuagCfuUfUfUfGfaaauggcas{invAb} | 1542 | asUfsgCfcAfuuucaaaAfgCfuagccsusu | 2274 |
| D-1203 | asusaaagAfcUfGfAfGfgugaccuus{invAb} | 1543 | asAfsaGfgUfcaccucaGfuCfuuuaususu | 2275 |
| D-1204 | csusgcagAfuAfUfUfAfauuuuccas{invAb} | 1544 | asUfsgGfaAfaauuaauAfuCfugcagsusu | 2276 |
| D-1205 | gsasuauuAfaUfUfUfUfccauagaus{invAb} | 1545 | asAfsuCfuAfuggaaaaUfuAfauaucsusu | 2277 |
| D-1206 | asusauuaAfuUfUfUfCfcauagaucs{invAb} | 1546 | asGfsaUfcUfauggaaaAfuUfaauaususu | 2278 |
| D-1207 | usasauuuUfcCfAfUfAfgaucuggas{invAb} | 1547 | asUfscCfaGfaucuaugGfaAfaauuasusu | 2279 |
| D-1208 | asasuuuuCfcAfUfAfGfaucuggaus{invAb} | 1548 | asAfsuCfcAfgaucuauGfgAfaaauususu | 2280 |
| D-1209 | asusuuucCfaUfAfGfAfucuggaucs{invAb} | 1549 | asGfsaUfcCfagaucuaUfgGfaaaaususu | 2281 |
| D-1210 | ususuccaUfaGfAfUfCfuggaucugs{invAb} | 1550 | asCfsaGfaUfccagaucUfaUfggaaasusu | 2282 |
| D-1211 | usgscuucUfcAfGfAfCfagcauuggs{invAb} | 1551 | usCfscAfaUfgcugucuGfaGfaagcasusu | 2283 |
| D-1212 | gscsuucuCfaGfAfCfAfgcauuggas{invAb} | 1552 | asUfscCfaAfugcugucUfgAfgaagcsusu | 2284 |
| D-1213 | uscsagacAfgCfAfUfUfggauuuccs{invAb} | 1553 | asGfsgAfaAfuccaaugCfuGfucugasusu | 2285 |
| D-1214 | csasgacaGfcAfUfUfGfgauuuccus{invAb} | 1554 | usAfsgGfaAfauccaauGfcUfgucugsusu | 2286 |
| D-1215 | asgsacagCfaUfUfGfGfauuuccuas{invAb} | 1555 | usUfsaGfgAfaauccaaUfgCfugucususu | 2287 |
| D-1216 | ususccuaAfaGfGfUfGfcucaggags{invAb} | 1556 | asCfsuCfcUfgagcaccUfuUfaggaasusu | 2288 |
| D-1217 | asgsgaccCfcUfGfGfAfuccuugccs{invAb} | 1557 | usGfsgCfaAfggauccaGfgGfguccususu | 2289 |
| D-1218 | cscscuggAfuCfCfUfUfgccauuccs{invAb} | 1558 | asGfsgAfaUfggcaaggAfuCfcagggsusu | 2290 |
| D-1219 | csusggauCfcUfUfGfCfcauuccccs{invAb} | 1559 | asGfsgGfgAfauggcaaGfgAfuccagsusu | 2291 |
| D-1220 | gsgsauccUfuGfCfCfAfuuccccucs{invAb} | 1560 | usGfsaGfgGfgaauggcAfaGfgauccsusu | 2292 |
| D-1221 | gsasuccuUfgCfCfAfUfuccccucas{invAb} | 1561 | asUfsgAfgGfggaauggCfaAfggaucsusu | 2293 |
| D-1222 | cscsuugcCfaUfUfCfCfccucagcus{invAb} | 1562 | usAfsgCfuGfaggggaaUfgGfcaaggsusu | 2294 |
| D-1223 | csusugccAfuUfCfCfCfcucagcuas{invAb} | 1563 | usUfsaGfcUfgaggggaAfuGfgcaagsusu | 2295 |
| D-1224 | gscscauuCfcCfCfUfCfagcuaaugs{invAb} | 1564 | usCfsaUfuAfgcugaggGfgAfauggcsusu | 2296 |
| D-1225 | csasuuccCfcUfCfAfGfcuaaugacs{invAb} | 1565 | asGfsuCfaUfuagcugaGfgGfgaaugsusu | 2297 |
| D-1226 | ascsggagUfgCfUfCfCfuucuccags{invAb} | 1566 | asCfsuGfgAfgaaggagCfaCfuccgususu | 2298 |
| D-1227 | gsasaaacCfuUfUfAfAfagggggaas{invAb} | 1567 | usUfsuCfcCfccuuuaaAfgGfuuuucsusu | 2299 |
| D-1228 | csasuaugUfcAfGfUfUfguuuaaaas{invAb} | 1568 | asUfsuUfuAfaacaacuGfaCfauaugsusu | 2300 |
| D-1229 | uscsaguuGfuUfUfAfAfaacccaaus{invAb} | 1569 | usAfsuUfgGfguuuuaaAfcAfacugasusu | 2301 |
| D-1230 | asgsuuguUfuAfAfAfAfcccaauaus{invAb} | 1570 | asAfsuAfuUfggguuuuAfaAfcaacususu | 2302 |
| D-1231 | asasggacGfcAfCfUfGfcucugauus{invAb} | 1571 | asAfsaUfcAfgagcaguGfcGfuccuususu | 2303 |
| D-1232 | csasagccCfcGfGfGfCfuagcuuuus{invAb} | 1572 | asAfsaAfaGfcuagcccGfgGfgcuugsusu | 2304 |
| D-1233 | cscsccggGfcUfAfGfCfuuuugaaas{invAb} | 1573 | asUfsuUfcAfaaagcuaGfcCfcggggsusu | 2305 |
| D-1234 | gsascugaGfgUfGfAfCfcuucaggas{invAb} | 1574 | usUfscCfuGfaaggucaCfcUfcagucsusu | 2306 |
| D-1235 | usasuuaaUfuUfUfCfCfauagaucus{invAb} | 1575 | asAfsgAfuCfuauggaaAfaUfuaauasusu | 2307 |
| D-1236 | ususuuccAfuAfGfAfUfcuggaucus{invAb} | 1576 | asAfsgAfuCfcagaucuAfuGfgaaaasusu | 2308 |
| D-1237 | ususcucaGfaCfAfGfCfauuggauus{invAb} | 1577 | asAfsaUfcCfaaugcugUfcUfgagaasusu | 2309 |
| D-1238 | ususuccuAfaAfGfGfUfgcucaggas{invAb} | 1578 | asUfscCfuGfagcaccuUfuAfggaaasusu | 2310 |
| D-1239 | cscsuggaUfcCfUfUfGfccauucccs{invAb} | 1579 | asGfsgGfaAfuggcaagGfaUfccaggsusu | 2311 |
| D-1240 | uscscuugCfcAfUfUfCfcccucagcs{invAb} | 1580 | asGfscUfgAfggggaauGfgCfaaggasusu | 2312 |
| D-1241 | cscsauucCfcCfUfCfAfgcuaaugas{invAb} | 1581 | asUfscAfuUfagcugagGfgGfaauggsusu | 2313 |
| D-1242 | asasaaccUfuUfAfAfAfgggggaaas{invAb} | 1582 | usUfsuUfcCfcccuuuaAfaGfguuuususu | 2314 |
| D-1243 | ususguuuAfaAfAfCfCfcaauaucus{invAb} | 1583 | usAfsgAfuAfuuggguuUfuAfaacaasusu | 2315 |
| D-1244 | csuscuaaGfaUfCfUfGfaugaaguas{invAb} | 1584 | asUfsaCfuUfcaucagaUfcUfuagagsusu | 2316 |
| D-1245 | csusaagaUfcUfGfAfUfgaaguauas{invAb} | 1585 | asUfsaUfaCfuucaucaGfaUfcuuagsusu | 2317 |
| D-1246 | usasagauCfuGfAfUfGfaaguauaus{invAb} | 1586 | asAfsuAfuAfcuucaucAfgAfucuuasusu | 2318 |
| D-1247 | asasgaucUfgAfUfGfAfaguauauus{invAb} | 1587 | asAfsaUfaUfacuucauCfaGfaucuususu | 2319 |
| D-1248 | gsasugaaGfuAfUfAfUfuuuuuauus{invAb} | 1588 | asAfsaUfaAfaaaauauAfcUfucaucsusu | 2320 |
| D-1249 | ususuuauUfgCfCfAfUfuuuguccus{invAb} | 1589 | asAfsgGfaCfaaaauggCfaAfuaaaasusu | 2321 |
| D-1250 | ususuauuGfcCfAfUfUfuuguccuus{invAb} | 1590 | asAfsaGfgAfcaaaaugGfcAfauaaasusu | 2322 |
| D-1251 | ususauugCfcAfUfUfUfuguccuuus{invAb} | 1591 | asAfsaAfgGfacaaaauGfgCfaauaasusu | 2323 |
| D-1252 | asusugccAfuUfUfUfGfuccuuugas{invAb} | 1592 | asUfscAfaAfggacaaaAfuGfgcaaususu | 2324 |
| D-1253 | asusauugGfgAfAfGfUfugacuaaas{invAb} | 1593 | asUfsuUfaGfucaacuuCfcCfaauaususu | 2325 |
| D-1254 | usgsggaaGfuUfGfAfCfuaaacuugs{invAb} | 1594 | usCfsaAfgUfuuagucaAfcUfucccasusu | 2326 |
| D-1255 | gsgsgaagUfuGfAfCfUfaaacuugas{invAb} | 1595 | usUfscAfaGfuuuagucAfaCfuucccsusu | 2327 |
| D-1256 | gsgsaaguUfgAfCfUfAfaacuugaas{invAb} | 1596 | usUfsuCfaAfguuuaguCfaAfcuuccsusu | 2328 |
| D-1257 | ascsugugAfaUfAfAfAfuggaagcus{invAb} | 1597 | usAfsgCfuUfccauuuaUfuCfacagususu | 2329 |
| D-1258 | usgsaauaAfaUfGfGfAfagcuacuus{invAb} | 1598 | asAfsaGfuAfgcuuccaUfuUfauucasusu | 2330 |
| D-1259 | usasaaugGfaAfGfCfUfacuuugacs{invAb} | 1599 | asGfsuCfaAfaguagcuUfcCfauuuasusu | 2331 |
| D-1260 | asasauggAfaGfCfUfAfcuuugacus{invAb} | 1600 | usAfsgUfcAfaaguagcUfuCfcauuususu | 2332 |
| D-1261 | asasgcuaCfuUfUfGfAfcuaguuucs{invAb} | 1601 | usGfsaAfaCfuagucaaAfgUfagcuususu | 2333 |
| D-1262 | asgscuacUfuUfGfAfCfuaguuucas{invAb} | 1602 | asUfsgAfaAfcuagucaAfaGfuagcususu | 2334 |
| D-1263 | gsgsagugCfuCfCfUfUfcuccaguus{invAb} | 1603 | asAfsaCfuGfgagaaggAfgCfacuccsusu | 2335 |
| D-1264 | asasccuuUfaAfAfGfGfgggaaaags{invAb} | 1604 | asCfsuUfuUfcccccuuUfaAfagguususu | 2336 |
| D-1265 | ascscuuuAfaAfGfGfGfggaaaaggs{invAb} | 1605 | usCfscUfuUfucccccuUfuAfaaggususu | 2337 |
| D-1266 | usasuugcCfaUfUfUfUfguccuuugs{invAb} | 1606 | usCfsaAfaGfgacaaaaUfgGfcaauasusu | 2338 |
| D-1267 | gsasaguuGfaCfUfAfAfacuugaaas{invAb} | 1607 | usUfsuUfcAfaguuuagUfcAfacuucsusu | 2339 |
| D-1268 | asasguugAfcUfAfAfAfcuugaaaas{invAb} | 1608 | usUfsuUfuCfaaguuuaGfuCfaacuususu | 2340 |
| D-1269 | gscsuacuUfuGfAfCfUfaguuucags{invAb} | 1609 | usCfsuGfaAfacuagucAfaAfguagcsusu | 2341 |
| D-1270 | usgsacucUfcAfGfUfGfcagccuacs{invAb} | 1610 | usGfsuAfgGfcugcacuGfaGfagucasusu | 2342 |
| D-1271 | ascsgcccAfcCfAfCfAfaaugcagus{invAb} | 1611 | asAfscUfgCfauuugugGfuGfggcgususu | 2343 |
| D-1272 | cscscaguGfgAfUfAfAfccagcuucs{invAb} | 1612 | asGfsaAfgCfugguuauCfcAfcugggsusu | 2344 |
| D-1273 | csasucaaAfuAfGfCfAfgacuuguus{invAb} | 1613 | asAfsaCfaAfgucugcuAfuUfugaugsusu | 2345 |
| D-1274 | uscsaaauAfgCfAfGfAfcuuguuccs{invAb} | 1614 | asGfsgAfaCfaagucugCfuAfuuugasusu | 2346 |
| D-1275 | csasggcuAfgAfGfAfAfgaaaguuas{invAb} | 1615 | usUfsaAfcUfuucuucuCfuAfgccugsusu | 2347 |
| D-1276 | asgsgcuaGfaGfAfAfGfaaaguuaas{invAb} | 1616 | usUfsuAfaCfuuucuucUfcUfagccususu | 2348 |
| D-1277 | ascscaacUfuCfAfGfGfcccaauaus{invAb} | 1617 | asAfsuAfuUfgggccugAfaGfuuggususu | 2349 |
| D-1278 | gsasgcuuCfuUfAfUfUfggugacgus{invAb} | 1618 | asAfscGfuCfaccaauaAfgAfagcucsusu | 2350 |
| D-1279 | csusucuuAfuUfGfGfUfgacguggas{invAb} | 1619 | usUfscCfaCfgucaccaAfuAfagaagsusu | 2351 |
| D-1280 | uscsuuauUfgGfUfGfAfcguggaacs{invAb} | 1620 | asGfsuUfcCfacgucacCfaAfuaagasusu | 2352 |
| D-1281 | asusugguGfaCfGfUfGfgaacugaas{invAb} | 1621 | usUfsuCfaGfuuccacgUfcAfccaaususu | 2353 |
| D-1282 | csusuguuCfcAfGfAfUfgcauuuuas{invAb} | 1622 | usUfsaAfaAfugcaucuGfgAfacaagsusu | 2354 |
| D-1283 | usgsuuccAfgAfUfGfCfauuuuaacs{invAb} | 1623 | asGfsuUfaAfaaugcauCfuGfgaacasusu | 2355 |
| D-1284 | csusggaaAfcAfCfUfGfaagaguuas{invAb} | 1624 | asUfsaAfcUfcuucaguGfuUfuccagsusu | 2356 |
| D-1285 | usgsgaaaCfaCfUfGfAfagaguuaus{invAb} | 1625 | asAfsuAfaCfucuucagUfgUfuuccasusu | 2357 |
| D-1286 | asasgaguUfaUfCfGfCfcagugugas{invAb} | 1626 | asUfscAfcAfcuggcgaUfaAfcucuususu | 2358 |
| D-1287 | asgsaguuAfuCfGfCfCfagugugacs{invAb} | 1627 | asGfsuCfaCfacuggcgAfuAfacucususu | 2359 |
| D-1288 | gsusuauaUfgGfAfAfAfaucaccacs{invAb} | 1628 | asGfsuGfgUfgauuuucCfaUfauaacsusu | 2360 |
| D-1289 | gsusgcugGfaAfAfAfCfccagggacs{invAb} | 1629 | asGfsuCfcCfuggguuuUfcCfagcacsusu | 2361 |
| D-1290 | usgscuggAfaAfAfCfCfcagggaccs{invAb} | 1630 | usGfsgUfcCfcuggguuUfuCfcagcasusu | 2362 |
| D-1291 | gscsuggaAfaAfCfCfCfagggaccas{invAb} | 1631 | asUfsgGfuCfccuggguUfuUfccagcsusu | 2363 |
| D-1292 | asasaaccCfaGfGfGfAfccaucaaas{invAb} | 1632 | asUfsuUfgAfuggucccUfgGfguuuususu | 2364 |
| D-1293 | asasacccAfgGfGfAfCfcaucaaags{invAb} | 1633 | asCfsuUfuGfaugguccCfuGfgguuususu | 2365 |
| D-1294 | asascccaGfgGfAfCfCfaucaaagus{invAb} | 1634 | asAfscUfuUfgauggucCfcUfggguususu | 2366 |
| D-1295 | ascsccagGfgAfCfCfAfucaaagugs{invAb} | 1635 | asCfsaCfuUfugaugguCfcCfugggususu | 2367 |
| D-1296 | gsusgggaGfaCfCfCfUfguguaccus{invAb} | 1636 | asAfsgGfuAfcacagggUfcUfcccacsusu | 2368 |
| D-1297 | gscsugggCfcAfGfUfAfaugggaacs{invAb} | 1637 | asGfsuUfcCfcauuacuGfgCfccagcsusu | 2369 |
| D-1298 | csasaaaaUfgAfCfAfAfcacuugaas{invAb} | 1638 | asUfsuCfaAfguguuguCfaUfuuuugsusu | 2370 |
| D-1299 | asusgacaAfcAfCfUfUfgaagcaugs{invAb} | 1639 | asCfsaUfgCfuucaaguGfuUfgucaususu | 2371 |
| D-1300 | ascsuugaAfgCfAfUfGfguguuucas{invAb} | 1640 | asUfsgAfaAfcaccaugCfuUfcaagususu | 2372 |
| D-1301 | asasauuuGfuGfAfUfUfuucacauus{invAb} | 1641 | asAfsaUfgUfgaaaaucAfcAfaauuususu | 2373 |
| D-1302 | asasugcuUfcAfAfUfGfucccagugs{invAb} | 1642 | asCfsaCfuGfggacauuGfaAfgcauususu | 2374 |
| D-1303 | asasaugaCfaAfGfAfCfaggauucus{invAb} | 1643 | asAfsgAfaUfccugucuUfgUfcauuususu | 2375 |
| D-1304 | ususauggAfaUfAfGfUfucuuucucs{invAb} | 1644 | asGfsaGfaAfagaacuaUfuCfcauaasusu | 2376 |
| D-1305 | usasuggaAfuAfGfUfUfcuuucuccs{invAb} | 1645 | asGfsgAfgAfaagaacuAfuUfccauasusu | 2377 |
| D-1306 | gsasauagUfuCfUfUfUfcuccugcus{invAb} | 1646 | asAfsgCfaGfgagaaagAfaCfuauucsusu | 2378 |
| D-1307 | asasuaguUfcUfUfUfCfuccugcuus{invAb} | 1647 | asAfsaGfcAfggagaaaGfaAfcuauususu | 2379 |
| D-1308 | usgscaucCfuGfUfCfAfcuaccacus{invAb} | 1648 | asAfsgUfgGfuagugacAfgGfaugcasusu | 2380 |
| D-1309 | gscsauccUfgUfCfAfCfuaccacucs{invAb} | 1649 | asGfsaGfuGfguagugaCfaGfgaugcsusu | 2381 |
| D-1310 | cscsgggcUfaGfCfUfUfuugaaaugs{invAb} | 1650 | asCfsaUfuUfcaaaagcUfaGfcccggsusu | 2382 |
| D-1311 | csgsggcuAfgCfUfUfUfugaaauggs{invAb} | 1651 | asCfscAfuUfucaaaagCfuAfgcccgsusu | 2383 |
| D-1312 | asasgacuGfaGfGfUfGfaccuucags{invAb} | 1652 | asCfsuGfaAfggucaccUfcAfgucuususu | 2384 |
| D-1313 | asgsgugaCfcUfUfCfAfggaagcacs{invAb} | 1653 | asGfsuGfcUfuccugaaGfgUfcaccususu | 2385 |
| D-1314 | cscsauagAfuCfUfGfGfaucuggccs{invAb} | 1654 | asGfsgCfcAfgauccagAfuCfuauggsusu | 2386 |
| D-1315 | usgsgauuUfcCfUfAfAfaggugcucs{invAb} | 1655 | usGfsaGfcAfccuuuagGfaAfauccasusu | 2387 |
| D-1316 | gsasuuucCfuAfAfAfGfgugcucags{invAb} | 1656 | asCfsuGfaGfcaccuuuAfgGfaaaucsusu | 2388 |
| D-1317 | uscscuaaAfgGfUfGfCfucaggaggs{invAb} | 1657 | usCfscUfcCfugagcacCfuUfuaggasusu | 2389 |
| D-1318 | usgsgaggAfcCfCfCfUfggauccuus{invAb} | 1658 | asAfsaGfgAfuccagggGfuCfcuccasusu | 2390 |
| D-1319 | gsgsaggaCfcCfCfUfGfgauccuugs{invAb} | 1659 | asCfsaAfgGfauccaggGfgUfccuccsusu | 2391 |
| D-1320 | gsasggacCfcCfUfGfGfauccuugcs{invAb} | 1660 | asGfscAfaGfgauccagGfgGfuccucsusu | 2392 |
| D-1321 | csgsgaguGfcUfCfCfUfucuccagus{invAb} | 1661 | asAfscUfgGfagaaggaGfcAfcuccgsusu | 2393 |
| D-1322 | asgsaaggAfcGfCfAfCfugcucugas{invAb} | 1662 | asUfscAfgAfgcagugcGfuCfcuucususu | 2394 |
| D-1323 | csuscugaUfuGfGfCfCfcggaagggs{invAb} | 1663 | asCfscCfuUfccgggccAfaUfcagagsusu | 2395 |
| D-1324 | uscsugauUfgGfCfCfCfggaagggus{invAb} | 1664 | asAfscCfcUfuccgggcCfaAfucagasusu | 2396 |
| D-1325 | csusgauuGfgCfCfCfGfgaaggguus{invAb} | 1665 | asAfsaCfcCfuuccgggCfcAfaucagsusu | 2397 |
| D-1326 | gsgsccaaAfgGfCfCfGfcaccuuccs{invAb} | 1666 | asGfsgAfaGfgugcggcCfuUfuggccsusu | 2398 |
| D-1327 | cscsucgcGfgAfGfAfAfgccagccas{invAb} | 1667 | asUfsgGfcUfggcuucuCfcGfcgaggsusu | 2399 |
| D-1328 | gsgsagaaGfcCfAfGfCfcaugggcgs{invAb} | 1668 | asCfsgCfcCfauggcugGfcUfucuccsusu | 2400 |
| D-1329 | gsasgaagCfcAfGfCfCfaugggcgcs{invAb} | 1669 | asGfscGfcCfcauggcuGfgCfuucucsusu | 2401 |
| D-1330 | gsasucaaCfcAfGfGfAfgggaaacas{invAb} | 1670 | asUfsgUfuUfcccuccuGfgUfugaucsusu | 2402 |
| D-1331 | ascsugcuCfgCfCfAfGfgaaccucgs{invAb} | 1671 | asCfsgAfgGfuuccuggCfgAfgcagususu | 2403 |
| D-1332 | uscsgccaGfgAfAfCfCfucgccuggs{invAb} | 1672 | asCfscAfgGfcgagguuCfcUfggcgasusu | 2404 |
| D-1333 | gscscaggAfaCfCfUfCfgccuggucs{invAb} | 1673 | asGfsaCfcAfggcgaggUfuCfcuggcsusu | 2405 |
| D-1334 | gsasaccuCfgCfCfUfGfguccugaus{invAb} | 1674 | asAfsuCfaGfgaccaggCfgAfgguucsusu | 2406 |
| D-1335 | asusggugAfcAfCfCfCfugacucucs{invAb} | 1675 | usGfsaGfaGfucaggguGfuCfaccaususu | 2407 |
| D-1336 | usgsgugaCfaCfCfCfUfgacucucas{invAb} | 1676 | asUfsgAfgAfgucagggUfgUfcaccasusu | 2408 |
| D-1337 | gsascaccCfuGfAfCfUfcucagugcs{invAb} | 1677 | usGfscAfcUfgagagucAfgGfgugucsusu | 2409 |
| D-1338 | ascsccugAfcUfCfUfCfagugcagcs{invAb} | 1678 | asGfscUfgCfacugagaGfuCfagggususu | 2410 |
| D-1339 | cscsgcccAfgUfGfGfAfuaaccagcs{invAb} | 1679 | asGfscUfgGfuuauccaCfuGfggcggsusu | 2411 |
| D-1340 | cscsgccuGfgUfGfCfAfcuucgagcs{invAb} | 1680 | asGfscUfcGfaagugcaCfcAfggcggsusu | 2412 |
| D-1341 | csgsccugGfuGfCfAfCfuucgagccs{invAb} | 1681 | asGfsgCfuCfgaagugcAfcCfaggcgsusu | 2413 |
| D-1342 | gscscuggUfgCfAfCfUfucgagccus{invAb} | 1682 | asAfsgGfcUfcgaagugCfaCfcaggcsusu | 2414 |
| D-1343 | cscsugguGfcAfCfUfUfcgagccucs{invAb} | 1683 | usGfsaGfgCfucgaaguGfcAfccaggsusu | 2415 |
| D-1344 | csusggugCfaCfUfUfCfgagccucas{invAb} | 1684 | asUfsgAfgGfcucgaagUfgCfaccagsusu | 2416 |
| D-1345 | usgsgugcAfcUfUfCfGfagccucacs{invAb} | 1685 | usGfsuGfaGfgcucgaaGfuGfcaccasusu | 2417 |
| D-1346 | gsgsugcaCfuUfCfGfAfgccucacas{invAb} | 1686 | asUfsgUfgAfggcucgaAfgUfgcaccsusu | 2418 |
| D-1347 | gsusgcacUfuCfGfAfGfccucacaus{invAb} | 1687 | asAfsuGfuGfaggcucgAfaGfugcacsusu | 2419 |
| D-1348 | usgscacuUfcGfAfGfCfcucacaugs{invAb} | 1688 | asCfsaUfgUfgaggcucGfaAfgugcasusu | 2420 |
| D-1349 | gscsacuuCfgAfGfCfCfucacaugcs{invAb} | 1689 | asGfscAfuGfugaggcuCfgAfagugcsusu | 2421 |
| D-1350 | csascuucGfaGfCfCfUfcacaugcgs{invAb} | 1690 | usCfsgCfaUfgugaggcUfcGfaagugsusu | 2422 |
| D-1351 | ascsuucgAfgCfCfUfCfacaugcgas{invAb} | 1691 | asUfscGfcAfugugaggCfuCfgaagususu | 2423 |
| D-1352 | csusucgaGfcCfUfCfAfcaugcgacs{invAb} | 1692 | asGfsuCfgCfaugugagGfcUfcgaagsusu | 2424 |
| D-1353 | ususcgagCfcUfCfAfCfaugcgaccs{invAb} | 1693 | asGfsgUfcGfcaugugaGfgCfucgaasusu | 2425 |
| D-1354 | uscsgagcCfuCfAfCfAfugcgaccgs{invAb} | 1694 | usCfsgGfuCfgcaugugAfgGfcucgasusu | 2426 |
| D-1355 | csgsagccUfcAfCfAfUfgcgaccgas{invAb} | 1695 | asUfscGfgUfcgcauguGfaGfgcucgsusu | 2427 |
| D-1356 | gscscucaCfaUfGfCfGfaccgagacs{invAb} | 1696 | asGfsuCfuCfggucgcaUfgUfgaggcsusu | 2428 |
| D-1357 | csusugauCfcUfUfUfCfugaggcgus{invAb} | 1697 | asAfscGfcCfucagaaaGfgAfucaagsusu | 2429 |
| D-1358 | csusggcgGfaUfCfUfCfaacuccags{invAb} | 1698 | asCfsuGfgAfguugagaUfcCfgccagsusu | 2430 |
| D-1359 | usgsgcggAfuCfUfCfAfacuccaggs{invAb} | 1699 | asCfscUfgGfaguugagAfuCfcgccasusu | 2431 |
| D-1360 | gscsgaugUfcUfAfUfGfcagaggaus{invAb} | 1700 | asAfsuCfcUfcugcauaGfaCfaucgcsusu | 2432 |
| D-1361 | gsasugucUfaUfGfCfAfgaggauucs{invAb} | 1701 | asGfsaAfuCfcucugcaUfaGfacaucsusu | 2433 |
| D-1362 | usgsucuaUfgCfAfGfAfggauucuus{invAb} | 1702 | asAfsaGfaAfuccucugCfaUfagacasusu | 2434 |
| D-1363 | gsuscuauGfcAfGfAfGfgauucuugs{invAb} | 1703 | asCfsaAfgAfauccucuGfcAfuagacsusu | 2435 |
| D-1364 | uscsuaugCfaGfAfGfGfauucuuggs{invAb} | 1704 | asCfscAfaGfaauccucUfgCfauagasusu | 2436 |
| D-1365 | csusaugcAfgAfGfGfAfuucuugggs{invAb} | 1705 | usCfscCfaAfgaauccuCfuGfcauagsusu | 2437 |
| D-1366 | gsgsugauGfgCfUfUfGfuuccagaus{invAb} | 1706 | asAfsuCfuGfgaacaagCfcAfucaccsusu | 2438 |
| D-1367 | gsusgaugGfcUfUfGfUfuccagaugs{invAb} | 1707 | asCfsaUfcUfggaacaaGfcCfaucacsusu | 2439 |
| D-1368 | usgsgcuuGfuUfCfCfAfgaugcauus{invAb} | 1708 | asAfsaUfgCfaucuggaAfcAfagccasusu | 2440 |
| D-1369 | csasuuuuAfaCfCfAfCfaguggaccs{invAb} | 1709 | asGfsgUfcCfacuguggUfuAfaaaugsusu | 2441 |
| D-1370 | ususuuaaCfcAfCfAfGfuggacccas{invAb} | 1710 | asUfsgGfgUfccacuguGfgUfuaaaasusu | 2442 |
| D-1371 | ususuaacCfaCfAfGfUfggacccags{invAb} | 1711 | usCfsuGfgGfuccacugUfgGfuuaaasusu | 2443 |
| D-1372 | csasccacUfcUfUfUfGfggcaguaus{invAb} | 1712 | asAfsuAfcUfgcccaaaGfaGfuggugsusu | 2444 |
| D-1373 | ascscacuCfuUfUfGfGfgcaguauus{invAb} | 1713 | asAfsaUfaCfugcccaaAfgAfguggususu | 2445 |
| D-1374 | csusuuggGfcAfGfUfAfuuuugugcs{invAb} | 1714 | asGfscAfcAfaaauacuGfcCfcaaagsusu | 2446 |
| D-1375 | ususugggCfaGfUfAfUfuuugugcus{invAb} | 1715 | asAfsgCfaCfaaaauacUfgCfccaaasusu | 2447 |
| D-1376 | ususgggcAfgUfAfUfUfuugugcugs{invAb} | 1716 | asCfsaGfcAfcaaaauaCfuGfcccaasusu | 2448 |
| D-1377 | usgsggcaGfuAfUfUfUfugugcuggs{invAb} | 1717 | usCfscAfgCfacaaaauAfcUfgcccasusu | 2449 |
| D-1378 | gsgscaguAfuUfUfUfGfugcuggaas{invAb} | 1718 | usUfsuCfcAfgcacaaaAfuAfcugccsusu | 2450 |
| D-1379 | usasuuuuGfuGfCfUfGfgaaaacccs{invAb} | 1719 | usGfsgGfuUfuuccagcAfcAfaaauasusu | 2451 |
| D-1380 | asusuuugUfgCfUfGfGfaaaacccas{invAb} | 1720 | asUfsgGfgUfuuuccagCfaCfaaaaususu | 2452 |
| D-1381 | ascscguaUfgUfCfCfUfggaauauus{invAb} | 1721 | usAfsaUfaUfuccaggaCfaUfacggususu | 2453 |
| D-1382 | cscsguauGfuCfCfUfGfgaauauuas{invAb} | 1722 | asUfsaAfuAfuuccaggAfcAfuacggsusu | 2454 |
| D-1383 | gsusauguCfcUfGfGfAfauauuagas{invAb} | 1723 | asUfscUfaAfuauuccaGfgAfcauacsusu | 2455 |
| D-1384 | usasugucCfuGfGfAfAfuauuagaus{invAb} | 1724 | asAfsuCfuAfauauuccAfgGfacauasusu | 2456 |
| D-1385 | asusguccUfgGfAfAfUfauuagaugs{invAb} | 1725 | asCfsaUfcUfaauauucCfaGfgacaususu | 2457 |
| D-1386 | usgsuccuGfgAfAfUfAfuuagaugcs{invAb} | 1726 | asGfscAfuCfuaauauuCfcAfggacasusu | 2458 |
| D-1387 | gsusccugGfaAfUfAfUfuagaugccs{invAb} | 1727 | asGfsgCfaUfcuaauauUfcCfaggacsusu | 2459 |
| D-1388 | uscscuggAfaUfAfUfUfagaugccus{invAb} | 1728 | asAfsgGfcAfucuaauaUfuCfcaggasusu | 2460 |
| D-1389 | cscsuggaAfuAfUfUfAfgaugccuus{invAb} | 1729 | asAfsaGfgCfaucuaauAfuUfccaggsusu | 2461 |
| D-1390 | csusggaaUfaUfUfAfGfaugccuuus{invAb} | 1730 | asAfsaAfgGfcaucuaaUfaUfuccagsusu | 2462 |
| D-1391 | csasugguGfuUfUfCfAfgaacugags{invAb} | 1731 | usCfsuCfaGfuucugaaAfcAfccaugsusu | 2463 |
| D-1392 | asusggugUfuUfCfAfGfaacugagas{invAb} | 1732 | asUfscUfcAfguucugaAfaCfaccaususu | 2464 |
| D-1393 | usgsguguUfuCfAfGfAfacugagacs{invAb} | 1733 | asGfsuCfuCfaguucugAfaAfcaccasusu | 2465 |
| D-1394 | gsgsuguuUfcAfGfAfAfcugagaccs{invAb} | 1734 | asGfsgUfcUfcaguucuGfaAfacaccsusu | 2466 |
| D-1395 | gsasggagAfaGfAfAfAfagugauucs{invAb} | 1735 | usGfsaAfuCfacuuuucUfuCfuccucsusu | 2467 |
| D-1396 | asgsaagaAfaAfGfUfGfauucagugs{invAb} | 1736 | usCfsaCfuGfaaucacuUfuUfcuucususu | 2468 |
| D-1397 | gsasagaaAfaGfUfGfAfuucagugas{invAb} | 1737 | asUfscAfcUfgaaucacUfuUfucuucsusu | 2469 |
| D-1398 | gsasaaagUfgAfUfUfCfagugauuus{invAb} | 1738 | asAfsaAfuCfacugaauCfaCfuuuucsusu | 2470 |
| D-1399 | asasagugAfuUfCfAfGfugauuucas{invAb} | 1739 | asUfsgAfaAfucacugaAfuCfacuuususu | 2471 |
| D-1400 | ascsuacuGfaAfAfAfCfcuuuaaags{invAb} | 1740 | asCfsuUfuAfaagguuuUfcAfguagususu | 2472 |
| D-1401 | usascugaAfaAfCfCfUfuuaaagggs{invAb} | 1741 | asCfscCfuUfuaaagguUfuUfcaguasusu | 2473 |
| D-1402 | usgsuauaAfcUfCfUfAfagaucugas{invAb} | 1742 | asUfscAfgAfucuuagaGfuUfauacasusu | 2474 |
| D-1403 | usasuaacUfcUfAfAfGfaucugaugs{invAb} | 1743 | usCfsaUfcAfgaucuuaGfaGfuuauasusu | 2475 |
| D-1404 | asusaacuCfuAfAfGfAfucugaugas{invAb} | 1744 | usUfscAfuCfagaucuuAfgAfguuaususu | 2476 |
| D-1405 | usasacucUfaAfGfAfUfcugaugaas{invAb} | 1745 | asUfsuCfaUfcagaucuUfaGfaguuasusu | 2477 |
| D-1406 | asascucuAfaGfAfUfCfugaugaags{invAb} | 1746 | asCfsuUfcAfucagaucUfuAfgaguususu | 2478 |
| D-1407 | ascsucuaAfgAfUfCfUfgaugaagus{invAb} | 1747 | usAfscUfuCfaucagauCfuUfagagususu | 2479 |
| D-1408 | gsasuuggCfcCfGfGfAfaggguucas{invAb} | 1748 | asUfsgAfaCfccuuccgGfgCfcaaucsusu | 2480 |
| D-1409 | cscsuuugGfgCfUfCfGfgggccaaas{invAb} | 1749 | asUfsuUfgGfccccgagCfcCfaaaggsusu | 2481 |
| D-1410 | ususgggcUfcGfGfGfGfccaaaggcs{invAb} | 1750 | asGfscCfuUfuggccccGfaGfcccaasusu | 2482 |
| D-1411 | csgscaccUfuCfCfCfCfcagcggccs{invAb} | 1751 | asGfsgCfcGfcugggggAfaGfgugcgsusu | 2483 |
| D-1412 | cscsgccgCfcAfCfCfUfcgcggagas{invAb} | 1752 | usUfscUfcCfgcgagguGfgCfggcggsusu | 2484 |
| D-1413 | uscscgcgCfuGfGfCfGfcgcuuugus{invAb} | 1753 | asAfscAfaAfgcgcgccAfgCfgcggasusu | 2485 |
| D-1414 | cscsgcgcUfgGfCfGfCfgcuuugucs{invAb} | 1754 | asGfsaCfaAfagcgcgcCfaGfcgcggsusu | 2486 |
| D-1415 | csgscgcuGfgCfGfCfGfcuuuguccs{invAb} | 1755 | asGfsgAfcAfaagcgcgCfcAfgcgcgsusu | 2487 |
| D-1416 | csgscgcuUfuGfUfCfCfuccucgcgs{invAb} | 1756 | asCfsgCfgAfggaggacAfaAfgcgcgsusu | 2488 |
| D-1417 | gscsgcuuUfgUfCfCfUfccucgcgcs{invAb} | 1757 | usGfscGfcGfaggaggaCfaAfagcgcsusu | 2489 |
| D-1418 | csgscuuuGfuCfCfUfCfcucgcgcas{invAb} | 1758 | usUfsgCfgCfgaggaggAfcAfaagcgsusu | 2490 |
| D-1419 | gscsuuugUfcCfUfCfCfucgcgcaas{invAb} | 1759 | asUfsuGfcGfcgaggagGfaCfaaagcsusu | 2491 |
| D-1420 | csusuuguCfcUfCfCfUfcgcgcaaus{invAb} | 1760 | asAfsuUfgCfgcgaggaGfgAfcaaagsusu | 2492 |
| D-1421 | ususugucCfuCfCfUfCfgcgcaaucs{invAb} | 1761 | asGfsaUfuGfcgcgaggAfgGfacaaasusu | 2493 |
| D-1422 | usgsuccuCfcUfCfGfCfgcaaucccs{invAb} | 1762 | asGfsgGfaUfugcgcgaGfgAfggacasusu | 2494 |
| D-1423 | gsusccucCfuCfGfCfGfcaaucccgs{invAb} | 1763 | asCfsgGfgAfuugcgcgAfgGfaggacsusu | 2495 |
| D-1424 | uscscuccUfcGfCfGfCfaaucccggs{invAb} | 1764 | asCfscGfgGfauugcgcGfaGfgaggasusu | 2496 |
| D-1425 | cscsuccuCfgCfGfCfAfaucccggcs{invAb} | 1765 | asGfscCfgGfgauugcgCfgAfggaggsusu | 2497 |
| D-1426 | csuscgcgCfaAfUfCfCfcggcccggs{invAb} | 1766 | asCfscGfgGfccgggauUfgCfgcgagsusu | 2498 |
| D-1427 | gscsgcaaUfcCfCfGfGfcccgggugs{invAb} | 1767 | asCfsaCfcCfgggccggGfaUfugcgcsusu | 2499 |
| D-1428 | csgscaauCfcCfGfGfCfccggguggs{invAb} | 1768 | asCfscAfcCfcgggccgGfgAfuugcgsusu | 2500 |
| D-1429 | gscsaaucCfcGfGfCfCfcggguggcs{invAb} | 1769 | asGfscCfaCfccgggccGfgGfauugcsusu | 2501 |
| D-1430 | gsgscccgGfgUfGfGfCfucgggguus{invAb} | 1770 | asAfsaCfcCfcgagccaCfcCfgggccsusu | 2502 |
| D-1431 | gscsccggGfuGfGfCfUfcgggguugs{invAb} | 1771 | asCfsaAfcCfccgagccAfcCfcgggcsusu | 2503 |
| D-1432 | cscscgggUfgGfCfUfCfgggguugcs{invAb} | 1772 | asGfscAfaCfcccgagcCfaCfccgggsusu | 2504 |
| D-1433 | uscsggggUfuGfCfCfGfcgcugggcs{invAb} | 1773 | asGfscCfcAfgcgcggcAfaCfcccgasusu | 2505 |
| D-1434 | gsgsuugcCfgCfGfCfUfgggccugas{invAb} | 1774 | asUfscAfgGfcccagcgCfgGfcaaccsusu | 2506 |
| D-1435 | gscscgcgCfuGfGfGfCfcugaccgcs{invAb} | 1775 | asGfscGfgUfcaggcccAfgCfgcggcsusu | 2507 |
| D-1436 | gscsgcugGfgCfCfUfGfaccgcggus{invAb} | 1776 | asAfscCfgCfggucaggCfcCfagcgcsusu | 2508 |
| D-1437 | usgsggccUfgAfCfCfGfcgguggcgs{invAb} | 1777 | asCfsgCfcAfccgcgguCfaGfgcccasusu | 2509 |
| D-1438 | gsgsgccuGfaCfCfGfCfgguggcgcs{invAb} | 1778 | asGfscGfcCfaccgcggUfcAfggcccsusu | 2510 |
| D-1439 | gsasgggaAfaCfAfUfGfguuacugcs{invAb} | 1779 | asGfscAfgUfaaccaugUfuUfcccucsusu | 2511 |
| D-1440 | gsgsaaacAfuGfGfUfUfacugcucgs{invAb} | 1780 | asCfsgAfgCfaguaaccAfuGfuuuccsusu | 2512 |
| D-1441 | gsasaacaUfgGfUfUfAfcugcucgcs{invAb} | 1781 | asGfscGfaGfcaguaacCfaUfguuucsusu | 2513 |
| D-1442 | asasacauGfgUfUfAfCfugcucgccs{invAb} | 1782 | usGfsgCfgAfgcaguaaCfcAfuguuususu | 2514 |
| D-1443 | asascaugGfuUfAfCfUfgcucgccas{invAb} | 1783 | asUfsgGfcGfagcaguaAfcCfauguususu | 2515 |
| D-1444 | ascsauggUfuAfCfUfGfcucgccags{invAb} | 1784 | asCfsuGfgCfgagcaguAfaCfcaugususu | 2516 |
| D-1445 | gsusuacuGfcUfCfGfCfcaggaaccs{invAb} | 1785 | asGfsgUfuCfcuggcgaGfcAfguaacsusu | 2517 |
| D-1446 | ususcccuGfaCfCfUfGfcgauggugs{invAb} | 1786 | usCfsaCfcAfucgcaggUfcAfgggaasusu | 2518 |
| D-1447 | ascscugcGfaUfGfGfUfgacacccus{invAb} | 1787 | asAfsgGfgUfgucaccaUfcGfcaggususu | 2519 |
| D-1448 | gsusgcagCfcUfAfCfAfcaaaggacs{invAb} | 1788 | asGfsuCfcUfuuguguaGfgCfugcacsusu | 2520 |
| D-1449 | usgscagcCfuAfCfAfCfaaaggaccs{invAb} | 1789 | asGfsgUfcCfuuuguguAfgGfcugcasusu | 2521 |
| D-1450 | csasgccuAfcAfCfAfAfaggaccuas{invAb} | 1790 | asUfsaGfgUfccuuuguGfuAfggcugsusu | 2522 |
| D-1451 | asgsccuaCfaCfAfAfAfggaccuacs{invAb} | 1791 | asGfsuAfgGfuccuuugUfgUfaggcususu | 2523 |
| D-1452 | gscscuacAfcAfAfAfGfgaccuacus{invAb} | 1792 | usAfsgUfaGfguccuuuGfuGfuaggcsusu | 2524 |
| D-1453 | cscsuacaCfaAfAfGfGfaccuacuas{invAb} | 1793 | asUfsaGfuAfgguccuuUfgUfguaggsusu | 2525 |
| D-1454 | csusacacAfaAfGfGfAfccuacuacs{invAb} | 1794 | asGfsuAfgUfagguccuUfuGfuguagsusu | 2526 |
| D-1455 | csascaaaGfgAfCfCfUfacuacugcs{invAb} | 1795 | asGfscAfgUfaguagguCfcUfuugugsusu | 2527 |
| D-1456 | asgsgaccUfaCfUfAfCfugccuaucs{invAb} | 1796 | usGfsaUfaGfgcaguagUfaGfguccususu | 2528 |
| D-1457 | gsgsaccuAfcUfAfCfUfgccuaucas{invAb} | 1797 | usUfsgAfuAfggcaguaGfuAfgguccsusu | 2529 |
| D-1458 | ascscuacUfaCfUfGfCfcuaucaaas{invAb} | 1798 | usUfsuUfgAfuaggcagUfaGfuaggususu | 2530 |
| D-1459 | usascuacUfgCfCfUfAfucaaaacgs{invAb} | 1799 | asCfsgUfuUfugauaggCfaGfuaguasusu | 2531 |
| D-1460 | csusacugCfcUfAfUfCfaaaacgccs{invAb} | 1800 | asGfsgCfgUfuuugauaGfgCfaguagsusu | 2532 |
| D-1461 | gscscuauCfaAfAfAfCfgcccaccas{invAb} | 1801 | asUfsgGfuGfggcguuuUfgAfuaggcsusu | 2533 |
| D-1462 | ascscacaAfaUfGfCfAfgugcacaas{invAb} | 1802 | asUfsuGfuGfcacugcaUfuUfguggususu | 2534 |
| D-1463 | csascaaaUfgCfAfGfUfgcacaagus{invAb} | 1803 | asAfscUfuGfugcacugCfaUfuugugsusu | 2535 |
| D-1464 | csasaaugCfaGfUfGfCfacaagugcs{invAb} | 1804 | usGfscAfcUfugugcacUfgCfauuugsusu | 2536 |
| D-1465 | asusgcagUfgCfAfCfAfagugcagas{invAb} | 1805 | asUfscUfgCfacuugugCfaCfugcaususu | 2537 |
| D-1466 | asgsugcaCfaAfGfUfGfcagagugcs{invAb} | 1806 | usGfscAfcUfcugcacuUfgUfgcacususu | 2538 |
| D-1467 | ascsaaguGfcAfGfAfGfugcacggcs{invAb} | 1807 | asGfscCfgUfgcacucuGfcAfcuugususu | 2539 |
| D-1468 | csasagugCfaGfAfGfUfgcacggccs{invAb} | 1808 | asGfsgCfcGfugcacucUfgCfacuugsusu | 2540 |
| D-1469 | usgscagaGfuGfCfAfCfggccuggas{invAb} | 1809 | asUfscCfaGfgccgugcAfcUfcugcasusu | 2541 |
| D-1470 | asgsagugCfaCfGfGfCfcuggagaus{invAb} | 1810 | usAfsuCfuCfcaggccgUfgCfacucususu | 2542 |
| D-1471 | gsasgugcAfcGfGfCfCfuggagauas{invAb} | 1811 | asUfsaUfcUfccaggccGfuGfcacucsusu | 2543 |
| D-1472 | usgsgagaUfaGfAfGfGfgcagggacs{invAb} | 1812 | asGfsuCfcCfugcccucUfaUfcuccasusu | 2544 |
| D-1473 | uscscugaAfgUfCfAfCfagcccuacs{invAb} | 1813 | asGfsuAfgGfgcugugaCfuUfcaggasusu | 2545 |
| D-1474 | csusgaagUfcAfCfAfGfcccuaccgs{invAb} | 1814 | asCfsgGfuAfgggcuguGfaCfuucagsusu | 2546 |
| D-1475 | gsasagucAfcAfGfCfCfcuaccgccs{invAb} | 1815 | asGfsgCfgGfuagggcuGfuGfacuucsusu | 2547 |
| D-1476 | cscsucacAfuGfCfGfAfccgagacgs{invAb} | 1816 | asCfsgUfcUfcggucgcAfuGfugaggsusu | 2548 |
| D-1477 | csuscacaUfgCfGfAfCfcgagacgus{invAb} | 1817 | asAfscGfuCfucggucgCfaUfgugagsusu | 2549 |
| D-1478 | uscsacauGfcGfAfCfCfgagacgucs{invAb} | 1818 | asGfsaCfgUfcucggucGfcAfugugasusu | 2550 |
| D-1479 | csascaugCfgAfCfCfGfagacguccs{invAb} | 1819 | asGfsgAfcGfucucgguCfgCfaugugsusu | 2551 |
| D-1480 | ascsaugcGfaCfCfGfAfgacguccus{invAb} | 1820 | asAfsgGfaCfgucucggUfcGfcaugususu | 2552 |
| D-1481 | usgscgacCfgAfGfAfCfguccucaus{invAb} | 1821 | asAfsuGfaGfgacgucuCfgGfucgcasusu | 2553 |
| D-1482 | gscsgaccGfaGfAfCfGfuccucaucs{invAb} | 1822 | usGfsaUfgAfggacgucUfcGfgucgcsusu | 2554 |
| D-1483 | cscsgagaCfgUfCfCfUfcaucaaaus{invAb} | 1823 | usAfsuUfuGfaugaggaCfgUfcucggsusu | 2555 |
| D-1484 | csgsagacGfuCfCfUfCfaucaaauas{invAb} | 1824 | asUfsaUfuUfgaugaggAfcGfucucgsusu | 2556 |
| D-1485 | asgsacguCfcUfCfAfUfcaaauagcs{invAb} | 1825 | usGfscUfaUfuugaugaGfgAfcgucususu | 2557 |
| D-1486 | uscscucaUfcAfAfAfUfagcagacus{invAb} | 1826 | asAfsgUfcUfgcuauuuGfaUfgaggasusu | 2558 |
| D-1487 | csuscaucAfaAfUfAfGfcagacuugs{invAb} | 1827 | asCfsaAfgUfcugcuauUfuGfaugagsusu | 2559 |
| D-1488 | csasgacaCfcAfGfCfCfcauucuugs{invAb} | 1828 | usCfsaAfgAfaugggcuGfgUfgucugsusu | 2560 |
| D-1489 | asgsacacCfaGfCfCfCfauucuugas{invAb} | 1829 | asUfscAfaGfaaugggcUfgGfugucususu | 2561 |
| D-1490 | gsascaccAfgCfCfCfAfuucuugaus{invAb} | 1830 | asAfsuCfaAfgaaugggCfuGfgugucsusu | 2562 |
| D-1491 | csasgcccAfuUfCfUfUfgauccuuus{invAb} | 1831 | asAfsaAfgGfaucaagaAfuGfggcugsusu | 2563 |
| D-1492 | cscsauucUfuGfAfUfCfcuuucugas{invAb} | 1832 | asUfscAfgAfaaggaucAfaGfaauggsusu | 2564 |
| D-1493 | gscsagagGfaUfUfCfUfugggaugas{invAb} | 1833 | asUfscAfuCfccaagaaUfcCfucugcsusu | 2565 |
| D-1494 | asusucuuGfgGfAfUfGfagcuucuus{invAb} | 1834 | usAfsaGfaAfgcucaucCfcAfagaaususu | 2566 |
| D-1495 | csusugggAfuGfAfGfCfuucuuauus{invAb} | 1835 | asAfsaUfaAfgaagcucAfuCfccaagsusu | 2567 |
| D-1496 | gsgsgaugAfgCfUfUfCfuuauuggus{invAb} | 1836 | asAfscCfaAfuaagaagCfuCfaucccsusu | 2568 |
| D-1497 | gsgsaugaGfcUfUfCfUfuauuggugs{invAb} | 1837 | usCfsaCfcAfauaagaaGfcUfcauccsusu | 2569 |
| D-1498 | gsusggaaCfuGfAfAfAfagggugaus{invAb} | 1838 | asAfsuCfaCfccuuuucAfgUfuccacsusu | 2570 |
| D-1499 | usgsgaacUfgAfAfAfAfgggugaugs{invAb} | 1839 | asCfsaUfcAfcccuuuuCfaGfuuccasusu | 2571 |
| D-1500 | gsasacugAfaAfAfGfGfgugauggcs{invAb} | 1840 | asGfscCfaUfcacccuuUfuCfaguucsusu | 2572 |
| D-1501 | csusgaaaAfgGfGfUfGfauggcuugs{invAb} | 1841 | asCfsaAfgCfcaucaccCfuUfuucagsusu | 2573 |
| D-1502 | usgsaaaaGfgGfUfGfAfuggcuugus{invAb} | 1842 | asAfscAfaGfccaucacCfcUfuuucasusu | 2574 |
| D-1503 | gsasaaagGfgUfGfAfUfggcuuguus{invAb} | 1843 | asAfsaCfaAfgccaucaCfcCfuuuucsusu | 2575 |
| D-1504 | asasaaggGfuGfAfUfGfgcuuguucs{invAb} | 1844 | asGfsaAfcAfagccaucAfcCfcuuuususu | 2576 |
| D-1505 | gsusggacCfcAfGfAfCfaccggugus{invAb} | 1845 | asAfscAfcCfggugucuGfgGfuccacsusu | 2577 |
| D-1506 | usgsgaccCfaGfAfCfAfccggugucs{invAb} | 1846 | usGfsaCfaCfcggugucUfgGfguccasusu | 2578 |
| D-1507 | gsgsacccAfgAfCfAfCfcggugucas{invAb} | 1847 | asUfsgAfcAfccgguguCfuGfgguccsusu | 2579 |
| D-1508 | ascsccagAfcAfCfCfGfgugucaugs{invAb} | 1848 | usCfsaUfgAfcaccgguGfuCfugggususu | 2580 |
| D-1509 | cscsagacAfcCfGfGfUfgucaugags{invAb} | 1849 | asCfsuCfaUfgacaccgGfuGfucuggsusu | 2581 |
| D-1510 | csasccggUfgUfCfAfUfgagcaggas{invAb} | 1850 | usUfscCfuGfcucaugaCfaCfcggugsusu | 2582 |
| D-1511 | gsuscaugAfgCfAfGfGfaaggaaccs{invAb} | 1851 | asGfsgUfuCfcuuccugCfuCfaugacsusu | 2583 |
| D-1512 | asusgagcAfgGfAfAfGfgaaccgcus{invAb} | 1852 | asAfsgCfgGfuuccuucCfuGfcucaususu | 2584 |
| D-1513 | asgsgaagGfaAfCfCfGfcuggaaacs{invAb} | 1853 | usGfsuUfuCfcagcgguUfcCfuuccususu | 2585 |
| D-1514 | gsgsaaggAfaCfCfGfCfuggaaacas{invAb} | 1854 | asUfsgUfuUfccagcggUfuCfcuuccsusu | 2586 |
| D-1515 | gsasaggaAfcCfGfCfUfggaaacacs{invAb} | 1855 | asGfsuGfuUfuccagcgGfuUfccuucsusu | 2587 |
| D-1516 | csusgggcCfaGfUfAfAfugggaaccs{invAb} | 1856 | asGfsgUfuCfccauuacUfgGfcccagsusu | 2588 |
| D-1517 | gsgsgccaGfuAfAfUfGfggaaccgus{invAb} | 1857 | usAfscGfgUfucccauuAfcUfggcccsusu | 2589 |
| D-1518 | gsgsccagUfaAfUfGfGfgaaccguas{invAb} | 1858 | asUfsaCfgGfuucccauUfaCfuggccsusu | 2590 |
| D-1519 | gscscaguAfaUfGfGfGfaaccguaus{invAb} | 1859 | asAfsuAfcGfguucccaUfuAfcuggcsusu | 2591 |
| D-1520 | cscsaguaAfuGfGfGfAfaccguaugs{invAb} | 1860 | asCfsaUfaCfgguucccAfuUfacuggsusu | 2592 |
| D-1521 | csasguaaUfgGfGfAfAfccguaugus{invAb} | 1861 | asAfscAfuAfcgguuccCfaUfuacugsusu | 2593 |
| D-1522 | asgsuaauGfgGfAfAfCfcguaugucs{invAb} | 1862 | asGfsaCfaUfacgguucCfcAfuuacususu | 2594 |
| D-1523 | usgsggaaCfcGfUfAfUfguccuggas{invAb} | 1863 | usUfscCfaGfgacauacGfgUfucccasusu | 2595 |
| D-1524 | gsgsaaccGfuAfUfGfUfccuggaaus{invAb} | 1864 | usAfsuUfcCfaggacauAfcGfguuccsusu | 2596 |
| D-1525 | gsasauauUfaGfAfUfGfccuuuuaas{invAb} | 1865 | usUfsuAfaAfaggcaucUfaAfuauucsusu | 2597 |
| D-1526 | gsasugccUfuUfUfAfAfaaauguucs{invAb} | 1866 | asGfsaAfcAfuuuuuaaAfaGfgcaucsusu | 2598 |
| D-1527 | gsusuucaGfaAfCfUfGfagaccucus{invAb} | 1867 | usAfsgAfgGfucucaguUfcUfgaaacsusu | 2599 |
| D-1528 | uscsagaaCfuGfAfGfAfccucuacas{invAb} | 1868 | asUfsgUfaGfaggucucAfgUfucugasusu | 2600 |
| D-1529 | ascsugagAfcCfUfCfUfacauuuucs{invAb} | 1869 | asGfsaAfaAfuguagagGfuCfucagususu | 2601 |
| D-1530 | csusgagaCfcUfCfUfAfcauuuucus{invAb} | 1870 | asAfsgAfaAfauguagaGfgUfcucagsusu | 2602 |
| D-1531 | usgsagacCfuCfUfAfCfauuuucuus{invAb} | 1871 | asAfsaGfaAfaauguagAfgGfucucasusu | 2603 |
| D-1532 | usgsauuuUfcAfCfAfUfuuuucgucs{invAb} | 1872 | asGfsaCfgAfaaaauguGfaAfaaucasusu | 2604 |
| D-1533 | gsasuuuuCfaCfAfUfUfuuucgucus{invAb} | 1873 | asAfsgAfcGfaaaaaugUfgAfaaaucsusu | 2605 |
| D-1534 | asusuuucAfcAfUfUfUfuucgucuus{invAb} | 1874 | asAfsaGfaCfgaaaaauGfuGfaaaaususu | 2606 |
| D-1535 | ususucacAfuUfUfUfUfcgucuuuus{invAb} | 1875 | asAfsaAfaGfacgaaaaAfuGfugaaasusu | 2607 |
| D-1536 | ususcacaUfuUfUfUfCfgucuuuugs{invAb} | 1876 | asCfsaAfaAfgacgaaaAfaUfgugaasusu | 2608 |
| D-1537 | uscsacauUfuUfUfCfGfucuuuuggs{invAb} | 1877 | usCfscAfaAfagacgaaAfaAfugugasusu | 2609 |
| D-1538 | ascsauuuUfuCfGfUfCfuuuuggacs{invAb} | 1878 | asGfsuCfcAfaaagacgAfaAfaaugususu | 2610 |
| D-1539 | ususuucgUfcUfUfUfUfggacuucus{invAb} | 1879 | asAfsgAfaGfuccaaaaGfaCfgaaaasusu | 2611 |
| D-1540 | ususucguCfuUfUfUfGfgacuucugs{invAb} | 1880 | asCfsaGfaAfguccaaaAfgAfcgaaasusu | 2612 |
| D-1541 | ususcgucUfuUfUfGfGfacuucuggs{invAb} | 1881 | asCfscAfgAfaguccaaAfaGfacgaasusu | 2613 |
| D-1542 | uscsgucuUfuUfGfGfAfcuucuggus{invAb} | 1882 | asAfscCfaGfaaguccaAfaAfgacgasusu | 2614 |
| D-1543 | csusuuugGfaCfUfUfCfuggugucus{invAb} | 1883 | asAfsgAfcAfccagaagUfcCfaaaagsusu | 2615 |
| D-1544 | ususggacUfuCfUfGfGfugucucaas{invAb} | 1884 | asUfsuGfaGfacaccagAfaGfuccaasusu | 2616 |
| D-1545 | gsgsacuuCfuGfGfUfGfucucaaugs{invAb} | 1885 | asCfsaUfuGfagacaccAfgAfaguccsusu | 2617 |
| D-1546 | gsascuucUfgGfUfGfUfcucaaugcs{invAb} | 1886 | asGfscAfuUfgagacacCfaGfaagucsusu | 2618 |
| D-1547 | ususcuggUfgUfCfUfCfaaugcuucs{invAb} | 1887 | usGfsaAfgCfauugagaCfaCfcagaasusu | 2619 |
| D-1548 | gscsuucaAfuGfUfCfCfcagugcaas{invAb} | 1888 | usUfsuGfcAfcugggacAfuUfgaagcsusu | 2620 |
| D-1549 | asusguccCfaGfUfGfCfaaaaaguas{invAb} | 1889 | usUfsaCfuUfuuugcacUfgGfgacaususu | 2621 |
| D-1550 | usgsucccAfgUfGfCfAfaaaaguaas{invAb} | 1890 | usUfsuAfcUfuuuugcaCfuGfggacasusu | 2622 |
| D-1551 | gsuscccaGfuGfCfAfAfaaaguaaas{invAb} | 1891 | asUfsuUfaCfuuuuugcAfcUfgggacsusu | 2623 |
| D-1552 | uscsccagUfgCfAfAfAfaaguaaags{invAb} | 1892 | usCfsuUfuAfcuuuuugCfaCfugggasusu | 2624 |
| D-1553 | asgsugcaAfaAfAfGfUfaaagaaaus{invAb} | 1893 | usAfsuUfuCfuuuacuuUfuUfgcacususu | 2625 |
| D-1554 | asasgaaaUfaUfAfGfUfcucaauaas{invAb} | 1894 | asUfsuAfuUfgagacuaUfaUfuucuususu | 2626 |
| D-1555 | asasauauAfgUfCfUfCfaauaacuus{invAb} | 1895 | usAfsaGfuUfauugagaCfuAfuauuususu | 2627 |
| D-1556 | asusauagUfcUfCfAfAfuaacuuags{invAb} | 1896 | asCfsuAfaGfuuauugaGfaCfuauaususu | 2628 |
| D-1557 | usasuaguCfuCfAfAfUfaacuuagus{invAb} | 1897 | usAfscUfaAfguuauugAfgAfcuauasusu | 2629 |
| D-1558 | asusagucUfcAfAfUfAfacuuaguas{invAb} | 1898 | asUfsaCfuAfaguuauuGfaGfacuaususu | 2630 |
| D-1559 | usasgucuCfaAfUfAfAfcuuaguags{invAb} | 1899 | asCfsuAfcUfaaguuauUfgAfgacuasusu | 2631 |
| D-1560 | asgsucucAfaUfAfAfCfuuaguaggs{invAb} | 1900 | usCfscUfaCfuaaguuaUfuGfagacususu | 2632 |
| D-1561 | uscsaauaAfcUfUfAfGfuaggacuus{invAb} | 1901 | asAfsaGfuCfcuacuaaGfuUfauugasusu | 2633 |
| D-1562 | csasauaaCfuUfAfGfUfaggacuucs{invAb} | 1902 | usGfsaAfgUfccuacuaAfgUfuauugsusu | 2634 |
| D-1563 | asusaacuUfaGfUfAfGfgacuucags{invAb} | 1903 | asCfsuGfaAfguccuacUfaAfguuaususu | 2635 |
| D-1564 | asascuuaGfuAfGfGfAfcuucaguas{invAb} | 1904 | usUfsaCfuGfaaguccuAfcUfaaguususu | 2636 |
| D-1565 | ascsuuagUfaGfGfAfCfuucaguaas{invAb} | 1905 | asUfsuAfcUfgaaguccUfaCfuaagususu | 2637 |
| D-1566 | ususaguaGfgAfCfUfUfcaguaagus{invAb} | 1906 | asAfscUfuAfcugaaguCfcUfacuaasusu | 2638 |
| D-1567 | usasguagGfaCfUfUfCfaguaagucs{invAb} | 1907 | usGfsaCfuUfacugaagUfcCfuacuasusu | 2639 |
| D-1568 | asgsuaggAfcUfUfCfAfguaagucas{invAb} | 1908 | asUfsgAfcUfuacugaaGfuCfcuacususu | 2640 |
| D-1569 | usasggacUfuCfAfGfUfaagucacus{invAb} | 1909 | asAfsgUfgAfcuuacugAfaGfuccuasusu | 2641 |
| D-1570 | usasaaugAfcAfAfGfAfcaggauucs{invAb} | 1910 | asGfsaAfuCfcugucuuGfuCfauuuasusu | 2642 |
| D-1571 | gsgsauucUfgAfAfAfAfcuccccgus{invAb} | 1911 | asAfscGfgGfgaguuuuCfaGfaauccsusu | 2643 |
| D-1572 | gsasuucuGfaAfAfAfCfuccccguus{invAb} | 1912 | asAfsaCfgGfggaguuuUfcAfgaaucsusu | 2644 |
| D-1573 | ususcugaAfaAfCfUfCfcccguuuas{invAb} | 1913 | usUfsaAfaCfggggaguUfuUfcagaasusu | 2645 |
| D-1574 | uscsugaaAfaCfUfCfCfccguuuaas{invAb} | 1914 | asUfsuAfaAfcggggagUfuUfucagasusu | 2646 |
| D-1575 | csusgaaaAfcUfCfCfCfcguuuaacs{invAb} | 1915 | asGfsuUfaAfacggggaGfuUfuucagsusu | 2647 |
| D-1576 | usgsaaaaCfuCfCfCfCfguuuaacus{invAb} | 1916 | asAfsgUfuAfaacggggAfgUfuuucasusu | 2648 |
| D-1577 | asasaacuCfcCfCfGfUfuuaacugas{invAb} | 1917 | asUfscAfgUfuaaacggGfgAfguuuususu | 2649 |
| D-1578 | ascsucccCfgUfUfUfAfacugauuas{invAb} | 1918 | asUfsaAfuCfaguuaaaCfgGfggagususu | 2650 |
| D-1579 | csusccccGfuUfUfAfAfcugauuaus{invAb} | 1919 | asAfsuAfaUfcaguuaaAfcGfgggagsusu | 2651 |
| D-1580 | uscscccgUfuUfAfAfCfugauuaugs{invAb} | 1920 | asCfsaUfaAfucaguuaAfaCfggggasusu | 2652 |
| D-1581 | cscsccguUfuAfAfCfUfgauuauggs{invAb} | 1921 | usCfscAfuAfaucaguuAfaAfcggggsusu | 2653 |
| D-1582 | ususcuccUfgCfUfUfCfuccguuuas{invAb} | 1922 | asUfsaAfaCfggagaagCfaGfgagaasusu | 2654 |
| D-1583 | uscsuccuGfcUfUfCfUfccguuuaus{invAb} | 1923 | asAfsuAfaAfcggagaaGfcAfggagasusu | 2655 |
| D-1584 | csusccugCfuUfCfUfCfcguuuaucs{invAb} | 1924 | asGfsaUfaAfacggagaAfgCfaggagsusu | 2656 |
| D-1585 | cscsugcuUfcUfCfCfGfuuuaucuas{invAb} | 1925 | asUfsaGfaUfaaacggaGfaAfgcaggsusu | 2657 |
| D-1586 | gscsuucuCfcGfUfUfUfaucuaccas{invAb} | 1926 | usUfsgGfuAfgauaaacGfgAfgaagcsusu | 2658 |
| D-1587 | csusucucCfgUfUfUfAfucuaccaas{invAb} | 1927 | asUfsuGfgUfagauaaaCfgGfagaagsusu | 2659 |
| D-1588 | ususcuccGfuUfUfAfUfcuaccaags{invAb} | 1928 | usCfsuUfgGfuagauaaAfcGfgagaasusu | 2660 |
| D-1589 | uscsuccgUfuUfAfUfCfuaccaagas{invAb} | 1929 | asUfscUfuGfguagauaAfaCfggagasusu | 2661 |
| D-1590 | csusccguUfuAfUfCfUfaccaagags{invAb} | 1930 | asCfsuCfuUfgguagauAfaAfcggagsusu | 2662 |
| D-1591 | uscscguuUfaUfCfUfAfccaagagcs{invAb} | 1931 | asGfscUfcUfugguagaUfaAfacggasusu | 2663 |
| D-1592 | csgsuuuaUfcUfAfCfCfaagagcgcs{invAb} | 1932 | usGfscGfcUfcuugguaGfaUfaaacgsusu | 2664 |
| D-1593 | ususaucuAfcCfAfAfGfagcgcagas{invAb} | 1933 | asUfscUfgCfgcucuugGfuAfgauaasusu | 2665 |
| D-1594 | asuscuacCfaAfGfAfGfcgcagacus{invAb} | 1934 | asAfsgUfcUfgcgcucuUfgGfuagaususu | 2666 |
| D-1595 | usasccaaGfaGfCfGfCfagacuugcs{invAb} | 1935 | usGfscAfaGfucugcgcUfcUfugguasusu | 2667 |
| D-1596 | ascscaagAfgCfGfCfAfgacuugcas{invAb} | 1936 | asUfsgCfaAfgucugcgCfuCfuuggususu | 2668 |
| D-1597 | csasagagCfgCfAfGfAfcuugcaucs{invAb} | 1937 | asGfsaUfgCfaagucugCfgCfucuugsusu | 2669 |
| D-1598 | asasgagcGfcAfGfAfCfuugcauccs{invAb} | 1938 | asGfsgAfuGfcaagucuGfcGfcucuususu | 2670 |
| D-1599 | gsasgcgcAfgAfCfUfUfgcauccugs{invAb} | 1939 | asCfsaGfgAfugcaaguCfuGfcgcucsusu | 2671 |
| D-1600 | gscsgcagAfcUfUfGfCfauccugucs{invAb} | 1940 | usGfsaCfaGfgaugcaaGfuCfugcgcsusu | 2672 |
| D-1601 | csgscagaCfuUfGfCfAfuccugucas{invAb} | 1941 | asUfsgAfcAfggaugcaAfgUfcugcgsusu | 2673 |
| D-1602 | csasgacuUfgCfAfUfCfcugucacus{invAb} | 1942 | usAfsgUfgAfcaggaugCfaAfgucugsusu | 2674 |
| D-1603 | csusugcaUfcCfUfGfUfcacuaccas{invAb} | 1943 | asUfsgGfuAfgugacagGfaUfgcaagsusu | 2675 |
| D-1604 | csasuccuGfuCfAfCfUfaccacucgs{invAb} | 1944 | asCfsgAfgUfgguagugAfcAfggaugsusu | 2676 |
| D-1605 | uscscuguCfaCfUfAfCfcacucguus{invAb} | 1945 | usAfsaCfgAfgugguagUfgAfcaggasusu | 2677 |
| D-1606 | cscsugucAfcUfAfCfCfacucguuas{invAb} | 1946 | asUfsaAfcGfagugguaGfuGfacaggsusu | 2678 |
| D-1607 | csusgucaCfuAfCfCfAfcucguuags{invAb} | 1947 | usCfsuAfaCfgagugguAfgUfgacagsusu | 2679 |
| D-1608 | usgsucacUfaCfCfAfCfucguuagas{invAb} | 1948 | asUfscUfaAfcgaguggUfaGfugacasusu | 2680 |
| D-1609 | ascsuaccAfcUfCfGfUfuagagaaas{invAb} | 1949 | asUfsuUfcUfcuaacgaGfuGfguagususu | 2681 |
| D-1610 | asasgaguGfgGfUfGfGfgcuggaags{invAb} | 1950 | usCfsuUfcCfagcccacCfcAfcucuususu | 2682 |
| D-1611 | uscscuagAfaUfGfUfGfuuauugccs{invAb} | 1951 | asGfsgCfaAfuaacacaUfuCfuaggasusu | 2683 |
| D-1612 | cscsuagaAfuGfUfGfUfuauugcccs{invAb} | 1952 | asGfsgGfcAfauaacacAfuUfcuaggsusu | 2684 |
| D-1613 | asasugugUfuAfUfUfGfccccuguus{invAb} | 1953 | asAfsaCfaGfgggcaauAfaCfacauususu | 2685 |
| D-1614 | gsusguuaUfuGfCfCfCfcuguucaus{invAb} | 1954 | asAfsuGfaAfcaggggcAfaUfaacacsusu | 2686 |
| D-1615 | ususauugCfcCfCfUfGfuucaugags{invAb} | 1955 | asCfsuCfaUfgaacaggGfgCfaauaasusu | 2687 |
| D-1616 | asusugccCfcUfGfUfUfcaugaggus{invAb} | 1956 | usAfscCfuCfaugaacaGfgGfgcaaususu | 2688 |
| D-1617 | asasugaaAfaUfUfAfAfauugcaccs{invAb} | 1957 | asGfsgUfgCfaauuuaaUfuUfucauususu | 2689 |
| D-1618 | asusgaaaAfuUfAfAfAfuugcacccs{invAb} | 1958 | asGfsgGfuGfcaauuuaAfuUfuucaususu | 2690 |
| D-1619 | asasauuaAfaUfUfGfCfaccccaaas{invAb} | 1959 | asUfsuUfgGfggugcaaUfuUfaauuususu | 2691 |
| D-1620 | asusuaaaUfuGfCfAfCfcccaaauas{invAb} | 1960 | asUfsaUfuUfggggugcAfaUfuuaaususu | 2692 |
| D-1621 | ususaaauUfgCfAfCfCfccaaauaus{invAb} | 1961 | asAfsuAfuUfuggggugCfaAfuuuaasusu | 2693 |
| D-1622 | asasuugcAfcCfCfCfAfaauauggcs{invAb} | 1962 | asGfscCfaUfauuugggGfuGfcaauususu | 2694 |
| D-1623 | asusugcaCfcCfCfAfAfauauggcus{invAb} | 1963 | asAfsgCfcAfuauuuggGfgUfgcaaususu | 2695 |
| D-1624 | asusauggCfuGfGfAfAfugccacuus{invAb} | 1964 | asAfsaGfuGfgcauuccAfgCfcauaususu | 2696 |
| D-1625 | usgsgaauGfcCfAfCfUfucccuuuus{invAb} | 1965 | asAfsaAfaGfggaagugGfcAfuuccasusu | 2697 |
| D-1626 | ususcccuUfuUfCfUfUfcucaagccs{invAb} | 1966 | asGfsgCfuUfgagaagaAfaAfgggaasusu | 2698 |
| D-1627 | uscsuucuCfaAfGfCfCfccgggcuas{invAb} | 1967 | asUfsaGfcCfcggggcuUfgAfgaagasusu | 2699 |
| D-1628 | uscsucaaGfcCfCfCfGfggcuagcus{invAb} | 1968 | asAfsgCfuAfgcccgggGfcUfugagasusu | 2700 |
| D-1629 | gscsuuuuGfaAfAfUfGfgcauaaags{invAb} | 1969 | usCfsuUfuAfugccauuUfcAfaaagcsusu | 2701 |
| D-1630 | ususugaaAfuGfGfCfAfuaaagacus{invAb} | 1970 | asAfsgUfcUfuuaugccAfuUfucaaasusu | 2702 |
| D-1631 | asasuggcAfuAfAfAfGfacugaggus{invAb} | 1971 | asAfscCfuCfagucuuuAfuGfccauususu | 2703 |
| D-1632 | usgsgcauAfaAfGfAfCfugaggugas{invAb} | 1972 | asUfscAfcCfucagucuUfuAfugccasusu | 2704 |
| D-1633 | gscsauaaAfgAfCfUfGfaggugaccs{invAb} | 1973 | asGfsgUfcAfccucaguCfuUfuaugcsusu | 2705 |
| D-1634 | gsasagcaCfuGfCfAfGfauauuaaus{invAb} | 1974 | asAfsuUfaAfuaucugcAfgUfgcuucsusu | 2706 |
| D-1635 | csusaaagGfuGfCfUfCfaggaggaus{invAb} | 1975 | asAfsuCfcUfccugagcAfcCfuuuagsusu | 2707 |
| D-1636 | asgsgugcUfcAfGfGfAfggaugguus{invAb} | 1976 | asAfsaCfcAfuccuccuGfaGfcaccususu | 2708 |
| D-1637 | gsusgcucAfgGfAfGfGfaugguugus{invAb} | 1977 | asAfscAfaCfcauccucCfuGfagcacsusu | 2709 |
| D-1638 | gsasggauGfgUfUfGfUfguagucaus{invAb} | 1978 | asAfsuGfaCfuacacaaCfcAfuccucsusu | 2710 |
| D-1639 | asgsgaugGfuUfGfUfGfuagucaugs{invAb} | 1979 | asCfsaUfgAfcuacacaAfcCfauccususu | 2711 |
| D-1640 | gsgsauggUfuGfUfGfUfagucauggs{invAb} | 1980 | usCfscAfuGfacuacacAfaCfcauccsusu | 2712 |
| D-1641 | ususguguAfgUfCfAfUfggaggaccs{invAb} | 1981 | asGfsgUfcCfuccaugaCfuAfcacaasusu | 2713 |
| D-1642 | uscsauggAfgGfAfCfCfccuggaucs{invAb} | 1982 | asGfsaUfcCfaggggucCfuCfcaugasusu | 2714 |
| D-1643 | asusucccCfuCfAfGfCfuaaugacgs{invAb} | 1983 | asCfsgUfcAfuuagcugAfgGfggaaususu | 2715 |
| D-1644 | ususccccUfcAfGfCfUfaaugacggs{invAb} | 1984 | usCfscGfuCfauuagcuGfaGfgggaasusu | 2716 |
| D-1645 | uscscccuCfaGfCfUfAfaugacggas{invAb} | 1985 | asUfscCfgUfcauuagcUfgAfggggasusu | 2717 |
| D-1646 | uscsagcuAfaUfGfAfCfggagugcus{invAb} | 1986 | asAfsgCfaCfuccgucaUfuAfgcugasusu | 2718 |
| D-1647 | gsasaaaaGfuUfCfUfGfaauucugus{invAb} | 1987 | asAfscAfgAfauucagaAfcUfuuuucsusu | 2719 |
| D-1648 | asgsuucuGfaAfUfUfCfuguggaggs{invAb} | 1988 | usCfscUfcCfacagaauUfcAfgaacususu | 2720 |
| D-1649 | asgsugauUfuCfAfGfAfuagacuacs{invAb} | 1989 | asGfsuAfgUfcuaucugAfaAfucacususu | 2721 |
| D-1650 | asusuucaGfaUfAfGfAfcuacugaas{invAb} | 1990 | usUfsuCfaGfuagucuaUfcUfgaaaususu | 2722 |
| D-1651 | csasgauaGfaCfUfAfCfugaaaaccs{invAb} | 1991 | asGfsgUfuUfucaguagUfcUfaucugsusu | 2723 |
| D-1652 | usasgacuAfcUfGfAfAfaaccuuuas{invAb} | 1992 | usUfsaAfaGfguuuucaGfuAfgucuasusu | 2724 |
| D-1653 | asgsacuaCfuGfAfAfAfaccuuuaas{invAb} | 1993 | usUfsuAfaAfgguuuucAfgUfagucususu | 2725 |
| D-1654 | asasggaaAfgCfAfUfAfugucaguus{invAb} | 1994 | asAfsaCfuGfacauaugCfuUfuccuususu | 2726 |
| D-1655 | asgsgaaaGfcAfUfAfUfgucaguugs{invAb} | 1995 | asCfsaAfcUfgacauauGfcUfuuccususu | 2727 |
| D-1656 | gsgsaaagCfaUfAfUfGfucaguugus{invAb} | 1996 | asAfscAfaCfugacauaUfgCfuuuccsusu | 2728 |
| D-1657 | asasagcaUfaUfGfUfCfaguuguuus{invAb} | 1997 | usAfsaAfcAfacugacaUfaUfgcuuususu | 2729 |
| D-1658 | asasgcauAfuGfUfCfAfguuguuuas{invAb} | 1998 | usUfsaAfaCfaacugacAfuAfugcuususu | 2730 |
| D-1659 | asgscauaUfgUfCfAfGfuuguuuaas{invAb} | 1999 | usUfsuAfaAfcaacugaCfaUfaugcususu | 2731 |
| D-1660 | usasaaacCfcAfAfUfAfucuauuuus{invAb} | 2000 | asAfsaAfaUfagauauuGfgGfuuuuasusu | 2732 |
| D-1661 | asascccaAfuAfUfCfUfauuuuuuas{invAb} | 2001 | usUfsaAfaAfaauagauAfuUfggguususu | 2733 |
| D-1662 | ususaacuGfaUfUfGfUfauaacucus{invAb} | 2002 | usAfsgAfgUfuauacaaUfcAfguuaasusu | 2734 |
| D-1663 | usasacugAfuUfGfUfAfuaacucuas{invAb} | 2003 | usUfsaGfaGfuuauacaAfuCfaguuasusu | 2735 |
| D-1664 | ascsugauUfgUfAfUfAfacucuaags{invAb} | 2004 | usCfsuUfaGfaguuauaCfaAfucagususu | 2736 |
| D-1665 | csusgauuGfuAfUfAfAfcucuaagas{invAb} | 2005 | asUfscUfuAfgaguuauAfcAfaucagsusu | 2737 |
| D-1666 | gsasuuguAfuAfAfCfUfcuaagaucs{invAb} | 2006 | asGfsaUfcUfuagaguuAfuAfcaaucsusu | 2738 |
| D-1667 | gscscauuUfuGfUfCfCfuuugauuas{invAb} | 2007 | asUfsaAfuCfaaaggacAfaAfauggcsusu | 2739 |
| D-1668 | usgsuccuUfuGfAfUfUfauauugggs{invAb} | 2008 | usCfscCfaAfuauaaucAfaAfggacasusu | 2740 |
| D-2000 | [GalNAc3]saggcccAfaUfAfUfUfguaauuucs{invAb} | 2009 | usGfsaaauUfacaauaUfuGfggccususu | 2741 |
| D-2001 | [GalNAc3]scagaacGfaAfAfGfUfuauauggas{invAb} | 2010 | usUfsccauAfuaacuuUfcGfuucugsusu | 2742 |
| D-2002 | [GalNAc3]suuccagAfuGfCfAfUfuuuaaccas{invAb} | 2011 | asUfsgguuAfaaaugcAfuCfuggaasusu | 2743 |
| D-2003 | [GalNAc3]sugcauuUfuAfAfCfCfacaguggas{invAb} | 2012 | asUfsccacUfgugguuAfaAfaugcasusu | 2744 |
| D-2004 | [GalNAc3]sccagugGfaUfAfAfCfcagcuuccs{invAb} | 2013 | asGfsgaagCfugguuaUfcCfacuggsusu | 2745 |
| D-2005 | [GalNAc3]sgcuggaAfaCfAfCfUfgaagaguus{invAb} | 2014 | usAfsacucUfucagugUfuUfccagcsusu | 2746 |
| D-2006 | [GalNAc3]sgaaacaCfuGfAfAfGfaguuaucgs{invAb} | 2015 | asCfsgauaAfcucuucAfgUfguuucsusu | 2747 |
| D-2007 | [GalNAc3]saacacuGfaAfGfAfGfuuaucgccs{invAb} | 2016 | usGfsgcgaUfaacucuUfcAfguguususu | 2748 |
| D-2008 | [GalNAc3]scccguuUfaAfCfUfGfauuauggas{invAb} | 2017 | usUfsccauAfaucaguUfaAfacgggsusu | 2749 |
| D-2009 | [GalNAc3]saaaugaCfaAfCfAfCfuugaagcas{invAb} | 2018 | asUfsgcuuCfaaguguUfgUfcauuususu | 2750 |
| D-2010 | [GalNAc3]scacuugAfaGfCfAfUfgguguuucs{invAb} | 2019 | usGfsaaacAfccaugcUfuCfaagugsusu | 2751 |
| D-2011 | [GalNAc3]sgauuauGfgAfAfUfAfguucuuucs{invAb} | 2020 | asGfsaaagAfacuauuCfcAfuaaucsusu | 2752 |
| D-2012 | [GalNAc3]sauuaugGfaAfUfAfGfuucuuucus{invAb} | 2021 | asAfsgaaaGfaacuauUfcCfauaaususu | 2753 |
| D-2013 | [GalNAc3]sugguguCfuCfAfAfUfgcuucaaus{invAb} | 2022 | asAfsuugaAfgcauugAfgAfcaccasusu | 2754 |
| D-2014 | [GalNAc3]sgacaagAfcAfGfGfAfuucugaaas{invAb} | 2023 | usUfsuucaGfaauccuGfuCfuugucsusu | 2755 |
| D-2015 | [GalNAc3]scauaugUfcAfGfUfUfguuuaaaas{invAb} | 2024 | asUfsuuuaAfacaacuGfaCfauaugsusu | 2756 |
| D-2016 | [GalNAc3]saguuguUfuAfAfAfAfcccaauaus{invAb} | 2025 | asAfsuauuGfgguuuuAfaAfcaacususu | 2757 |
| D-2017 | [GalNAc3]suuguuuAfaAfAfCfCfcaauaucus{invAb} | 2026 | usAfsgauaUfuggguuUfuAfaacaasusu | 2758 |
| D-2018 | [GalNAc3]sgaugaaGfuAfUfAfUfuuuuuauus{invAb} | 2027 | asAfsauaaAfaaauauAfcUfucaucsusu | 2759 |
| D-2019 | [GalNAc3]suuuuauUfgCfCfAfUfuuuguccus{invAb} | 2028 | asAfsggacAfaaauggCfaAfuaaaasusu | 2760 |
| D-2020 | [GalNAc3]sauugccAfuUfUfUfGfuccuuugas{invAb} | 2029 | asUfscaaaGfgacaaaAfuGfgcaaususu | 2761 |
| D-2021 | [GalNAc3]sauauugGfgAfAfGfUfugacuaaas{invAb} | 2030 | asUfsuuagUfcaacuuCfcCfaauaususu | 2762 |
| D-2022 | [GalNAc3]sggaaguUfgAfCfUfAfaacuugaas{invAb} | 2031 | usUfsucaaGfuuuaguCfaAfcuuccsusu | 2763 |
| D-2023 | [GalNAc3]sacugugAfaUfAfAfAfuggaagcus{invAb} | 2032 | usAfsgcuuCfcauuuaUfuCfacagususu | 2764 |
| D-2024 | [GalNAc3]scagauuGfcUfUfAfCfucagacacs{invAb} | 2033 | asGfsugucUfgaguaaGfcAfaucugsusu | 2765 |
| D-2025 | [GalNAc3]scugaagAfgUfUfAfUfcgccagugs{invAb} | 2034 | asCfsacugGfcgauaaCfuCfuucagsusu | 2766 |
| D-2026 | [GalNAc3]sacccuuCfaGfAfAfCfgaaaguuas{invAb} | 2035 | asUfsaacuUfucguucUfgAfagggususu | 2767 |
| D-2027 | [GalNAc3]sgugaccCfuUfCfAfGfaacgaaags{invAb} | 2036 | asCfsuuucGfuucugaAfgGfgucacsusu | 2768 |
| D-2028 | [GalNAc3]sucagaaCfgAfAfAfGfuuauauggs{invAb} | 2037 | usCfscauaUfaacuuuCfgUfucugasusu | 2769 |
| D-2029 | [GalNAc3]suucuuaUfuGfGfUfGfacguggaas{invAb} | 2038 | asUfsuccaCfgucaccAfaUfaagaasusu | 2770 |
| D-2030 | [GalNAc3]saguuauAfuGfGfAfAfaaucaccas{invAb} | 2039 | asUfsggugAfuuuuccAfuAfuaacususu | 2771 |
| D-2031 | [GalNAc3]scccuucAfgAfAfCfGfaaaguuaus{invAb} | 2040 | usAfsuaacUfuucguuCfuGfaagggsusu | 2772 |
| D-2032 | [GalNAc3]sccuucaGfaAfCfGfAfaaguuauas{invAb} | 2041 | asUfsauaaCfuuucguUfcUfgaaggsusu | 2773 |
| D-2033 | [GalNAc3]scuucagAfaCfGfAfAfaguuauaus{invAb} | 2042 | asAfsuauaAfcuuucgUfuCfugaagsusu | 2774 |
| D-2034 | [GalNAc3]scaacuuCfaGfGfCfCfcaauauugs{invAb} | 2043 | asCfsaauaUfugggccUfgAfaguugsusu | 2775 |
| D-2035 | [GalNAc3]sggaaacAfcUfGfAfAfgaguuaucs{invAb} | 2044 | asGfsauaaCfucuucaGfuGfuuuccsusu | 2776 |
| D-2036 | [GalNAc3]sacuucaGfgCfCfCfAfauauuguas{invAb} | 2045 | usUfsacaaUfauugggCfcUfgaagususu | 2777 |
| D-2037 | [GalNAc3]saaguuaAfaGfCfAfAfccaacuucs{invAb} | 2046 | usGfsaaguUfgguugcUfuUfaacuususu | 2778 |
| D-2038 | [GalNAc3]scuucagGfcCfCfAfAfuauuguaas{invAb} | 2047 | asUfsuacaAfuauuggGfcCfugaagsusu | 2779 |
| D-2039 | [GalNAc3]sgaccagAfuUfGfCfUfuacucagas{invAb} | 2048 | asUfscugaGfuaagcaAfuCfuggucsusu | 2780 |
| D-2040 | [GalNAc3]sacugauUfaUfGfGfAfauaguucus{invAb} | 2049 | asAfsgaacUfauuccaUfaAfucagususu | 2781 |
| D-2041 | [GalNAc3]sauauggAfaAfAfUfCfaccacucus{invAb} | 2050 | asAfsgaguGfgugauuUfuCfcauaususu | 2782 |
| D-2042 | [GalNAc3]scaaugcUfuCfAfAfUfgucccagus{invAb} | 2051 | asAfscuggGfacauugAfaGfcauugsusu | 2783 |
| D-2043 | [GalNAc3]sugcuucAfaUfGfUfCfccagugcas{invAb} | 2052 | usUfsgcacUfgggacaUfuGfaagcasusu | 2784 |
| D-2044 | [GalNAc3]saaugacAfaGfAfCfAfggauucugs{invAb} | 2053 | usCfsagaaUfccugucUfuGfucauususu | 2785 |
| D-2045 | [GalNAc3]scuaagaUfcUfGfAfUfgaaguauas{invAb} | 2054 | asUfsauacUfucaucaGfaUfcuuagsusu | 2786 |
| D-2046 | [GalNAc3]scuggugUfcUfCfAfAfugcuucaas{invAb} | 2055 | asUfsugaaGfcauugaGfaCfaccagsusu | 2787 |
| D-2047 | [GalNAc3]sugucucAfaUfGfCfUfucaaugucs{invAb} | 2056 | asGfsacauUfgaagcaUfuGfagacasusu | 2788 |
| D-2048 | [GalNAc3]suuuuccAfuAfGfAfUfcuggaucus{invAb} | 2057 | asAfsgaucCfagaucuAfuGfgaaaasusu | 2789 |
| D-2049 | [GalNAc3]sugugacCfcUfUfCfAfgaacgaaas{invAb} | 2058 | asUfsuucgUfucugaaGfgGfucacasusu | 2790 |
| D-2050 | [GalNAc3]scaguguGfaCfCfCfUfucagaacgs{invAb} | 2059 | usCfsguucUfgaagggUfcAfcacugsusu | 2791 |
| D-2051 | [GalNAc3]scaccccAfaAfUfAfUfggcuggaas{invAb} | 2060 | asUfsuccaGfccauauUfuGfgggugsusu | 2792 |
| D-2052 | [GalNAc3]scucaauGfcUfUfCfAfaugucccas{invAb} | 2061 | asUfsgggaCfauugaaGfcAfuugagsusu | 2793 |
| D-2053 | [GalNAc3]sacaggaUfuCfUfGfAfaaacucccs{invAb} | 2062 | asGfsggagUfuuucagAfaUfccugususu | 2794 |
| D-2054 | [GalNAc3]sggauccUfuGfCfCfAfuuccccucs{invAb} | 2063 | usGfsagggGfaauggcAfaGfgauccsusu | 2795 |
| D-2055 | [GalNAc3]scugcagAfuAfUfUfAfauuuuccas{invAb} | 2064 | asUfsggaaAfauuaauAfuCfugcagsusu | 2796 |
| D-2056 | [GalNAc3]saagaucUfgAfUfGfAfaguauauus{invAb} | 2065 | asAfsauauAfcuucauCfaGfaucuususu | 2797 |
| D-2057 | [GalNAc3]saauagcAfgAfCfUfUfguuccgacs{invAb} | 2066 | asGfsucggAfacaaguCfuGfcuauususu | 2798 |
| D-2058 | [GalNAc3]sgacaacAfcUfUfGfAfagcauggus{invAb} | 2067 | asAfsccauGfcuucaaGfuGfuugucsusu | 2799 |
| D-2059 | [GalNAc3]sucagacAfgCfAfUfUfggauuuccs{invAb} | 2068 | asGfsgaaaUfccaaugCfuGfucugasusu | 2800 |
| D-2060 | [GalNAc3]suggaaaAfuCfAfCfCfacucuuugs{invAb} | 2069 | asCfsaaagAfguggugAfuUfuuccasusu | 2801 |
| D-2061 | [GalNAc3]sagacagCfaUfUfGfGfauuuccuas{invAb} | 2070 | usUfsaggaAfauccaaUfgCfugucususu | 2802 |
| D-2062 | [GalNAc3]scugauuAfuGfGfAfAfuaguucuus{invAb} | 2071 | asAfsagaaCfuauuccAfuAfaucagsusu | 2803 |
| D-2063 | [GalNAc3]sguauguCfcUfGfGfAfauauuagas{invAb} | 3062 | asUfscuaaUfauuccaGfgAfcauacsusu | 3321 |
| D-2064 | [GalNAc3]saggcuaGfaGfAfAfGfaaaguuaas{invAb} | 3063 | usUfsuaacUfuucuucUfcUfagccususu | 3322 |
| D-2065 | [GalNAc3]suguauaAfcUfCfUfAfagaucugas{invAb} | 3064 | asUfscagaUfcuuagaGfuUfauacasusu | 3323 |
| D-2066 | [GalNAc3]sccguauGfuCfCfUfGfgaauauuas{invAb} | 3065 | asUfsaauaUfuccaggAfcAfuacggsusu | 3324 |
| D-2067 | [GalNAc3]scaaaaaUfgAfCfAfAfcacuugaas{invAb} | 3066 | asUfsucaaGfuguuguCfaUfuuuugsusu | 3325 |
| D-2068 | [GalNAc3]suacugaAfaAfCfCfUfuuaaagggs{invAb} | 3067 | asCfsccuuUfaaagguUfuUfcaguasusu | 3326 |
| D-2069 | [GalNAc3]sacuacuGfaAfAfAfCfcuuuaaags{invAb} | 3068 | asCfsuuuaAfagguuuUfcAfguagususu | 3327 |
| D-2070 | [GalNAc3]sagaagaAfaAfGfUfGfauucagugs{invAb} | 3069 | usCfsacugAfaucacuUfuUfcuucususu | 3328 |
| D-2071 | [GalNAc3]suuugggCfaGfUfAfUfuuugugcus{invAb} | 3070 | asAfsgcacAfaaauacUfgCfccaaasusu | 3329 |
| D-2072 | [GalNAc3]suaccaaGfaGfCfGfCfagacuugcs{invAb} | 3071 | usGfscaagUfcugcgcUfcUfugguasusu | 3330 |
| D-2073 | [GalNAc3]scgggcuAfgCfUfUfUfugaaauggs{invAb} | 3072 | asCfscauuUfcaaaagCfuAfgcccgsusu | 3331 |
| D-2074 | [GalNAc3]scuggaaAfcAfCfUfGfaagaguuas{invAb} | 3073 | asUfsaacuCfuucaguGfuUfuccagsusu | 3332 |
| D-2075 | [GalNAc3]sauguccCfaGfUfGfCfaaaaaguas{invAb} | 3074 | usUfsacuuUfuugcacUfgGfgacaususu | 3333 |
| D-2076 | [GalNAc3]scuggaaUfaUfUfAfGfaugccuuus{invAb} | 3075 | asAfsaaggCfaucuaaUfaUfuccagsusu | 3334 |
| D-2077 | [GalNAc3]sccuggaAfuAfUfUfAfgaugccuus{invAb} | 3076 | asAfsaggcAfucuaauAfuUfccaggsusu | 3335 |
| D-2078 | [GalNAc3]succuggAfaUfAfUfUfagaugccus{invAb} | 3077 | asAfsggcaUfcuaauaUfuCfcaggasusu | 3336 |
| D-2079 | [GalNAc3]sacucuaAfgAfUfCfUfgaugaagus{invAb} | 3078 | usAfscuucAfucagauCfuUfagagususu | 3337 |
| D-2080 | [GalNAc3]sguccugGfaAfUfAfUfuagaugccs{invAb} | 3079 | asGfsgcauCfuaauauUfcCfaggacsusu | 3338 |
| D-2081 | [GalNAc3]suguccuGfgAfAfUfAfuuagaugcs{invAb} | 3080 | asGfscaucUfaauauuCfcAfggacasusu | 3339 |
| D-2082 | [GalNAc3]suaacucUfaAfGfAfUfcugaugaas{invAb} | 3081 | asUfsucauCfagaucuUfaGfaguuasusu | 3340 |
| D-2083 | [GalNAc3]sgugaugGfcUfUfGfUfuccagaugs{invAb} | 3082 | asCfsaucuGfgaacaaGfcCfaucacsusu | 3341 |
| D-2084 | [GalNAc3]sgucuauGfcAfGfAfGfgauucuugs{invAb} | 3083 | asCfsaagaAfuccucuGfcAfuagacsusu | 3342 |
| D-2085 | [GalNAc3]sacccugAfcUfCfUfCfagugcagcs{invAb} | 3084 | asGfscugcAfcugagaGfuCfagggususu | 3343 |
| D-2086 | [GalNAc3]sagccuaCfaCfAfAfAfggaccuacs{invAb} | 3085 | asGfsuaggUfccuuugUfgUfaggcususu | 3344 |
| D-2087 | [GalNAc3]saccaagAfgCfGfCfAfgacuugcas{invAb} | 3086 | asUfsgcaaGfucugcgCfuCfuuggususu | 3345 |
| D-2088 | [GalNAc3]sgaaggaAfcCfGfCfUfggaaacacs{invAb} | 3087 | asGfsuguuUfccagcgGfuUfccuucsusu | 3346 |
| D-2089 | [GalNAc3]suucccuUfuUfCfUfUfcucaagccs{invAb} | 3088 | asGfsgcuuGfagaagaAfaAfgggaasusu | 3347 |
| D-2090 | [GalNAc3]sccugucAfcUfAfCfCfacucguuas{invAb} | 3089 | asUfsaacgAfgugguaGfuGfacaggsusu | 3348 |
| D-2091 | [GalNAc3]scgcgcuUfuGfUfCfCfuccucgcgs{invAb} | 3090 | asCfsgcgaGfgaggacAfaAfgcgcgsusu | 3349 |
| D-2092 | [GalNAc3]sgaaacaUfgGfUfUfAfcugcucgcs{invAb} | 3091 | asGfscgagCfaguaacCfaUfguuucsusu | 3350 |
| D-2093 | [GalNAc3]scuuuguCfcUfCfCfUfcgcgcaaus{invAb} | 3092 | asAfsuugcGfcgaggaGfgAfcaaagsusu | 3351 |
| D-2094 | [GalNAc3]sgcauaaAfgAfCfUfGfaggugaccs{invAb} | 3093 | asGfsgucaCfcucaguCfuUfuaugcsusu | 3352 |
| D-2095 | [GalNAc3]suuugucCfuCfCfUfCfgcgcaaucs{invAb} | 3094 | asGfsauugCfgcgaggAfgGfacaaasusu | 3353 |
| D-2096 | [GalNAc3]sgaaaagGfgUfGfAfUfggcuuguus{invAb} | 3095 | asAfsacaaGfccaucaCfcCfuuuucsusu | 3354 |
| D-2097 | [GalNAc3]saaaaggGfuGfAfUfGfgcuuguucs{invAb} | 3096 | asGfsaacaAfgccaucAfcCfcuuuususu | 3355 |
| D-2098 | [GalNAc3]scuacugCfcUfAfUfCfaaaacgccs{invAb} | 3097 | asGfsgcguUfuugauaGfgCfaguagsusu | 3356 |
| D-2099 | [GalNAc3]scucaucAfaAfUfAfGfcagacuugs{invAb} | 3098 | asCfsaaguCfugcuauUfuGfaugagsusu | 3357 |
| D-2100 | [GalNAc3]sagacacCfaGfCfCfCfauucuugas{invAb} | 3099 | asUfscaagAfaugggcUfgGfugucususu | 3358 |
| D-2101 | [GalNAc3]scagcccAfuUfCfUfUfgauccuuus{invAb} | 3100 | asAfsaaggAfucaagaAfuGfggcugsusu | 3359 |
| D-2102 | [GalNAc3]sgcagagGfaUfUfCfUfugggaugas{invAb} | 3101 | asUfscaucCfcaagaaUfcCfucugcsusu | 3360 |
| D-2103 | [GalNAc3]scuaaagGfuGfCfUfCfaggaggaus{invAb} | 3102 | asAfsuccuCfcugagcAfcCfuuuagsusu | 3361 |
| D-2104 | [GalNAc3]saaagcaUfaUfGfUfCfaguuguuus{invAb} | 3103 | usAfsaacaAfcugacaUfaUfgcuuususu | 3362 |
| D-2105 | [GalNAc3]suuaacuGfaUfUfGfUfauaacucus{invAb} | 3104 | usAfsgaguUfauacaaUfcAfguuaasusu | 3363 |
| D-2106 | [GalNAc3]suaacugAfuUfGfUfAfuaacucuas{invAb} | 3105 | usUfsagagUfuauacaAfuCfaguuasusu | 3364 |
| D-2107 | [GalNAc3]sugaaaaCfuCfCfCfCfguuuaacus{invAb} | 3106 | asAfsguuaAfacggggAfgUfuuucasusu | 3365 |
| D-2108 | [GalNAc3]sgaugccUfuUfUfAfAfaaauguucs{invAb} | 3107 | asGfsaacaUfuuuuaaAfaGfgcaucsusu | 3366 |
| D-2109 | [GalNAc3]saaguugAfcUfAfAfAfcuugaaaas{invAb} | 3108 | usUfsuuucAfaguuuaGfuCfaacuususu | 3367 |
| D-2110 | [GalNAc3]scacaaaGfgAfCfCfUfacuacugcs{invAb} | 3109 | asGfscaguAfguagguCfcUfuugugsusu | 3368 |
| D-2111 | [GalNAc3]sgauuuuCfaCfAfUfUfuuucgucus{invAb} | 3110 | asAfsgacgAfaaaaugUfgAfaaaucsusu | 3369 |
| D-2112 | [GalNAc3]sccauucUfuGfAfUfCfcuuucugas{invAb} | 3111 | asUfscagaAfaggaucAfaGfaauggsusu | 3370 |
| D-2113 | [GalNAc3]sgaauauUfaGfAfUfGfccuuuuaas{invAb} | 3112 | usUfsuaaaAfggcaucUfaAfuauucsusu | 3371 |
| D-2114 | [GalNAc3]saaggaaAfgCfAfUfAfugucaguus{invAb} | 3113 | asAfsacugAfcauaugCfuUfuccuususu | 3372 |
| D-2115 | [GalNAc3]saggaaaGfcAfUfAfUfgucaguugs{invAb} | 3114 | asCfsaacuGfacauauGfcUfuuccususu | 3373 |
| D-2116 | [GalNAc3]sccccguUfuAfAfCfUfgauuauggs{invAb} | 3115 | usCfscauaAfucaguuAfaAfcggggsusu | 3374 |
| D-2117 | [GalNAc3]sggaaagCfaUfAfUfGfucaguugus{invAb} | 3116 | asAfscaacUfgacauaUfgCfuuuccsusu | 3375 |
| D-2118 | [GalNAc3]sgccauuUfuGfUfCfCfuuugauuas{invAb} | 3117 | asUfsaaucAfaaggacAfaAfauggcsusu | 3376 |
| D-2119 | [GalNAc3]saugacaAfcAfCfUfUfgaagcaugs{invAb} | 3118 | asCfsaugcUfucaaguGfuUfgucaususu | 3377 |
| D-2120 | [GalNAc3]scuccugCfuUfCfUfCfcguuuaucs{invAb} | 3119 | asGfsauaaAfcggagaAfgCfaggagsusu | 3378 |
| D-2121 | [GalNAc3]scagacuUfgCfAfUfCfcugucacus{invAb} | 3120 | usAfsgugaCfaggaugCfaAfgucugsusu | 3379 |
| D-2122 | [GalNAc3]sgagggaAfaCfAfUfGfguuacugcs{invAb} | 3121 | asGfscaguAfaccaugUfuUfcccucsusu | 3380 |
| D-2123 | [GalNAc3]suggcauAfaAfGfAfCfugaggugas{invAb} | 3122 | asUfscaccUfcagucuUfuAfugccasusu | 3381 |
| D-2124 | [GalNAc3]succuagAfaUfGfUfGfuuauugccs{invAb} | 3123 | asGfsgcaaUfaacacaUfuCfuaggasusu | 3382 |
| D-2125 | [GalNAc3]suggaaaCfaCfUfGfAfagaguuaus{invAb} | 3124 | asAfsuaacUfcuucagUfgUfuuccasusu | 3383 |
| D-2130 | [GalNAc3]suggauuUfcCfUfAfAfaggugcucs{invAb} | 3125 | usGfsagcaCfcuuuagGfaAfauccasusu | 3384 |
| D-2131 | [GalNAc3]sgaggagAfaGfAfAfAfagugauucs{invAb} | 3126 | usGfsaaucAfcuuuucUfuCfuccucsusu | 3385 |
| D-2134 | [GalNAc3]saccuacUfaCfUfGfCfcuaucaaas{invAb} | 3127 | usUfsuugaUfaggcagUfaGfuaggususu | 3386 |
| D-2135 | [GalNAc3]suacuacUfgCfCfUfAfucaaaacgs{invAb} | 3128 | asCfsguuuUfgauaggCfaGfuaguasusu | 3387 |
| D-2136 | [GalNAc3]saaauauAfgUfCfUfCfaauaacuus{invAb} | 3129 | usAfsaguuAfuugagaCfuAfuauuususu | 3388 |
| D-2137 | [GalNAc3]saacucuAfaGfAfUfCfugaugaags{invAb} | 3130 | asCfsuucaUfcagaucUfuAfgaguususu | 3389 |
| D-2138 | [GalNAc3]saagaguUfaUfCfGfCfcagugugas{invAb} | 3131 | asUfscacaCfuggcgaUfaAfcucuususu | 3390 |
| D-2139 | [GalNAc3]sccgggcUfaGfCfUfUfuugaaaugs{invAb} | 3132 | asCfsauuuCfaaaagcUfaGfcccggsusu | 3391 |
| D-2140 | [GalNAc3]sguuauaUfgGfAfAfAfaucaccacs{invAb} | 3133 | asGfsugguGfauuuucCfaUfauaacsusu | 3392 |
| D-2141 | [GalNAc3]saaagugAfuUfCfAfGfugauuucas{invAb} | 3134 | asUfsgaaaUfcacugaAfuCfacuuususu | 3393 |
| D-2142 | [GalNAc3]suaugucCfuGfGfAfAfuauuagaus{invAb} | 3135 | asAfsucuaAfuauuccAfgGfacauasusu | 3394 |
| D-2143 | [GalNAc3]sauaacuCfuAfAfGfAfucugaugas{invAb} | 3136 | usUfscaucAfgaucuuAfgAfguuaususu | 3395 |
| D-2144 | [GalNAc3]scuuguuCfcAfGfAfUfgcauuuuas{invAb} | 3137 | usUfsaaaaUfgcaucuGfgAfacaagsusu | 3396 |
| D-2145 | [GalNAc3]suauugcCfaUfUfUfUfguccuuugs{invAb} | 3138 | usCfsaaagGfacaaaaUfgGfcaauasusu | 3397 |
| D-2146 | [GalNAc3]sugauuuUfcAfCfAfUfuuuucgucs{invAb} | 3139 | asGfsacgaAfaaauguGfaAfaaucasusu | 3398 |
| D-2147 | [GalNAc3]scaagagCfgCfAfGfAfcuugcaucs{invAb} | 3140 | asGfsaugcAfagucugCfgCfucuugsusu | 3399 |
| D-2148 | [GalNAc3]scagauaGfaCfUfAfCfugaaaaccs{invAb} | 3141 | asGfsguuuUfcaguagUfcUfaucugsusu | 3400 |
| D-2149 | [GalNAc3]suuauggAfaUfAfGfUfucuuucucs{invAb} | 3142 | asGfsagaaAfgaacuaUfuCfcauaasusu | 3401 |
| D-2150 | [GalNAc3]scaugguGfuUfUfCfAfgaacugags{invAb} | 3143 | usCfsucagUfucugaaAfcAfccaugsusu | 3402 |
| D-2151 | [GalNAc3]sgaagaaAfaGfUfGfAfuucagugas{invAb} | 3144 | asUfscacuGfaaucacUfuUfucuucsusu | 3403 |
| D-2152 | [GalNAc3]sgucccaGfuGfCfAfAfaaaguaaas{invAb} | 3145 | asUfsuuacUfuuuugcAfcUfgggacsusu | 3404 |
| D-2153 | [GalNAc3]sacugauUfgUfAfUfAfacucuaags{invAb} | 3146 | usCfsuuagAfguuauaCfaAfucagususu | 3405 |
| D-2154 | [GalNAc3]saccguaUfgUfCfCfUfggaauauus{invAb} | 3147 | usAfsauauUfccaggaCfaUfacggususu | 3406 |
| D-2155 | [GalNAc3]succccgUfuUfAfAfCfugauuaugs{invAb} | 3148 | asCfsauaaUfcaguuaAfaCfggggasusu | 3407 |
| D-2156 | [GalNAc3]sauauagUfcUfCfAfAfuaacuuags{invAb} | 3149 | asCfsuaagUfuauugaGfaCfuauaususu | 3408 |
| D-2157 | [GalNAc3]succcagUfgCfAfAfAfaaguaaags{invAb} | 3150 | usCfsuuuaCfuuuuugCfaCfugggasusu | 3409 |
| D-2158 | [GalNAc3]saugcuuCfaAfUfGfUfcccaguuus{invAb} | 3151 | asAfscuggGfacauugAfaGfcaususu | 3410 |
| D-2159 | [GalNAc3]sgaacgaAfaGfUfUfAfuauggaaus{invAb} | 3152 | usUfsccauAfuaacuuUfcGfuucsusu | 3411 |
| D-2160 | [GalNAc3]sgauugcUfuAfCfUfCfagacacuus{invAb} | 3153 | asGfsugucUfgaguaaGfcAfaucsusu | 3412 |
| D-2161 | [GalNAc3]scuucagGfcCfCfAfAfuauuguaas{invAb} | 2047 | asUfsuAfcAfauauuggGfcCfugaagsusu | 2212 |
| D-2162 | [GalNAc3]scaaugcUfuCfAfAfUfgucccagus{invAb} | 2051 | asAfscUfgGfgacauugAfaGfcauugsusu | 2249 |
| D-2163 | [GalNAc3]scagaacGfaAfAfGfUfuauauggas{invAb} | 2010 | usUfscCfaUfauaacuuUfcGfuucugsusu | 2168 |
| D-2164 | [GalNAc3]sggaaguUfgAfCfUfAfaacuugaas{invAb} | 2031 | usUfsuCfaAfguuuaguCfaAfcuuccsusu | 2328 |
| D-2165 | [GalNAc3]sccagugGfaUfAfAfCfcagcuuccs{invAb} | 2013 | asGfsgAfaGfcugguuaUfcCfacuggsusu | 2116 |
| D-2166 | [GalNAc3]scuaagaUfcUfGfAfUfgaaguauas{invAb} | 2054 | asUfsaUfaCfuucaucaGfaUfcuuagsusu | 2317 |
| D-2167 | [GalNAc3]scagauuGfcUfUfAfCfucagacacs{invAb} | 2033 | asGfsuGfuCfugaguaaGfcAfaucugsusu | 2185 |
| D-2168 | [GalNAc3]scuucaggcCfcAfAfUfAfuuguaas{invAb} | 3154 | asUfsuacaAfuauuGfgGfccugaagsusu | 3413 |
| D-2169 | [GalNAc3]scaaugcuuCfaAfUfGfUfcccagus{invAb} | 3155 | asAfscuggGfacauUfgAfagcauugsusu | 3414 |
| D-2170 | [GalNAc3]scagaacgaAfaGfUfUfAfuauggas{invAb} | 3156 | usUfsccauAfuaacUfuUfcguucugsusu | 3415 |
| D-2171 | [GalNAc3]sggaaguugAfcUfAfAfAfcuugaas{invAb} | 3157 | usUfsucaaGfuuuaGfuCfaacuuccsusu | 3416 |
| D-2172 | [GalNAc3]sccaguggaUfaAfCfCfAfgcuuccs{invAb} | 3158 | asGfsgaagCfugguUfaUfccacuggsusu | 3417 |
| D-2173 | [GalNAc3]scuaagaucUfgAfUfGfAfaguauas{invAb} | 3159 | asUfsauacUfucauCfaGfaucuuagsusu | 3418 |
| D-2174 | [GalNAc3]scagauugcUfuAfCfUfCfagacacs{invAb} | 3160 | asGfsugucUfgaguAfaGfcaaucugsusu | 3419 |
| D-2175 | [GalNAc3]scuucagGfcCfCfAfAfuauuguaas{invAb} | 2047 | asUfsuacaAfuauuggGfcCfugasasgsusg | 3420 |
| D-2176 | [GalNAc3]scaaugcUfuCfAfAfUfgucccagus{invAb} | 2051 | asAfscuggGfacauugAfaGfcaususgsusg | 3421 |
| D-2177 | [GalNAc3]scagaacGfaAfAfGfUfuauauggas{invAb} | 2010 | usUfsccauAfuaacuuUfcGfuucsusgsusg | 3422 |
| D-2178 | [GalNAc3]sggaaguUfgAfCfUfAfaacuugaas{invAb} | 2031 | usUfsucaaGfuuuaguCfaAfcuuscscsusg | 3423 |
| D-2179 | [GalNAc3]sccagugGfaUfAfAfCfcagcuuccs{invAb} | 2013 | asGfsgaagCfugguuaUfcCfacusgsgsusg | 3424 |
| D-2180 | [GalNAc3]scuaagaUfcUfGfAfUfgaaguauas{invAb} | 2054 | asUfsauacUfucaucaGfaUfcuusasgsusg | 3425 |
| D-2181 | [GalNAc3]scagauuGfcUfUfAfCfucagacacs{invAb} | 2033 | asGfsugucUfgaguaaGfcAfaucsusgsusg | 3426 |
| D-2182 | [GalNAc3]scaaugcUfuCfAfAfUfgucccagus{invAb} | 2051 | asAfscuggGfacauugAfaGfcauugsasg | 3427 |
| D-2183 | [GalNAc3]scaaugcUfuCfaAfugucccagus{invAb} | 3161 | asAfscugggacauUfgAfaGfcauugsusu | 3428 |
| D-2184 | [GalNAc3]scaaugcUfuCfAfAfUfgucccagus{invAb} | 2051 | asAfscUfggGfacauugAfaGfcauugsusu | 3429 |
| D-2185 | [GalNAc3]scaaugcUfuCfAfAfUfgucccagus{invAb} | 2051 | asAfscugGfGfacauugAfaGfcauugsusu | 3430 |
| D-2186 | [GalNAc3]scaaugcUfuCfAfAfUfgucccagus{invAb} | 2051 | asAfscuggGfacauUfgAfaGfcauugsusu | 3431 |
| D-2187 | [GalNAc3]scaaugcUfuCfAfAfUfgucccagus{invAb} | 2051 | asAfscUfgGfGfacauugAfaGfcauugsusu | 3432 |
| D-2188 | [GalNAc3]scuucagGfcCfCfAfAfuauuguaas{invAb} | 2047 | asUfsuacaAfuauuggGfcCfugasasg | 3433 |
| D-2189 | [GalNAc3]scaaugcUfuCfAfAfUfgucccagus{invAb} | 2051 | asAfscuggGfacauugAfaGfcaususg | 3434 |
| D-2190 | [GalNAc3]scagaacGfaAfAfGfUfuauauggas{invAb} | 2010 | usUfsccauAfuaacuuUfcGfuucsusg | 3435 |
| D-2191 | [GalNAc3]sggaaguUfgAfCfUfAfaacuugaas{invAb} | 2031 | usUfsucaaGfuuuaguCfaAfcuuscsc | 3436 |
| D-2192 | [GalNAc3]sccagugGfaUfAfAfCfcagcuuccs{invAb} | 2013 | asGfsgaagCfugguuaUfcCfacusgsg | 3437 |
| D-2193 | [GalNAc3]scuaagaUfcUfGfAfUfgaaguauas{invAb} | 2054 | asUfsauacUfucaucaGfaUfcuusasg | 3438 |
| D-2194 | [GalNAc3]scagauuGfcUfUfAfCfucagacacs{invAb} | 2033 | asGfsugucUfgaguaaGfcAfaucsusg | 3439 |
| D-2195 | [GalNAc3]scaaugcUfuCfAfAfUfgucccauus{invAb} | 3162 | asUfsgggaCfauugaaGfcAfuugsusu | 3440 |
| D-2196 | [GalNAc3]sguuuaaAfaCfCfCfAfauaucuaus{invAb} | 3163 | usAfsgauaUfuggguuUfuAfaacsusu | 3441 |
| D-2197 | [GalNAc3]scuucaaUfgUfCfCfCfagugcaaus{invAb} | 3164 | usUfsgcacUfgggacaUfuGfaagsusu | 3442 |
| D-2198 | [GalNAc3]sagacagCfaUfUfGfGfauuuccuus{invAb} | 3165 | asGfsgaaaUfccaaugCfuGfucususu | 3443 |
| D-2199 | [GalNAc3]sgaaaauCfaCfCfAfCfucuuuguus{invAb} | 3166 | asCfsaaagAfguggugAfuUfuucsusu | 3444 |
| D-2200 | [GalNAc3]sacagcaUfuGfGfAfUfuuccuaaus{invAb} | 3167 | usUfsaggaAfauccaaUfgCfugususu | 3445 |
| D-2201 | [GalNAc3]saggauuCfuGfAfAfAfacucccuus{invAb} | 3168 | asGfsggagUfuuucagAfaUfccususu | 3446 |
| D-2202 | [GalNAc3]scaacacUfuGfAfAfGfcaugguuus{invAb} | 3169 | asAfsccauGfcuucaaGfuGfuugsusu | 3447 |
| D-2203 | [GalNAc3]scucaaugcUfuCfAfAfUfgucccas{invAb} | 3170 | asUfsgggaCfauugAfaGfcauugagsusu | 3448 |
| D-2204 | [GalNAc3]suuguuuaaAfaCfCfCfAfauaucus{invAb} | 3171 | usAfsgauaUfugggUfuUfuaaacaasusu | 3449 |
| D-2205 | [GalNAc3]sugcuucaaUfgUfCfCfCfagugcas{invAb} | 3172 | usUfsgcacUfgggaCfaUfugaagcasusu | 3450 |
| D-2206 | [GalNAc3]sucagacagCfaUfUfGfGfauuuccs{invAb} | 3173 | asGfsgaaaUfccaaUfgCfugucugasusu | 3451 |
| D-2207 | [GalNAc3]suggaaaauCfaCfCfAfCfucuuugs{invAb} | 3174 | asCfsaaagAfguggUfgAfuuuuccasusu | 3452 |
| D-2208 | [GalNAc3]sagacagcaUfuGfGfAfUfuuccuas{invAb} | 3175 | usUfsaggaAfauccAfaUfgcugucususu | 3453 |
| D-2209 | [GalNAc3]sacaggauuCfuGfAfAfAfacucccs{invAb} | 3176 | asGfsggagUfuuucAfgAfauccugususu | 3454 |
| D-2210 | [GalNAc3]sgacaacacUfuGfAfAfGfcauggus{invAb} | 3177 | asAfsccauGfcuucAfaGfuguugucsusu | 3455 |
| D-2211 | [GalNAc3]scucaauGfcUfUfCfAfaugucccas{invAb} | 2061 | asUfsgGfgAfcauugaaGfcAfuugagsusu | 2248 |
| D-2212 | [GalNAc3]suuguuuAfaAfAfCfCfcaauaucus{invAb} | 2026 | usAfsgAfuAfuuggguuUfuAfaacaasusu | 2315 |
| D-2213 | [GalNAc3]sugcuucAfaUfGfUfCfccagugcas{invAb} | 2052 | usUfsgCfaCfugggacaUfuGfaagcasusu | 2251 |
| D-2214 | [GalNAc3]sucagacAfgCfAfUfUfggauuuccs{invAb} | 2068 | asGfsgAfaAfuccaaugCfuGfucugasusu | 2285 |
| D-2215 | [GalNAc3]suggaaaAfuCfAfCfCfacucuuugs{invAb} | 2069 | asCfsaAfaGfaguggugAfuUfuuccasusu | 2222 |
| D-2216 | [GalNAc3]sagacagCfaUfUfGfGfauuuccuas{invAb} | 2070 | usUfsaGfgAfaauccaaUfgCfugucususu | 2287 |
| D-2217 | [GalNAc3]sacaggaUfuCfUfGfAfaaacucccs{invAb} | 2062 | asGfsgGfaGfuuuucagAfaUfccugususu | 2256 |
| D-2218 | [GalNAc3]sgacaacAfcUfUfGfAfagcauggus{invAb} | 2067 | asAfscCfaUfgcuucaaGfuGfuugucsusu | 2238 |
| D-2219 | [GalNAc3]scucaauGfcUfUfCfAfaugucccas{invAb} | 2061 | asUfsgggaCfauugaaGfcAfuugsasg | 3456 |
| D-2220 | [GalNAc3]suuguuuAfaAfAfCfCfcaauaucus{invAb} | 2026 | usAfsgauaUfuggguuUfuAfaacsasa | 3457 |
| D-2221 | [GalNAc3]sugcuucAfaUfGfUfCfccagugcas{invAb} | 2052 | usUfsgcacUfgggacaUfuGfaagscsa | 3458 |
| D-2222 | [GalNAc3]sucagacAfgCfAfUfUfggauuuccs{invAb} | 2068 | asGfsgaaaUfccaaugCfuGfucusgsa | 3459 |
| D-2223 | [GalNAc3]suggaaaAfuCfAfCfCfacucuuugs{invAb} | 2069 | asCfsaaagAfguggugAfuUfuucscsa | 3460 |
| D-2224 | [GalNAc3]sagacagCfaUfUfGfGfauuuccuas{invAb} | 2070 | usUfsaggaAfauccaaUfgCfuguscsu | 3461 |
| D-2225 | [GalNAc3]sacaggaUfuCfUfGfAfaaacucccs{invAb} | 2062 | asGfsggagUfuuucagAfaUfccusgsu | 3462 |
| D-2226 | [GalNAc3]sgacaacAfcUfUfGfAfagcauggus{invAb} | 2067 | asAfsccauGfcuucaaGfuGfuugsusc | 3463 |
| D-2227 | [GalNAc3]sucagaaCfgAfAfAfGfuuauauggs{invAb} | 2037 | usCfscAfuAfuaacuuuCfgUfucugasusu | 2167 |
| D-2228 | [GalNAc3]suuccagAfuGfCfAfUfuuuaaccas{invAb} | 2011 | asUfsgGfuUfaaaaugcAfuCfuggaasusu | 2133 |
| D-2229 | [GalNAc3]sccuucaGfaAfCfGfAfaaguuauas{invAb} | 2041 | asUfsaUfaAfcuuucguUfcUfgaaggsusu | 2164 |
| D-2230 | [GalNAc3]scuucagAfaCfGfAfAfaguuauaus{invAb} | 2042 | asAfsuAfuAfacuuucgUfuCfugaagsusu | 2165 |
| D-2231 | [GalNAc3]scaacuuCfaGfGfCfCfcaauauugs{invAb} | 2043 | asCfsaAfuAfuugggccUfgAfaguugsusu | 2210 |
| D-2232 | [GalNAc3]sacuucaGfgCfCfCfAfauauuguas{invAb} | 2045 | usUfsaCfaAfuauugggCfcUfgaagususu | 2211 |
| D-2233 | [GalNAc3]scuguuuAfaAfAfCfCfcaauaucus{invAb} | 3178 | usAfsgauaUfuggguuUfuAfaacagsusu | 3464 |
| D-2234 | [GalNAc3]scgcuucAfaUfGfUfCfccagugcas{invAb} | 3179 | usUfsgcacUfgggacaUfuGfaagcgsusu | 3465 |
| D-2235 | [GalNAc3]sccagacAfgCfAfUfUfggauuuccs{invAb} | 3180 | asGfsgaaaUfccaaugCfuGfucuggsusu | 3466 |
| D-2236 | [GalNAc3]scggaaaAfuCfAfCfCfacucuuugs{invAb} | 3181 | asCfsaaagAfguggugAfuUfuuccgsusu | 3467 |
| D-2237 | [GalNAc3]sggacagCfaUfUfGfGfauuuccuas{invAb} | 3182 | usUfsaggaAfauccaaUfgCfuguccsusu | 3468 |
| D-2238 | [GalNAc3]sgcaggaUfuCfUfGfAfaaacucccs{invAb} | 3183 | asGfsggagUfuuucagAfaUfccugcsusu | 3469 |
| D-2239 | [GalNAc3]sgauggcUfuGfUfUfCfcagauguus{invAb} | 3184 | asCfsaucuGfgaacaaGfcCfaucsusu | 3470 |
| D-2240 | [GalNAc3]succuggAfaUfAfUfUfagaugcuus{invAb} | 3185 | asGfscaucUfaauauuCfcAfggasusu | 3471 |
| D-2241 | [GalNAc3]sccuggaAfuAfUfUfAfgaugccuus{invAb} | 3076 | asGfsgcauCfuaauauUfcCfaggsusu | 3472 |
| D-2242 | [GalNAc3]sucuaagAfuCfUfGfAfugaaguaus{invAb} | 3186 | usAfscuucAfucagauCfuUfagasusu | 3473 |
| D-2243 | [GalNAc3]scuggaaUfaUfUfAfGfaugccuuus{invAb} | 3075 | asAfsggcaUfcuaauaUfuCfcagsusu | 3474 |
| D-2244 | [GalNAc3]sgugauggcUfuGfUfUfCfcagaugs{invAb} | 3187 | asCfsaucuGfgaacAfaGfccaucacsusu | 3475 |
| D-2245 | [GalNAc3]suguccuggAfaUfAfUfUfagaugcs{invAb} | 3188 | asGfscaucUfaauaUfuCfcaggacasusu | 3476 |
| D-2246 | [GalNAc3]sguccuggaAfuAfUfUfAfgaugccs{invAb} | 3189 | asGfsgcauCfuaauAfuUfccaggacsusu | 3477 |
| D-2247 | [GalNAc3]sacucuaagAfuCfUfGfAfugaagus{invAb} | 3190 | usAfscuucAfucagAfuCfuuagagususu | 3478 |
| D-2248 | [GalNAc3]succuggaaUfaUfUfAfGfaugccus{invAb} | 3191 | asAfsggcaUfcuaaUfaUfuccaggasusu | 3479 |
| D-2249 | [GalNAc3]sgugaugGfcUfUfGfUfuccagaugs{invAb} | 3082 | asCfsaUfcUfggaacaaGfcCfaucacsusu | 2439 |
| D-2250 | [GalNAc3]suguccuGfgAfAfUfAfuuagaugcs{invAb} | 3080 | asGfscAfuCfuaauauuCfcAfggacasusu | 2458 |
| D-2251 | [GalNAc3]sguccugGfaAfUfAfUfuagaugccs{invAb} | 3079 | asGfsgCfaUfcuaauauUfcCfaggacsusu | 2459 |
| D-2252 | [GalNAc3]sacucuaAfgAfUfCfUfgaugaagus{invAb} | 3078 | usAfscUfuCfaucagauCfuUfagagususu | 2479 |
| D-2253 | [GalNAc3]succuggAfaUfAfUfUfagaugccus{invAb} | 3077 | asAfsgGfcAfucuaauaUfuCfcaggasusu | 2460 |
| D-2254 | [GalNAc3]sgugaugGfcUfUfGfUfuccagaugs{invAb} | 3082 | asCfsaucuGfgaacaaGfcCfaucsasc | 3480 |
| D-2255 | [GalNAc3]suguccuGfgAfAfUfAfuuagaugcs{invAb} | 3080 | asGfscaucUfaauauuCfcAfggascsa | 3481 |
| D-2256 | [GalNAc3]sguccugGfaAfUfAfUfuagaugccs{invAb} | 3079 | asGfsgcauCfuaauauUfcCfaggsasc | 3482 |
| D-2257 | [GalNAc3]sacucuaAfgAfUfCfUfgaugaagus{invAb} | 3078 | usAfscuucAfucagauCfuUfagasgsu | 3483 |
| D-2258 | [GalNAc3]succuggAfaUfAfUfUfagaugccus{invAb} | 3077 | asAfsggcaUfcuaauaUfuCfcagsgsa | 3484 |
| D-2259 | [GalNAc3]scccuggAfaUfAfUfUfagaugccus{invAb} | 3192 | asAfsggcaUfcuaauaUfuCfcagggsusu | 3485 |
| D-2260 | +GalNAcThgcucuaAfgAfUfCfUfgaugaagus{invAb} | 3193 | usAfscuucAfucagauCfuUfagagcsusu | 3486 |
| D-2261 | [GalNAc3]scguccuGfgAfAfUfAfuuagaugcs{invAb} | 3194 | asGfscaucUfaauauuCfcAfggacgsusu | 3487 |
| D-2262 | [GalNAc3]scaacucUfaAfGfAfUfcugaugaas{invAb} | 3195 | asUfsucauCfagaucuUfaGfaguugsusu | 3488 |
| D-2263 | [GalNAc3]sggaagaAfaAfGfUfGfauucagugs{invAb} | 3196 | usCfsacugAfaucacuUfuUfcuuccsusu | 3489 |
| D-2264 | [GalNAc3]s[invAb]guccugGfaAfUfAfUfuagaugcscs{invAb} | 3197 | asGfsgcauCfuaauauUfcCfaggacsusu | 3338 |
| D-2265 | [GalNAc3]s[invAb]uccuggAfaUfAfUfUfagaugccsus{invAb} | 3198 | asAfsggcaUfcuaauaUfuCfcaggasusu | 3336 |
| D-2266 | [GalNAc3]s[invAb]acucuaAfgAfUfCfUfgaugaagsus{invAb} | 3199 | usAfscuucAfucagauCfuUfagagususu | 3337 |
| D-2267 | [GalNAc3]s[invAb]agacagCfaUfUfGfGfauuuccusas{invAb} | 3200 | usUfsaggaAfauccaaUfgCfugucususu | 2802 |
| D-2268 | [GalNAc3]sguccagGfaAfUfAfUfuagaugccs{invAb} | 3201 | asGfsgcauCfuaauauUfcCfuggacsusu | 3490 |
| D-2269 | [GalNAc3]sgugcugGfaAfUfAfUfuagaugccs{invAb} | 3202 | asGfsgcauCfuaauauUfcCfagcacsusu | 3491 |
| D-2270 | [GalNAc3]succucgAfaUfAfUfUfagaugccus{invAb} | 3203 | asAfsggcaUfcuaauaUfuCfgaggasusu | 3492 |
| D-2271 | [GalNAc3]sucguggAfaUfAfUfUfagaugccus{invAb} | 3204 | asAfsggcaUfcuaauaUfuCfcacgasusu | 3493 |
| D-2272 | [GalNAc3]sacacuaAfgAfUfCfUfgaugaagus{invAb} | 3205 | usAfscuucAfucagauCfuUfagugususu | 3494 |
| D-2273 | [GalNAc3]sagucuaAfgAfUfCfUfgaugaagus{invAb} | 3206 | usAfscuucAfucagauCfuUfagacususu | 3495 |
| D-2274 | [GalNAc3]sagucagCfaUfUfGfGfauuuccuas{invAb} | 3207 | usUfsaggaAfauccaaUfgCfugacususu | 3496 |
| D-2275 | [GalNAc3]sacacagCfaUfUfGfGfauuuccuas{invAb} | 3208 | usUfsaggaAfauccaaUfgCfugugususu | 3497 |
| D-2276 | [GalNAc3]s{invAb}guccuggaAfuAfUfUfAfgaugcscs{invAb} | 3209 | asGfsgcauCfuaauauUfcCfaggacsusu | 3338 |
| D-2277 | [GalNAc3]s{invAb}guccugGfaAfuAfuuagaugcscs{invAb} | 3210 | asGfsgcauCfuaauauUfcCfaggacsusu | 3338 |
| D-2278 | [GalNAc3]s{invAb}guccugGfaAfUfAfUfuagaugcscs{invAb} | 3211 | asGfsgcauCfuaauAfuUfccaggacsusu | 3477 |
| D-2279 | [GalNAc3]s{invAb}guccuggaAfuAfUfUfAfgaugcscs{invAb} | 3209 | asGfsgcauCfuaauAfuUfccaggacsusu | 3477 |
| D-2280 | [GalNAc3]s{invAb}guccugGfaAfuAfuuagaugcscs{invAb} | 3210 | asGfsgcauCfuaauAfuUfccaggacsusu | 3477 |
| D-2281 | [GalNAc3]s{invAb}guccugGfaAfUfAfUfuagaugcscs{invAb} | 3211 | asGfsgcaucuaauauUfcCfaggacsusu | 3498 |
| D-2282 | [GalNAc3]s{invAb}guccuggaAfuAfUfUfAfgaugcscs{invAb} | 3209 | asGfsgcaucuaauauUfcCfaggacsusu | 3498 |
| D-2283 | [GalNAc3]s{invAb}guccugGfaAfuAfuuagaugcscs{invAb} | 3210 | asGfsgcaucuaauauUfcCfaggacsusu | 3498 |
| D-2284 | [GalNAc3]s{invAb}guccugGfaAfUfAfUfuagaugcscs{invAb} | 3211 | asGfsgcaUfCfuaauauUfcCfaggacsusu | 3499 |
| D-2285 | [GalNAc3]s{invAb}guccuggaAfuAfUfUfAfgaugcscs{invAb} | 3209 | asGfsgcaUfCfuaauauUfcCfaggacsusu | 3499 |
| D-2286 | [GalNAc3]s{invAb}guccugGfaAfuAfuuagaugcscs{invAb} | 3210 | asGfsgcaUfCfuaauauUfcCfaggacsusu | 3499 |
| D-2287 | [GalNAc3]s{invAb}guccugGfaAfUfAfUfuagaugcscs{invAb} | 3211 | asGfsgcauCfuaauAfuUfcCfaggacsusu | 3500 |
| D-2288 | [GalNAc3]s{invAb}guccuggaAfuAfUfUfAfgaugcscs{invAb} | 3209 | asGfsgcauCfuaauAfuUfcCfaggacsusu | 3500 |
| D-2289 | [GalNAc3]s{invAb}guccugGfaAfuAfuuagaugcscs{invAb} | 3210 | asGfsgcauCfuaauAfuUfcCfaggacsusu | 3500 |
| D-2291 | [GalNAc3]scaaugcUfuCfaAfugucccagus{invAb} | 3161 | asAfscuggGfacauugAfaGfcauugsusu | 2783 |
| D-2292 | [GalNAc3]scaaugcUfuCfAfAfUfgucccagus{invAb} | 2051 | asAfscuggGfacauUfgAfagcauugsusu | 3414 |
| D-2293 | [GalNAc3]scaaugcUfuCfaAfugucccagus{invAb} | 3161 | asAfscuggGfacauUfgAfagcauugsusu | 3414 |
| D-2294 | [GalNAc3]scaaugcUfuCfAfAfUfgucccagus{invAb} | 2051 | asAfscugggacauUfgAfaGfcauugsusu | 3428 |
| D-2295 | [GalNAc3]scaaugcuuCfaAfUfGfUfcccagus{invAb} | 3155 | asAfscugggacauUfgAfaGfcauugsusu | 3428 |
| D-2296 | [GalNAc3]scaaugcuuCfaAfUfGfUfcccagus{invAb} | 3155 | asAfscugGfGfacauugAfaGfcauugsusu | 3430 |
| D-2297 | [GalNAc3]scaaugcUfuCfaAfugucccagus{invAb} | 3161 | asAfscugGfGfacauugAfaGfcauugsusu | 3430 |
| D-2298 | [GalNAc3]scaaugcuuCfaAfUfGfUfcccagus{invAb} | 3155 | asAfscuggGfacauUfgAfaGfcauugsusu | 3431 |
| D-2299 | [GalNAc3]scaaugcUfuCfaAfugucccagus{invAb} | 3161 | asAfscuggGfacauUfgAfaGfcauugsusu | 3431 |
| D-2301 | [GalNAc3]sauguccUfgGfAfAfUfauuagaugs{invAb} | 3212 | asCfsaucuAfauauucCfaGfgacaususu | 3501 |
| D-2302 | [GalNAc3]suauaacUfcUfAfAfGfaucugaugs{invAb} | 3213 | usCfsaucaGfaucuuaGfaGfuuauasusu | 3502 |
| D-2303 | [GalNAc3]suaagauCfuGfAfUfGfaaguauaus{invAb} | 3214 | asAfsuauaCfuucaucAfgAfucuuasusu | 3503 |
| D-2304 | [GalNAc3]sggugucUfcAfAfUfGfcuucaaugs{invAb} | 3215 | asCfsauugAfagcauuGfaGfacaccsusu | 3504 |
| D-2305 | [GalNAc3]sgugucuCfaAfUfGfCfuucaaugus{invAb} | 3216 | asAfscauuGfaagcauUfgAfgacacsusu | 3505 |
| D-2306 | [GalNAc3]sucucaaUfgCfUfUfCfaaugucccs{invAb} | 3217 | usGfsggacAfuugaagCfaUfugagasusu | 3506 |
| D-2307 | [GalNAc3]saaugcuUfcAfAfUfGfucccagugs{invAb} | 3218 | asCfsacugGfgacauuGfaAfgcauususu | 3507 |
| D-2308 | [GalNAc3]saugcuuCfaAfUfGfUfcccagugcs{invAb} | 3219 | usGfscacuGfggacauUfgAfagcaususu | 3508 |
| D-2309 | [GalNAc3]succuggAfaUfaUfuagaugccus{invAb} | 3220 | asAfsggcaUfcuaauaUfuCfcaggasusu | 3336 |
| D-2310 | [GalNAc3]sacucuaAfgAfuCfugaugaagus{invAb} | 3221 | usAfscuucAfucagauCfuUfagagususu | 3337 |
| D-2311 | [GalNAc3]sguccugGfaAfuAfuuagaugccs{invAb} | 3222 | asGfsgcauCfuaauauUfcCfaggacsusu | 3338 |
| D-2312 | [GalNAc3]suguccuGfgAfaUfauuagaugcs{invAb} | 3223 | asGfscaucUfaauauuCfcAfggacasusu | 3339 |
| D-2313 | [GalNAc3]suaacucUfaAfgAfucugaugaas{invAb} | 3224 | asUfsucauCfagaucuUfaGfaguuasusu | 3340 |
| D-2314 | [GalNAc3]succuggaaUfaUfUfAfGfaugccus{invAb} | 3191 | asAfsggcaUfcuaauaUfuCfcaggasusu | 3336 |
| D-2315 | [GalNAc3]sacucuaagAfuCfUfGfAfugaagus{invAb} | 3190 | usAfscuucAfucagauCfuUfagagususu | 3337 |
| D-2316 | [GalNAc3]sguccuggaAfuAfUfUfAfgaugccs{invAb} | 3189 | asGfsgcauCfuaauauUfcCfaggacsusu | 3338 |
| D-2317 | [GalNAc3]suguccuggAfaUfAfUfUfagaugcs{invAb} | 3188 | asGfscaucUfaauauuCfcAfggacasusu | 3339 |
| D-2318 | [GalNAc3]suaacucuaAfgAfUfCfUfgaugaas{invAb} | 3225 | asUfsucauCfagaucuUfaGfaguuasusu | 3340 |
| D-2319 | [GalNAc3]succuggAfaUfaUfuagaugccus{invAb} | 3220 | asAfsggcaUfcuaaUfaUfuccaggasusu | 3479 |
| D-2320 | [GalNAc3]sacucuaAfgAfuCfugaugaagus{invAb} | 3221 | usAfscuucAfucagAfuCfuuagagususu | 3478 |
| D-2321 | [GalNAc3]sguccugGfaAfuAfuuagaugccs{invAb} | 3222 | asGfsgcauCfuaauAfuUfccaggacsusu | 3477 |
| D-2322 | [GalNAc3]suguccuGfgAfaUfauuagaugcs{invAb} | 3223 | asGfscaucUfaauaUfuCfcaggacasusu | 3476 |
| D-2323 | [GalNAc3]suaacucUfaAfgAfucugaugaas{invAb} | 3224 | asUfsucauCfagauCfuUfagaguuasusu | 3509 |
| D-2324 | [GalNAc3]succuggAfaUfAfUfUfagaugccus{invAb} | 3077 | asAfsggcaucuaaUfaUfuCfcaggasusu | 3510 |
| D-2325 | [GalNAc3]sacucuaAfgAfUfCfUfgaugaagus{invAb} | 3078 | usAfscuucaucagAfuCfuUfagagususu | 3511 |
| D-2326 | [GalNAc3]sguccugGfaAfUfAfUfuagaugccs{invAb} | 3079 | asGfsgcaucuaauAfuUfcCfaggacsusu | 3512 |
| D-2327 | [GalNAc3]suguccuGfgAfAfUfAfuuagaugcs{invAb} | 3080 | asGfscaucuaauaUfuCfcAfggacasusu | 3513 |
| D-2328 | [GalNAc3]suaacucUfaAfGfAfUfcugaugaas{invAb} | 3081 | asUfsucaucagauCfuUfaGfaguuasusu | 3514 |
| D-2329 | [GalNAc3]succuggAfaUfAfUfUfagaugccus{invAb} | 3077 | asAfsggcaUfcuaaUfaUfuccaggasusu | 3479 |
| D-2330 | [GalNAc3]sacucuaAfgAfUfCfUfgaugaagus{invAb} | 3078 | usAfscuucAfucagAfuCfuuagagususu | 3478 |
| D-2331 | [GalNAc3]sguccugGfaAfUfAfUfuagaugccs{invAb} | 3079 | asGfsgcauCfuaauAfuUfccaggacsusu | 3477 |
| D-2332 | [GalNAc3]suguccuGfgAfAfUfAfuuagaugcs{invAb} | 3080 | asGfscaucUfaauaUfuCfcaggacasusu | 3476 |
| D-2333 | [GalNAc3]suaacucUfaAfGfAfUfcugaugaas{invAb} | 3081 | asUfsucauCfagauCfuUfagaguuasusu | 3509 |
| D-2334 | [GalNAc3]succuggaaUfaUfUfAfGfaugccus{invAb} | 3191 | asAfsggcaucuaaUfaUfuCfcaggasusu | 3510 |
| D-2335 | [GalNAc3]sacucuaagAfuCfUfGfAfugaagus{invAb} | 3190 | usAfscuucaucagAfuCfuUfagagususu | 3511 |
| D-2336 | [GalNAc3]sguccuggaAfuAfUfUfAfgaugccs{invAb} | 3189 | asGfsgcaucuaauAfuUfcCfaggacsusu | 3512 |
| D-2337 | [GalNAc3]suguccuggAfaUfAfUfUfagaugcs{invAb} | 3188 | asGfscaucuaauaUfuCfcAfggacasusu | 3513 |
| D-2338 | [GalNAc3]suaacucuaAfgAfUfCfUfgaugaas{invAb} | 3225 | asUfsucaucagauCfuUfaGfaguuasusu | 3514 |
| D-2339 | [GalNAc3]succuggaaUfaUfUfAfGfaugccus{invAb} | 3191 | asAfsggcaUfcuaaUfaUfuCfcaggasusu | 3515 |
| D-2340 | [GalNAc3]sacucuaagAfuCfUfGfAfugaagus{invAb} | 3190 | usAfscuucAfucagAfuCfuUfagagususu | 3516 |
| D-2341 | [GalNAc3]sguccuggaAfuAfUfUfAfgaugccs{invAb} | 3189 | asGfsgcauCfuaauAfuUfcCfaggacsusu | 3500 |
| D-2342 | [GalNAc3]suguccuggAfaUfAfUfUfagaugcs{invAb} | 3188 | asGfscaucUfaauaUfuCfcAfggacasusu | 3517 |
| D-2343 | [GalNAc3]suaacucuaAfgAfUfCfUfgaugaas{invAb} | 3225 | asUfsucauCfagauCfuUfaGfaguuasusu | 3518 |
| D-2344 | [GalNAc3]succuggAfaUfaUfuagaugccus{invAb} | 3220 | asAfsggcAfUfcuaauaUfuCfcaggasusu | 3519 |
| D-2345 | [GalNAc3]sacucuaAfgAfuCfugaugaagus{invAb} | 3221 | usAfscuuCfAfucagauCfuUfagagususu | 3520 |
| D-2346 | [GalNAc3]sguccugGfaAfuAfuuagaugccs{invAb} | 3222 | asGfsgcaUfCfuaauauUfcCfaggacsusu | 3499 |
| D-2347 | [GalNAc3]suguccuGfgAfaUfauuagaugcs{invAb} | 3223 | asGfscauCfUfaauauuCfcAfggacasusu | 3521 |
| D-2348 | [GalNAc3]suaacucUfaAfgAfucugaugaas{invAb} | 3224 | asUfsucaUfCfagaucuUfaGfaguuasusu | 3522 |
| D-2349 | [GalNAc3]succuggaaUfaUfUfAfGfaugccus{invAb} | 3191 | asAfsggcAfUfcuaauaUfuCfcaggasusu | 3519 |
| D-2350 | [GalNAc3]sacucuaagAfuCfUfGfAfugaagus{invAb} | 3190 | usAfscuuCfAfucagauCfuUfagagususu | 3520 |
| D-2351 | [GalNAc3]sguccuggaAfuAfUfUfAfgaugccs{invAb} | 3189 | asGfsgcaUfCfuaauauUfcCfaggacsusu | 3499 |
| D-2352 | [GalNAc3]suguccuggAfaUfAfUfUfagaugcs{invAb} | 3188 | asGfscauCfUfaauauuCfcAfggacasusu | 3521 |
| D-2353 | [GalNAc3]suaacucuaAfgAfUfCfUfgaugaas{invAb} | 3225 | asUfsucaUfCfagaucuUfaGfaguuasusu | 3522 |
| D-2354 | [GalNAc3]succuggAfaUfaUfuagaugccus{invAb} | 3220 | asAfsggcaUfcuaaUfaUfuCfcaggasusu | 3515 |
| D-2355 | [GalNAc3]sacucuaAfgAfuCfugaugaagus{invAb} | 3221 | usAfscuucAfucagAfuCfuUfagagususu | 3516 |
| D-2356 | [GalNAc3]sguccugGfaAfuAfuuagaugccs{invAb} | 3222 | asGfsgcauCfuaauAfuUfcCfaggacsusu | 3500 |
| D-2357 | [GalNAc3]suguccuGfgAfaUfauuagaugcs{invAb} | 3223 | asGfscaucUfaauaUfuCfcAfggacasusu | 3517 |
| D-2358 | [GalNAc3]suaacucUfaAfgAfucugaugaas{invAb} | 3224 | asUfsucauCfagauCfuUfaGfaguuasusu | 3518 |
| D-2359 | [GalNAc3]sugauggCfuUfGfUfUfccagaugcs{invAb} | 3226 | usGfscaucUfggaacaAfgCfcaucasusu | 3523 |
| D-2360 | [GalNAc3]sgauggcUfuGfUfUfCfcagaugcas{invAb} | 3227 | asUfsgcauCfuggaacAfaGfccaucsusu | 3524 |
| D-2361 | [GalNAc3]scucagaCfaGfCfAfUfuggauuucs{invAb} | 3228 | asGfsaaauCfcaaugcUfgUfcugagsusu | 3525 |
| D-2362 | [GalNAc3]sugccauUfuUfGfUfCfcuuugauus{invAb} | 3229 | usAfsaucaAfaggacaAfaAfuggcasusu | 3526 |
| D-2363 | [GalNAc3]sccauuuUfgUfCfCfUfuugauuaus{invAb} | 3230 | usAfsuaauCfaaaggaCfaAfaauggsusu | 3527 |
| D-2364 | [GalNAc3]sugauggcuUfgUfUfCfCfagaugcs{invAb} | 3231 | usGfscaucUfggaaCfaAfgccaucasusu | 3528 |
| D-2365 | [GalNAc3]sgauggcuuGfuUfCfCfAfgaugcas{invAb} | 3232 | asUfsgcauCfuggaAfcAfagccaucsusu | 3529 |
| D-2366 | [GalNAc3]scucagacaGfcAfUfUfGfgauuucs{invAb} | 3233 | asGfsaaauCfcaauGfcUfgucugagsusu | 3530 |
| D-2367 | [GalNAc3]sugccauuuUfgUfCfCfUfuugauus{invAb} | 3234 | usAfsaucaAfaggaCfaAfaauggcasusu | 3531 |
| D-2368 | [GalNAc3]sccauuuugUfcCfUfUfUfgauuaus{invAb} | 3235 | usAfsuaauCfaaagGfaCfaaaauggsusu | 3532 |
| D-2369 | [GalNAc3]sugauggCfuUfGfUfUfccagaugcs{invAb} | 3226 | usGfscAfuCfuggaacaAfgCfcaucasusu | 3533 |
| D-2370 | [GalNAc3]sgauggcUfuGfUfUfCfcagaugcas{invAb} | 3227 | asUfsgCfaUfcuggaacAfaGfccaucsusu | 3534 |
| D-2371 | [GalNAc3]scucagaCfaGfCfAfUfuggauuucs{invAb} | 3228 | asGfsaAfaUfccaaugcUfgUfcugagsusu | 3535 |
| D-2372 | [GalNAc3]sugccauUfuUfGfUfCfcuuugauus{invAb} | 3229 | usAfsaUfcAfaaggacaAfaAfuggcasusu | 3536 |
| D-2373 | [GalNAc3]sccauuuUfgUfCfCfUfuugauuaus{invAb} | 3230 | usAfsuAfaUfcaaaggaCfaAfaauggsusu | 3537 |
| D-2374 | [GalNAc3]sgsauuuuCfaCfAfUfUfuuucgucus{invAb} | 3236 | asAfsgacgAfaaaaugUfgAfaaaucsusu | 3369 |
| D-2375 | [GalNAc3]sgauuuucaCfaUfUfUfUfucgucus{invAb} | 3237 | asAfsgacgAfaaaaUfgUfgaaaaucsusu | 3538 |
| D-2376 | [GalNAc3]sgaauauuaGfaUfGfCfCfuuuuaas{invAb} | 3238 | usUfsuaaaAfggcaUfcUfaauauucsusu | 3539 |
| D-2377 | [GalNAc3]saggaaagcAfuAfUfGfUfcaguugs{invAb} | 3239 | asCfsaacuGfacauAfuGfcuuuccususu | 3540 |
| D-2378 | [GalNAc3]sgccauuuuGfuCfCfUfUfugauuas{invAb} | 3240 | asUfsaaucAfaaggAfcAfaaauggcsusu | 3541 |
| D-2379 | [GalNAc3]sgauuuuCfaCfAfUfUfuuucgucus{invAb} | 3110 | asAfsgAfcGfaaaaaugUfgAfaaaucsusu | 2605 |
| D-2380 | [GalNAc3]sgaauauUfaGfAfUfGfccuuuuaas{invAb} | 3112 | usUfsuAfaAfaggcaucUfaAfuauucsusu | 2597 |
| D-2381 | [GalNAc3]saggaaaGfcAfUfAfUfgucaguugs{invAb} | 3114 | asCfsaAfcUfgacauauGfcUfuuccususu | 2727 |
| D-2382 | [GalNAc3]sgccauuUfuGfUfCfCfuuugauuas{invAb} | 3117 | asUfsaAfuCfaaaggacAfaAfauggcsusu | 2739 |
| D-2383 | [GalNAc3]sgsasuuuuCfaCfAfUfUfuuucgucus{invAb} | 3241 | asAfsgacgAfaaaaugUfgAfaaaucsusu | 3369 |
| D-2384 | [GalNAc3]scaaugcuuCfaAfUfGfUfcccagus{invAb} | 3155 | asAfscuggGfacauugAfaGfcaususg | 3434 |
| D-2385 | [GalNAc3]scaaugcUfuCfAfAfUfgucccagus{invAb} | 2051 | asAfscugGfGfacauugAfaGfcaususg | 3542 |
| D-2386 | [GalNAc3]saugcuuCfaAfUfGfUfcccaguuus{invAb} | 3151 | asAfscuggGfacauugAfaGfcaususg | 3434 |
| D-2387 | [GalNAc3]saugcuuCfaAfUfGfUfcccaguuus{invAb} | 3151 | asAfscugGfGfacauugAfaGfcaususu | 3543 |
| D-2388 | [GalNAc3]scaaugcuuCfaAfUfGfUfcccagus{invAb} | 3155 | asAfscuggGfacauugAfaGfcauugsusu | 2783 |
| D-2389 | [GalNAc3]scaaugcuuCfaAfUfGfUfcccagus{invAb} | 3155 | asAfscuggGfacauugAfaGfcauugsasg | 3427 |
| D-2390 | [GalNAc3]saugcUfuCfAfAfUfgucccaguuus{invAb} | 3242 | asAfscuggGfacauugAfaGfcaususu | 3410 |
| D-2391 | [GalNAc3]scaaugcUfuCfAfAfUfgucccagus{invAb} | 2051 | asAfscugGfGfacauugAfaGfcauugsasg | 3544 |
| D-2392 | [GalNAc3]saugcUfuCfAfAfUfgucccaguuus{invAb} | 3242 | asAfscuggGfacauugAfaGfcaususg | 3434 |
| D-2393 | [GalNAc3]succuggAfaUfaUfuagaugccus{invAb} | 3220 | asAfsggcaucuaaUfaUfuCfcaggasusu | 3510 |
| D-2394 | [GalNAc3]sacucuaAfgAfuCfugaugaagus{invAb} | 3221 | usAfscuucaucagAfuCfuUfagagususu | 3511 |
| D-2395 | [GalNAc3]sguccugGfaAfuAfuuagaugccs{invAb} | 3222 | asGfsgcaucuaauAfuUfcCfaggacsusu | 3512 |
| D-2396 | [GalNAc3]suguccuGfgAfaUfauuagaugcs{invAb} | 3223 | asGfscaucuaauaUfuCfcAfggacasusu | 3513 |
| D-2397 | [GalNAc3]suaacucUfaAfgAfucugaugaas{invAb} | 3224 | asUfsucaucagauCfuUfaGfaguuasusu | 3514 |
| D-2399 | [GalNAc3]scaaugcUfuCfAfAfUfgucccagus{invAb} | 2051 | asAfscuggGfacauUfgAfaGfcaususg | 3545 |
| D-2400 | [GalNAc3]saugcuuCfaAfUfGfUfcccaguuus{invAb} | 3151 | asAfscuggGfacauUfgAfaGfcaususu | 3546 |
| D-2401 | [GalNAc3]scaaugcUfuCfAfAfUfgucccagus{invAb} | 2051 | asAfscuggGfacauUfgAfaGfcauugsasg | 3547 |
| D-2402 | [GalNAc3]scaaugcuuCfaAfUfGfUfcccagus{invAb} | 3155 | asAfscugGfGfacauugAfaGfcauugsasg | 3544 |
| D-2403 | [GalNAc3]scaaugcuuCfaAfUfGfUfcccagus{invAb} | 3155 | asAfscuggGfacauUfgAfaGfcauugsasg | 3547 |
| D-2430 | [GalNAc3]sgauuuuCfaCfAfUfUfuuucgucus{invAb} | 3110 | asAfsgacgAfaaaaugUfgAfaaasusc | 3548 |
| D-2431 | [GalNAc3]sgaauauUfaGfAfUfGfccuuuuaas{invAb} | 3112 | usUfsuaaaAfggcaucUfaAfuaususc | 3549 |
| D-2432 | [GalNAc3]saggaaaGfcAfUfAfUfgucaguugs{invAb} | 3114 | asCfsaacuGfacauauGfcUfuucscsu | 3550 |
| D-2433 | [GalNAc3]sgccauuUfuGfUfCfCfuuugauuas{invAb} | 3117 | asUfsaaucAfaaggacAfaAfaugsgsc | 3551 |
| D-2434 | [GalNAc3]saaauauAfgUfCfUfCfaauaacuus{invAb} | 3129 | usAfsaguuAfuugagaCfuAfuaususu | 3552 |
| D-2435 | [GalNAc3]suaccaaGfaGfCfGfCfagacuugcs{invAb} | 3071 | usGfscaagUfcugcgcUfcUfuggsusa | 3553 |
| D-2436 | [GalNAc3]sgaugccUfuUfUfAfAfaaauguucs{invAb} | 3107 | asGfsaacaUfuuuuaaAfaGfgcasusc | 3554 |
| D-2437 | [GalNAc3]sauguccUfgGfAfAfUfauuagaugs{invAb} | 3212 | asCfsaucuAfauauucCfaGfgacsasu | 3555 |
| D-2438 | [GalNAc3]saaauauagUfcUfCfAfAfuaacuus{invAb} | 3243 | usAfsaguuAfuugaGfaCfuauauuususu | 3556 |
| D-2439 | [GalNAc3]suaccaagaGfcGfCfAfGfacuugcs{invAb} | 3244 | usGfscaagUfcugcGfcUfcuugguasusu | 3557 |
| D-2440 | [GalNAc3]sgaugccuuUfuAfAfAfAfauguucs{invAb} | 3245 | asGfsaacaUfuuuuAfaAfaggcaucsusu | 3558 |
| D-2441 | [GalNAc3]sauguccugGfaAfUfAfUfuagaugs{invAb} | 3246 | asCfsaucuAfauauUfcCfaggacaususu | 3559 |
| D-2442 | [GalNAc3]saaauauAfgUfCfUfCfaauaacuus{invAb} | 3129 | usAfsaGfuUfauugagaCfuAfuauuususu | 2627 |
| D-2443 | [GalNAc3]suaccaaGfaGfCfGfCfagacuugcs{invAb} | 3071 | usGfscAfaGfucugcgcUfcUfugguasusu | 2667 |
| D-2444 | [GalNAc3]sgaugccUfuUfUfAfAfaaauguucs{invAb} | 3107 | asGfsaAfcAfuuuuuaaAfaGfgcaucsusu | 2598 |
| D-2445 | [GalNAc3]sauguccUfgGfAfAfUfauuagaugs{invAb} | 3212 | asCfsaUfcUfaauauucCfaGfgacaususu | 2457 |
| D-2446 | [GalNAc3]suuuucaCfaUfUfUfUfucgucuuus{invAb} | 3247 | asAfsgacgAfaaaaugUfgAfaaasusu | 3560 |
| D-2447 | [GalNAc3]sauauuaGfaUfGfCfCfuuuuaaaus{invAb} | 3248 | usUfsuaaaAfggcaucUfaAfuaususu | 3561 |
| D-2448 | [GalNAc3]sgaaagcAfuAfUfGfUfcaguuguus{invAb} | 3249 | asCfsaacuGfacauauGfcUfuucsusu | 3562 |
| D-2449 | [GalNAc3]scauuuuGfuCfCfUfUfugauuauus{invAb} | 3250 | asUfsaaucAfaaggacAfaAfaugsusu | 3563 |
| D-2450 | [GalNAc3]sauauagUfcUfCfAfAfuaacuuaus{invAb} | 3251 | usAfsaguuAfuugagaCfuAfuaususu | 3552 |
| D-2451 | [GalNAc3]sccaagaGfcGfCfAfGfacuugcaus{invAb} | 3252 | usGfscaagUfcugcgcUfcUfuggsusu | 3564 |
| D-2452 | [GalNAc3]sugccuuUfuAfAfAfAfauguucuus{invAb} | 3253 | asGfsaacaUfuuuuaaAfaGfgcasusu | 3565 |
| D-2453 | [GalNAc3]sguccugGfaAfUfAfUfuagauguus{invAb} | 3254 | asCfsaucuAfauauucCfaGfgacsusu | 3566 |
| D-2454 | [GalNAc3]sgcucuaAfgAfUfCfUfgaugaagus{invAb} | 3193 | usAfscUfuCfaucagauCfuUfagagcsusu | 3567 |
| D-2455 | [GalNAc3]sgcucuaagAfuCfUfGfAfugaagus{invAb} | 3255 | usAfscuucAfucagAfuCfuuagagcsusu | 3568 |
| D-2456 | [GalNAc3]sgcucuaagAfuCfUfGfAfugaagus{invAb} | 3255 | usAfscUfuCfaucagAfuCfuuagagcsusu | 3569 |
| D-2457 | [GalNAc3]sccuaagAfuCfUfGfAfugaaguaus{invAb} | 3256 | usAfscuucAfucagauCfuUfaggsusu | 3570 |
| D-2458 | [GalNAc3]sccuaagAfuCfUfGfAfugaaguaus{invAb} | 3256 | usAfscUfuCfaucagauCfuUfaggsusu | 3571 |
| D-2459 | [GalNAc3]sccuaAfgAfUfCfUfgaugaaguaus{invAb} | 3257 | usAfscuucAfucagauCfuUfaggsusu | 3570 |
| D-2460 | [GalNAc3]sccuaAfgAfUfCfUfgaugaaguaus{invAb} | 3257 | usAfscUfuCfaucagauCfuUfaggsusu | 3571 |
| D-2461 | [GalNAc3]suuguuuAfaAfAfCfCfcaauaucus{invAb} | 2026 | usAfsgauaUfuggguuUfuAfaacaascsu | 3572 |
| D-2462 | [GalNAc3]scagaacGfaAfAfGfUfuauauggas{invAb} | 2010 | usUfsccauAfuaacuuUfcGfuucugsasa | 3573 |
| D-2463 | [GalNAc3]scuaagaUfcUfGfAfUfgaaguauas{invAb} | 2054 | asUfsauacUfucaucaGfaUfcuuagsasg | 3574 |
| D-2464 | [GalNAc3]suggaaaAfuCfAfCfCfacucuuugs{invAb} | 2069 | asCfsaaagAfguggugAfuUfuuccasusa | 3575 |
| D-2465 | [GalNAc3]suaacucUfaAfGfAfUfcugaugaas{invAb} | 3081 | asUfsucauCfagaucuUfaGfagususa | 3576 |
| D-2466 | [GalNAc3]sagaagaAfaAfGfUfGfauucagugs{invAb} | 3069 | usCfsacugAfaucacuUfuUfcuuscsu | 3577 |
| D-2467 | [GalNAc3]sgauuuuCfaCfAfUfUfuuucgucus{invAb} | 3110 | asAfsgacgAfaaaaugUfgAfaaaucsasc | 3578 |
| D-2468 | [GalNAc3]suaacucUfaAfGfAfUfcugaugaas{invAb} | 3081 | asUfsucauCfagaucuUfaGfaguuasusa | 3579 |
| D-2469 | [GalNAc3]sagaagaAfaAfGfUfGfauucagugs{invAb} | 3069 | usCfsacugAfaucacuUfuUfcuucuscsc | 3580 |
| D-2470 | [GalNAc3]suaacucuaAfgAfUfCfUfgaugaas{invAb} | 3225 | asUfsucauCfagauCfuUfagaguuasusu | 3509 |
| D-2471 | [GalNAc3]sagaagaaaAfgUfGfAfUfucagugs{invAb} | 3258 | usCfsacugAfaucaCfuUfuucuucususu | 3581 |
| D-2472 | [GalNAc3]saagaucUfgAfUfGfAfaguauauus{invAb} | 2065 | asUfsauacUfucaucaGfaUfcuusasg | 3438 |
| D-2473 | [GalNAc3]sacucuaAfgAfUfCfUfgaugaauus{invAb} | 3259 | asUfsucauCfagaucuUfaGfagususu | 3582 |
| D-2474 | [GalNAc3]saagaaaAfgUfGfAfUfucagugaus{invAb} | 3260 | usCfsacugAfaucacuUfuUfcuususu | 3583 |
| D-2475 | [GalNAc3]sgaaaauCfaCfCfAfCfucuuuguus{invAb} | 3166 | asCfsaaagAfguggugAfuUfuucscsa | 3460 |
| D-2476 | [GalNAc3]suuuucaCfaUfUfUfUfucgucuuus{invAb} | 3247 | asAfsgacgAfaaaaugUfgAfaaasusc | 3548 |
| D-2477 | [GalNAc3]sacucuaAfgAfUfCfUfgaugaauus{invAb} | 3259 | asUfsucauCfagaucuUfaGfagususa | 3576 |
| D-2478 | [GalNAc3]saagaaaAfgUfGfAfUfucagugaus{invAb} | 3260 | usCfsacugAfaucacuUfuUfcuuscsu | 3577 |
| D-2479 | [GalNAc3]sgaacgaAfaGfUfUfAfuauggaaus{invAb} | 3152 | usUfsccauAfuaacuuUfcGfuucsusg | 3435 |
| D-2480 | [GalNAc3]suaccaaGfaGfCfGfCfagacuugcs{invAb} | 3071 | usGfscaagUfcugcgcUfcUfugguasgsa | 3584 |
| D-2481 | [GalNAc3]sccaagaGfcGfCfAfGfacuugcaus{invAb} | 3252 | usGfscaagUfcugcgcUfcUfuggsusa | 3553 |
| D-2482 | [GalNAc3]sccuggaAfuAfUfUfAfgaugccusus{invAb} | 3261 | asGfsgcauCfuaauauUfcCfaggsusu | 3472 |
| D-2483 | [GalNAc3]saauagcAfgAfCfUfUfguuccgacs{invAb} | 2066 | asGfsucggAfacaaguCfuGfcuasusu | 3585 |
| D-2484 | [GalNAc3]sccccguUfuAfAfCfUfgauuauggs{invAb} | 3115 | usCfscauaAfucaguuAfaAfcggsgsg | 3586 |
| D-2485 | [GalNAc3]saugacaAfcAfCfUfUfgaagcaugs{invAb} | 3118 | asCfsaugcUfucaaguGfuUfgucsasu | 3587 |
| D-2486 | [GalNAc3]suauugcCfaUfUfUfUfguccuuugs{invAb} | 3138 | usCfsaaagGfacaaaaUfgGfcaasusa | 3588 |
| D-2487 | [GalNAc3]suauaacUfcUfAfAfGfaucugaugs{invAb} | 3213 | usCfsaucaGfaucuuaGfaGfuuasusa | 3589 |
| D-2488 | [GalNAc3]sgugucuCfaAfUfGfCfuucaaugus{invAb} | 3216 | asAfscauuGfaagcauUfgAfgacsasc | 3590 |
| D-2489 | [GalNAc3]saauagcAfgAfCfUfUfguuccgacs{invAb} | 2066 | asGfsuCfgGfaacaaguCfuGfcuauususu | 2171 |
| D-2490 | [GalNAc3]sccccguUfuAfAfCfUfgauuauggs{invAb} | 3115 | usCfscAfuAfaucaguuAfaAfcggggsusu | 2653 |
| D-2491 | [GalNAc3]saugacaAfcAfCfUfUfgaagcaugs{invAb} | 3118 | asCfsaUfgCfuucaaguGfuUfgucaususu | 2371 |
| D-2492 | [GalNAc3]suauugcCfaUfUfUfUfguccuuugs{invAb} | 3138 | usCfsaAfaGfgacaaaaUfgGfcaauasusu | 2338 |
| D-2493 | [GalNAc3]suauaacUfcUfAfAfGfaucugaugs{invAb} | 3213 | usCfsaUfcAfgaucuuaGfaGfuuauasusu | 2475 |
| D-2494 | [GalNAc3]sgugucuCfaAfUfGfCfuucaaugus{invAb} | 3216 | asAfscAfuUfgaagcauUfgAfgacacsusu | 2245 |
| D-2495 | [GalNAc3]suagcagAfcUfUfGfUfuccgacusus{invAb} | 3262 | asGfsucggAfacaaguCfuGfcuasusu | 3585 |
| D-2496 | [GalNAc3]sccguuuAfaCfUfGfAfuuauggasus{invAb} | 3263 | usCfscauaAfucaguuAfaAfcggsusu | 3591 |
| D-2497 | [GalNAc3]sgacaacAfcUfUfGfAfagcaugusus{invAb} | 3264 | asCfsaugcUfucaaguGfuUfgucsusu | 3592 |
| D-2498 | [GalNAc3]suugccaUfuUfUfGfUfccuuugasus{invAb} | 3265 | usCfsaaagGfacaaaaUfgGfcaasusu | 3593 |
| D-2499 | [GalNAc3]suaacucUfaAfGfAfUfcugaugasus{invAb} | 3266 | usCfsaucaGfaucuuaGfaGfuuasusu | 3594 |
| D-2500 | [GalNAc3]sgucucaAfuGfCfUfUfcaauguusus{invAb} | 3267 | asAfscauuGfaagcauUfgAfgacsusu | 3595 |
| D-2501 | [GalNAc3]saauagcagAfcUfUfGfUfuccgacs{invAb} | 3268 | asGfsucggAfacaaGfuCfugcuauususu | 3596 |
| D-2502 | [GalNAc3]sccccguuuAfaCfUfGfAfuuauggs{invAb} | 3269 | usCfscauaAfucagUfuAfaacggggsusu | 3597 |
| D-2503 | [GalNAc3]saugacaacAfcUfUfGfAfagcaugs{invAb} | 3270 | asCfsaugcUfucaaGfuGfuugucaususu | 3598 |
| D-2504 | [GalNAc3]suauugccaUfuUfUfGfUfccuuugs{invAb} | 3271 | usCfsaaagGfacaaAfaUfggcaauasusu | 3599 |
| D-2505 | [GalNAc3]suauaacucUfaAfGfAfUfcugaugs{invAb} | 3272 | usCfsaucaGfaucuUfaGfaguuauasusu | 3600 |
| D-2506 | [GalNAc3]sgugucucaAfuGfCfUfUfcaaugus{invAb} | 3273 | asAfscauuGfaagcAfuUfgagacacsusu | 3601 |
| D-2507 | [GalNAc3]saauagcagAfcUfUfGfUfuccgacs{invAb} | 3268 | asGfsucggAfacaaGfuCfuGfcuauususu | 3602 |
| D-2508 | [GalNAc3]sccccguuuAfaCfUfGfAfuuauggs{invAb} | 3269 | usCfscauaAfucagUfuAfaAfcggggsusu | 3603 |
| D-2509 | [GalNAc3]saugacaacAfcUfUfGfAfagcaugs{invAb} | 3270 | asCfsaugcUfucaaGfuGfuUfgucaususu | 3604 |
| D-2510 | [GalNAc3]suauugccaUfuUfUfGfUfccuuugs{invAb} | 3271 | usCfsaaagGfacaaAfaUfgGfcaauasusu | 3605 |
| D-2511 | [GalNAc3]suauaacucUfaAfGfAfUfcugaugs{invAb} | 3272 | usCfsaucaGfaucuUfaGfaGfuuauasusu | 3606 |
| D-2512 | [GalNAc3]sgugucucaAfuGfCfUfUfcaaugus{invAb} | 3273 | asAfscauuGfaagcAfuUfgAfgacacsusu | 3607 |
| D-2514 | gsusgaugGfcUfUfGfUfuccggaugs{invAb} | 3274 | asCfsauccGfgaacaaGfcCfaucsasc | 3608 |
| D-2515 | gsusgaugGfcUfUfGfUfugcagaugs{invAb} | 3275 | asCfsaucuGfcaacaaGfcCfaucsasc | 3609 |
| D-2516 | csasgaacGfaAfAfGfUfuaugugga{invAb} | 3276 | usUfsccacAfuaacuuUfcGfuucugsasa | 3610 |
| D-2517 | csasgaacGfaAfAfGfUfuguaugga{invAb} | 3277 | usUfsccauAfcaacuuUfcGfuucugsasa | 3611 |
| D-2518 | usasugucCfuGfGfAfAfuauaagaus{invAb} | 3278 | asAfsucuuAfuauuccAfgGfacauasusu | 3612 |
| D-2519 | usasugucCfuGfGfAfAfuguuagaus{invAb} | 3279 | asAfsucuaAfcauuccAfgGfacauasusu | 3613 |
| D-2520 | asusguccUfgGfAfAfUfauuggaugs{invAb} | 3280 | asCfsauccAfauauucCfaGfgacaususu | 3614 |
| D-2521 | asusguccUfgGfAfAfUfaauagaugs{invAb} | 3281 | asCfsaucuAfuuauucCfaGfgacaususu | 3615 |
| D-2522 | usgsuccuGfgAfAfUfAfuuaaaugcs{invAb} | 3282 | asGfscauuUfaauauuCfcAfggacasusu | 3616 |
| D-2523 | usgsuccuGfgAfAfUfAfuaagaugcs{invAb} | 3283 | asGfscaucUfuauauuCfcAfggacasusu | 3617 |
| D-2524 | cscsuggaAfuAfUfUfAfggugccuus{invAb} | 3284 | asGfsgcacCfuaauauUfcCfaggsusu | 3618 |
| D-2525 | cscsuggaAfuAfUfUfGfgaugccuus{invAb} | 3285 | asGfsgcauCfcaauauUfcCfaggsusu | 3619 |
| D-2526 | uscscuggAfaUfAfUfUfagaagccus{invAb} | 3286 | asAfsggcuUfcuaauaUfuCfcagsgsa | 3620 |
| D-2527 | uscscuggAfaUfAfUfUfaaaugccus{invAb} | 3287 | asAfsggcaUfuuaauaUfuCfcagsgsa | 3621 |
| D-2528 | cscsuggaAfuAfUfUfAfgauaccuus{invAb} | 3288 | asAfsagguAfucuaauAfuUfccaggsusu | 3622 |
| D-2529 | cscsuggaAfuAfUfUfAfggugccuus{invAb} | 3284 | asAfsaggcAfccuaauAfuUfccaggsusu | 3623 |
| D-2530 | csusggaaUfaUfUfAfGfauggcuuus{invAb} | 3289 | asAfsaagcCfaucuaaUfaUfuccagsusu | 3624 |
| D-2531 | csusggaaUfaUfUfAfGfaagccuuus{invAb} | 3290 | asAfsaaggCfuucuaaUfaUfuccagsusu | 3625 |
| D-2532 | gsasauauUfaGfAfUfGfccuauuaa{invAb} | 3291 | usUfsuaauAfggcaucUfaAfuauucsusu | 3626 |
| D-2533 | gsasauauUfaGfAfUfGfcguuuuaa{invAb} | 3292 | usUfsuaaaAfcgcaucUfaAfuauucsusu | 3627 |
| D-2534 | gsasugccUfuUfUfAfAfaaaaguucs{invAb} | 3293 | asGfsaacuUfuuuuaaAfaGfgcaucsusu | 3628 |
| D-2535 | gsasugccUfuUfUfAfAfagauguucs{invAb} | 3294 | asGfsaacaUfcuuuaaAfaGfgcaucsusu | 3629 |
| D-2536 | gsasuuuuCfaCfAfUfUfuuuggucus{invAb} | 3295 | asAfsgaccAfaaaaugUfgAfaaaucsusu | 3630 |
| D-2537 | gsasuuuuCfaCfAfUfUfuaucgucus{invAb} | 3296 | asAfsgacgAfuaaaugUfgAfaaaucsusu | 3631 |
| D-2538 | csuscaauGfcUfUfCfAfaugaccca{invAb} | 3297 | asUfsggguCfauugaaGfcAfuugagsusu | 3632 |
| D-2539 | csuscaauGfcUfUfCfAfaaguccca{invAb} | 3298 | asUfsgggaCfuuugaaGfcAfuugagsusu | 3633 |
| D-2540 | csasaugcUfuCfAfAfUfgucgcagus{invAb} | 3299 | asAfscugcGfacauUfgAfaGfcaususg | 3634 |
| D-2541 | csasaugcUfuCfAfAfUfgacccagus{invAb} | 3300 | asAfscuggGfucauUfgAfaGfcaususg | 3635 |
| D-2542 | asasauauAfgUfCfUfCfaaugacuus{invAb} | 3301 | usAfsagucAfuugagaCfuAfuauuususu | 3636 |
| D-2543 | asasauauAfgUfCfUfCfaguaacuus{invAb} | 3302 | usAfsaguuAfcugagaCfuAfuauuususu | 3637 |
| D-2544 | gscsaggaUfuCfUfGfAfaaagucccs{invAb} | 3303 | asGfsggacUfuuucagAfaUfccugcsusu | 3638 |
| D-2545 | gscsaggaUfuCfUfGfAfagacucccs{invAb} | 3304 | asGfsggagUfcuucagAfaUfccugcsusu | 3639 |
| D-2546 | usasccaaGfaGfCfGfCfagaguugcs{invAb} | 3305 | usGfscaacUfcugcgcUfcUfugguasusu | 3640 |
| D-2547 | usasccaaGfaGfCfGfCfaaacuugcs{invAb} | 3306 | usGfscaagUfuugcgcUfcUfugguasusu | 3641 |
| D-2548 | asgsgaaaGfcAfUfAfUfgucgguugs{invAb} | 3307 | asCfsaaccGfacauauGfcUfuuccususu | 3642 |
| D-2549 | asgsgaaaGfcAfUfAfUfgacaguugs{invAb} | 3308 | asCfsaacuGfucauauGfcUfuuccususu | 3643 |
| D-2550 | gsusuuaaAfaCfCfCfAfaaaucuaus{invAb} | 3309 | usAfsgauuUfuggguuUfuAfaacsusu | 3644 |
| D-2551 | gsusuuaaAfaCfCfCfGfauaucuaus{invAb} | 3310 | usAfsgauaUfcggguuUfuAfaacsusu | 3645 |
| D-2552 | ususaacuGfaUfUfGfUfauagcucus{invAb} | 3311 | usAfsgagcUfauacaaUfcAfguuaasusu | 3646 |
| D-2553 | ususaacuGfaUfUfGfUfaaaacucus{invAb} | 3312 | usAfsgaguUfuuacaaUfcAfguuaasusu | 3647 |
| D-2554 | usasacucUfaAfGfAfUfcuggugaa{invAb} | 3313 | asUfsucacCfagaucuUfaGfagususa | 3648 |
| D-2555 | usasacucUfaAfGfAfUfcagaugaa{invAb} | 3314 | asUfsucauCfugaucuUfaGfagususa | 3649 |
| D-2556 | ascsucuaAfgAfUfCfUfgauaaagus{invAb} | 3315 | usAfscuuuAfucagauCfuUfagagususu | 3650 |
| D-2557 | ascsucuaAfgAfUfCfUfggugaagus{invAb} | 3316 | usAfscuucAfccagauCfuUfagagususu | 3651 |
| D-2558 | usasuugccaUfuUfUfGfUfcguuugs{invAb} | 3317 | usCfsaaacGfacaaAfaUfgGfcaauausu | 3652 |
| D-2559 | usasuugccaUfuUfUfGfAfccuuugs{invAb} | 3318 | usCfsaaagGfucaaAfaUfgGfcaauausu | 3653 |
| D-2560 | gscscauuUfuGfUfCfCfuuuaauua{invAb} | 3319 | asUfsaauuAfaaggacAfaAfauggcsusu | 3654 |
| D-2561 | gscscauuUfuGfUfCfCfuaugauua{invAb} | 3320 | asUfsaaucAfuaggacAfaAfauggcsusu | 3655 |

### Example 3. In Vitro Evaluation of mARC1 siRNA Molecules in a Cell-Based Assay

The mARC1 siRNA molecules having different sequences prioritized from the bioinformatics analyses described in Example 2 were screened for efficacy in reducing human mARC1 mRNA using an RNA FISH (fluorescence in situ hybridization) assay. Hep3B cells (purchased from ATCC) were cultured in Eagle's Minimum Essential Medium (EMEM) (ATCC 30-2003) supplemented with 10% fetal bovine serum (FBS, Sigma) and 1% penicillin-streptomycin (P-S, Corning). siRNAs were transfected into cells by reverse transfection using Lipofectamine RNAiMAX transfection reagent (Thermo Fisher Scientific). The mARC1 siRNA molecules were tested in a 10-point dose response format, 3-fold dilutions, ranging from 500 nM to 25 pM (run 1), 25 nM to 1 pM (run 2), or 100 nM to 5 pM (run 3), final concentrations. 1 μL of the test siRNA molecule or phosphate-buffered saline (PBS) vehicle and 4 μL of base EMEM without supplements were added to PDL-coated CellCarrier-384 Ultra assay plates (PerkinElmer) by a Bravo automated liquid handling platform (Agilent). 5 μL of Lipofectamine RNAiMAX (Thermo Fisher Scientific), pre-diluted in base EMEM without supplements (0.035 μL of RNAiMAX in 5 μL EMEM), was then dispensed into the assay plates by a Multidrop Combi reagent dispenser (Thermo Fisher Scientific). After 20-minute incubation of the siRNA/RNAiMAX mixture at room temperature (RT), 30 μL of Hep3B cells (2000 cells per well) in EMEM supplemented with 10% FBS and 1% P-S were added to the transfection complex using a Multidrop Combi reagent dispenser. The assay plates were incubated at RT for 20 mins prior to being placed in an incubator. Cells were incubated for 72 hrs at 37° C. and 5% CO₂. RNA FISH assay was performed 72 hours after siRNA transfection using the manufacturer's assay reagents and protocol (QuantiGene® ViewRNA HC Screening Assay from Thermo Fisher Scientific) on an in-house assembled automated FISH assay platform. In brief, cells were fixed in 4% formaldehyde (Thermo Fisher Scientific) for 15 mins at RT, permeabilized with detergent for 3 mins at RT and then treated with protease solution for 10 mins at RT. Target-specific probes (Thermo Fisher Scientific) or vehicle (target probe diluent without target probes as negative control) were incubated for 3 hours, whereas preamplifiers, amplifiers, and label probes were incubated for 1 hour each. All hybridization steps were carried out at 40° C. in a Cytomat 2 C-LIN automated incubator (Thermo Fisher Scientific). After hybridization reactions, cells were stained for 30 mins with Hoechst and CellMask Blue (Thermo Fisher Scientific) and then imaged on an Opera Phenix high-content screening system (PerkinElmer). The images were analyzed using a Columbus image data storage and analysis system (PerkinElmer) to obtain the mean spot count per cell. The mean spot count per cell was normalized using the high (PBS with target probes) and low (PBS without target probes) control wells. The normalized values against the total siRNA concentrations were plotted and the data were fit to a four-parameter sigmoidal model using Genedata Screener data analysis software (Genedata) to obtain IC50 and maximum activity values. If the data could not be fit to the model, an IC50 value was not calculated and only a maximum activity value was reported.

The mARC1 siRNA molecules were initially screened in a first run at ten different concentrations ranging from 500 nM to 25 pM. siRNA molecules exhibiting significant activity in the first run were screened in second and third runs at ten different concentrations over narrower concentration ranges (run 2: 25 nM to 1 pM; run 3: 100 nM to 5 pM). The results of the assays for all three runs are shown in Table 3 below.

TABLE 3
In vitro inhibition of human mARC1 mRNA in Hep3B cells

| | Run 1 | | Run 2 | | Run 3 | |
|---|---|---|---|---|---|---|
| | (500 nM to 25 pM) | | (25 nM to 1 pM) | | (100 nM to 5 pM) | |
| Duplex | IC50 | Max | IC50 | Max | IC50 | Max |
| No. | [M] | Activity | [M] | Activity | [M] | Activity |
| D-1092 | 1.64E−10 | −96.0 | 1.60E−09 | −93.0 | 2.43E−09 | −96.5 |
| D-1093 | 1.03E−10 | −89.5 | 1.19E−09 | −90.9 | 9.30E−10 | −95.8 |
| D-1139 | 3.44E−10 | −87.1 | 2.43E−09 | −93.7 | 2.35E−09 | −90.6 |
| D-1061 | 3.44E−11 | −89.0 | 9.04E−10 | −90.5 | 1.79E−09 | −93.0 |
| D-1138 | 1.13E−10 | −89.9 | 2.19E−09 | −87.9 | 1.60E−09 | −92.4 |
| D-1095 | 1.27E−10 | −86.9 | 1.34E−09 | −86.5 | 1.28E−09 | −92.4 |
| D-1191 | — | −93.5 | 1.06E−09 | −91.7 | 8.45E−10 | −86.4 |
| D-1180 | 1.52E−10 | −86.2 | 1.41E−09 | −88.3 | 2.15E−09 | −89.6 |
| D-1090 | 1.26E−10 | −88.1 | 1.42E−09 | −87.6 | 2.10E−09 | −89.5 |
| D-1062 | 3.36E−11 | −88.5 | 1.15E−09 | −87.9 | 1.01E−09 | −89.0 |
| D-1177 | 5.02E−11 | −81.0 | 1.43E−09 | −90.2 | 2.33E−09 | −86.5 |
| D-1083 | 6.31E−10 | −87.8 | 1.88E−09 | −84.8 | 1.19E−09 | −91.8 |
| D-1245 | 1.04E−10 | −83.9 | 4.46E−10 | −87.6 | 2.19E−09 | −88.5 |
| D-1067 | — | −79.8 | 1.41E−09 | −85.5 | 9.69E−10 | −90.3 |
| D-1143 | — | −92.8 | 1.49E−09 | −85.2 | 2.41E−09 | −90.3 |
| D-1170 | 1.87E−10 | −86.4 | 1.21E−09 | −86.0 | 9.50E−10 | −89.1 |
| D-1044 | 4.69E−11 | −81.3 | 1.45E−09 | −89.4 | 1.03E−09 | −85.6 |
| D-1096 | 7.11E−11 | −91.0 | 5.60E−10 | −82.5 | 8.71E−10 | −91.8 |
| D-1113 | 1.15E−10 | −85.9 | 1.56E−09 | −87.1 | 1.39E−09 | −85.5 |
| D-1086 | 2.40E−10 | −83.5 | 2.26E−09 | −84.0 | 2.28E−09 | −88.5 |
| D-1256 | — | −88.9 | 6.08E−10 | −87.1 | 8.37E−10 | −85.2 |
| D-1189 | 1.50E−10 | −84.7 | 1.36E−09 | −85.4 | 2.08E−09 | −86.7 |
| D-1091 | 9.38E−11 | −88.8 | 2.12E−09 | −87.8 | 1.42E−09 | −84.2 |
| D-1174 | 1.50E−10 | −84.1 | 1.57E−09 | −85.5 | 2.53E−09 | −86.5 |
| D-1185 | 3.25E−11 | −86.6 | 4.67E−10 | −82.6 | 2.18E−09 | −88.6 |
| D-1066 | 4.91E−11 | −80.3 | 1.33E−09 | −86.7 | 1.21E−09 | −84.3 |
| D-1171 | — | −83.8 | 1.10E−09 | −88.0 | 1.01E−09 | −83.0 |
| D-1140 | 3.09E−10 | −87.8 | 2.64E−09 | −86.5 | 2.97E−09 | −84.0 |
| D-1130 | 1.76E−10 | −77.0 | 2.94E−09 | −88.9 | 2.36E−09 | −81.5 |
| D-1068 | 4.98E−11 | −80.1 | 1.40E−09 | −82.9 | 1.08E−09 | −87.5 |
| D-1243 | — | −90.2 | 6.70E−10 | −84.9 | 8.36E−10 | −85.4 |
| D-1074 | 6.75E−11 | −76.5 | 1.01E−09 | −85.8 | 1.41E−09 | −83.7 |
| D-1150 | 1.78E−10 | −87.5 | 1.21E−09 | −84.3 | 2.11E−09 | −84.7 |
| D-1249 | 2.48E−11 | −85.1 | 1.03E−09 | −84.2 | 2.11E−09 | −84.5 |
| D-1111 | 6.71E−11 | −87.3 | 1.10E−09 | −85.4 | 1.52E−09 | −82.9 |
| D-1230 | 3.95E−11 | −83.7 | 9.01E−10 | −84.7 | 1.20E−09 | −83.5 |
| D-1087 | 2.03E−10 | −83.3 | 1.75E−09 | −85.3 | 1.76E−09 | −82.9 |
| D-1099 | 4.39E−11 | −79.6 | 1.82E−09 | −84.8 | 1.16E−09 | −82.9 |
| D-1190 | 1.47E−10 | −82.9 | 1.15E−09 | −84.8 | 2.01E−09 | −82.8 |
| D-1236 | 2.72E−10 | −85.2 | 1.20E−09 | −85.3 | 2.14E−09 | −82.2 |
| D-1184 | 1.62E−10 | −81.3 | 2.24E−09 | −81.8 | 2.43E−09 | −85.6 |
| D-1228 | — | −83.3 | 5.73E−10 | −78.9 | 5.66E−10 | −87.3 |
| D-1220 | 5.60E−10 | −86.0 | 2.05E−09 | −80.8 | 9.99E−10 | −85.1 |
| D-1204 | 4.99E−11 | −79.7 | 1.84E−09 | −78.9 | 2.51E−09 | −87.0 |
| D-1179 | 7.45E−11 | −81.8 | 1.64E−09 | −86.5 | 1.26E−09 | −78.1 |
| D-1147 | 3.64E−10 | −85.0 | 8.60E−10 | −89.7 | 2.85E−09 | −73.8 |
| D-1097 | 3.39E−10 | −86.6 | 2.97E−09 | −85.2 | 3.27E−09 | −78.0 |
| D-1194 | 1.31E−10 | −78.9 | 1.40E−09 | −71.2 | 2.49E−09 | −86.3 |
| D-1054 | 1.63E−10 | −66.3 | 1.89E−09 | −85.2 | 4.36E−09 | −71.2 |
| D-1176 | 4.61E−11 | −74.7 | 1.34E−09 | −67.1 | 1.55E−09 | −85.1 |
| D-1215 | 1.08E−10 | −85.0 | 1.53E−09 | −84.5 | 2.35E−09 | −82.4 |
| D-1166 | 8.24E−11 | −84.0 | 1.94E−09 | −84.2 | 2.39E−09 | −82.4 |
| D-1213 | 4.13E−11 | −73.9 | 5.89E−10 | −84.3 | 1.23E−09 | −82.1 |
| D-1187 | 1.55E−10 | −87.9 | 2.30E−09 | −82.7 | 2.25E−09 | −83.3 |
| D-1210 | — | — | 2.12E−09 | −82.4 | 1.93E−09 | −83.5 |
| D-1209 | 1.10E−10 | −78.7 | 1.81E−09 | −82.3 | 1.84E−09 | −83.0 |
| D-1246 | 5.28E−11 | −77.0 | 5.07E−10 | −84.1 | 2.19E−09 | −80.4 |
| D-1257 | — | −82.1 | 9.40E−10 | −80.7 | 2.03E−09 | −83.4 |
| D-1020 | 1.18E−10 | −78.3 | 2.53E−09 | −84.8 | 1.79E−09 | −78.9 |
| D-1168 | 3.87E−11 | −84.5 | 1.32E−09 | −82.0 | 8.52E−10 | −81.5 |
| D-1241 | 2.42E−10 | −81.0 | 1.92E−09 | −81.2 | 2.63E−09 | −81.9 |
| D-1255 | 2.93E−11 | −80.7 | 1.27E−09 | −80.4 | 1.24E−09 | −82.5 |
| D-1181 | 2.85E−10 | −85.0 | 1.30E−09 | −78.1 | 1.69E−09 | −84.6 |
| D-1252 | — | −81.6 | 8.95E−10 | −78.4 | 2.22E−09 | −84.3 |
| D-1172 | 9.64E−11 | −78.7 | 1.51E−09 | −83.3 | 1.04E−09 | −79.2 |
| D-1175 | 6.64E−11 | −76.8 | 1.28E−09 | −80.9 | 2.21E−09 | −81.5 |
| D-1235 | 2.60E−10 | −83.0 | 1.66E−09 | −81.3 | 2.63E−09 | −80.8 |
| D-1229 | 1.30E−10 | −82.5 | 6.74E−10 | −84.3 | 1.29E−09 | −77.9 |
| D-1070 | — | −80.2 | 1.32E−09 | −81.7 | 1.48E−09 | −79.9 |
| D-1203 | 9.51E−11 | −80.8 | 1.73E−09 | −82.0 | 2.51E−09 | −78.4 |
| D-1183 | 1.14E−10 | −76.4 | 9.71E−10 | −81.9 | 2.10E−09 | −78.3 |
| D-1050 | 5.15E−11 | −87.0 | 1.30E−09 | −83.6 | 1.22E−09 | −76.7 |
| D-1167 | 2.87E−11 | −76.6 | 5.21E−10 | −79.4 | 1.14E−09 | −80.8 |
| D-1164 | 3.14E−10 | −87.8 | 1.75E−09 | −81.6 | 2.49E−09 | −78.4 |
| D-1237 | 9.94E−10 | −78.1 | 1.88E−09 | −80.7 | 2.19E−09 | −79.0 |
| D-1247 | 2.30E−11 | −78.7 | 8.40E−10 | −77.1 | 2.03E−09 | −82.3 |
| D-1075 | 1.17E−10 | −73.8 | 1.90E−09 | −84.5 | 1.00E−09 | −74.9 |
| D-1211 | 6.67E−11 | −72.9 | 1.52E−09 | −75.0 | 3.10E−09 | −84.0 |
| D-1248 | 3.22E−11 | −83.3 | 8.91E−10 | −80.6 | 1.37E−09 | −77.8 |
| D-1250 | 5.19E−11 | −77.3 | 6.63E−10 | −77.5 | 2.30E−09 | −80.8 |
| D-1069 | 8.02E−11 | −78.9 | 1.81E−09 | −78.2 | 2.22E−09 | −80.1 |
| D-1253 | — | −81.2 | 4.63E−10 | −77.9 | 2.21E−09 | −80.3 |
| D-1056 | 6.68E−11 | −76.7 | 2.02E−09 | −78.4 | 1.48E−09 | −79.6 |
| D-1079 | 8.81E−11 | −66.6 | 1.23E−09 | −78.5 | 1.70E−09 | −79.2 |
| D-1162 | 6.89E−11 | −77.8 | 2.08E−09 | −83.7 | 2.24E−09 | −73.5 |
| D-1045 | 7.98E−11 | −71.8 | 2.14E−09 | −78.6 | 1.63E−09 | −78.5 |
| D-1173 | 1.41E−10 | −84.6 | 1.70E−09 | −77.0 | 2.35E−09 | −80.0 |
| D-1182 | 6.61E−11 | −90.2 | 3.09E−09 | −81.5 | 2.92E−09 | −75.4 |
| D-1146 | 1.18E−10 | −77.7 | 3.39E−09 | −77.3 | 3.03E−09 | −79.2 |
| D-1244 | 7.71E−11 | −74.6 | 8.53E−10 | −78.7 | 2.23E−09 | −77.8 |
| D-1186 | 1.07E−10 | −71.3 | 1.03E−09 | −77.5 | 2.35E−09 | −78.9 |
| D-1258 | 1.60E−10 | −76.9 | 1.42E−09 | −77.4 | 2.26E−09 | −78.7 |
| D-1043 | 8.57E−10 | −81.2 | 3.33E−09 | −71.8 | 9.87E−09 | −83.9 |
| D-1163 | 3.85E−11 | −87.7 | 1.85E−10 | −80.1 | 2.11E−09 | −75.4 |
| D-1206 | 1.35E−10 | −78.8 | 1.62E−09 | −77.8 | 1.91E−09 | −77.5 |
| D-1089 | 2.34E−10 | −84.6 | 2.27E−09 | −81.6 | 2.51E−09 | −73.7 |
| D-1207 | 2.94E−11 | −74.0 | 1.54E−09 | −77.6 | 1.40E−09 | −77.6 |
| D-1202 | 3.74E−11 | −67.6 | 1.31E−09 | −77.0 | 1.47E−09 | −78.3 |
| D-1221 | 1.77E−10 | −84.4 | 2.08E−09 | −79.4 | 3.46E−09 | −75.7 |
| D-1212 | 6.59E−11 | −77.9 | 2.20E−09 | −77.9 | 2.18E−09 | −77.0 |
| D-1188 | 2.07E−10 | −84.5 | 1.62E−09 | −70.6 | 1.57E−09 | −83.7 |
| D-1037 | 3.48E−10 | −80.6 | 2.28E−09 | −76.3 | 2.12E−09 | −77.5 |
| D-1251 | 3.53E−11 | −78.2 | 6.32E−10 | −77.3 | 2.00E−09 | −76.0 |
| D-1148 | 9.64E−11 | −86.5 | 1.80E−09 | −76.0 | 1.41E−09 | −77.0 |
| D-1214 | 4.88E−11 | −71.7 | 1.72E−09 | −75.4 | 6.69E−10 | −77.6 |
| D-1046 | 2.18E−10 | −70.4 | 3.35E−09 | −84.0 | 4.63E−09 | −68.4 |
| D-1051 | 1.12E−10 | −72.4 | 1.53E−09 | −78.6 | 1.05E−09 | −73.6 |
| D-1112 | 6.34E−11 | −67.5 | 1.80E−09 | −77.6 | 2.24E−09 | −74.7 |
| D-1114 | 7.18E−11 | −75.2 | 1.66E−09 | −75.0 | 2.27E−09 | −77.1 |
| D-1149 | 2.18E−10 | −76.5 | 1.86E−09 | −80.9 | 2.15E−09 | −71.0 |
| D-1119 | 8.91E−11 | −73.1 | 3.58E−09 | −78.6 | 5.56E−09 | −73.4 |
| D-1126 | 1.04E−10 | −82.6 | 2.07E−09 | −70.7 | 2.02E−09 | −81.1 |
| D-1254 | 6.11E−11 | −81.4 | 1.45E−09 | −73.5 | 2.37E−09 | −78.2 |
| D-1219 | 5.51E−11 | −72.2 | 1.85E−09 | −77.9 | 2.36E−09 | −73.8 |
| D-1134 | 2.91E−10 | −75.9 | 1.69E−09 | −77.5 | 2.11E−09 | −73.4 |
| D-1023 | 1.49E−10 | −86.2 | 2.22E−09 | −74.7 | 2.97E−09 | −76.1 |
| D-1201 | 9.75E−11 | −69.6 | 2.02E−09 | −71.0 | 2.77E−09 | −79.0 |
| D-1059 | — | −66.9 | 2.32E−09 | −77.5 | 1.80E−09 | −72.2 |
| D-1195 | — | — | 1.48E−09 | −71.7 | 5.53E−10 | −77.5 |
| D-1160 | 1.39E−10 | −78.9 | 5.70E−10 | −66.1 | 1.70E−09 | −82.5 |
| D-1141 | 4.31E−10 | −81.0 | 4.03E−09 | −74.1 | 3.60E−09 | −73.9 |
| D-1137 | 1.93E−10 | −81.6 | 3.29E−09 | −65.3 | 6.20E−09 | −82.5 |
| D-1260 | 6.31E−11 | −69.6 | 1.51E−09 | −74.8 | 5.40E−09 | −72.4 |
| D-1073 | — | −77.7 | 1.78E−09 | −75.5 | 1.90E−09 | −71.6 |
| D-1178 | 4.29E−10 | −73.4 | 4.35E−09 | −67.2 | 8.66E−09 | −79.1 |
| D-1157 | 3.87E−10 | −74.0 | — | −70.6 | 7.10E−09 | −75.3 |
| D-1047 | 1.26E−10 | −74.5 | 2.58E−09 | −76.4 | 2.69E−09 | −69.0 |
| D-1161 | 5.65E−11 | −76.8 | 1.10E−09 | −61.9 | 2.17E−09 | −83.5 |
| D-1098 | 4.18E−10 | −86.1 | — | −68.2 | 3.05E−09 | −75.3 |
| D-1081 | 7.18E−11 | −67.3 | 2.33E−09 | −73.4 | 2.35E−09 | −69.6 |
| D-1240 | 8.69E−10 | −72.9 | 1.68E−09 | −69.4 | 1.86E−09 | −73.2 |
| D-1259 | 2.52E−11 | −70.1 | 7.91E−10 | −73.1 | 7.64E−10 | −69.3 |
| D-1120 | 3.48E−10 | −83.6 | 2.24E−09 | −71.1 | 2.52E−09 | −69.3 |
| D-1104 | 9.89E−11 | −64.0 | 2.73E−09 | −69.8 | 2.86E−09 | −70.5 |
| D-1225 | — | −68.8 | 1.17E−09 | −65.3 | 2.11E−09 | −74.4 |
| D-1052 | 3.31E−11 | −70.1 | 1.64E−09 | −69.5 | 9.63E−10 | −70.2 |
| D-1072 | — | −75.6 | 2.14E−09 | −71.8 | 1.53E−09 | −67.7 |
| D-1082 | 1.54E−10 | −70.1 | 2.11E−09 | −72.4 | 2.47E−09 | −67.2 |
| D-1224 | 9.53E−11 | −80.0 | 2.19E−09 | −67.3 | 2.87E−09 | −70.3 |
| D-1032 | 1.35E−10 | −89.8 | 3.18E−09 | −73.1 | 2.64E−09 | −64.3 |
| D-1017 | 1.16E−10 | −69.3 | 2.49E−09 | −69.6 | 1.90E−09 | −66.5 |
| D-1208 | 1.46E−10 | −67.5 | 2.45E−09 | −64.3 | 3.96E−09 | −71.7 |
| D-1048 | 2.86E−10 | −67.2 | 3.12E−09 | −70.4 | 2.60E−09 | −65.6 |
| D-1080 | 1.19E−10 | −55.0 | 3.84E−09 | −75.4 | 1.82E−09 | −59.2 |
| D-1102 | 4.22E−11 | −64.6 | 2.07E−09 | −70.8 | 1.97E−09 | −63.8 |
| D-1076 | 1.18E−10 | −57.7 | 2.38E−09 | −62.5 | 2.29E−09 | −71.5 |
| D-1055 | — | −47.6 | 2.89E−09 | −64.5 | 5.23E−09 | -69.2 |
| D-1216 | 1.83E−10 | −70.6 | 4.14E−09 | −67.1 | 3.59E−09 | −66.2 |
| D-1193 | 1.79E−10 | −82.0 | 1.40E−09 | −60.6 | 4.45E−09 | −72.5 |
| D-1217 | 2.58E−10 | −67.5 | 3.12E−09 | −60.9 | 4.77E−09 | −71.1 |
| D-1200 | 1.40E−10 | −70.4 | 3.63E−09 | −64.5 | 4.11E−09 | −67.1 |
| D-1058 | 1.46E−10 | −52.8 | 2.63E−09 | −65.4 | 1.94E−09 | −65.7 |
| D-1084 | 1.13E−10 | −75.6 | 1.98E−09 | −71.5 | 2.19E−09 | −59.4 |
| D-1118 | 3.51E−10 | −75.1 | 2.94E−09 | −65.1 | 6.26E−09 | −65.0 |
| D-1136 | 1.63E−10 | −65.7 | 3.06E−09 | −65.6 | 3.20E−09 | −63.3 |
| D-1116 | 3.67E−10 | −76.6 | 3.95E−09 | −58.9 | 3.33E−09 | −69.4 |
| D-1169 | 1.79E−10 | −72.8 | 1.99E−09 | −53.5 | 4.33E−09 | −74.5 |
| D-1065 | 1.57E−10 | −60.6 | 1.91E−09 | −64.1 | 2.14E−09 | −63.5 |
| D-1063 | 2.80E−11 | −65.2 | 1.38E−09 | −63.2 | 1.26E−09 | −63.1 |
| D-1034 | 1.45E−10 | −68.7 | 1.87E−09 | −62.6 | 1.70E−09 | −60.7 |
| D-1218 | 2.05E−10 | −62.9 | 2.21E−09 | −58.8 | 2.67E−09 | −63.7 |
| D-1154 | 1.84E−10 | −69.2 | 2.55E−09 | −55.5 | 3.44E−09 | −66.0 |
| D-1049 | 3.04E−10 | −70.7 | 2.10E−09 | −69.9 | 2.53E−09 | −51.4 |
| D-1088 | 5.85E−10 | −73.7 | 3.65E−09 | −58.6 | 3.15E−09 | −62.6 |
| D-1199 | 3.95E−10 | −73.2 | 2.70E−09 | −55.1 | 4.45E−09 | −64.4 |
| D-1165 | 2.02E−10 | −72.7 | — | −52.5 | 4.14E−09 | −66.5 |
| D-1028 | 1.82E−09 | −84.0 | 5.79E−09 | −51.0 | 1.80E−08 | −64.3 |
| D-1078 | 1.53E−10 | −55.4 | 1.97E−09 | −54.0 | 4.95E−09 | −60.8 |
| D-1222 | 1.82E−10 | −59.9 | 2.33E−09 | −60.4 | 2.18E−09 | −54.4 |
| D-1131 | 6.25E−10 | −75.0 | 5.53E−09 | −56.9 | 5.17E−09 | −57.8 |
| D-1027 | 6.87E−11 | −66.4 | 1.94E−09 | −51.6 | 2.22E−09 | −63.1 |
| D-1151 | 1.39E−10 | −59.0 | 2.21E−09 | −52.8 | 1.89E−09 | −61.2 |
| D-1135 | 2.33E−10 | −61.2 | 4.03E−09 | −56.0 | 5.09E−09 | −56.9 |
| D-1038 | — | −35.4 | 3.59E−09 | −73.4 | 7.37E−09 | −36.7 |
| D-1196 | 1.37E−10 | −57.4 | 3.05E−09 | −49.5 | 3.84E−09 | −58.0 |
| D-1223 | 1.38E−10 | −63.6 | 2.96E−09 | −54.8 | 2.75E−09 | −44.6 |
| D-1100 | 8.61E−11 | −46.0 | 2.94E−09 | −54.5 | 1.94E−09 | −43.5 |
| D-1197 | 1.96E−10 | −53.9 | 3.68E−09 | −49.1 | 5.22E−09 | −46.9 |
| D-1205 | 2.98E−10 | −67.1 | 2.83E−09 | −50.0 | 4.84E−09 | −43.7 |
| D-1192 | — | −84.6 | >25E−9 | −5.1 | 2.17E−09 | −82.6 |
| D-1024 | 8.25E−10 | −71.0 | 3.63E−09 | −52.2 | >100E−9 | −34.5 |
| D-1231 | 2.40E−09 | −74.7 | 6.04E−09 | −37.4 | 1.15E−08 | −45.5 |
| D-1031 | 9.42E−11 | −43.6 | — | −38.0 | 6.12E−09 | −41.0 |
| D-1103 | 6.03E−11 | −67.1 | >25E−9 | −2.0 | 1.77E−09 | −60.0 |
| D-1132 | 2.93E−10 | −79.2 | >25E−9 | −2.4 | 2.87E−09 | −50.1 |
| D-1025 | — | −27.8 | >25E−9 | −21.9 | >100E−9 | −20.8 |
| D-1004 | >500E−9 | −5.3 | — | — | — | — |
| D-1005 | >500E−9 | −8.5 | — | — | — | — |
| D-1006 | >500E−9 | 17.0 | — | — | — | — |
| D-1007 | >500E−9 | 38.0 | — | — | — | — |
| D-1008 | >500E−9 | 4.8 | — | — | — | — |
| D-1009 | >500E−9 | −2.1 | — | — | — | — |
| D-1010 | >500E−9 | 9.8 | — | — | — | — |
| D-1011 | >500E−9 | −6.4 | — | — | — | — |
| D-1012 | >500E−9 | 39.7 | — | — | — | — |
| D-1013 | >500E−9 | 37.7 | — | — | — | — |
| D-1014 | >500E−9 | −23.4 | — | — | — | — |
| D-1015 | 3.51E−10 | −57.4 | — | — | — | — |
| D-1016 | 2.06E−10 | −51.2 | — | — | — | — |
| D-1018 | >500E−9 | 1.9 | — | — | — | — |
| D-1019 | 2.61E−10 | −36.5 | — | — | — | — |
| D-1021 | 1.86E−10 | −39.0 | — | — | — | — |
| D-1022 | 2.63E−09 | −47.8 | — | — | — | — |
| D-1026 | >500E−9 | 21.9 | — | — | — | — |
| D-1029 | 2.61E−10 | −57.4 | — | — | — | — |
| D-1030 | 2.66E−10 | −55.4 | — | — | — | — |
| D-1033 | >500E−9 | 37.1 | — | — | — | — |
| D-1035 | 7.68E-10 | −57.2 | — | — | — | — |
| D-1036 | 5.91E-10 | −55.7 | — | — | — | — |
| D-1039 | >500E−9 | −18.2 | — | — | — | — |
| D-1040 | >500E−9 | −14.3 | — | — | — | — |
| D-1041 | >500E−9 | −18.8 | — | — | — | — |
| D-1042 | 3.74E−10 | −45.5 | — | — | — | — |
| D-1053 | >500E−9 | −17.6 | — | — | — | — |
| D-1057 | >500E−9 | −5.1 | — | — | — | — |
| D-1060 | >500E−9 | −3.6 | — | — | — | — |
| D-1064 | >500E−9 | 22.6 | — | — | — | — |
| D-1071 | >500E−9 | −6.8 | — | — | — | — |
| D-1077 | >500E−9 | −4.7 | — | — | — | — |
| D-1085 | >500E−9 | −0.4 | — | — | — | — |
| D-1094 | 3.76E−10 | −66.8 | — | — | — | — |
| D-1101 | >500E−9 | −3.5 | — | — | — | — |
| D-1105 | >500E−9 | −10.8 | — | — | — | — |
| D-1106 | 4.64E−10 | −53.1 | — | — | — | — |
| D-1107 | >500E−9 | −0.1 | — | — | — | — |
| D-1108 | >500E−9 | −7.8 | — | — | — | — |
| D-1109 | 7.32E−10 | −36.0 | — | — | — | — |
| D-1110 | >500E−9 | 1.0 | — | — | — | — |
| D-1115 | >500E−9 | −5.2 | — | — | — | — |
| D-1117 | 2.06E−10 | −41.7 | — | — | — | — |
| D-1121 | 2.62E−09 | −54.1 | — | — | — | — |
| D-1122 | 3.22E−10 | −65.9 | — | — | — | — |
| D-1123 | >500E−9 | 16.9 | — | — | — | — |
| D-1124 | 4.33E−10 | −56.1 | — | — | — | — |
| D-1128 | 3.74E−10 | −51.3 | — | — | — | — |
| D-1129 | >500E−9 | −24.9 | — | — | — | — |
| D-1133 | 6.33E−10 | −39.4 | — | — | — | — |
| D-1142 | 4.80E−10 | −65.9 | — | — | — | — |
| D-1144 | >500E−9 | 6.1 | — | — | — | — |
| D-1145 | >500E−9 | 2.0 | — | — | — | — |
| D-1152 | 7.09E−10 | −44.4 | — | — | — | — |
| D-1153 | 8.57E−08 | −48.1 | — | — | — | — |
| D-1155 | 1.48E−10 | −32.2 | — | — | — | — |
| D-1156 | >500E−9 | −8.3 | — | — | — | — |
| D-1158 | 1.94E−09 | −35.0 | — | — | — | — |
| D-1159 | 7.15E−10 | −67.2 | — | — | — | — |
| D-1198 | 3.37E−10 | −69.0 | — | — | — | — |
| D-1226 | >500E−9 | −2.5 | — | — | — | — |
| D-1227 | >500E−9 | 3.4 | — | — | — | — |
| D-1232 | 8.99E−10 | −61.4 | — | — | — | — |
| D-1233 | 1.19E−09 | −68.5 | — | — | — | — |
| D-1234 | 5.48E−10 | −65.1 | — | — | — | — |
| D-1238 | 1.51E−09 | −45.6 | — | — | — | — |
| D-1239 | 6.25E−10 | −67.1 | — | — | — | — |
| D-1242 | 6.22E−10 | −63.4 | — | — | — | — |
| D-1261 | >500E−9 | −19.1 | — | — | — | — |
| D-1262 | >500E−9 | −21.5 | — | — | — | — |

Of the initial 257 mARC1 siRNA molecules evaluated in the RNA FISH assay, 74 molecules exhibited an average of 80% or greater knockdown of human mARC1 mRNA and had IC50 values at least in the single-digit nanomolar range in assay runs 2 and 3. In particular, 32 molecules (duplex nos. D-1092; D-1093; D-1139; D-1061; D-1138; D-1095; D-1191; D-1180; D-1090; D-1062; D-1177; D-1083; D-1245; D-1067; D-1143; D-1170; D-1044; D-1096; D-1113; D-1086; D-1256; D-1189; D-1091; D-1174; D-1185; D-1066; D-1171; D-1140; D-1130; D-1068; D-1243; D-1074) reduced human mARC1 mRNA by at least 85% in one or both assay runs 2 and 3.

In a second series of experiments, additional mARC1 siRNA molecules were evaluated in the RNA FISH assay at ten different concentrations ranging from 100 nM to 5 pM, and IC50 and maximum activity values were calculated as described above. The results of the assays from this second series of experiments are shown in Table 4 below. Assays were repeated for a subset of molecules. For such molecules, the IC50 and maximum activity values for both runs are shown.

TABLE 4
In vitro inhibition of human mARC1 mRNA by select mARC1
siRNA molecules in Hep3B cells

| Duplex No. | IC50 [M] | Max Activity | Duplex No. | IC50 [M] | Max Activity |
|---|---|---|---|---|---|
| D-1061 | 663E−12 | −99.13 | D-1267-run 1 | 3.4E−9 | −80.64 |
| D-1093 | 720E−12 | −90.29 | D-1267-run 2 | 1.33E−09 | −83.64 |
| D-1139 | 1.72E−09 | −97.32 | D-1268-run 1 | 1.23E−9 | −88.75 |
| D-1220 | 3.26E−9 | −97.17 | D-1268-run 2 | 3.98E−10 | −89.45 |
| D-1245 | 93.3E−12 | −88.21 | D-1269 | >100E−9 | −32.26 |
| D-1263 | 3.9E−9 | −74.11 | D-1270 | 1.38E−9 | −56.38 |
| D-1264 | 1.4E−9 | −79.53 | D-1271 | >100E−9 | −26.78 |
| D-1265 | 2.68E−9 | −77.10 | D-1272-run 1 | 941E−12 | −84.38 |
| D-1266-run 1 | 541E−12 | −85.44 | D-1272-run 2 | 1.36E−09 | −89.35 |
| D-1266-run 2 | 1.61E−10 | −93.80 | D-1273-run 1 | 1.22E−9 | −85.29 |
| D-1274-run 1 | 638E−12 | −86.28 | D-1273-run 2 | 1.17E−09 | −90.43 |
| D-1274-run 2 | 8.95E−10 | −89.55 | D-1281-run 1 | 2.58E−9 | −86.39 |
| D-1275 | 428E−12 | −79.99 | D-1281-run 2 | 1.43E−09 | −88.37 |
| D-1276-run 1 | 1.54E−9 | −92.38 | D-1282-run 1 | 638E−12 | −87.53 |
| D-1276-run 2 | 1.59E−09 | −88.32 | D-1282-run 2 | 5.56E−10 | −95.34 |
| D-1277 | 2E−9 | −79.86 | D-1283-run 1 | 1.97E−9 | −80.62 |
| D-1278-run 1 | 1.21E−9 | −81.71 | D-1283-run 2 | 1.90E−09 | −81.20 |
| D-1278-run 2 | 1.55E−09 | −84.13 | D-1284-run 1 | 1.94E−9 | −91.35 |
| D-1279 | 323E−12 | −76.50 | D-1284-run 2 | 3.09E−09 | −91.61 |
| D-1280 | >100E−9 | 1.94 | D-1285-run 1 | 1E−9 | −88.21 |
| D-1286-run 1 | 2.04E−9 | −89.49 | D-1285-run 2 | 1.54E−09 | −86.72 |
| D-1286-run 2 | 2.42E−09 | −94.63 | D-1293 | — | −56.99 |
| D-1287-run 1 | 1.31E−9 | −82.87 | D-1294 | 2.72E−9 | −40.67 |
| D-1287-run 2 | 1.11E−09 | −83.35 | D-1295-run 1 | 3.86E−9 | −81.47 |
| D-1288-run 1 | 3.58E−9 | −88.81 | D-1295-run 2 | 4.77E−09 | −79.64 |
| D-1288-run 2 | 3.36E−09 | −90.32 | D-1296-run 1 | 1.29E−9 | −87.96 |
| D-1289 | 2.17E−9 | −73.71 | D-1296-run 2 | 1.97E−09 | −89.76 |
| D-1290 | 29.3E−9 | −50.69 | D-1297 | >100E−9 | 0.97 |
| D-1291 | 2.37E−9 | −62.67 | D-1298-run 1 | 636E−12 | −94.52 |
| D-1292-run 1 | 6.56E−9 | −81.82 | D-1298-run 2 | 4.99E−10 | −94.67 |
| D-1292-run 2 | 5.35E−09 | −75.91 | D-1299-run 1 | 293E−12 | −86.71 |
| D-1300 | 2.7E−9 | −79.68 | D-1299-run 2 | 6.23E−10 | −90.92 |
| D-1301 | >100E−9 | −46.76 | D-1308-run 1 | 1.61E−9 | −83.41 |
| D-1302 | 2.15E−9 | −80.01 | D-1308-run 2 | 1.28E−09 | −85.58 |
| D-1303-run 1 | 1.44E−9 | −85.61 | D-1309 | 405E−12 | −78.77 |
| D-1303-run 2 | 1.05E−09 | −88.46 | D-1310-run 1 | 1.83E−9 | −89.68 |
| D-1304-run 1 | 490E−12 | −85.23 | D-1310-run 2 | 1.91E−09 | −95.65 |
| D-1304-run 2 | 6.56E−10 | −88.62 | D-1311-run 1 | 1.05E−9 | −90.99 |
| D-1305 | 802E−12 | −79.34 | D-1311-run 2 | 8.00E−10 | −87.63 |
| D-1306 | 2.4E−9 | −77.95 | D-1312-run 1 | 2.26E−9 | −87.43 |
| D-1307 | 2.43E−9 | −77.80 | D-1312-run 2 | 1.65E−09 | −82.70 |
| D-1314 | 768E−12 | −76.88 | D-1313 | 1.85E−9 | −78.77 |
| D-1315-run 1 | 2.2E−9 | −88.75 | D-1322-run 1 | >100E−9 | −6.77 |
| D-1315-run 2 | 2.49E−09 | −83.37 | D-1322-run 2 | >100E−9 | −1.46 |
| D-1316 | 3.66E−9 | −71.22 | D-1323 | >100E−9 | 2.99 |
| D-1317 | 3.03E−9 | −69.28 | D-1324 | >100E−9 | −6.04 |
| D-1318 | 8.65E−9 | −59.48 | D-1325 | >100E−9 | 15.82 |
| D-1319-run 1 | 6.1E−9 | −82.28 | D-1326-run 1 | >100E−9 | −16.18 |
| D-1319-run 2 | 3.94E−09 | −79.12 | D-1326-run 2 | >100E−9 | −6.29 |
| D-1320 | 4.95E−9 | −70.44 | D-1327 | 6.21E−9 | −56.80 |
| D-1321-run 1 | 2.21E−9 | −84.41 | D-1328 | >100E−9 | −40.40 |
| D-1321-run 2 | 1.74E−09 | −79.23 | D-1329 | >100E−9 | −39.95 |
| D-1330-run 1 | 7.48E−9 | −82.11 | D-1335-run 1 | 1.15E−9 | −87.64 |
| D-1330-run 2 | 7.14E−09 | −78.69 | D-1335-run 2 | 9.61E−10 | −86.06 |
| D-1331-run 1 | 3.59E−9 | −65.41 | D-1336-run 1 | 2.62E−9 | −85.53 |
| D-1331-run 2 | 4.38E−9 | −65.82 | D-1336-run 2 | 1.58E−09 | −80.06 |
| D-1332-run 1 | >100E−9 | −11.88 | D-1337 | 4.88E−9 | −68.99 |
| D-1332-run 2 | >100E−9 | −8.78 | D-1338-run 1 | 2.16E−9 | −95.31 |
| D-1333-run 1 | 5.73E−9 | −60.02 | D-1338-run 2 | 2.44E−09 | −95.49 |
| D-1333-run 2 | 20.5E−9 | −72.67 | D-1339 | 5.6E−9 | −77.24 |
| D-1334-run 1 | 5.96E−9 | −86.77 | D-1340 | 3.77E−9 | −67.14 |
| D-1334-run 2 | 5.74E−9 | −70.29 | D-1341-run 1 | 1.27E−9 | −82.33 |
| D-1334-run 3 | 6.24E−09 | −79.62 | D-1341-run 2 | 1.22E−09 | −85.14 |
| D-1342 | 3.79E−9 | −79.64 | D-1351-run 1 | 2.49E−9 | −83.34 |
| D-1343 | 7.69E−9 | −76.38 | D-1351-run 2 | 2.06E−09 | −84.99 |
| D-1344 | 4.7E−9 | −80.14 | D-1352 | 2.29E−9 | −78.03 |
| D-1345 | >100E−9 | −33.98 | D-1353 | 2.4E−9 | −51.04 |
| D-1346 | 1.8E−9 | −67.30 | D-1354 | 1.99E−9 | −69.59 |
| D-1347 | 3.71E−9 | −72.82 | D-1355 | 3.05E−9 | −60.02 |
| D-1348 | 21.7E−9 | −58.27 | D-1356 | 5.45E−9 | −41.04 |
| D-1349 | 1.7E−9 | −78.63 | D-1357 | 2.85E−9 | −57.34 |
| D-1350-run 1 | 438E−12 | −86.65 | D-1358-run 1 | 967E−12 | −82.23 |
| D-1350-run 2 | 4.23E−10 | −82.90 | D-1358-run 2 | 1.17E−09 | −90.43 |
| D-1359 | 2.03E−9 | −70.00 | D-1366 | 4.75E−9 | −76.16 |
| D-1360-run 1 | 3.62E−9 | −87.34 | D-1367-run 1 | 2.26E−9 | −93.08 |
| D-1360-run 2 | 3.10E−09 | −83.45 | D-1367-run 2 | 1.98E−09 | −93.44 |
| D-1361 | 632E−12 | −76.77 | D-1368-run 1 | 2.82E−9 | −83.59 |
| D-1362 | 2.58E−9 | −76.67 | D-1368-run 2 | 1.12E−09 | −88.39 |
| D-1363-run 1 | 1.29E−9 | −91.72 | D-1369 | 2.11E−9 | −75.09 |
| D-1363-run 2 | 2.30E−09 | −91.91 | D-1370 | 1.96E−9 | −79.61 |
| D-1364-run 1 | 1.11E−9 | −87.19 | D-1371-run 1 | 1.19E−9 | −84.84 |
| D-1364-run 2 | 1.14E−09 | −91.01 | D-1371-run 2 | 1.14E−09 | −86.39 |
| D-1365-run 1 | 1.42E−9 | −85.38 | D-1372 | 1.38E−9 | −69.85 |
| D-1365-run 2 | 1.80E−09 | −88.72 | D-1373 | 2.62E−9 | −68.36 |
| D-1374 | 2.83E−9 | −78.50 | D-1380-run 1 | 2.43E−9 | −81.50 |
| D-1375-run 1 | 754E−12 | −91.87 | D-1380-run 2 | 2.13E−09 | −84.97 |
| D-1375-run 2 | 8.44E−10 | −89.51 | D-1381-run 1 | 202E−12 | −89.58 |
| D-1376-run 1 | 2.47E−9 | −85.68 | D-1381-run 2 | 3.79E−10 | −89.29 |
| D-1376-run 2 | 2.26E−09 | −85.67 | D-1382-run 1 | 429E−12 | −97.54 |
| D-1377-run 1 | 1.24E−9 | −83.02 | D-1382-run 2 | 2.17E−10 | −90.44 |
| D-1377-run 2 | 1.45E−09 | −88.30 | D-1383-run 1 | 939E−12 | −92.11 |
| D-1378 | 4.05E−9 | −53.31 | D-1383-run 2 | 7.75E−10 | −90.60 |
| D-1379-run 1 | 2.45E−9 | −85.07 | D-1384-run 1 | 29.6E−9 | −81.72 |
| D-1379-run 2 | 1.58E−09 | −87.92 | D-1384-run 2 | 1.62E−10 | −93.78 |
| D-1385-run 1 | 470E−12 | −84.34 | D-1390-run 1 | 587E−12 | −101.09 |
| D-1385-run 2 | 1.96E−10 | −85.42 | D-1390-run 2 | 2.46E−10 | −93.89 |
| D-1386-run 1 | 508E−12 | −93.47 | D-1391-run 1 | 206E−12 | −86.03 |
| D-1386-run 2 | 3.20E−10 | −93.01 | D-1391-run 2 | 1.91E−10 | −89.58 |
| D-1387-run 1 | 564E−12 | −93.18 | D-1392-run 1 | 602E−12 | −87.07 |
| D-1387-run 2 | 3.07E−10 | −90.08 | D-1392-run 2 | 8.32E−10 | −83.70 |
| D-1388-run 1 | 632E−12 | −92.58 | D-1393-run 1 | 1.28E−9 | −80.15 |
| D-1388-run 2 | 7.64E−10 | −95.22 | D-1393-run 2 | 8.95E−10 | −74.95 |
| D-1389-run 1 | 227E−12 | −94.67 | D-1394-run 1 | 1.72E−9 | −80.33 |
| D-1389-run 2 | 3.90E−10 | −95.16 | D-1394-run 2 | 1.05E−09 | −80.33 |
| D-1395-run 1 | 746E−12 | −88.44 | D-1400-run 1 | 1.17E−9 | −93.27 |
| D-1395-run 2 | 5.06E−10 | −77.24 | D-1400-run 2 | 6.99E−10 | −86.44 |
| D-1396-run 1 | 784E−12 | −92.23 | D-1401-run 1 | 753E−12 | −92.66 |
| D-1396-run 2 | 9.88E−10 | −86.55 | D-1401-run 2 | 6.27E−10 | −85.65 |
| D-1397-run 1 | 551E−12 | −86.58 | D-1402-run 1 | 411E−12 | −90.34 |
| D-1397-run 2 | 5.51E−10 | −84.90 | D-1402-run 2 | 1.29E−10 | −91.80 |
| D-1398-run 1 | 489E−12 | −80.69 | D-1403-run 1 | 771E−12 | −88.84 |
| D-1398-run 2 | 2.09E−10 | −86.47 | D-1403-run 2 | 4.78E−10 | −86.79 |
| D-1399-run 1 | 369E−12 | −86.49 | D-1404-run 1 | 421E−12 | −86.45 |
| D-1399-run 2 | 1.34E−10 | −93.52 | D-1404-run 2 | 4.48E−10 | −93.17 |
| D-1405-run 1 | 187E−12 | −91.82 | D-1412 | >100E−9 | −20.63 |
| D-1405-run 2 | 2.50E−10 | −95.68 | D-1413-run 1 | 5.18E−9 | −81.68 |
| D-1406-run 1 | 282E−12 | −88.00 | D-1413-run 2 | 3.13E−09 | −76.27 |
| D-1406-run 2 | 1.25E−10 | −91.00 | D-1414 | 2.02E−9 | −63.97 |
| D-1407-run 1 | 403E−12 | −91.27 | D-1415 | 3.84E−9 | −56.01 |
| D-1407-run 2 | 2.01E−10 | −82.75 | D-1416-run 1 | 1.94E−9 | −94.76 |
| D-1408 | >100E−9 | 19.37 | D-1416-run 2 | 1.47E−09 | −85.86 |
| D-1409 | >100E−9 | 44.69 | D-1417 | 5.26E−9 | −50.98 |
| D-1410 | >100E−9 | −16.05 | D-1418 | 5.94E−9 | −63.89 |
| D-1411 | >100E−9 | 10.66 | D-1419 | >100E−9 | −19.86 |
| D-1420-run 1 | 2.68E−9 | −94.55 | D-1428 | >100E−9 | 4.40 |
| D-1420-run 2 | 1.24E−09 | −86.57 | D-1429 | >100E−9 | −9.19 |
| D-1421-run 1 | 5.7E−9 | −89.95 | D-1430 | >100E−9 | 2.73 |
| D-1421-run 2 | 6.19E−09 | −84.56 | D-1431 | >100E−9 | −19.92 |
| D-1422 | 25.5E−9 | −53.28 | D-1432 | >100E−9 | −2.75 |
| D-1423 | 31.8E−9 | −62.33 | D-1433 | >100E−9 | −12.03 |
| D-1424 | >100E−9 | −30.24 | D-1434 | 4.58E−9 | −53.99 |
| D-1425 | >100E−9 | 19.38 | D-1435 | >100E−9 | 2.85 |
| D-1426 | >100E−9 | 1.22 | D-1436 | >100E−9 | −21.73 |
| D-1427 | >100E−9 | −8.72 | D-1437 | 9.37E−9 | −53.74 |
| D-1438 | 33.3E−9 | −33.44 | D-1445 | 7.92E−9 | −71.51 |
| D-1439-run 1 | 3.21E−9 | −88.80 | D-1446 | 100E−9 | −28.05 |
| D-1439-run 2 | 2.07E−09 | −86.97 | D-1447-run 1 | 4.6E−9 | −81.98 |
| D-1440 | 15.8E−9 | −53.11 | D-1447-run 2 | 1.92E−09 | −74.90 |
| D-1441-run 1 | 860E−12 | −93.62 | D-1448 | 754E−12 | −75.31 |
| D-1441-run 2 | 1.41E−09 | −92.60 | D-1449 | 1.61E−9 | −73.73 |
| D-1442 | 15.3E−9 | −60.48 | D-1450 | 1.91E−9 | −78.64 |
| D-1443 | 4.55E−9 | −61.60 | D-1451-run 1 | 1.41E−9 | −92.19 |
| D-1444-run 1 | 3.64E−9 | −80.44 | D-1451-run 2 | 1.61E−09 | −88.76 |
| D-1444-run 2 | 1.88E−09 | −75.71 | D-1452-run 1 | 477E−12 | −84.31 |
| D-1453 | 1.64E−9 | −77.22 | D-1452-run 2 | 5.50E−10 | −81.90 |
| D-1454-run 1 | 1.39E−9 | −87.14 | D-1459-run 1 | 845E−12 | −85.31 |
| D-1454-run 2 | 2.24E−09 | −87.40 | D-1459-run 2 | 1.24E−09 | −93.61 |
| D-1455-run 1 | 341E−12 | −86.11 | D-1460-run 1 | 702E−12 | −88.96 |
| D-1455-run 2 | 2.42E−10 | −95.21 | D-1460-run 2 | 9.15E−10 | −89.53 |
| D-1456-run 1 | 2.03E−9 | −86.90 | D-1461 | 1.49E−9 | −71.01 |
| D-1456-run 2 | 1.41E−09 | −84.06 | D-1462 | 7.37E−9 | −66.33 |
| D-1457 | 1.92E−9 | −78.57 | D-1463 | >100E−9 | −17.28 |
| D-1458-run 1 | 6.71E−9 | −86.68 | D-1464 | 7.58E−9 | −61.74 |
| D-1458-run 2 | 2.24E−09 | −94.33 | D-1465 | 5.16E−9 | −75.36 |
| D-1466 | >100E−9 | −17.33 | D-1475 | 9.72E−9 | −59.40 |
| D-1467 | 10.9E−9 | −56.04 | D-1476 | 1.08E−9 | −54.33 |
| D-1468 | 3.78E−9 | −61.93 | D-1477 | 2.27E−9 | −55.97 |
| D-1469-run 1 | 953E−12 | −81.14 | D-1478 | 1.15E−9 | −54.86 |
| D-1469-run 2 | 1.59E−09 | −83.63 | D-1479 | 1.33E−9 | −44.54 |
| D-1470 | 19.6E−9 | −41.41 | D-1480-run 1 | 2.03E−9 | −85.44 |
| D-1471 | 46.7E−9 | −37.18 | D-1480-run 2 | 8.61E−10 | −88.52 |
| D-1472 | 4.45E−9 | −50.43 | D-1481-run 1 | 1.57E−9 | −80.97 |
| D-1473 | 2.17E−9 | −78.87 | D-1481-run 2 | 1.33E−09 | −90.57 |
| D-1474 | 8.29E−9 | −61.78 | D-1482 | 6.26E−9 | −53.31 |
| D-1483 | 10.8E−9 | −73.64 | D-1489-run 1 | 2.49E−9 | −92.70 |
| D-1484 | 1.63E−9 | −80.04 | D-1489-run 2 | 2.02E−09 | −100.75 |
| D-1485-run 1 | 614E−12 | −86.35 | D-1490 | 7.08E−9 | −77.03 |
| D-1485-run 2 | 7.55E−10 | −90.60 | D-1491-run 1 | 1.31E−9 | −95.30 |
| D-1486-run 1 | 2.44E−9 | −81.67 | D-1491-run 2 | 9.32E−10 | −92.84 |
| D-1486-run 2 | 5.61E−10 | −85.95 | D-1492-run 1 | 470E−12 | −88.29 |
| D-1487-run 1 | 3.14E−9 | −92.01 | D-1492-run 2 | 6.96E−10 | −91.86 |
| D-1487-run 2 | 4.26E−10 | −90.53 | D-1493-run 1 | 2.72E−9 | −89.31 |
| D-1488-run 1 | 4.58E−9 | −82.33 | D-1493-run 2 | 2.25E−09 | −89.06 |
| D-1488-run 2 | 2.65E−09 | −80.98 | D-1494 | 2.08E−9 | −79.36 |
| D-1495 | 3.7E−9 | −63.78 | D-1502 | 12.3E−9 | −60.25 |
| D-1496-run 1 | 2.26E−9 | −82.93 | D-1503-run 1 | 1.35E−9 | −94.10 |
| D-1496-run 2 | 6.17E−10 | −86.34 | D-1503-run 2 | 8.46E−10 | −88.39 |
| D-1497-run 1 | 3.09E−9 | −83.24 | D-1504-run 1 | 2.62E−9 | −92.42 |
| D-1497-run 2 | 1.20E−09 | −89.93 | D-1504-run 2 | 2.39E−09 | −84.78 |
| D-1498 | 958E−12 | −79.10 | D-1505 | 12.1E−9 | −44.23 |
| D-1499-run 1 | 434E−12 | −82.58 | D-1506-run 1 | 2.33E−9 | −82.44 |
| D-1499-run 2 | 7.85E−10 | −86.02 | D-1506-run 2 | 2.43E−09 | −74.70 |
| D-1500 | 2.94E−9 | −64.87 | D-1507-run 1 | 7.22E−9 | −85.49 |
| D-1501 | 3.79E−9 | −67.10 | D-1507-run 2 | 3.07E−09 | −78.73 |
| D-1508 | 9.61E−9 | −69.25 | D-1516 | 4.11E−9 | −72.86 |
| D-1509-run 1 | 1.84E−9 | −87.02 | D-1517 | 1.86E−9 | −70.15 |
| D-1509-run 2 | 1.54E−09 | −90.33 | D-1518 | 5.19E−9 | −80.44 |
| D-1510-run 1 | 2E−9 | −84.70 | D-1519-run 1 | 3.16E−9 | −87.18 |
| D-1510-run 2 | 1.33E−09 | −74.09 | D-1519-run 2 | 1.85E−09 | −81.61 |
| D-1511 | 4.91E−9 | −70.77 | D-1520 | 2.61E−9 | −75.37 |
| D-1512 | 13.7E−9 | −50.81 | D-1521 | 8.95E−9 | −71.81 |
| D-1513 | 10.4E−9 | −61.35 | D-1522 | 1.05E−9 | −77.55 |
| D-1514 | 8.52E−9 | −58.65 | D-1523 | 5.61E−9 | −56.61 |
| D-1515-run 1 | 3.02E−9 | −94.07 | D-1524 | 8.18E−9 | −70.51 |
| D-1515-run 2 | 2.11E−09 | −88.32 | D-1525-run 1 | 187E−12 | −85.31 |
| D-1526-run 1 | 724E−12 | −88.90 | D-1525-run 2 | 3.09E−10 | −89.32 |
| D-1526-run 2 | 1.09E−10 | −93.08 | D-1533-run 1 | 160E−12 | −87.58 |
| D-1527 | 969E−12 | −79.47 | D-1533-run 2 | 1.77E−10 | −89.17 |
| D-1528-run 1 | 476E−12 | −81.98 | D-1534 | 4.12E−9 | −77.89 |
| D-1528-run 2 | 5.16E−10 | −86.43 | D-1535-run 1 | 3.91E−9 | −81.08 |
| D-1529 | 275E−12 | −80.46 | D-1535-run 2 | 2.53E−09 | −83.97 |
| D-1530 | 603E−12 | −79.76 | D-1536-run 1 | 1.17E−9 | −85.54 |
| D-1531-run 1 | 465E−12 | −81.21 | D-1536-run 2 | 5.45E−10 | −84.54 |
| D-1531-run 2 | 4.26E−10 | −86.14 | D-1537 | 5.53E−9 | −72.51 |
| D-1532-run 1 | 234E−12 | −82.79 | D-1538 | 869E−12 | −74.09 |
| D-1532-run 2 | 2.09E−10 | −88.87 | D-1539 | 6.85E−9 | −74.08 |
| D-1540-run 1 | 2.09E−9 | −83.12 | D-1548 | 27.6E−9 | −37.78 |
| D-1540-run 2 | 1.27E−09 | −88.11 | D-1549-run 1 | 1.04E−9 | −93.06 |
| D-1541 | 932E−12 | −76.06 | D-1549-run 2 | 8.95E−10 | −88.71 |
| D-1542-run 1 | 3.16E−9 | −81.16 | D-1550 | 652E−12 | −79.25 |
| D-1542-run 2 | 1.48E−09 | −83.09 | D-1551-run 1 | 500E−12 | −86.59 |
| D-1543 | 8.54E−9 | −79.76 | D-1551-run 2 | 5.64E−10 | −84.20 |
| D-1544 | 8.28E−9 | −73.28 | D-1552-run 1 | 228E−12 | −81.86 |
| D-1545 | 1.7E−9 | −75.05 | D-1552-run 2 | 3.22E−10 | −89.73 |
| D-1546 | 1.45E−9 | −79.08 | D-1553-run 1 | 931E−12 | −80.68 |
| D-1547 | 22.3E−9 | −69.31 | D-1553-run 2 | 8.28E−10 | −89.65 |
| D-1554 | 1.27E−9 | −78.63 | D-1560 | 456E−12 | −74.69 |
| D-1555-run 1 | 663E−12 | −88.13 | D-1561-run 1 | 2.39E−9 | −83.41 |
| D-1555-run 2 | 4.61E−10 | −90.35 | D-1561-run 2 | 3.83E−9 | −80.47 |
| D-1556-run 1 | 165E−12 | −83.51 | D-1561-run 3 | 2.15E−09 | −83.36 |
| D-1556-run 2 | 2.53E−10 | −87.06 | D-1562-run 1 | 1.1E−9 | −72.98 |
| D-1557-run 1 | 440E−12 | −87.71 | D-1562-run 2 | 1.26E−9 | −74.09 |
| D-1557-run 2 | 2.50E−10 | −85.00 | D-1563-run 1 | 898E−12 | −78.70 |
| D-1558-run 1 | 762E−12 | −81.35 | D-1563-run 2 | 1.07E−9 | −76.14 |
| D-1558-run 2 | 2.60E−10 | −80.18 | D-1564-run 1 | 906E−12 | −85.03 |
| D-1559-run 1 | 668E−12 | −87.94 | D-1564-run 2 | 1.75E−9 | −76.80 |
| D-1559-run 2 | 5.42E−10 | −86.43 | D-1564-run 3 | 1.01E−09 | −81.88 |
| D-1565-run 1 | 467E−12 | −79.91 | D-1570-run 1 | 925E−12 | −78.14 |
| D-1565-run 2 | 1.27E−9 | −78.07 | D-1570-run 2 | 992E−12 | −76.81 |
| D-1566-run 1 | 1.5E−9 | −80.84 | D-1571-run 1 | 779E−12 | −80.29 |
| D-1566-run 2 | 4.17E−9 | −85.05 | D-1571-run 2 | 2.7E−9 | −80.52 |
| D-1566-run 3 | 8.64E−10 | −84.74 | D-1571-run 3 | 8.27E−10 | −84.74 |
| D-1567-run 1 | 3.71E−9 | −75.99 | D-1572 | 820E−12 | −76.58 |
| D-1567-run 2 | 2.34E−9 | −72.76 | D-1573-run 1 | 1.52E−9 | −79.66 |
| D-1568 | 7.49E−9 | −49.04 | D-1573-run 2 | 3.19E−9 | −79.88 |
| D-1569-run 1 | 2.74E−9 | −78.08 | D-1574-run 1 | 4.19E−9 | −60.81 |
| D-1569-run 2 | 3.13E−9 | −74.70 | D-1574-run 2 | >100E−9 | −15.89 |
| D-1575-run 1 | 1.59E−9 | −76.35 | D-1579-run 1 | 515E−12 | −84.80 |
| D-1575-run 2 | 2.88E−9 | −71.95 | D-1579-run 2 | 207E−12 | −79.57 |
| D-1576-run 1 | 1.07E−9 | −90.18 | D-1579-run 3 | 2.98E−09 | −83.77 |
| D-1576-run 2 | 1.37E−09 | −94.66 | D-1580-run 1 | 233E−12 | −83.48 |
| D-1577-run 1 | 1.3E−9 | −83.26 | D-1580-run 2 | 3.19E−10 | −90.62 |
| D-1577-run 2 | 1.52E−9 | −80.99 | D-1581-run 1 | 376E−12 | −89.56 |
| D-1577-run 3 | 1.14E−09 | −85.26 | D-1581-run 2 | 164E−12 | −84.43 |
| D-1578-run 1 | 1.34E−9 | −84.03 | D-1581-run 3 | 4.10E−10 | −88.78 |
| D-1578-run 2 | 571E−12 | −77.18 | D-1582-run 1 | 1.25E−9 | −77.75 |
| D-1578-run 3 | 6.20E−10 | −79.39 | D-1582-run 2 | 1.38E−9 | −73.23 |
| D-1583-run 1 | 3.97E−9 | −79.12 | D-1589 | 1.89E−9 | −79.52 |
| D-1583-run 2 | 2.2E−9 | −78.20 | D-1590-run 1 | 1.56E−9 | −86.60 |
| D-1584-run 1 | 377E−12 | −84.12 | D-1590-run 2 | 9.85E−10 | −86.66 |
| D-1584-run 2 | 5.41E−10 | −86.04 | D-1591 | 1.01E−9 | −78.77 |
| D-1585-run 1 | 854E−12 | −83.31 | D-1592 | 1.67E−9 | −78.16 |
| D-1585-run 2 | 1.15E−09 | −89.54 | D-1593 | 3.47E−9 | −58.82 |
| D-1586 | 1.24E−9 | −76.28 | D-1594 | 2.08E−9 | −71.26 |
| D-1587-run 1 | 1.04E−9 | −87.24 | D-1595-run 1 | 531E−12 | −92.76 |
| D-1587-run 2 | 1.22E−09 | −90.08 | D-1595-run 2 | 4.46E−10 | −93.09 |
| D-1588-run 1 | 1.24E−9 | −82.16 | D-1596-run 1 | 655E−12 | −95.03 |
| D-1588-run 2 | 2.00E−09 | −84.12 | D-1596-run 2 | 6.71E−10 | −88.34 |
| D-1597-run 1 | 566E−12 | −87.01 | D-1603-run 1 | 868E−12 | −84.49 |
| D-1597-run 2 | 9.25E−10 | −86.25 | D-1603-run 2 | 1.25E−09 | −89.46 |
| D-1598-run 1 | 1.07E−9 | −81.80 | D-1604-run 1 | 1.03E−9 | −85.81 |
| D-1598-run 2 | 1.18E−09 | −77.57 | D-1604-run 2 | 8.37E−10 | −82.91 |
| D-1599-run 1 | 844E−12 | −85.25 | D-1605-run 1 | 831E−12 | −87.43 |
| D-1599-run 2 | 7.40E−10 | −79.77 | D-1605-run 2 | 7.85E−10 | −84.90 |
| D-1600 | 1.34E−9 | −65.29 | D-1606-run 1 | 617E−12 | −91.60 |
| D-1601 | 646E−12 | −71.11 | D-1606-run 2 | 9.69E−10 | −83.11 |
| D-1602-run 1 | 668E−12 | −88.45 | D-1607-run 1 | 549E−12 | −85.52 |
| D-1602-run 2 | 8.97E−10 | −91.07 | D-1607-run 2 | 7.47E−10 | −77.90 |
| D-1608 | 1.35E−9 | −75.97 | D-1614-run 1 | 803E−12 | −80.05 |
| D-1609-run 1 | 1.54E−9 | −81.34 | D-1614-run 2 | 6.78E−10 | −86.26 |
| D-1609-run 2 | 1.63E−09 | −77.49 | D-1615 | 2.12E−9 | −67.39 |
| D-1610-run 1 | 2.58E−9 | −84.25 | D-1616 | 38.3E−9 | −57.61 |
| D-1610-run 2 | 2.56E−09 | −80.89 | D-1617 | 4.8E−9 | −57.99 |
| D-1611-run 1 | 865E−12 | −89.50 | D-1618-run 1 | 1.18E−9 | −85.32 |
| D-1611-run 2 | 6.04E−10 | −86.80 | D-1618-run 2 | 2.42E−09 | −85.91 |
| D-1612 | 1.89E−9 | −77.95 | D-1619 | >100E−9 | 2.12 |
| D-1613-run 1 | 1.29E−9 | −84.46 | D-1620 | — | −47.40 |
| D-1613-run 2 | 1.44E−09 | −80.67 | D-1621 | >100E−9 | 1.40 |
| D-1622 | 1.37E−9 | −79.11 | D-1630-run 1 | 1.67E−9 | −83.08 |
| D-1623 | 3.43E−9 | −66.62 | D-1630-run 2 | 2.29E−09 | −83.88 |
| D-1624-run 1 | 6.9E−9 | −85.54 | D-1631-run 1 | 1.42E−9 | −87.66 |
| D-1624-run 2 | 3.15E−09 | −84.79 | D-1631-run 2 | 1.12E−09 | −86.08 |
| D-1625 | 2.07E−9 | −79.25 | D-1632-run 1 | 1.3E−9 | −88.34 |
| D-1626-run 1 | 2.14E−9 | −98.64 | D-1632-run 2 | 8.33E−10 | −85.76 |
| D-1626-run 2 | 2.02E−09 | −87.31 | D-1633-run 1 | 1.79E−9 | −90.59 |
| D-1627 | 6E−9 | −67.49 | D-1633-run 2 | 1.66E−09 | −82.90 |
| D-1628 | >100E−9 | 3.55 | D-1634-run 1 | 837E−12 | −82.47 |
| D-1629-run 1 | 1.13E−9 | −87.48 | D-1634-run 2 | 7.69E−10 | −84.53 |
| D-1629-run 2 | 1.43E−09 | −81.66 | D-1635-run 1 | 1.04E−9 | −88.75 |
| D-1636 | 964E−12 | −69.05 | D-1635-run 2 | 1.58E−09 | −93.88 |
| D-1637 | 2.36E−9 | −75.45 | D-1646 | 517E−12 | −80.04 |
| D-1638-run 1 | 583E−12 | −82.46 | D-1647 | 319E−12 | −73.09 |
| D-1638-run 2 | 7.20E−10 | −85.90 | D-1648-run 1 | 1.57E−9 | −81.70 |
| D-1639 | 411E−12 | −68.37 | D-1648-run 2 | 1.30E−09 | −84.65 |
| D-1640 | 788E−12 | −79.76 | D-1649 | 275E−12 | −79.59 |
| D-1641 | 1.85E−9 | −69.30 | D-1650 | 1.63E−9 | −80.12 |
| D-1642 | 2.52E−9 | −75.03 | D-1651-run 1 | 415E−12 | −83.10 |
| D-1643 | 3.97E−9 | −73.66 | D-1651-run 2 | 3.97E−10 | −90.33 |
| D-1644 | 886E−12 | −67.60 | D-1652-run 1 | 445E−12 | −81.69 |
| D-1645 | 822E−12 | −70.30 | D-1652-run 2 | 1.61E−10 | −86.02 |
| D-1653 | 197E−12 | −79.25 | D-1663-run 1 | 144E−12 | −87.88 |
| D-1654-run 1 | 279E−12 | −85.62 | D-1663-run 2 | 1.92E−10 | −91.58 |
| D-1654-run 2 | 3.82E−10 | −89.31 | D-1664-run 1 | 155E−12 | −81.73 |
| D-1655-run 1 | 380E−12 | −88.31 | D-1664-run 2 | 2.87E−10 | −89.13 |
| D-1655-run 2 | 3.35E−10 | −96.53 | D-1665-run 1 | 164E−12 | −81.33 |
| D-1656-run 1 | 200E−12 | −87.91 | D-1665-run 2 | 2.62E−10 | −87.08 |
| D-1656-run 2 | 1.86E−10 | −85.63 | D-1666-run 1 | 484E−12 | −84.08 |
| D-1657-run 1 | 144E−12 | −85.58 | D-1666-run 2 | 2.86E−10 | −82.03 |
| D-1657-run 2 | 3.53E−10 | −88.57 | D-1667-run 1 | 408E−12 | −85.17 |
| D-1658 | 197E−12 | −80.34 | D-1667-run 2 | 2.66E−10 | −87.72 |
| D-1659 | 255E−12 | −80.45 | D-1668-run 1 | 650E−12 | −83.77 |
| D-1660 | 597E−12 | −78.68 | D-1668-run 2 | 2.74E−10 | −86.81 |
| D-1661 | 219E−12 | −78.29 | | | |
| D-1662-run 1 | 369E−12 | −89.73 | | | |
| D-1662-run 2 | 2.84E−10 | −96.38 | | | |

Of the additional 406 mARC1 siRNA molecules targeting different regions of the human mARC1 transcript, 128 molecules produced a reduction of human mARC1 mRNA in Hep3B cells of 85% or greater. Forty-six molecules (duplex nos. D-1061; D-1093; D-1220; D-1276; D-1284; D-1298; D-1310; D-1311; D-1338; D-1363; D-1367; D-1375; D-1381; D-1382; D-1383; D-1386; D-1387; D-1388; D-1389; D-1390; D-1396; D-1400; D-1401; D-1402; D-1405; D-1407; D-1416; D-1420; D-1421; D-1441; D-1451; D-1487; D-1489; D-1491; D-1503; D-1504; D-1515; D-1549; D-1576; D-1581; D-1595; D-1596; D-1606; D-1626; D-1633; and D-1662) reduced human mARC1 mRNA by at least 90% with the majority of the molecules having IC50 values below 1 nM.

### Example 4. In Vivo Efficacy of siRNA Molecules in AAV Human mARC1 Mouse Model

To assess the efficacy of the mARC1 siRNA molecules in vivo, the sense strand in each siRNA molecule was conjugated to the trivalent GalNAc moiety shown in Formula VII by the methods described in Example 2 and the mARC1 siRNA molecules were administered to mice expressing the human MARC1 gene. 10-12-week-old C57BL/6 mice (The Jackson Laboratory) were fed standard chow (Harlan, 2020× Teklad global soy protein-free extruded rodent diet). Mice were intraperitoneally (i.p.) injected with an adeno-associated virus (AAV) encoding the human MARC1 gene (AAV-hmARC1) at a dose of 1×10¹¹ genome copies (GC) per animal. One week following AAV-hmARC1 injection, mice received a single subcutaneous (s.c.) injection of buffer or the mARC1 siRNA molecule at a dose of 0.5 mg/kg, 1 mg/kg, or 3 mg/kg body weight in buffer (n=3 each group). Animals were fasted and harvested four weeks following siRNA administration for further analysis. Liver total RNA from harvested animals was processed for qPCR analysis and serum parameters were measured by clinical analyzer (AU400 Chemistry Analyzer, Olympus). A percentage change in human mARC1 mRNA in liver for each animal was calculated relative to human mARC1 mRNA liver levels in control animals which expressed human mARC1 mRNA and received the buffer only injection (i.e. AAV-hmARC1 only animals).

The top performing mARC1 siRNA molecules from the in vitro activity assays described in Example 3 were evaluated for in vivo efficacy in this model. mARC1 siRNA molecules that exhibited significant in vivo knockdown activity were further evaluated in SAR studies to further improve in vivo potency and durability by altering chemical modification patterns. Results of 18 separate studies in the AAV-hmARC1 mouse model with different mARC1 siRNA molecules are shown in Tables 5-22 below. Data are expressed as average percent change from control at week 5 of study (4 weeks after siRNA injection) for each treatment group (n=3 animals/group). If a mARC1 siRNA molecule has the same trigger family designation as another mARC1 siRNA molecule, then the two molecules have the same core sequence (i.e. target the same region of the mARC1 transcript) but differ in chemical modification pattern.

TABLE 5
In vivo inhibition of human mARC1 mRNA in AAV-hmARC1 mice-Study 1

| | | | Avg. % | | | | Avg. % |
|---|---|---|---|---|---|---|---|
| | | | Change | | | | Change |
| | | | in | | | | in |
| Treatment | | Trigger | human | Treatment | | Trigger | human |
| (duplex | Dose | Family | mARC1 | (duplex | Dose | Family | mARC1 |
| no.) | (mg/kg) | Designation | mRNA | no.) | (mg/kg) | Designation | mRNA |
| D-2000 | 1 | T918 | −58 | D-2032 | 1 | T1110 | 6.8 |
| D-2001 | 1 | T1114 | −71.6 | D-2033 | 1 | T1111 | 8.4 |
| D-2002 | 1 | T1016 | −50.6 | D-2034 | 1 | T911 | −14.1 |
| D-2003 | 1 | T1023 | −51.5 | D-2035 | 1 | T1079 | −25 |
| D-2004 | 1 | T704 | −67.4 | D-2036 | 1 | T913 | −23.6 |
| D-2005 | 1 | T1076 | −43.3 | D-2038 | 1 | T914 | −67.2 |
| D-2008 | 1 | T1476 | −45.3 | D-2040 | 1 | T1484 | −54.7 |
| D-2011 | 1 | T1487 | −54.2 | D-2042 | 1 | T1372 | −76.6 |
| D-2013 | 1 | T1364 | −17.3 | D-2044 | 1 | T1449 | −62.6 |
| D-2022 | 1 | T2131 | −67.6 | D-2045 | 1 | T2077 | −69.2 |
| D-2024 | 1 | T816 | −72.8 | D-2046 | 1 | T1363 | −39.7 |
| D-2026 | 1 | T1108 | −10.5 | D-2047 | 1 | T1367 | −58.4 |
| D-2028 | 1 | T1113 | −4.8 | D-2049 | 1 | T1104 | −26 |
| D-2031 | 1 | T1109 | −27.8 | D-2050 | 1 | T1101 | −42.4 |

TABLE 6
In vivo inhibition of human mARC1 mRNA in AAV-hmARC1 mice-Study 2

| | | | Avg. % | | | | Avg. % |
|---|---|---|---|---|---|---|---|
| | | | Change | | | | Change |
| | | | in | | | | in |
| Treatment | | Trigger | human | Treatment | | Trigger | human |
| (duplex | Dose | Family | mARC1 | (duplex | Dose | Family | mARC1 |
| no.) | (mg/kg) | Designation | mRNA | no.) | (mg/kg) | Designation | mRNA |
| D-2006 | 1 | T1080 | −60.12 | D-2048 | 1 | T1780 | −48.24 |
| D-2015 | 1 | T2024 | −59.3 | D-2051 | 1 | T1670 | −61.04 |
| D-2016 | 1 | T2032 | −63.1 | D-2052 | 1 | T1370 | −89.53 |
| D-2017 | 1 | T2034 | −70.1 | D-2053 | 1 | T1458 | −81.49 |
| D-2019 | 1 | T2099 | −43.07 | D-2054 | 1 | T1878 | −36.34 |
| D-2025 | 1 | T1086 | −53.07 | D-2055 | 1 | T1767 | −66.47 |
| D-2027 | 1 | T1105 | −22.88 | D-2057 | 1 | T788 | −69.06 |
| D-2029 | 1 | T976 | −42.62 | D-2058 | 1 | T1275 | −84.33 |
| D-2030 | 1 | T1123 | −28.6 | D-2059 | 1 | T1814 | −78.71 |
| D-2037 | 1 | T898 | −18.98 | D-2060 | 1 | T1130 | −74.14 |
| D-2039 | 1 | T813 | −69.93 | D-2061 | 1 | T1816 | −75.23 |
| D-2041 | 1 | T1127 | −21.84 | D-2062 | 1 | T1485 | −58.27 |
| D-2043 | 1 | T1375 | −72.11 | | | | |

TABLE 7
In vivo inhibition of human mARC1 mRNA in AAV-hmARC1 mice-Study 3

| | | | Avg. % | | | | Avg. % |
|---|---|---|---|---|---|---|---|
| | | | Change | | | | Change |
| | | | in | | | | in |
| Treatment | | Trigger | human | Treatment | | Trigger | human |
| (duplex | Dose | Family | mARC1 | (duplex | Dose | Family | mARC1 |
| no.) | (mg/kg) | Designation | mRNA | no.) | (mg/kg) | Designation | mRNA |
| D-2063 | 1 | T1229 | −69.57 | D-2075 | 1 | T1382 | −52.85 |
| D-2064 | 1 | T885 | −26.21 | D-2076 | 1 | T1236 | −86.21 |
| D-2065 | 1 | T2068 | −73.42 | D-2077 | 1 | T1235 | −87.22 |
| D-2066 | 1 | T1227 | −54.74 | D-2078 | 1 | T1234 | −91.7 |
| D-2067 | 1 | T1268 | −27.15 | D-2079 | 1 | T2074 | −85.91 |
| D-2068 | 1 | T1992 | −56.89 | D-2080 | 1 | T1233 | −88.99 |
| D-2069 | 1 | T1990 | −17.92 | D-2081 | 1 | T1232 | −89.64 |
| D-2070 | 1 | T1959 | −72.22 | D-2082 | 1 | T2072 | −74.89 |
| D-2071 | 1 | T1146 | −55.69 | D-2083 | 1 | T1005 | −78.85 |
| D-2072 | 1 | T1526 | −81.87 | D-2084 | 1 | T948 | −45.28 |
| D-2073 | 1 | T1717 | −57.1 | D-2085 | 1 | T573 | −34.79 |
| D-2074 | 1 | T1077 | −25.26 | D-2101 | 1 | T836 | −57 |

TABLE 8
In vivo inhibition of human mARC1 mRNA in AAV-hmARC1 mice-Study 4

| | | | Avg. % | | | | Avg. % |
|---|---|---|---|---|---|---|---|
| | | | Change | | | | Change |
| | | | in | | | | in |
| Treatment | | Trigger | human | Treatment | | Trigger | human |
| (duplex | Dose | Family | mARC1 | (duplex | Dose | Family | mARC1 |
| no.) | (mg/kg) | Designation | mRNA | no.) | (mg/kg) | Designation | mRNA |
| D-2042 | 1 | T1372 | −72.75 | D-2097 | 1 | T999 | −11.78 |
| D-2086 | 1 | T590 | −18.09 | D-2098 | 1 | T609 | −44.23 |
| D-2087 | 1 | T1527 | −51.27 | D-2099 | 1 | T781 | −60.5 |
| D-2088 | 1 | T1067 | −23.35 | D-2100 | 1 | T830 | −41.23 |
| D-2089 | 1 | T1696 | −40.51 | D-2102 | 1 | T954 | −61.93 |
| D-2090 | 1 | T1548 | −51.00 | D-2103 | 1 | T1833 | −35.41 |
| D-2091 | 1 | T235 | −20.06 | D-2104 | 1 | T2020 | −36.31 |
| D-2092 | 1 | T508 | −5.28 | D-2105 | 1 | T2059 | −70.02 |
| D-2093 | 1 | T239 | −11.41 | D-2106 | 1 | T2060 | −64.34 |
| D-2094 | 1 | T1736 | −50.26 | D-2107 | 1 | T1467 | −37.67 |
| D-2095 | 1 | T240 | 13.33 | D-2108 | 1 | T1247 | −79.83 |
| D-2096 | 1 | T998 | −7.13 | D-2109 | 1 | T2133 | −31.79 |

TABLE 9
In vivo inhibition of human mARC1 mRNA in AAV-hmARC1 mice-Study 5

| | | | Avg. % | | | | Avg. % |
|---|---|---|---|---|---|---|---|
| | | | Change | | | | Change |
| | | | in | | | | in |
| Treatment | | Trigger | human | Treatment | | Trigger | human |
| (duplex | Dose | Family | mARC1 | (duplex | Dose | Family | mARC1 |
| no.) | (mg/kg) | Designation | mRNA | no.) | (mg/kg) | Designation | mRNA |
| D-2042 | 1 | T1372 | −75.98 | D-2110 | 1 | T596 | −67.89 |
| D-2052 | 3 | T1370 | −91.31 | D-2111 | 1 | T1334 | −88.01 |
| D-2052 | 1 | T1370 | −79.79 | D-2112 | 1 | T840 | −57.49 |
| D-2052 | 0.5 | T1370 | −51.84 | D-2113 | 1 | T1239 | −76.41 |
| D-2053 | 3 | T1458 | −91.83 | D-2114 | 1 | T2016 | −59.91 |
| D-2053 | 1 | T1458 | −85.54 | D-2115 | 1 | T2017 | −80.5 |
| D-2053 | 0.5 | T1458 | −30.4 | D-2116 | 1 | T1475 | −71.84 |
| D-2058 | 3 | T1275 | −85.79 | D-2117 | 1 | T2018 | −59.18 |
| D-2058 | 1 | T1275 | −74.59 | D-2118 | 1 | T2106 | −82.04 |
| D-2058 | 0.5 | T1275 | −52.58 | D-2119 | 1 | T1273 | −70.63 |
| D-2059 | 1 | T1814 | −71.24 | D-2120 | 1 | T1506 | −53.72 |
| D-2061 | 1 | T1816 | −67.34 | D-2121 | 1 | T1537 | −67.99 |

TABLE 10
In vivo inhibition of human mARC1 mRNA in AAV-hmARC1 mice-Study 6

| | | | Avg. % | | | | Avg. % |
|---|---|---|---|---|---|---|---|
| | | | Change | | | | Change |
| | | | in | | | | in |
| Treatment | | Trigger | human | Treatment | | Trigger | human |
| (duplex | Dose | Family | mARC1 | (duplex | Dose | Family | mARC1 |
| no.) | (mg/kg) | Designation | mRNA | no.) | (mg/kg) | Designation | mRNA |
| D-2042 | 1 | T1372 | −70.56 | D-2137 | 1 | T2073 | −28.29 |
| D-2078 | 3 | T1234 | −95.31 | D-2138 | 1 | T1089 | −42.53 |
| D-2078 | 1 | T1234 | −81.28 | D-2139 | 1 | T1716 | −60.34 |
| D-2078 | 0.5 | T1234 | −78.05 | D-2140 | 1 | T1124 | −46.49 |
| D-2079 | 3 | T2074 | −87.42 | D-2141 | 1 | T1965 | −51.64 |
| D-2079 | 1 | T2074 | −71.78 | D-2142 | 1 | T1230 | −73.09 |
| D-2079 | 0.5 | T2074 | −68.17 | D-2143 | 1 | T2071 | −47.83 |
| D-2080 | 1 | T1233 | −87.66 | D-2144 | 1 | T1012 | −17.18 |
| D-2081 | 3 | T1232 | −96.49 | D-2145 | 1 | T2102 | −73.86 |
| D-2081 | 1 | T1232 | −85.71 | D-2158 | 1 | T1372 | −80.83 |
| D-2081 | 0.5 | T1232 | −70.37 | D-2169 | 1 | T1372 | −71.01 |
| D-2083 | 1 | T1005 | −64.01 | D-2182 | 1 | T1372 | −80.03 |
| D-2134 | 1 | T604 | 15.84 | D-2185 | 1 | T1372 | −76.36 |
| D-2135 | 1 | T607 | −47.29 | D-2189 | 1 | T1372 | −82.41 |
| D-2136 | 1 | T1405 | −76.24 | | | | |

TABLE 11
In vivo inhibition of human mARC1 mRNA in
AAV-hmARC1 mice - Study 7

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2042 | 1 | T1372 | −70.27 |
| D-2161 | 1 | T914 | −29.66 |
| D-2162 | 1 | T1372 | −53.65 |
| D-2163 | 1 | T1114 | −60.02 |
| D-2166 | 1 | T2077 | −9.73 |
| D-2167 | 1 | T816 | −9.79 |
| D-2183 | 1 | T1372 | −64.17 |
| D-2184 | 1 | T1372 | −68.3 |
| D-2186 | 1 | T1372 | −73.66 |
| D-2187 | 1 | T1372 | −65.74 |
| D-2201 | 1 | T1458 | −46.02 |
| D-2206 | 1 | T1814 | −32.35 |
| D-2207 | 1 | T1130 | −24.97 |
| D-2208 | 1 | T1816 | 24.09 |
| D-2209 | 1 | T1458 | −71.78 |
| D-2210 | 1 | T1275 | −40.33 |
| D-2211 | 1 | T1370 | −80.39 |
| D-2212 | 1 | T2034 | −66.09 |
| D-2213 | 1 | T1375 | −72.26 |
| D-2214 | 1 | T1814 | −66.69 |
| D-2215 | 1 | T1130 | −24.86 |
| D-2216 | 1 | T1816 | −44.88 |
| D-2217 | 1 | T1458 | −53.44 |
| D-2218 | 1 | T1275 | −67.9 |
| D-2222 | 1 | T1814 | −61.29 |
| D-2223 | 1 | T1130 | −71.27 |
| D-2224 | 1 | T1816 | −41.34 |
| D-2225 | 1 | T1458 | −82.05 |
| D-2226 | 1 | T1275 | −66.42 |

TABLE 12
In vivo inhibition of human mARC1 mRNA in AAV-
hmARC1 mice - Study 8

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2081 | 1 | T1232 | −89 |
| D-2081 | 1 | T1232 | −84.81 |
| D-2081 | 0.5 | T1232 | −81.83 |
| D-2239 | 0.5 | T1005 | −43.57 |
| D-2240 | 0.5 | T1232 | −70.09 |
| D-2241 | 0.5 | T1233 | −91.21 |
| D-2242 | 0.5 | T2074 | −72.33 |
| D-2243 | 0.5 | T1234 | −82.12 |
| D-2244 | 0.5 | T1005 | −61.6 |
| D-2245 | 0.5 | T1232 | −70.32 |
| D-2246 | 0.5 | T1233 | −82.34 |
| D-2247 | 0.5 | T2074 | −71.57 |
| D-2248 | 0.5 | T1234 | −78.58 |
| D-2249 | 0.5 | T1005 | −33.8 |
| D-2250 | 0.5 | T1232 | −68.42 |
| D-2251 | 0.5 | T1233 | −61.68 |
| D-2252 | 0.5 | T2074 | −73.55 |
| D-2253 | 0.5 | T1234 | −75.93 |
| D-2254 | 0.5 | T1005 | −72.84 |
| D-2255 | 0.5 | T1232 | −85.73 |
| D-2256 | 0.5 | T1233 | −73.31 |
| D-2257 | 0.5 | T2074 | −57.42 |
| D-2258 | 0.5 | T1234 | −86.98 |
| D-2259 | 0.5 | T1234 | −76.42 |
| D-2260 | 0.5 | T2074 | −72.66 |
| D-2261 | 0.5 | T1232 | −78.9 |
| D-2262 | 0.5 | T2072 | −43.95 |

TABLE 13
In vivo inhibition of human mARC1 mRNA
in AAV-hmARC1 mice - Study 9

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2042 | 1 | T1372 | −70.46 |
| D-2081 | 0.5 | T1232 | −73.32 |
| D-2168 | 1 | T914 | −25.57 |
| D-2170 | 1 | T1114 | −59.16* |
| D-2173 | 1 | T2077 | −29.63 |
| D-2190 | 1 | T1114 | −1.89 |
| D-2193 | 1 | T2077 | −52.59* |
| D-2204 | 1 | T2034 | −37.36 |
| D-2220 | 1 | T2034 | −65.89 |
| D-2227 | 1 | T1113 | −39.03 |
| D-2229 | 1 | T1110 | −67.96 |
| D-2232 | 1 | T913 | −63.89 |
| D-2233 | 1 | T2034 | −62.85 |
| D-2236 | 1 | T1130 | −61.02 |
| D-2264 | 0.5 | T1233 | −63.96 |
| D-2265 | 0.5 | T1234 | −70 |
| D-2266 | 0.5 | T2074 | −47.47 |
| D-2268 | 0.5 | T1233 | −63.89 |
| D-2269 | 0.5 | T1233 | −58.41 |
| D-2270 | 0.5 | T1234 | −53.36 |
| D-2271 | 0.5 | T1234 | −57.52 |
| D-2272 | 0.5 | T2074 | −65.81 |
| D-2273 | 0.5 | T2074 | −62.06 |
| D-2301 | 1 | T1231 | −81.85 |
| D-2302 | 1 | T2070 | −72.03 |
| D-2303 | 1 | T2078 | −54.08 |
| D-2304 | 1 | T1365 | −67.08 |
| D-2305 | 1 | T1366 | −71.92 |
| D-2306 | 1 | T1369 | −64.17 |

*averages include one outlier; if outlier removed, average % change would be −79.41% (D-2170) and −70.68% (D-2193).

TABLE 14
In vivo inhibition of human mARC1 mRNA in
AAV-hmARC1 mice - Study 10

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2042 | 1 | T1372 | −74.85 |
| D-2081 | 0.5 | T1232 | −79.33 |
| D-2307 | 1 | T1373 | −57.28 |
| D-2308 | 1 | T1374 | −54.28 |
| D-2309 | 0.5 | T1234 | −68.23 |
| D-2310 | 0.5 | T2074 | −49.9 |
| D-2311 | 0.5 | T1233 | −71.87 |
| D-2312 | 0.5 | T1232 | −56.57 |
| D-2313 | 0.5 | T2072 | −46.01 |
| D-2314 | 0.5 | T1234 | −72.33 |
| D-2315 | 0.5 | T2074 | −61.11 |
| D-2316 | 0.5 | T1233 | −80.59 |
| D-2317 | 0.5 | T1232 | −79.12 |
| D-2318 | 0.5 | T2072 | −60.72 |
| D-2344 | 0.5 | T1234 | −79.85 |
| D-2345 | 0.5 | T2074 | −66.64 |
| D-2346 | 0.5 | T1233 | −71.49 |
| D-2347 | 0.5 | T1232 | −67.88 |
| D-2348 | 0.5 | T2072 | −29.7 |
| D-2349 | 0.5 | T1234 | −63.39 |
| D-2350 | 0.5 | T2074 | −53.12 |
| D-2351 | 0.5 | T1233 | −67.77 |
| D-2352 | 0.5 | T1232 | −36.82 |
| D-2353 | 0.5 | T2072 | −37.66 |
| D-2393 | 0.5 | T1234 | −47.75 |
| D-2394 | 0.5 | T2074 | −65.84 |
| D-2395 | 0.5 | T1233 | −70.01 |
| D-2396 | 0.5 | T1232 | −54.78 |
| D-2397 | 0.5 | T2072 | −28.06 |

TABLE 15
In vivo inhibition of human mARC1 mRNA in
AAV-hmARC1 mice - Study 11

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2081 | 0.5 | T1232 | −79.43 |
| D-2319 | 0.5 | T1234 | −63.31 |
| D-2320 | 0.5 | T2074 | −61.86 |
| D-2321 | 0.5 | T1233 | −75.48 |
| D-2322 | 0.5 | T1232 | −59.65 |
| D-2323 | 0.5 | T2072 | −34.97 |
| D-2324 | 0.5 | T1234 | −72.96 |
| D-2325 | 0.5 | T2074 | −66.78 |
| D-2326 | 0.5 | T1233 | −66.35 |
| D-2327 | 0.5 | T1232 | −55.48 |
| D-2328 | 0.5 | T2072 | −32.95 |
| D-2329 | 0.5 | T1234 | −79.94 |
| D-2330 | 0.5 | T2074 | −35.52 |
| D-2331 | 0.5 | T1233 | −55.59 |
| D-2332 | 0.5 | T1232 | −77.85 |
| D-2333 | 0.5 | T2072 | −28.56 |
| D-2334 | 0.5 | T1234 | −69.42 |
| D-2335 | 0.5 | T2074 | −40.1 |
| D-2336 | 0.5 | T1233 | −59.06 |
| D-2337 | 0.5 | T1232 | −53.38 |
| D-2338 | 0.5 | T2072 | −56.97 |
| D-2339 | 0.5 | T1234 | −72.93 |
| D-2340 | 0.5 | T2074 | −46.96 |
| D-2341 | 0.5 | T1233 | −83.39 |
| D-2342 | 0.5 | T1232 | −64.03 |
| D-2354 | 0.5 | T1234 | −66.85 |
| D-2355 | 0.5 | T2074 | −49.63 |
| D-2356 | 0.5 | T1233 | −80.84 |
| D-2357 | 0.5 | T1232 | −76.56 |

TABLE 16
In vivo inhibition of human mARC1 mRNA
in AAV-hmARC1 mice - Study 12

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2042 | 1 | T1372 | −78.96 |
| D-2080 | 0.5 | T1233 | −76.6 |
| D-2081 | 0.5 | T1232 | −80.5 |
| D-2241 | 0.5 | T1233 | −79.44 |
| D-2258 | 0.5 | T1234 | −79.5 |
| D-2374 | 1 | T1334 | −76.39 |
| D-2375 | 1 | T1334 | −84.32 |
| D-2376 | 1 | T1239 | −77.92 |
| D-2377 | 1 | T2017 | −68.11 |
| D-2378 | 1 | T2106 | −73.92 |
| D-2379 | 1 | T1334 | −81.46 |
| D-2380 | 1 | T1239 | −60.94 |
| D-2381 | 1 | T2017 | −73.06 |
| D-2382 | 1 | T2106 | −72.66 |
| D-2384 | 1 | T1372 | −87.07 |
| D-2385 | 1 | T1372 | −86.66 |
| D-2386 | 1 | T1372 | −82.75 |
| D-2387 | 1 | T1372 | −85.64 |
| D-2388 | 1 | T1372 | −85.26 |
| D-2389 | 1 | T1372 | −82.12 |
| D-2390 | 1 | T1372 | −78.41 |
| D-2391 | 1 | T1372 | −88.62 |
| D-2392 | 1 | T1372 | −79.8 |
| D-2399 | 1 | T1372 | −90.61 |
| D-2400 | 1 | T1372 | −82.94 |
| D-2401 | 1 | T1372 | −92.12 |
| D-2402 | 1 | T1372 | −73.56 |
| D-2403 | 1 | T1372 | −89.72 |

TABLE 17
In vivo inhibition of human mARC1 mRNA in
AAV-hmARC1 mice - Study 13

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2042 | 1 | T1372 | −72.06 |
| D-2045 | 1 | T2077 | −70.1 |
| D-2053 | 0.5 | T1458 | −43.98 |
| D-2079 | 0.5 | T2074 | −74.22 |
| D-2079 | 0.5 | T2074 | −67.06 |
| D-2081 | 0.5 | T1232 | −75.85 |
| D-2158 | 0.5 | T1372 | −77.49 |
| D-2159 | 1 | T1114 | −73.23 |
| D-2170 | 1 | T1114 | −69.02 |
| D-2182 | 0.5 | T1372 | −80.47 |
| D-2188 | 1 | T914 | −78.98 |
| D-2189 | 0.5 | T1372 | −76.83 |
| D-2193 | 1 | T2077 | −69.63 |
| D-2196 | 1 | T2034 | −87.29 |
| D-2200 | 1 | T1816 | −69.25 |
| D-2225 | 0.5 | T1458 | −65.93 |
| D-2228 | 1 | T1016 | −81.59 |
| D-2230 | 1 | T1111 | −28.82 |
| D-2231 | 1 | T911 | −41.89 |
| D-2237 | 1 | T1816 | −50.64 |
| D-2238 | 1 | T1458 | −80.45 |
| D-2242 | 0.5 | T2074 | −69.91 |
| D-2247 | 0.5 | T2074 | −59.81 |
| D-2252 | 0.5 | T2074 | −63.16 |
| D-2254 | 0.5 | T1005 | −34.25 |
| D-2260 | 0.5 | T2074 | −71.08 |
| D-2267 | 1 | T1816 | −21.42 |
| D-2343 | 0.5 | T2072 | −37.41 |
| D-2358 | 0.5 | T2072 | −36.05 |

TABLE 18
In vivo inhibition of human mARC1 mRNA
in AAV-hmARC1 mice - Study 14

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2081 | 0.5 | T1232 | −76.37 |
| D-2108 | 0.5 | T1247 | −75.82 |
| D-2111 | 0.5 | T1334 | −78.2 |
| D-2113 | 0.5 | T1239 | −69.06 |
| D-2115 | 0.5 | T2017 | −69.6 |
| D-2118 | 0.5 | T2106 | −66.06 |
| D-2430 | 0.5 | T1334 | −72.94 |
| D-2431 | 0.5 | T1239 | −66.56 |
| D-2432 | 0.5 | T2017 | −76.11 |
| D-2433 | 0.5 | T2106 | −67.19 |
| D-2434 | 0.5 | T1405 | −24.95 |
| D-2435 | 0.5 | T1526 | −68.17 |
| D-2436 | 0.5 | T1247 | −69.01 |
| D-2437 | 0.5 | T1231 | −75.43 |
| D-2438 | 0.5 | T1405 | −35.85 |
| D-2439 | 0.5 | T1526 | −64.23 |
| D-2440 | 0.5 | T1247 | −78.51 |
| D-2441 | 0.5 | T1231 | −72.52 |
| D-2442 | 0.5 | T1405 | −9.8 |
| D-2443 | 0.5 | T1526 | −48.33 |
| D-2444 | 0.5 | T1247 | −62.49 |
| D-2445 | 0.5 | T1231 | −56.81 |
| D-2446 | 0.5 | T1334 | −68.94 |
| D-2447 | 0.5 | T1239 | −56.33 |
| D-2448 | 0.5 | T2017 | −67.27 |
| D-2449 | 0.5 | T2106 | −75.69 |
| D-2450 | 0.5 | T1405 | −45.34 |
| D-2451 | 0.5 | T1526 | −64.83 |
| D-2453 | 0.5 | T1231 | −65.09 |

TABLE 19
In vivo inhibition of human mARC1 mRNA in
AAV-hmARC1 mice - Study 15

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2081 | 0.5 | T1232 | −78.97 |
| D-2199 | 0.5 | T1130 | −59.09 |
| D-2454 | 0.5 | T2074 | −65.23 |
| D-2455 | 0.5 | T2074 | −67.42 |
| D-2456 | 0.5 | T2074 | −61.94 |
| D-2457 | 0.5 | T2074 | −65.7 |
| D-2458 | 0.5 | T2074 | −46.41 |
| D-2459 | 0.5 | T2074 | −50.02 |
| D-2460 | 0.5 | T2074 | −60.38 |
| D-2461 | 0.5 | T2034 | −58.32 |
| D-2462 | 0.5 | T1114 | −77.64 |
| D-2463 | 0.5 | T2077 | −70.9 |
| D-2464 | 0.5 | T1130 | −58.04 |
| D-2465 | 0.5 | T2072 | −65.15 |
| D-2466 | 0.5 | T1959 | −55.27 |
| D-2467 | 0.5 | T1334 | −75.26 |
| D-2468 | 0.5 | T2072 | −60.05 |
| D-2469 | 0.5 | T1959 | −44.15 |
| D-2470 | 0.5 | T2072 | −51.87 |
| D-2471 | 0.5 | T1959 | −34.89 |
| D-2472 | 0.5 | T2077 | −43.85 |
| D-2473 | 0.5 | T2072 | −60.33 |
| D-2474 | 0.5 | T1959 | −38.56 |
| D-2475 | 0.5 | T1130 | −38.23 |
| D-2476 | 0.5 | T1334 | −65.84 |
| D-2477 | 0.5 | T2072 | −45.84 |
| D-2478 | 0.5 | T1959 | −50.33 |
| D-2479 | 0.5 | T1114 | −62.99 |
| D-2480 | 0.5 | T1526 | −79.32 |

TABLE 20
In vivo inhibition of human mARC1 mRNA
in AAV-hmARC1 mice - Study 16

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2078 | 0.5 | T1234 | −80.46 |
| D-2080 | 0.5 | T1233 | −78.74 |
| D-2081 | 0.5 | T1232 | −71.47 |
| D-2082 | 0.5 | T2072 | −63.84 |
| D-2105 | 0.5 | T2059 | −62.69 |
| D-2136 | 0.5 | T1405 | −32.32 |
| D-2241 | 3 | T1233 | −96.62 |
| D-2241 | 1 | T1233 | −92.47 |
| D-2241 | 0.5 | T1233 | −84.8 |
| D-2243 | 3 | T1234 | −97.39 |
| D-2243 | 1 | T1234 | −94.88 |
| D-2243 | 0.5 | T1234 | −83.38 |
| D-2246 | 3 | T1233 | −95.55 |
| D-2246 | 1 | T1233 | −92.55 |
| D-2246 | 0.5 | T1233 | −81.55 |
| D-2255 | 3 | T1232 | −95.38 |
| D-2255 | 1 | T1232 | −84.2 |
| D-2255 | 0.5 | T1232 | −76.6 |
| D-2258 | 3 | T1234 | −96.65 |
| D-2258 | 1 | T1234 | −89.16 |
| D-2258 | 0.5 | T1234 | −79.98 |
| D-2301 | 0.5 | T1231 | −87.12 |
| D-2316 | 0.5 | T1233 | −76.86 |
| D-2317 | 0.5 | T1232 | −66.58 |
| D-2318 | 0.5 | T2072 | −54.2 |
| D-2341 | 0.5 | T1233 | −90.21 |
| D-2344 | 0.5 | T1234 | −72.37 |
| D-2481 | 0.5 | T1526 | −78.98 |
| D-2072 | 0.5 | T1526 | −71.36 |

TABLE 21
In vivo inhibition of human mARC1 mRNA
in AAV-hmARC1 mice - Study 17

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2057 | 0.5 | T788 | −36.45 |
| D-2060 | 0.5 | T1130 | −49.77 |
| D-2081 | 0.5 | T1232 | −78.72 |
| D-2188 | 0.5 | T914 | −41.93 |
| D-2196 | 3 | T2034 | −94.08 |
| D-2196 | 1 | T2034 | −78.27 |
| D-2196 | 0.5 | T2034 | −67.92 |
| D-2225 | 3 | T1458 | −91.32 |
| D-2225 | 1 | T1458 | −79.05 |
| D-2225 | 0.5 | T1458 | −57.61 |
| D-2238 | 0.5 | T1458 | −74.65 |
| D-2260 | 3 | T2074 | −91.09 |
| D-2260 | 1 | T2074 | −74.13 |
| D-2260 | 0.5 | T2074 | −56.43 |
| D-2384 | 0.5 | T1372 | −76.06 |
| D-2391 | 0.5 | T1372 | −69.13 |
| D-2399 | 0.5 | T1372 | −75.08 |
| D-2399 | 1 | T1372 | −78.92 |
| D-2399 | 3 | T1372 | −95.3 |
| D-2401 | 0.5 | T1372 | −56.74 |
| D-2401 | 1 | T1372 | −84.24 |
| D-2401 | 3 | T1372 | −91.75 |
| D-2403 | 0.5 | T1372 | −58.73 |
| D-2462 | 3 | T1114 | −86.71 |
| D-2462 | 1 | T1114 | −55.52 |
| D-2462 | 0.5 | T1114 | −35.15 |
| D-2465 | 3 | T2072 | −91.63 |
| D-2465 | 1 | T2072 | −67.47 |
| D-2465 | 0.5 | T2072 | −62.5 |

TABLE 22
In vivo inhibition of human mARC1 mRNA
in AAV-hmARC1 mice - Study 18

| | | | Avg. % |
|---|---|---|---|
| | | | Change |
| | | | in |
| Treatment | | Trigger | human |
| (duplex | Dose | Family | mARC1 |
| no.) | (mg/kg) | Designation | mRNA |
| D-2081 | 0.5 | T1232 | −80.49 |
| D-2483 | 0.5 | T788 | −19.16 |
| D-2484 | 0.5 | T1475 | −48.32 |
| D-2485 | 0.5 | T1273 | −34.62 |
| D-2486 | 0.5 | T2102 | −50.34 |
| D-2487 | 0.5 | T2070 | −44.11 |
| D-2488 | 0.5 | T1366 | −55.46 |
| D-2489 | 0.5 | T788 | −15.04 |
| D-2490 | 0.5 | T1475 | −71.42 |
| D-2491 | 0.5 | T1273 | −47.55 |
| D-2492 | 0.5 | T2102 | −59.06 |
| D-2493 | 0.5 | T2070 | −51.01 |
| D-2494 | 0.5 | T1366 | −65.43 |
| D-2495 | 0.5 | T788 | −46.33 |
| D-2496 | 0.5 | T1475 | −35.85 |
| D-2497 | 0.5 | T1273 | −59.29 |
| D-2498 | 0.5 | T2102 | −21.14 |
| D-2499 | 0.5 | T2070 | −15.52 |
| D-2500 | 0.5 | T1366 | −35.23 |
| D-2501 | 0.5 | T788 | −23.95 |
| D-2502 | 0.5 | T1475 | −53.81 |
| D-2503 | 0.5 | T1273 | −52.52 |
| D-2504 | 0.5 | T2102 | −66.42 |
| D-2505 | 0.5 | T2070 | −37.75 |
| D-2506 | 0.5 | T1366 | −62.14 |
| D-2507 | 0.5 | T788 | −45.32 |
| D-2509 | 0.5 | T1273 | −15.16 |
| D-2510 | 0.5 | T2102 | −80.41 |
| D-2511 | 0.5 | T2070 | −65.62 |
| D-2512 | 0.5 | T1366 | −68.19 |

Two mARC1 siRNA molecules, which exhibited significant silencing activity in early in vivo studies (duplex nos. D-2042 and D-2081), were used as benchmark compounds in later in vivo studies. Seventy mARC1 siRNA molecules produced a 75% or greater reduction of human mARC1 mRNA in the AAV-hmARC1 mice at four weeks following a single s.c. injection at a dose of 1 mg/kg. Some of the tested mARC1 siRNA molecules, including D-2081, D-2241, D-2255, and D-2258, were particularly potent as evidenced by an 85% or greater reduction of human mARC1 mRNA at four weeks with just a single s.c. injection of 0.5 mg/kg. In addition, mARC1 siRNA molecules targeting certain regions of the human mARC1 transcript were observed to produce greater reductions of human mARC1 mRNA in vivo as compared to mARC1 siRNA molecules targeting other regions of the transcript. For example, mARC1 siRNA molecules with antisense strands having a sequence complementary to a region of the human mARC1 transcript (SEQ ID NO: 1) between nucleotides 1205 to 1250, nucleotides 1345 to 1375, or nucleotides 2039 to 2078 exhibited significant knockdown activity four weeks after a single s.c. injection at 1 mg/kg (Table 23). Table 23 summarizes the average percent change in human mARC1 mRNA liver levels from the studies described above for siRNA molecules having the same chemical modification pattern and targeting the human transcript at the indicated nucleotide range. mARC1 siRNA molecules targeting the human transcript between nucleotides 1211 to 1236 were especially efficacious as administration of a single s.c. dose of 1 mg/kg of such siRNA molecules reduced human mARC1 mRNA levels by greater than 80% for at least four weeks following dosing.

TABLE 23
Summary of in vivo efficacy for mARC1 siRNA molecules targeting specific
transcript regions

| | | | | |
|---|---|---|---|---|
| | | | | Avg. % |
| | Target site | | | change in |
| | within | | | human |
| | human MARC1 | | | mARC1 |
| | transcript | | | mRNA at 4 |
| Duplex | (SEQ ID | Antisense sequence | Antisense sequence | weeks |
| No. | NO: 1) | (unmodified) | (modified) | (1 mg/kg) |

Human MARC1 transcript region 1

| | | | | |
|---|---|---|---|---|
| D-2066 | 1207- | AUAAUAUUCCAGGACAUACGGUU | asUfsaauaUfuccaggAfcAfuacggsusu | -54.74 |
| | 1227 | (SEQ ID NO: 1053) | (SEQ ID NO: 3324) | |
| D-2063 | 1209- | AUCUAAUAUUCCAGGACAUACUU | asUfscuaaUfauuccaGfgAfcauacsusu | -69.57 |
| | 1229 | (SEQ ID NO: 1054) | (SEQ ID NO: 3321) | |
| D-2142 | 1210- | AAUCUAAUAUUCCAGGACAUAUU | asAfsucuaAfuauuccAfgGfacauasusu | -73.09 |
| | 1230 | (SEQ ID NO: 1055) | (SEQ ID NO: 3394) | |
| D-2301 | 1211- | ACAUCUAAUAUUCCAGGACAUUU | asCfsaucuAfauauucCfaGfgacaususu | -81.85 |
| | 1231 | (SEQ ID NO: 1055) | (SEQ ID NO: 3501) | |
| D-2081 | 1212- | AGCAUCUAAUAUUCCAGGACAUU | asGfscaucUfaauauuCfcAfggacasusu | -87.30¹ |
| | 1232 | (SEQ ID NO: 1057) | (SEQ ID NO: 3339) | |
| D-2080 | 1213- | AGGCAUCUAAUAUUCCAGGACUU | asGfsgcauCfuaauauUfcCfaggacsusu | -88.32² |
| | 1233 | (SEQ ID NO: 1058) | (SEQ ID NO: 3338) | |
| D-2078 | 1214- | AAGGCAUCUAAUAUUCCAGGAUU | asAfsggcaUfcuaauaUfuCfcaggasusu | -86.49² |
| | 1234 | (SEQ ID NO: 1059) | (SEQ ID NO: 3336) | |
| D-2077 | 1215- | AAAGGCAUCUAAUAUUCCAGGUU | asAfsaggcAfucuaauAfuUfccaggsusu | -87.22 |
| | 1235 | (SEQ ID NO: 1060) | (SEQ ID NO: 3335) | |
| D-2076 | 1216- | AAAAGGCAUCUAAUAUUCCAGUU | asAfsaaggCfaucuaaUfaUfuccagsusu | -86.21 |
| | 1236 | (SEQ ID NO: 1061) | (SEQ ID NO: 3334) | |
| D-2113 | 1219- | UUUAAAAGGCAUCUAAUAUUCUU | usUfsuaaaAfggcaucUfaAfuauucsusu | -76.41 |
| | 1239 | (SEQ ID NO: 1196) | (SEQ ID NO: 3371) | |
| D-2108 | 1227- | AGAACAUUUUUAAAAGGCAUCUU | asGfsaacaUfuuuuaaAfaGfgcaucsusu | -79.83 |
| | 1247 | (SEQ ID NO: 1197) | (SEQ ID NO: 3366) | |
| D-2067 | 1248- | AUUCAAGUGUUGUCAUUUUUGUU | asUfsucaaGfuguuguCfaUfuuuugsusu | -27.15 |
| | 1268 | (SEQ ID NO: 969) | (SEQ ID NO: 3325) | |

Human MARC1 transcript region 2

| | | | | |
|---|---|---|---|---|
| D-2013 | 1344- | AAUUGAAGCAUUGAGACACCAUU | asAfsuugaAfgcauugAfgAfcaccasusu | -17.3 |
| | 1364 | (SEQ ID NO: 842) | (SEQ ID NO: 2754) | |
| D-2304 | 1345- | ACAUUGAAGCAUUGAGACACCUU | asCfsauugAfagcauuGfaGfacaccsusu | -67.08 |
| | 1365 | (SEQ ID NO: 843) | (SEQ ID NO: 3504) | |
| D-2305 | 1346- | AACAUUGAAGCAUUGAGACACUU | asAfscauuGfaagcauUfgAfgacacsusu | -71.92 |
| | 1366 | (SEQ ID NO: 844) | (SEQ ID NO: 3505) | |
| D-2047 | 1347- | AGACAUUGAAGCAUUGAGACAUU | asGfsacauUfgaagcaUfuGfagacasusu | -58.4 |
| | 1367 | (SEQ ID NO: 845) | (SEQ ID NO: 2788) | |
| D-2306 | 1349- | UGGGACAUUGAAGCAUUGAGAUU | usGfsggacAfuugaagCfaUfugagasusu | -64.17 |
| | 1369 | (SEQ ID NO: 846) | (SEQ ID NO: 3506) | |
| D-2052 | 1350- | AUGGGACAUUGAAGCAUUGAGUU | asUfsgggaCfauugaaGfcAfuugagsusu | -84.66³ |
| | 1370 | (SEQ ID NO: 847) | (SEQ ID NO: 2793) | |
| D-2042 | 1352- | AACUGGGACAUUGAAGCAUUGUU | asAfscuggGfacauugAfaGfcauugsusu | -73.61⁴ |
| | 1372 | (SEQ ID NO: 848) | (SEQ ID NO: 2783) | |
| D-2307 | 1353- | ACACUGGGACAUUGAAGCAUUUU | asCfsacugGfgacauuGfaAfgcauususu | -57.28 |
| | 1373 | (SEQ ID NO: 973) | (SEQ ID NO: 3507) | |
| D-2308 | 1354- | UGCACUGGGACAUUGAAGCAUUU | usGfscacuGfggacauUfgAfagcaususu | -54.28 |
| | 1374 | (SEQ ID NO: 849) | (SEQ ID NO: 3508) | |
| D-2043 | 1355- | UUGCACUGGGACAUUGAAGCAUU | usUfsgcacUfgggacaUfuGfaagcasusu | -72.11 |
| | 1375 | (SEQ ID NO: 850) | (SEQ ID NO: 2784) | |
| D-2075 | 1362- | UUACUUUUUGCACUGGGACAUUU | usUfsacuuUfuugcacUfgGfgacaususu | -52.85 |
| | 1382 | (SEQ ID NO: 1220) | (SEQ ID NO: 3333) | |

Human MARC1 transcript region 3

| | | | | |
|---|---|---|---|---|
| D-2017 | 2014- | UAGAUAUUGGGUUUUAAACAAUU | usAfsgauaUfuggguuUfuAfaacaasusu | -70.1 |
| | 2034 | (SEQ ID NO: 914) | (SEQ ID NO: 2758) | |
| D-2105 | 2039- | UAGAGUUAUACAAUCAGUUAAUU | usAfsgaguUfauacaaUfcAfguuaasusu | -70.02 |
| | 2059 | (SEQ ID NO: 1333) | (SEQ ID NO: 3363) | |
| D-2106 | 2040- | UUAGAGUUAUACAAUCAGUUAUU | usUfsagagUfuauacaAfuCfaguuasusu | -64.34 |
| | 2060 | (SEQ ID NO: 1334) | (SEQ ID NO: 3364) | |
| D-2065 | 2048- | AUCAGAUCUUAGAGUUAUACAUU | asUfscagaUfcuuagaGfuUfauacasusu | -73.42 |
| | 2068 | (SEQ ID NO: 1073) | (SEQ ID NO: 3323) | |
| D-2302 | 2050- | UCAUCAGAUCUUAGAGUUAUAUU | usCfsaucaGfaucuuaGfaGfuuauasusu | -72.03 |
| | 2070 | (SEQ ID NO: 1074) | (SEQ ID NO: 3502) | |
| D-2143 | 2051- | UUCAUCAGAUCUUAGAGUUAUUU | usUfscaucAfgaucuuAfgAfguuaususu | -47.83 |
| | 2071 | (SEQ ID NO: 1075) | (SEQ ID NO: 3395) | |
| D-2082 | 2052- | AUUCAUCAGAUCUUAGAGUUAUU | asUfsucauCfagaucuUfaGfaguuasusu | -74.89 |
| | 2072 | (SEQ ID NO: 1075) | (SEQ ID NO: 3340) | |
| D-2137 | 2053- | ACUUCAUCAGAUCUUAGAGUUUU | asCfsuucaUfcagaucUfuAfgaguususu | -28.29 |
| | 2073 | (SEQ ID NO: 1077) | (SEQ ID NO: 3389) | |
| D-2079 | 2054- | UACUUCAUCAGAUCUUAGAGUUU | usAfscuucAfucagauCfuUfagagususu | -78.84² |
| | 2074 | (SEQ ID NO: 1078) | (SEQ ID NO: 3337) | |
| D-2045 | 2057- | AUAUACUUCAUCAGAUCUUAGUU | asUfsauacUfucaucaGfaUfcuuagsusu | -69.65⁵ |
| | 2077 | (SEQ ID NO: 916) | (SEQ ID NO: 2786) | |
| D-2303 | 2058- | AAUAUACUUCAUCAGAUCUUAUU | asAfsuauaCfuucaucAfgAfucuuasusu | -54.08 |
| | 2078 | (SEQ ID NO: 917) | (SEQ ID NO: 3503) | |
| D-2019 | 2079- | AAGGACAAAAUGGCAAUAAAAUU | asAfsggacAfaaauggCfaAfuaaaasusu | -43.07 |
| | 2099 | (SEQ ID NO: 920) | (SEQ ID NO: 2760) | |

¹Average from 1 mg/kg dose groups in studies 3, 6, and 8 (Tables 7, 10, and 12, respectively)
²Average from 1 mg/kg dose groups in studies 3 and 6 (Tables 7 and 10, respectively)
³Average from 1 mg/kg dose groups in studies 2 and 5 (Tables 6 and 9, respectively)
⁴Average from 1 mg/kg dose groups in studies 1, 4, 5, 6, 7, 9, 10, 12, and 13 (Tables 5, 8, 9, 10, 11, 13, 14, 16 and 17, respectively)
⁵Average from 1 mg/kg dose groups in studies 1 and 13 (Tables 5 and 17, respectively)

### Example 5. Efficacy of mARC1 siRNA in Treatment of NASH in a Mouse Model

To determine whether inhibition of mARC1 expression may be therapeutic for fatty liver diseases, mice on a 0.2% cholesterol diet (TD190883 diet) were administered an siRNA molecule targeting the mouse Marc1 gene or a control siRNA molecule. The TD190883 diet contains 0.2% cholesterol, 20% fructose, 12% sucrose, and 22% hydrogenated vegetable oil (HVO). Similar diets have been shown to induce features of NAFLD and NASH in mice placed on the diet over several weeks (see, e.g., Zhong et al., Digestion, Vol. 101:522-535, 2020 and Kroh et al., Gastroenterol Res Pract. Vol. 2020:7347068, 2020, doi:10.1155/2020/7347068).

6-week-old male c57BL/6 mice (Charles River Laboratories) were fed standard chow (Harlan, 2020× Teklad global soy protein-free extruded rodent diet) or 0.2% cholesterol diet (TD190883, Envigo). Mice on the 0.2% cholesterol diet received, by subcutaneous injection, buffer alone (phosphate-buffered saline), mARC1-targeted siRNA (duplex no. D-1000), or a control siRNA (duplex no. D-1002) at 3 mg/kg body weight in 0.2 ml buffer once every two weeks for 24 weeks. The siRNA molecules were synthesized and conjugated to a trivalent GalNAc moiety (structure shown in Formula VII) as described in Example 2. The structure of each of the siRNA molecules is provided in Tables 1 and 2. Animals were fasted and harvested on week 24 for further analysis. Liver total RNA from harvested animals was processed for qPCR analysis and serum parameters were measured by clinical analyzer (AU400 Chemistry Analyzer, Olympus). mRNA levels were first normalized to 18S ribosomal RNA levels in each liver sample, and then compared to the expression levels in the chow control group. Data were presented as relative fold over expression in the chow control group. Liver tissues were homogenized and extracted by isopropanol for total cholesterol and total triglyceride measurement (ThermoFisher, Infinity cholesterol and Infinity triglyceride). All animal housing conditions and research protocols were approved by the Amgen Institutional Animal Care and Use Committee (IACUC). Mice were housed in a specified-pathogen free, AAALAC, Intl-accredited facility in ventilated microisolators. Procedures and housing rooms were positively pressured and regulated on a 12:12 dark:light cycle. All animals received reverse-osmosis purified water ad libitum via an automatic watering system.

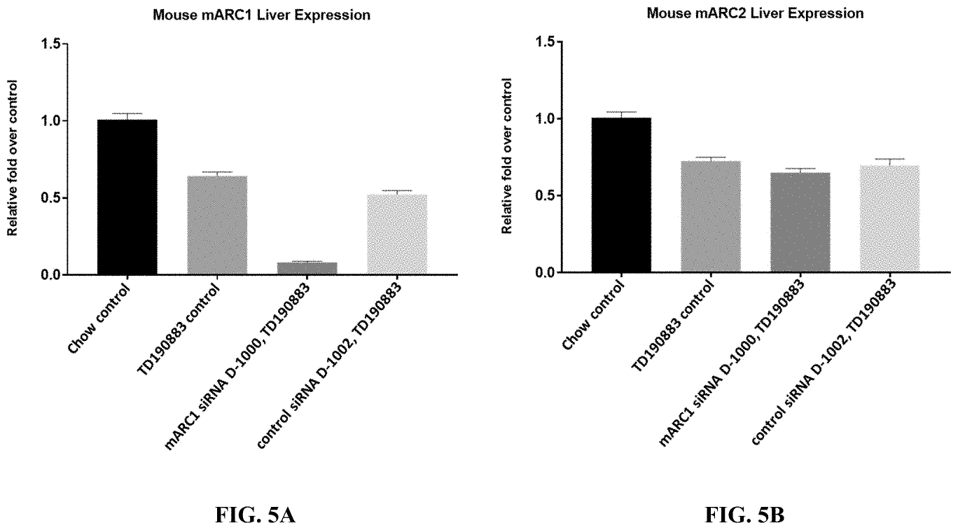

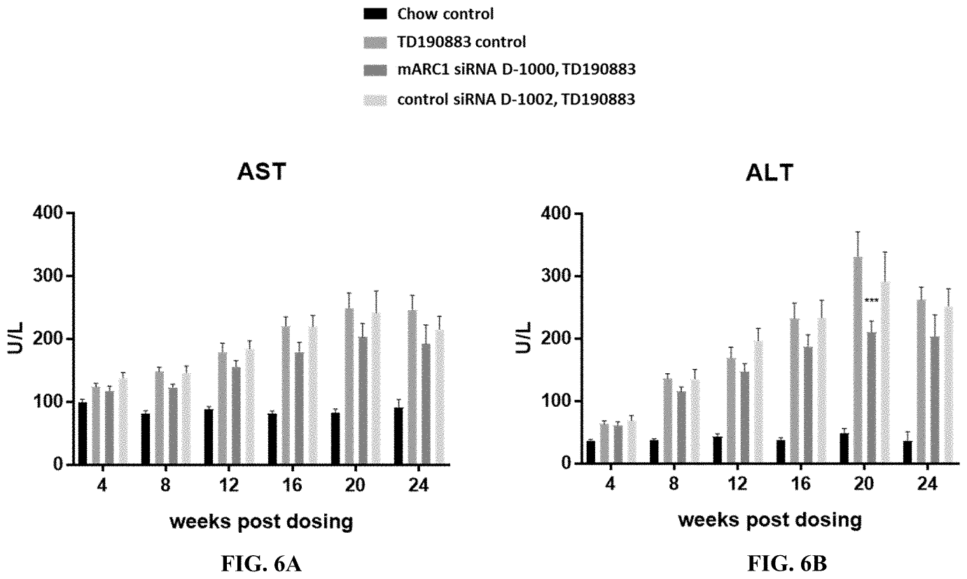

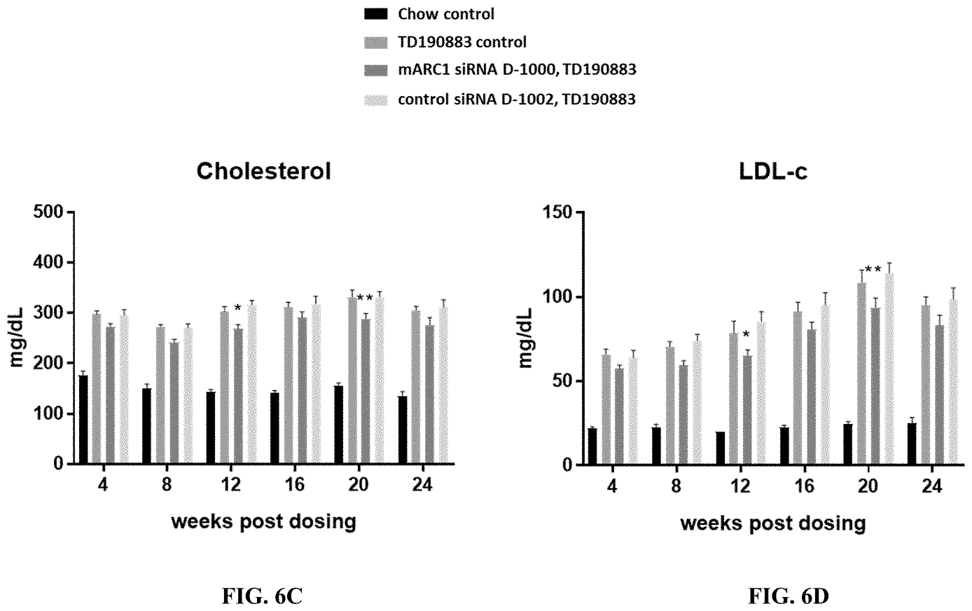

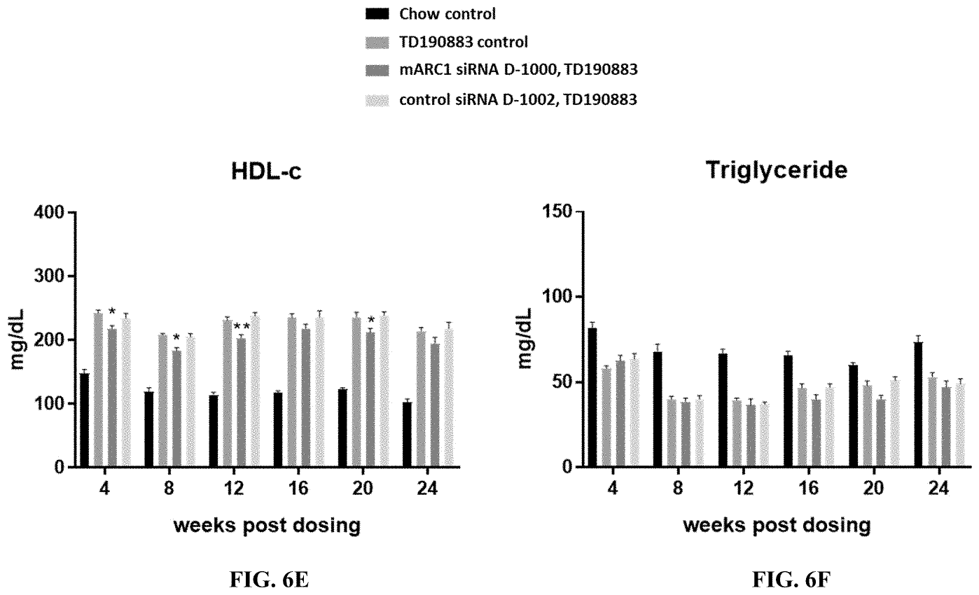

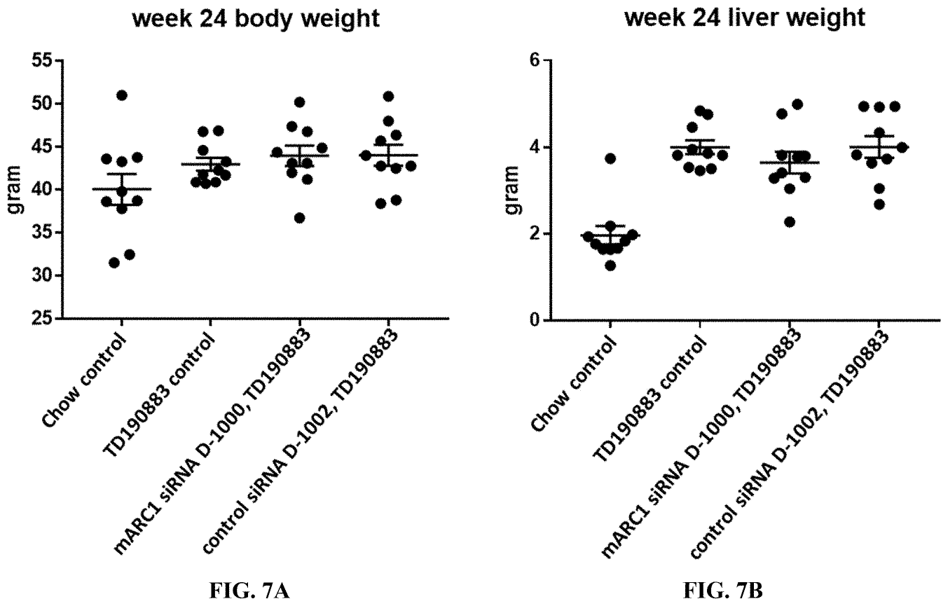

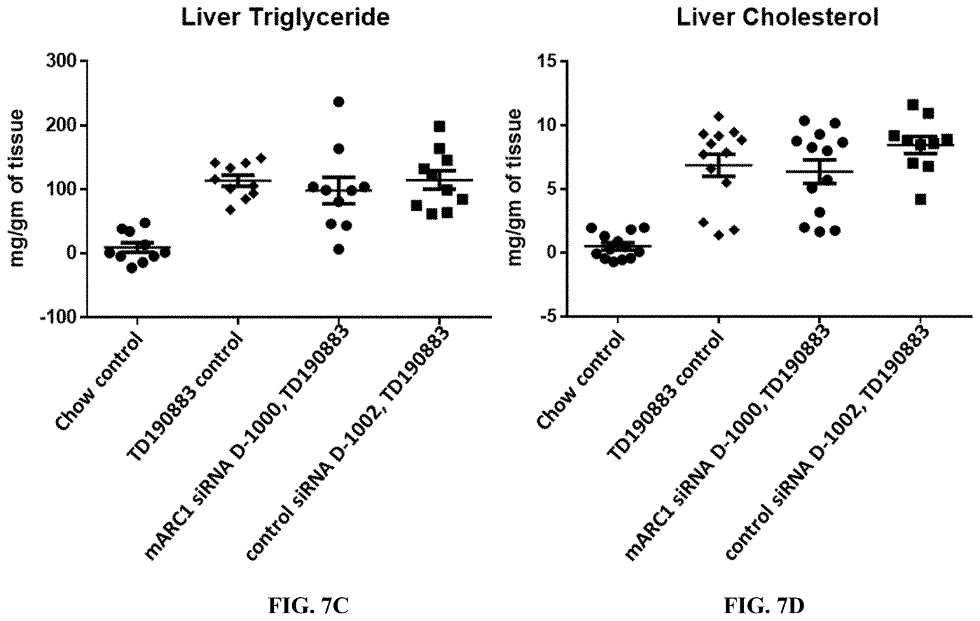

Liver expression of both mARC1 and mARC2 was reduced in mice fed the 0.2% cholesterol diet. mARC1 expression, but not mARC2 expression, was further reduced in animals treated with the mARC1-targeted siRNA (FIGS. 5A and 5B). As expected, mice on the 0.2% cholesterol diet had increased serum levels of liver enzymes (AST and ALT), cholesterol, LDL-cholesterol (LDL-C) and HDL-cholesterol (HDL-C) over the course of the study (FIGS. 6A-6E). Treatment with the mARC1-targeted siRNA reduced the diet-induced increases in serum cholesterol, LDL-C and HDL-C (FIGS. 6C-6E). The mARC1 siRNA treatment also showed a trend in reducing diet-induced serum levels of liver enzymes (FIGS. 6A-6B). Animals on the 0.2% cholesterol diet had increased body and liver weight after 24 weeks (FIGS. 7A and 7B). Triglyceride and cholesterol levels in the liver were also increased in animals on the 0.2% cholesterol diet at 24 weeks (FIGS. 7C and 7D). mARC1 siRNA treatment did not significantly reduce the diet-induced increases in body weight, liver weight, liver triglyceride levels or liver cholesterol levels (FIGS. 7A-7D).

In sum, the results of this study show that inhibition of mARC1 liver expression with a mARC1-targeted siRNA molecule reduces serum cholesterol, LDL-C, HDL-C, and liver enzymes in a mouse model of NASH, suggesting that mARC1 siRNA molecules may be a novel therapeutic approach for treating this disease and other fatty liver disorders.

### Example 6. Impact of Mismatches on Potency of mARC1 siRNA Molecules

To assess the effect of base pair mismatches on the potency of mARC1 siRNA molecules, analogs of a subset of the most potent siRNA molecules were synthesized to have a different nucleotide at positions 6 or 8 from the 5′ end of the antisense strand such that a base pair mismatch was created at that position when the antisense strand hybridized to its target region of the mARC1 mRNA transcript. However, in each analog, the sequence of the sense strand was designed to be fully complementary to the sequence of the antisense strand so no mismatches were created between the sense and antisense strands in the siRNA duplex. The unmodified and modified sequences for each of the mismatch analogs (duplex nos. D-2514 to D-2561) and the parental siRNA molecules (duplex nos. D-2052, D-2072, D-2076, D-2077, D-2079, D-2081, D-2105, D-2108, D-2111, D-2113, D-2115, D-2118, D-2142, D-2136, D-2189, D-2196, D-2238, D-2241, D-2254, D-2258, D-2301, D-2462, D-2465, and D-2510) are provided in Tables 1 and 2, respectively. The efficacy of the mismatch analogs and the parental siRNA molecules in reducing human mARC1 mRNA levels was evaluated in Hep3B cells using the in vitro RNA FISH assay described in Example 3 above. Ten different concentrations of each of the siRNA molecules ranging from 100 nM to 5 pM were tested, and IC50 and maximum activity values were calculated from the dose response curves as described in Example 3. The results of these assays are shown in Table 24 below.

TABLE 24
In vitro efficacy of mARC1 siRNA mismatch analogs in Hep3B cells

| | Target site | Mismatch | | |
|---|---|---|---|---|
| | within human | Position | | |
| | MARC1 | from 5′ end | | |
| | transcript (SEQ | of antisense | | Max |
| Duplex No. | ID NO: 1) | strand | IC50 [M] | Activity |
| D-2254 | 985-1005 | none | 4.17E−09 | −83.59 |
| D-2514 | 985-1005 | 6 | — | −74.91 |
| D-2515 | 985-1005 | 8 | 2.97E−08 | −78.35 |
| D-2462 | 1092-1114 | none | 6.9E−10 | −93.14 |
| D-2516 | 1092-1114 | 6 | 2.00E−08 | −79.30 |
| D-2517 | 1092-1114 | 8 | 1.54E−09 | −87.85 |
| D-2142 | 1210-1230 | none | 6.82E−10 | −90.23 |
| D-2518 | 1210-1230 | 6 | 3.34E−09 | −82.58 |
| D-2519 | 1210-1230 | 8 | 4.63E−09 | −85.78 |
| D-2301 | 1211-1231 | none | 3.3E−10 | −84.34 |
| D-2520 | 1211-1231 | 6 | 7.59E−09 | −79.51 |
| D-2521 | 1211-1231 | 8 | 1.49E−08 | −70.30 |
| D-2081 | 1212-1232 | none | 5.88E−10 | −86.84 |
| D-2522 | 1212-1232 | 6 | 2.91E−09 | −85.61 |
| D-2523 | 1212-1232 | 8 | 2.33E−09 | −89.43 |
| D-2241 | 1215-1233 | none | 1.26E−09 | −86.29 |
| D-2524 | 1215-1233 | 6 | 2.51E−08 | −82.90 |
| D-2525 | 1215-1233 | 8 | 5.49E−09 | −85.28 |
| D-2258 | 1214-1234 | none | 7.55E−10 | −81.90 |
| D-2526 | 1214-1234 | 6 | 3.37E−09 | −86.17 |
| D-2527 | 1214-1234 | 8 | 2.24E−08 | −73.80 |
| D-2077 | 1215-1235 | none | 4.42E−10 | −87.32 |
| D-2528 | 1215-1235 | 6 | 5.33E−09 | −86.59 |
| D-2529 | 1215-1235 | 8 | 5.6E−09 | −86.43 |
| D-2076 | 1216-1236 | none | 5.41E−10 | −89.64 |
| D-2530 | 1216-1236 | 6 | 1.79E−08 | −82.25 |
| D-2531 | 1216-1236 | 8 | 2.52E−09 | −82.91 |
| D-2113 | 1219-1239 | none | 5.86E−10 | −86.53 |
| D-2532 | 1219-1239 | 6 | 1.10E−08 | −82.08 |
| D-2533 | 1219-1239 | 8 | 6.44E−09 | −76.61 |
| D-2108 | 1227-1247 | none | 1.44E−09 | −85.90 |
| D-2534 | 1227-1247 | 6 | 4.7E−09 | −78.40 |
| D-2535 | 1227-1247 | 8 | 3.69E−09 | −85.20 |
| D-2111 | 1314-1334 | none | 2.78E−10 | −88.86 |
| D-2536 | 1314-1334 | 6 | — | −31.51 |
| D-2537 | 1314-1334 | 8 | 4.7E−09 | −83.69 |
| D-2052 | 1350-1370 | none | 5.75E−10 | −80.89 |
| D-2538 | 1350-1370 | 6 | 1.49E−08 | −75.03 |
| D-2539 | 1350-1370 | 8 | 2.19E−09 | −81.35 |
| D-2189 | 1352-1372 | none | 1.49E−09 | −85.52 |
| D-2540 | 1352-1372 | 6 | — | −76.77 |
| D-2541 | 1352-1372 | 8 | 4.1E−09 | −88.64 |
| D-2136 | 1385-1405 | none | 9.11E−10 | −84.91 |
| D-2542 | 1385-1405 | 6 | — | −16.91 |
| D-2543 | 1385-1405 | 8 | 3.21E−08 | −70.17 |
| D-2238 | 1438-1458 | none | 7.37E−10 | −77.36 |
| D-2544 | 1438-1458 | 6 | 1.12E−08 | −61.11 |
| D-2545 | 1438-1458 | 8 | 7.51E−09 | −82.10 |
| D-2072 | 1506-1526 | none | 8.57E−10 | −87.83 |
| D-2546 | 1506-1526 | 6 | 8.49E−09 | −83.30 |
| D-2547 | 1506-1526 | 8 | 2.68E−09 | −87.92 |
| D-2115 | 1997-2017 | none | 5.67E−10 | −82.42 |
| D-2548 | 1997-2017 | 6 | 8.32E−09 | −84.98 |
| D-2549 | 1997-2017 | 8 | 2.82E−09 | −83.58 |
| D-2196 | 2016-2034 | none | 1.38E−09 | −82.91 |
| D-2550 | 2016-2034 | 6 | 1.85E−08 | −78.12 |
| D-2551 | 2016-2034 | 8 | — | −75.52 |
| D-2105 | 2039-2059 | none | 7.52E−10 | −89.10 |
| D-2552 | 2039-2059 | 6 | 1.45E−08 | −83.79 |
| D-2553 | 2039-2059 | 8 | 4.00E−09 | −82.31 |
| D-2465 | 2052-2072 | none | 5.98E−10 | −84.77 |
| D-2554 | 2052-2072 | 6 | 6.74E−09 | −77.83 |
| D-2555 | 2052-2072 | 8 | 2.05E−09 | −86.44 |
| D-2079 | 2054-2074 | none | 4.03E−10 | −85.54 |
| D-2556 | 2054-2074 | 6 | 2.74E−09 | −71.14 |
| D-2557 | 2054-2074 | 8 | 3.57E−09 | −84.85 |
| D-2510 | 2082-2102 | none | 4.08E−10 | −81.51 |
| D-2558 | 2082-2102 | 6 | 2.35E−08 | −62.54 |
| D-2559 | 2082-2102 | 8 | 1.61E−09 | −84.40 |
| D-2118 | 2086-2106 | none | 4.64E−10 | −82.20 |
| D-2560 | 2086-2106 | 6 | 9.57E−09 | −75.61 |
| D-2561 | 2086-2106 | 8 | 7.37E−09 | −83.48 |

For the majority of the molecules, the mismatches at positions 6 and 8, which are located within the seed region of the antisense strand, did not significantly affect the maximum knockdown activity or the potency of the siRNA molecules as compared to the parental molecules in which the antisense strand was fully complementary to the target mARC1 mRNA sequence. These results are somewhat surprising as the seed region of the antisense strand (i.e. nucleotides 2 to 8 from the 5′ end) is believed to be important for on-target efficacy.

### Example 7. In Vivo Efficacy of mARC1 siRNA Molecules in Non-Human Primates

Efficacy and pharmacokinetic profile of three different mARC1 siRNA molecules (duplex nos. D-2241, D-2081, or D-2258) were evaluated in cynomolgus monkeys. Each of the three different mARC1 siRNA molecules had antisense strand sequences that cross-reacted with the cynomolgus monkey (Macaca fascicularis) MARC1 gene. Female treatment-naïve cynomolgus macaque monkeys, ages 22 to 48 months, of Mauritius origin were sourced from Charles River Laboratories, Inc. Research Model Services (Houston, Tex.). Animals (n=3 per treatment group) were administered a single 3 mg/kg subcutaneous (s.c.) injection into the scapular and mid-dorsal region of GalNAc-conjugated mARC1 siRNA molecule, either duplex no. D-2241, D-2081, or D-2258, formulated in 1× phosphate buffered saline. Serum was prepared from whole blood collected at the following time points post-dose: 0.083, 0.25, 1, 2, 4, 24, 28, 96, 168, 264, 336, 456, 528, 576, 720, 864, and 1056 hours. Surgical liver biopsies (approximately 100 mg tissue per left and right liver lobe) were collected under anesthesia at pre-treatment (either days −13 or −7) and days 14 and 30 post-dose. Day 44 post-dose liver samples were collected at necropsy.

### Serum and Liver Pharmacokinetics

To determine the serum and liver pharmacokinetic profiles of each of the GalNAc-conjugated mARC1 siRNA molecules, serum and liver samples collected at different time points following treatment with a single 3 mg/kg s.c. dose of the mARC1 siRNA molecules were analyzed for each of the mARC1 siRNA molecules (antisense and sense strands) using a plate-based oligonucleotide electro-chemiluminescent (POE) immunoassay similar to that described in Thayer et al., Sci. Rep., Vol. 10(1): 10425, 2020. Oligonucleotide capture (biotin) and detection (digoxygenin) probes were custom synthesized from Qiagen Inc. (Hilden, Germany), the sequences for which are listed in Table 25 below. Liver samples were homogenized in lysis buffer containing 50 mM Tris HCl, 100 nM NaCl, 0.1% Triton X100, and Roche protease inhibitor cocktail (11836170001) to a final concentration of 200 mg/mL. For the bioanalysis, GalNAc-mARC1 siRNA standards were spiked into serum or liver homogenate over a concentration range of 0.13 to 2500 ng/mL. Standards and biological samples were then diluted 1:10 in a 96 well PCR plate to a final volume of 50 μL. Oligonucleotide capture and detection probes were prepared in a hybridization buffer consisting of 60 mM Na₂PO₄ (pH 7.0, dibasic), 1 M NaCl, 5 mM EDTA, and 0.02% Tween 20. Probes were combined and added to the PCR plate at a final concentration of 10 nM bringing the total sample volume to 100 μL per well. Hybridization was performed using a thermal cycler under the following conditions: 90° C. for 5 minutes, 40° C. for 30 minutes, and a final hold at 12° C. After hybridization, 45 μL of samples were transferred to a Meso Scale Diagnostics, LLC MSD Gold 96-well Streptavidin SECTOR plate (L15SA) and incubated at room temperature for 30 minutes while shaking. The plates were washed with SerCare Life Sciences 1×KPL immunoassay wash solution (5150-0011). After washing, plates were incubated for 1 hour with 50 μL of 0.5 μg/mL ruthenium labeled anti-digoxygenin antibody diluted in ThermoFisher Scientific SuperBlock T20 TBS Blocking Buffer (37536). A final wash was performed prior to the addition of Meso Scale Diagnostics, LLC 1×MSD Read Buffer T (R92TC; 150 μL) and read on a Meso Scale Diagnostics, LLC Meso Sector S 600 instrument. Serum and liver concentrations of the mARC1 siRNA molecules were interpolated from a standard curve using a 4-parameter logistic model and a weighting factor of 1/Y2 in Watson LIMS bioanalytical software version 7.5 (ThermoFisher Scientific). Liver concentrations were converted from units of ng/mL to ng/mg by dividing by 200 mg/mL. Serum pharmacokinetic parameters from 0.083 to 24 hours post-dose were determined using noncompartmental analysis in Phoenix WinNonlin software version 8.3.2.116 (Pharsight).

TABLE 25
POE immunoassay capture and detection probes

| Duplex | | | SEQ |
|---|---|---|---|
| No. | Strand | Sequence (5′→3′)¹ | ID NO: |
| D-2241 | Antisense | /5Biosg/ACCTGGAATA | 3659 |
| D-2241 | Antisense | TTAGATGCCT/3Di_N/ | 3660 |
| D-2241 | Sense | /5Biosg/AAGGCATCTA | 3661 |
| D-2241 | Sense | ATATTCCAGG/3Dig_N/ | 3662 |
| D-2081 | Antisense | /5Biosg/ATGTCCTGGAA | 3663 |
| D-2081 | Antisense | TATTAGATGCT/3Dig_N/ | 3664 |
| D-2081 | Sense | /5Biosg/GCATCTAATA | 3665 |
| D-2081 | Sense | TTCCAGGACA/3Dig_N/ | 3666 |
| D-2258 | Antisense | /5Biosg/CCTGGAATAT | 3667 |
| D-2258 | Antisense | TAGATGCCTT/3Dig_N/ | 3668 |
| D-2258 | Sense | /5Biosg/AGGCATCTAA | 3669 |
| D-2258 | Sense | TATTCCAGGA/3Dig_N/ | 3670 |

¹Underlined base = locked nucleic acid modification; /5Biosg/ = biotin conjugation via a six-carbon linker;
/3Dig_N/ = digoxygenin conjugation via a N-hydroxysuccinimide ester.

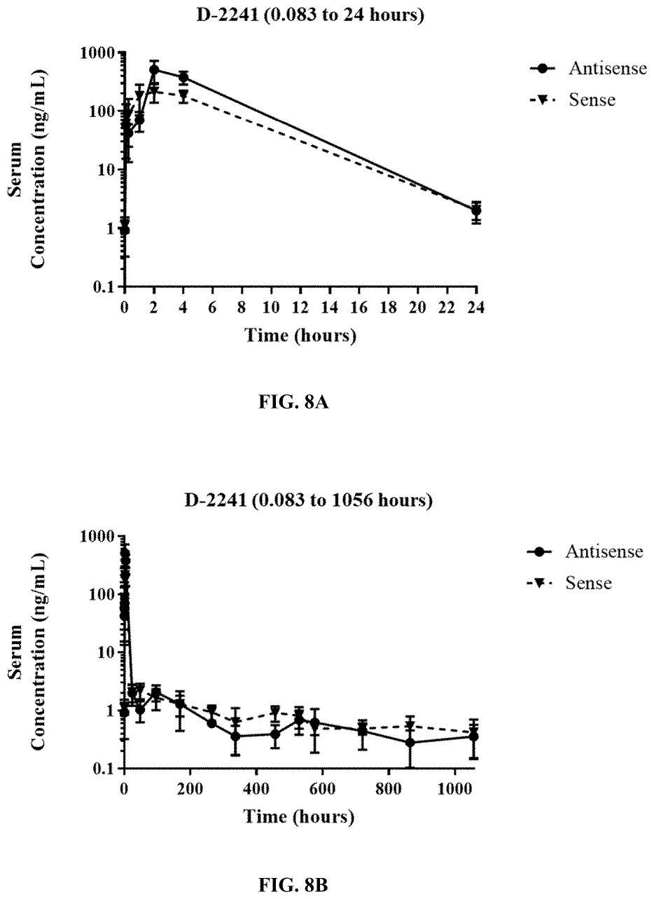

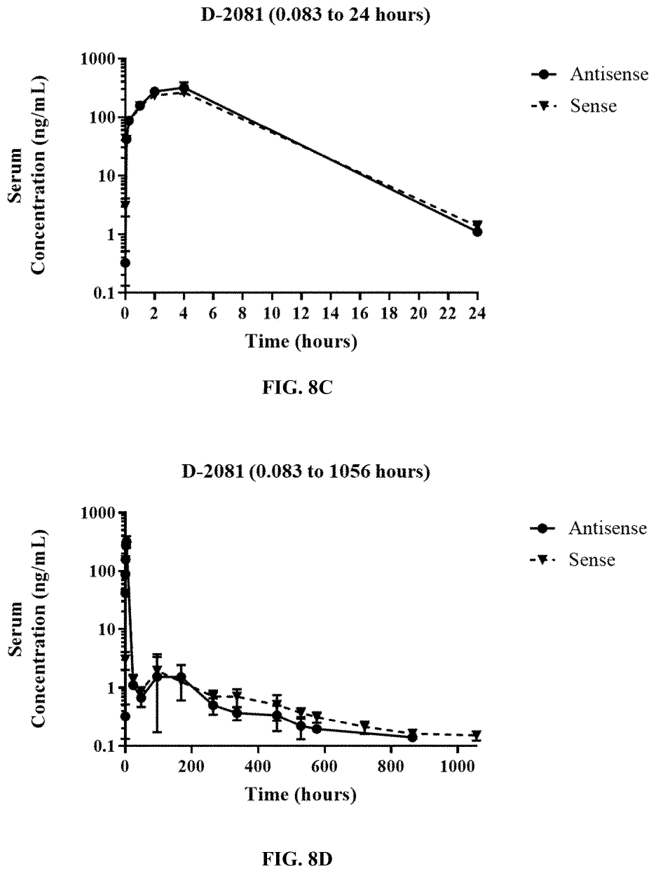

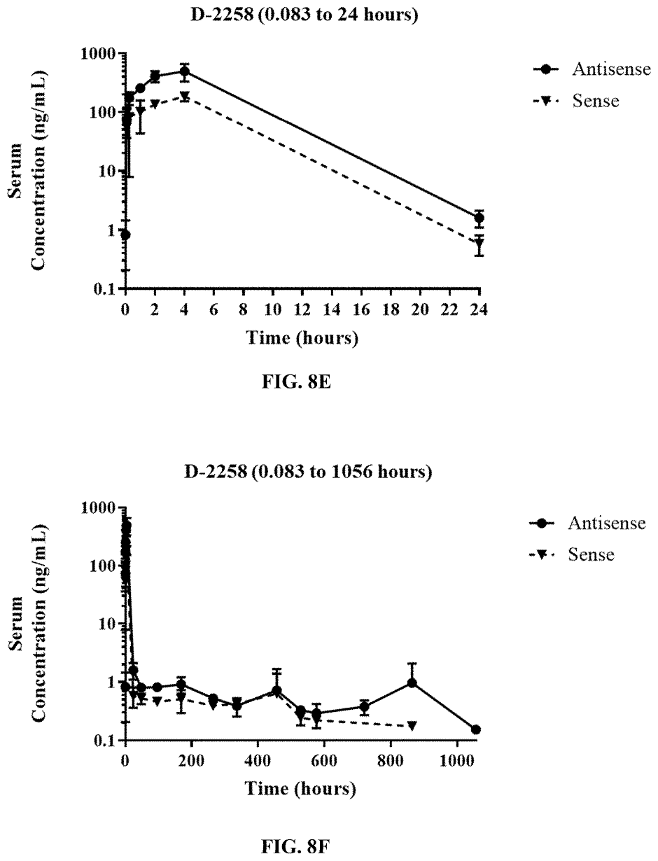

Serum concentration-time profiles for antisense and sense strand concentrations for each of the three different mARC1 siRNA molecules are shown in FIGS. 8A-8F. The mean maximum observed antisense strand concentration (Cmax) in serum was 511, 496, and 321 ng/mL for D-2241, D-2258, and D-2081, respectively, at 2.0 to 4.0 hours post-dose as summarized in Table 26. The mean area under the concentration time curve from the start of dose administration to 24 hours post-dose (AUC_{0-24 hour}) for serum antisense strands was 6399, 5040, and 4137 h*ng/mL for D-2258, D-2241, and D-2081, respectively. The ratio of the serum concentrations of the sense strand to antisense strand for duplex no. D-2258 indicates a potential instability of the duplex with strand separation possibly occurring at the site of injection or in systemic circulation. siRNA liver concentrations for antisense and sense strands on days 14, 30 and 44 post-dose are reported in Table 27. Day 14 liver antisense strand concentrations were greatest for duplex no. D-2081 followed by D-2241 and then D-2258. Consistent with the serum pharmacokinetic profile, the ratio of the liver concentrations of the sense and antisense strands for duplex no. D-2258 indicates strand separation.

TABLE 26
Antisense strand serum pharmacokinetic parameters with a single 3 mg/kg s.c.
dose of mARC1 siRNA molecules in cynomolgus macaque monkeys

| | GalNAc-conjugated mARC1 | | |
|---|---|---|---|
| Pharmacokinetic | siRNA Treatment (duplex no.) | | |
| Parameter¹ | D-2241 | D-2081 | D-2258 |
| Tₘₐₓ (h) | 2.0 | 4.0 | 4.0 |
| Cₘₐₓ (ng/mL) | 511 | 321 | 496 |
| AUC_{0-24 hour} | 5040 | 4137 | 6399 |
| (h*ng/mL) | | | |

¹Tₘₐₓ = the time after dosing at which the maximum observed concentration was observed;
Cₘₐₓ = the maximum observed concentration measured after dosing;
AUC_{0-24 hour} = the area under the concentration versus time curve using the linear trapezoidal method from the start of dose administration to 24 hours post-dose.
N = 3 animals per treatment group.

TABLE 27
Antisense and sense strand liver concentrations with a single 3 mg/kg
s.c. dose of mARC1 siRNA molecules in cynomolgus macaque monkeys

| | GaINAc-conjugated mARC1 siRNA Treatment (duplex no.) | | | | | |
|---|---|---|---|---|---|---|
| | D-2241 | | D-2081 | | D-2258 | |
| | (Mean ± SD; ng/mg) | | (Mean ± SD; ng/mg) | | (Mean ± SD; ng/mg) | |
| | Antisense | Sense | Antisense | Sense | Antisense | Sense |
| Day 14 | 28 ± 11 | 29 ± 6.7 | 39 ± 10 | 20 ± 3.5 | 14 ± 1.1 | 42 ± 3.7 |
| Post-Dose | | | | | | |
| Day 30 | 11 ± 4.5 | 12 ± 1.7 | 4.3 ± 0.37 | 11 ± 1.9 | 7.4 ± 0.60 | 29 ± 1.4 |
| Post-Dose | | | | | | |
| Day 44 | 5.9 ± 3.0 | 0.69 ± 0.29 | 2.4 ± 0.33 | 5.1 ± 0.95 | 5.1 ± 0.57 | 16 ± 3.6 |
| Post-Dose | | | | | | |

SD = standard deviation

Liver mARC1 mRNA Silencing

The three GalNAc-conjugated mARC1 siRNA molecules (duplex nos. D-2241, D-2081, and D-2258) were evaluated for efficacy in knocking down mARC1 mRNA levels in the liver of cynomolgus macaque monkeys following a 3 mg/kg s.c. dose. RNA was purified from snap frozen liver using the ThermoFisher Scientific MagMAX-96 Total RNA Isolation Kit (AM1830) of which sample integrity (260/280 ratio) and RNA concentrations were determined with a ThermoFisher Scientific NanoDrop 2000 Spectrophotometer (ND-2000). One step reverse transcription-polymerase chain reaction (RT-PCR) was performed using ThermoFisher Scientific's TaqMan™ RNA-to-CT 1-Step Kit (4392938). Reactions were assembled into a 96 well PCR plate by mixing 50 ng of RNA template with 2× TaqMan RT-PCR Mix, 40× TaqMan RT Enzyme Mix, 20× mARC1 primer-probe (IDT, forward primer 5′-TTCAGGATGCGATGT CTATGC-3′ (SEQ ID NO: 3671), reverse primer 5′-TGCCCAAAGAGTGGTGATTT-3′ (SEQ ID NO: 3672), probe 5′-/56-FAM/AGCCGCTGG (SEQ ID NO: 3673)/ZEN/AAACACT GAAGAGTT (SEQ ID NO: 3674)/3IABkFQ/-3′), and 20× glyceraldehyde-3-phosphate dehydrogenase primer-probe (GAPDH; ThermoFisher Scientific, Mf04392546_g1 VIC-MGB). RT-PCR was performed using the ThermoFisher Scientific QuantStudio 7 Flex Real-Time PCR System (4485701) under the following conditions: 48° C. for 30 minutes, and 90° C. for 10 minutes followed by 40 cycles of 90° C. for 15 seconds and 60° C. for 1 minute. mRNA expression for each sample was normalized by taking a ratio of the concentration of the gene of interest (mARC1) over the concentration of the housekeeping gene (GAPDH). Percent (%) of mARC1 mRNA expression post-siRNA dose (days 14, 30, and 44) was then calculated relative to the pre-treatment (days −13 or −7) time point for each animal replicate per treatment group, which was expressed as % remaining of pre-treatment. Percent (%) silencing of mARC1 mRNA transcript was ultimately calculated by subtracting the % remaining of pre-treatment value from 100%. Both mRNA % remaining of pre-treatment and % silencing values are summarized below in Table 28. Duplex no. D-2241 was the most potent GalNAc-conjugated mARC1 siRNA molecule tested, reducing cynomolgus mARC1 liver mRNA to <20% remaining of pre-treatment (>80% silencing) on days 14, 30, and 44 following a single subcutaneous injection.

TABLE 28
Cynomolgus macaque liver mARC1 mRNA silencing with
a single 3 mg/kg s.c. dose of GaINAc-conjugated mARC
siRNA molecules

| | GaINAc- | | | | | | | | | |
|---|---|---|---|---|---|---|---|---|---|---|
| | conjugated | | | | | | | | | |
| | mARC1 siRNA | | | | | | | | | |
| | Treatment | | | | | | | | | |
| | (duplex no.) | D-2241 | | | D-2081 | | | D-2258 | | |
| | Animal Replicate | 1 | 2 | 3 | 1 | 2 | 3 | 1 | 2 | 3 |
| Day 14 | % Remaining of | ND | 0.67 | 0.57 | 29 | 22 | 14 | 20 | 38 | 1.0 |
| Post-Dose | Pre-treatment | (0) | | | | | | | | |
| | % Silencing | 100 | 99 | 99 | 71 | 78 | 86 | 80 | 62 | 99 |
| | % Silencing; | 99 ± 0.58 | | | 78 ± 7.5 | | | 80 ± 19 | | |
| | Mean ± SD | | | | | | | | | |
| Day 30 | % Remaining of | 3.7 | 23 | 22 | 36 | 52 | 21 | 37 | 40 | 57 |
| Post-Dose | Pre-treatment | | | | | | | | | |
| | % Silencing | 96 | 77 | 78 | 64 | 48 | 79 | 63 | 60 | 43 |
| | % Silencing; | 84 ± 11 | | | 64 ± 16 | | | 55 ± 11 | | |
| | Mean ± SD | | | | | | | | | |
| Day 44 | % Remaining of | 0.20 | 23 | 21 | 47 | 41 | 8.8 | 30 | 30 | 28 |
| Post-Dose | Pre-treatment | | | | | | | | | |
| | % Silencing | 100 | 78 | 79 | 53 | 59 | 91 | 70 | 70 | 72 |

| | | | |
|---|---|---|---|
| % Silencing; | 86 ± 12 | 68 ± 21 | 71 ± 1.0 |
| Mean ± SD | | | |

ND = not detected;
SC = subcutaneous;
SD = standard deviation;
Samples in which mARC1 mRNA expression was below the limit of assay detection were denoted as “ND” (not detected) and set to zero.

Liver mARC1 Protein Silencing

Efficacy of the three GalNAc-conjugated mARC1 siRNA molecules (duplex nos. D-2241, D-2081, and D-2258) in knocking down mARC1 protein levels in the liver of cynomolgus macaque following a 3 mg/kg s.c. dose was also assessed. Snap frozen liver tissue was homogenized at 200 mg/mL in Boston Bioproduct NP-40 Lysis Buffer (BP-119) containing ThermoFisher Scientific Protease Inhibitor Tablets (A32963). Homogenates were then spun down at 10,000×g under 4° C. for 10 minutes and supernatants were transferred to a 2 mL 96 deep-well plate. Supernatants were treated with 1% trifluoroacetic acid in methanol while incubating for 15 minutes at room temperature and shaking at 1400 rpm. Precipitated proteins were pelleted for 15 minutes at 4,000 rpm from which the supernatants were aspirated and the pellets were washed twice with methanol. Resulting proteins were reduced and denatured in a solution containing 10 mM tris(2-carboxyethyl)phosphine (ThermoFisher Scientific, 77720) and 8 M urea for 30 minutes at 37° C. Iodoacetamide (20 mM; ThermoFisher Scientific, A39271) was then added to the samples in 20 mM ammonium bicarbonate buffer and incubated for 30 minutes at room temperature. Tryptic digestion was performed overnight at 37° C. with the addition of 30 μg trypsin (ThermoFisher Scientific, A90058) and 10 pmol of the stable isotopically labeled (SIL) peptide (ThermoFisher Scientific custom peptide; SPLFGQYFVLENPGTIK (SEQ ID NO: 3675)). The digestion reaction was terminated with 20% formic acid and the samples were prepared for solid phase extraction (SPE) desalting (Waters Corporation, 186008052). Prior to loading samples, the SPE plate was conditioned with methanol and washed once with 1% acetonitrile. Samples were added to the conditioned SPE plate and analytes were eluted using 70% acetonitrile. Eluates were resuspended in 10 mM ammonium formate at pH 10 and injected onto an Agilent 1260 Infinity Bio-inert Analytical-scale Fraction Collector (G5664A). The fractionated samples (11th fraction) were resuspended in 0.1% formic acid solution for analysis on a ThermoFisher Scientific Ultimate 3000 ultra-high performance liquid chromatography (LC) system coupled to an Orbitrap Lumos mass spectrometer (MS). The LC method was performed as follows: trapping at 3% acetonitrile/water, 8 μL/minute and analytical gradient at 3.0 to 36% acetonitrile/water over 1.0 to 12.1 minutes, 350 nL/minute, with a column temperature at 45° C. A parallel reaction monitoring experiment was performed on the Orbitrap Fusion Lumos instrument monitoring light- and heavy-labeled peptides SPLFGQYFVLENPGTIK (SEQ ID NO: 3675) at m/z=955.5066 and SPLFGQYFVLENPGTIK (SEQ ID NO: 3675) at m/z=959.5137, respectively. Data was then imported into Skyline 21.1 software (Pino L K et al. The Skyline ecosystem: Informatics for quantitative mass spectrometry proteomics. Mass Spectrom Rev. 2020 May; 39(3):229-244. doi: 10.1002/mas.21540. Epub 2017 Jul. 9.), where the SPLFGQYFVLENPGTIK (SEQ ID NO: 3675) peptide peak area from each sample was normalized to the peak area of the spiked-in SIL peptide SPLFGQYFVLENPGTIK (SEQ ID NO: 3675). The measurement of GAPDH housekeeping protein was performed using the same starting tissue homogenate and precipitated with ice-cold acetone followed by mixing at 1250 rpm for 10 minutes and centrifugation at 3220×g for 15 minutes. The supernatants were aspirated and protein pellets were washed with methanol, dissolved in 50 mM ammonium bicarbonate buffer containing 10 μg trypsin, and digested overnight at 37° C. with mixing at 1000 rpm. The digestion reaction was terminated with 20% formic acid and injected for LC-MS/MS analysis monitoring the GAPDH peptide: LISWYDNEFGYSNR (SEQ ID NO: 3676) at 588.61 and 743.35 m/z. The GAPDH peptide peak area was integrated using SCIEX Analyst software. Protein expression for each sample was normalized by taking a ratio of the concentration of the protein of interest (mARC1) as determined relative to the SIL peptide over the concentration of the housekeeping protein (GAPDH). Percent (%) of mARC1 protein expression post-siRNA dose (days 14, 30, and 44) was then calculated relative to the pre-treatment (days −13 or −7) time point for each animal replicate per treatment group, which was expressed as % remaining of pre-treatment. Percent (%) silencing of mARC1 protein expression was ultimately calculated by subtracting the % remaining of pre-treatment value from 100%. Both protein % remaining of pre-treatment and % silencing values are summarized in Table 29. Duplex no. D-2081 showed the greatest reduction in cynomolgus mARC1 liver protein expression on day 14 post-dose with 89±0.71% silencing following a single subcutaneous injection. On day 30 post-dose, duplex nos. D-2081 and D-2241 decreased protein expression to <20% remaining of pre-treatment with 82±7.8% and 87±11% silencing, respectively, which was maintained or increased through day 44 post-dose.

TABLE 29
Cynomolgus macaque liver mARC1 protein silencing with a single
3 mg/kg s.c. dose of GaINAc-conjugated mARC siRNA molecules

| | GaINAc- | | | | | | | | | |
|---|---|---|---|---|---|---|---|---|---|---|
| | conjugated | | | | | | | | | |
| | mARC1 | | | | | | | | | |
| | siRNA | | | | | | | | | |
| | Treatment | | | | | | | | | |
| | (duplex no.) | D-2241 | | | D-2081 | | | D-2258 | | |
| | Animal Replicate | 1 | 2 | 3 | 1 | 2 | 3 | 1 | 2 | 3 |
| Day | % Remaining of | 34 | 35 | 22 | ND | 11 | 12 | 56 | 68 | 25 |
| 14 | Pre-treatment | | | | (0) | | | | | |
| Post- | % Silencing | 66 | 65 | 78 | N/A | 89 | 88 | 44 | 32 | 75 |
| Dose | % Silencing; | 70 ± 7.2 | | | 89 ± 0.71 | | | 50 ± 22 | | |
| | Mean ± SD | | | | | | | | | |
| Day | % Remaining of | 0.99 | 14 | 22 | ND | 24 | 13 | 39 | 54 | 3.6 |
| 30 | Pre-treatment | | | | (0) | | | | | |
| Post- | % Silencing | 99 | 86 | 78 | N/A | 76 | 87 | 61 | 46 | 96 |
| Dose | % Silencing; | 87 ± 11 | | | 82 ± 7.8 | | | 68 ± 26 | | |
| | Mean ± SD | | | | | | | | | |
| Day | % Remaining of | 10 | 16 | ND | ND | 9 | 12 | 44 | 52 | 14 |
| 44 | Pre-treatment | | | (0) | (0) | | | | | |
| Post- | % Silencing | 90 | 84 | N/A | N/A | 91 | 88 | 56 | 48 | 86 |

| | | | | |
|---|---|---|---|---|
| Dose | % Silencing; | 86 ± 4.2 | 90 ± 2.1 | 64 ± 20 |
| | Mean ± SD | | | |

N/A = not applicable;
ND = not detected;
SC = subcutaneous;
SD = standard deviation; Samples in which mARC1 protein expression was below the limit of assay detection were denoted as “ND” (not detected) and set to zero.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the following claims.

1. An RNAi construct comprising a sense strand and an antisense strand, wherein the antisense strand comprises a region having a sequence that is substantially complementary to a mARC1 mRNA sequence, and wherein said region comprises at least 15 contiguous nucleotides from an antisense sequence listed in Table 1 or Table 2.

2. The RNAi construct of claim 1, wherein the sense strand comprises a sequence that is sufficiently complementary to the sequence of the antisense strand to form a duplex region of about 15 to about 30 base pairs in length.

3. The RNAi construct of claim 2, wherein the duplex region is about 17 to about 24 base pairs in length.

4. (canceled)

5. The RNAi construct of claim 1, wherein the sense strand and the antisense strand are each independently about 19 to about 30 nucleotides in length.

6. The RNAi construct of claim 5, wherein the sense strand and the antisense strand are each independently about 19 to about 23 nucleotides in length.

7. The RNAi construct of claim 1, wherein the RNAi construct comprises one or two blunt ends.

8. The RNAi construct of claim 1, wherein the RNAi construct comprises one or two nucleotide overhangs of 1 to 4 unpaired nucleotides.

9. (canceled)

10. The RNAi construct of claim 8, wherein the RNAi construct comprises a nucleotide overhang at the 3′ end of the sense strand, the 3′ end of the antisense strand, or the 3′ end of both the sense strand and the antisense strand.

11. The RNAi construct of claim 1, wherein the RNAi construct comprises at least one modified nucleotide.

12. (canceled)

13. The RNAi construct of claim 11, wherein the modified nucleotide is a 2′-fluoro modified nucleotide, a 2′-O-methyl modified nucleotide, a 2′-O-methoxyethyl modified nucleotide, 2′-O-alkyl modified nucleotide, a 2′-O-allyl modified nucleotide, a bicyclic nucleic acid (BNA), a deoxyribonucleotide, or combinations thereof.

14. (canceled)

15. (canceled)

16. The RNAi construct of claim 1, wherein the sense strand comprises an abasic nucleotide as the terminal nucleotide at its 3′ end, its 5′ end, or both its 3′ and 5′ ends.

17. The RNAi construct of claim 16, wherein the abasic nucleotide is linked to the adjacent nucleotide through a 3′-3′ internucleotide linkage or a 5′-5′ internucleotide linkage.

18. The RNAi construct of claim 1, wherein the sense strand, the antisense strand, or both the sense and antisense strands comprise one or more phosphorothioate internucleotide linkages.

19. The RNAi construct of claim 18, wherein the antisense strand comprises two consecutive phosphorothioate internucleotide linkages between the terminal nucleotides at both the 3′ and 5′ ends.

20. The RNAi construct of claim 18, wherein the sense strand comprises a single phosphorothioate internucleotide linkage between the terminal nucleotides at the 3′ end.

21. (canceled)

22. The RNAi construct of claim 1, wherein the antisense strand comprises or consists of a sequence selected from the antisense sequences listed in Table 1 or Table 2.

23. The RNAi construct of claim 1, wherein the antisense strand comprises or consists of a sequence selected from SEQ ID NO: 715; SEQ ID NO: 732; SEQ ID NO: 733; SEQ ID NO: 738; SEQ ID NO: 754; SEQ ID NO: 761; SEQ ID NO: 763; SEQ ID NO: 764; SEQ ID NO: 766; SEQ ID NO: 809; SEQ ID NO: 810; SEQ ID NO: 814; SEQ ID NO: 841; SEQ ID NO: 848; SEQ ID NO: 851; SEQ ID NO: 862; SEQ ID NO: 916; SEQ ID NO: 1057; SEQ ID NO: 1078; SEQ ID NO: 2919; SEQ ID NO: 2926; SEQ ID NO: 2946; SEQ ID NO: 2949; SEQ ID NO: 2953; and SEQ ID NO: 2956.

24-26. (canceled)

27. The RNAi construct of claim 1, wherein:
(i) the sense strand comprises or consists of the sequence of SEQ ID NO: 409 and the antisense strand comprises or consists of the sequence of SEQ ID NO: 1078;
(ii) the sense strand comprises or consists of the sequence of SEQ ID NO: 388 and the antisense strand comprises or consists of the sequence of SEQ ID NO: 1057;
(iii) the sense strand comprises or consists of the sequence of SEQ ID NO: 2808 and the antisense strand comprises or consists of the sequence of SEQ ID NO: 2926;
(iv) the sense strand comprises or consists of the sequence of SEQ ID NO: 2820 and the antisense strand comprises or consists of the sequence of SEQ ID NO: 2946;
(v) the sense strand comprises or consists of the sequence of SEQ ID NO: 391 and the antisense strand comprises or consists of the sequence of SEQ ID NO: 2949;
(vi) the sense strand comprises or consists of the sequence of SEQ ID NO: 390 and the antisense strand comprises or consists of the sequence of SEQ ID NO: 2956;
(vii) the sense strand comprises or consists of the sequence of SEQ ID NO: 179 and the antisense strand comprises or consists of the sequence of SEQ ID NO: 2919;
(viii) the sense strand comprises or consists of the sequence of SEQ ID NO: 388 and the antisense strand comprises or consists of the sequence of SEQ ID NO: 2953; or
(ix) the sense strand comprises or consists of the sequence of SEQ ID NO: 388 and the antisense strand comprises or consists of the sequence of SEQ ID NO: 1057.

28. The RNAi construct of claim 27, wherein:
(i) the sense strand comprises or consists of the sequence of modified nucleotides according to SEQ ID NO: 3078 and the antisense strand comprises or consists of the sequence of modified nucleotides according to SEQ ID NO: 3337;
(ii) the sense strand comprises or consists of the sequence of modified nucleotides according to SEQ ID NO: 3080 and the antisense strand comprises or consists of the sequence of modified nucleotides according to SEQ ID NO: 3339;
(iii) the sense strand comprises or consists of the sequence of modified nucleotides according to SEQ ID NO: 3163 and the antisense strand comprises or consists of the sequence of modified nucleotides according to SEQ ID NO: 3441;
(iv) the sense strand comprises or consists of the sequence of modified nucleotides according to SEQ ID NO: 3183 and the antisense strand comprises or consists of the sequence of modified nucleotides according to SEQ ID NO: 3469;
(v) the sense strand comprises or consists of the sequence of modified nucleotides according to SEQ ID NO: 3076 and the antisense strand comprises or consists of the sequence of modified nucleotides according to SEQ ID NO: 3472;
(vi) the sense strand comprises or consists of the sequence of modified nucleotides according to SEQ ID NO: 3077 and the antisense strand comprises or consists of the sequence of modified nucleotides according to SEQ ID NO: 3484;
(vii) the sense strand comprises or consists of the sequence of modified nucleotides according to SEQ ID NO: 2051 and the antisense strand comprises or consists of the sequence of modified nucleotides according to SEQ ID NO: 3545;
(viii) the sense strand comprises or consists of the sequence of modified nucleotides according to SEQ ID NO: 3080 and the antisense strand comprises or consists of the sequence of modified nucleotides according to SEQ ID NO: 3481;
(ix) the sense strand comprises or consists of the sequence of modified nucleotides according to SEQ ID NO: 3188 and the antisense strand comprises or consists of the sequence of modified nucleotides according to SEQ ID NO: 3339;
(x) the sense strand comprises or consists of the sequence of modified nucleotides according to SEQ ID NO: 3080 and the antisense strand comprises or consists of the sequence of modified nucleotides according to SEQ ID NO: 3476; or
(xi) the sense strand comprises or consists of the sequence of modified nucleotides according to SEQ ID NO: 3223 and the antisense strand comprises or consists of the sequence of modified nucleotides according to SEQ ID NO: 3517.

29-31. (canceled)

32. An RNAi construct for inhibiting expression of a human MARC1 gene in a cell, said RNAi construct comprising a sense strand and an antisense strand that hybridize to form a duplex region of about 15 to about 30 base pairs in length, and wherein the antisense strand comprises a region having a sequence that is substantially complementary to the sequence of at least 15 contiguous nucleotides of nucleotides 1205 to 1250 of SEQ ID NO: 1.

33. The RNAi construct of claim 32, wherein the region of the antisense strand comprises a sequence that is substantially complementary to the sequence of at least 15 contiguous nucleotides of nucleotides 1209 to 1239 of SEQ ID NO: 1.

34. The RNAi construct of claim 32, wherein the region of the antisense strand comprises a sequence of CAUCUAAUAUUCCAG (SEQ ID NO: 3656).

35. (canceled)

36. (canceled)

37. An RNAi construct for inhibiting expression of a human MARC1 gene in a cell, said RNAi construct comprising a sense strand and an antisense strand that hybridize to form a duplex region of about 15 to about 30 base pairs in length, and wherein the antisense strand comprises a region having a sequence that is substantially complementary to the sequence of at least 15 contiguous nucleotides of nucleotides 1345 to 1375 of SEQ ID NO: 1.

38. The RNAi construct of claim 37, wherein the region of the antisense strand comprises a sequence of UGGGACAUUGAAGCA (SEQ ID NO: 3657).

39. (canceled)

40. (canceled)

41. An RNAi construct for inhibiting expression of a human MARC1 gene in a cell, said RNAi construct comprising a sense strand and an antisense strand that hybridize to form a duplex region of about 15 to about 30 base pairs in length, and wherein the antisense strand comprises a region having a sequence that is substantially complementary to the sequence of at least 15 contiguous nucleotides of nucleotides 2039 to 2078 of SEQ ID NO: 1.

42. The RNAi construct of claim 41, wherein the region of the antisense strand comprises a sequence that is substantially complementary to the sequence of at least 15 contiguous nucleotides of nucleotides 2048 to 2074 of SEQ ID NO: 1.

43. The RNAi construct of claim 41, wherein the region of the antisense strand comprises a sequence of AUCAGAUCUUAGAGU (SEQ ID NO: 3658).

44-63. (canceled)

64. The RNAi construct of claim 1, wherein the RNAi construct further comprises a ligand.

65. The RNAi construct of claim 64, wherein the ligand comprises a cholesterol moiety, a vitamin, a steroid, a bile acid, a folate moiety, a fatty acid, a carbohydrate, a glycoside, or antibody or antigen-binding fragment thereof.

66. The RNAi construct of claim 64, wherein the ligand comprises galactose, galactosamine, or N-acetyl-galactosamine.

67. The RNAi construct of claim 66, wherein the ligand comprises a multivalent galactose moiety or multivalent N-acetyl-galactosamine moiety.

68. The RNAi construct of claim 67, wherein the multivalent galactose moiety or multivalent N-acetyl-galactosamine moiety is trivalent or tetravalent.

69. The RNAi construct of claim 64, wherein the ligand is covalently attached to the sense strand optionally through a linker.

70. The RNAi construct of claim 69, wherein the ligand is covalently attached to the 5′ end of the sense strand.

71. A pharmaceutical composition comprising the RNAi construct of claim 1 and a pharmaceutically acceptable carrier or excipient.

72. A method for reducing the expression of mARC1 protein in a patient in need thereof comprising administering to the patient the RNAi construct of claim 1.

73-76. (canceled)

77. A method for treating, preventing, or reducing the risk of developing fatty liver disease in a patient in need thereof comprising administering to the patient the RNAi construct of claim 1.

78. The method of claim 77, wherein the fatty liver disease is nonalcoholic fatty liver disease or nonalcoholic steatohepatitis.

79. (canceled)

80. (canceled)

81. A method for treating, preventing, or reducing liver fibrosis in a patient in need thereof comprising administering to the patient the RNAi construct of claim 1.

82-97. (canceled)
